(19) United States
(12) Patent Application Publication
(10) Pub. No.: US 2026/0226117 A1
(43) Pub. Date: Aug. 6, 2026

(54) PROTEIN COMPOSITIONS AND METHODS OF USE THEREOF IN MODULATING PER1 FUNCTION

(71) Applicant: Washington University, St. Louis, MO (US)

(72) Inventors: Brian J. DeBosch, St. Louis, MO (US); Cassandra Higgins, St. Louis, MO (US); Jiameng Sun, St. Louis, MO (US)

(73) Assignee: Washington University, St. Louis, MO (US)

(21) Appl. No.: 19/451,578

(22) Filed: Jan. 16, 2026

Related U.S. Application Data
(60) Provisional application No. 63/746,462, filed on Jan. 17, 2025.

(51) Int. Cl. C07K 14/47; A61K 38/00; A61K 47/26; A61P 3/04; C12N 15/86

(52) U.S. Cl. CPC C07K 14/4702; A61K 47/26; A61P 3/04; C12N 15/86; A61K 38/00; C07K 2319/10; C07K 2319/30; C07K 2319/42; C12N 2750/14143

(57) ABSTRACT

Protein compositions, such as fusion protein compositions, and methods of use thereof for modulating PER1 function are provided. Exemplary embodiments include methods for inducing PER1 expression, protecting against glucose intolerance, and improving glucose homeostasis. Exemplary compositions include at least one of: a deltaCBD fusion protein, a CBD fragment fusion protein, a TAT-CBD fusion protein, an Fc-CBD fusion protein, and an AAV8-native CBD fusion protein.

### CROSS-REFERENCE TO RELATED APPLICATIONS

This application claims the benefit of priority to U.S. Provisional Application Ser. No. 63/746,462 filed 17 Jan. 2025, which is incorporated herein by reference in its entirety.

### STATEMENT REGARDING FEDERALLY SPONSORED RESEARCH OR DEVELOPMENT

This invention was made with government support under HL147968 and DK126622 awarded by the National Institutes of Health (NIH). The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “020193-US-NP_Sequence-Listing” created on 15 Jan. 2026; 50,222 bytes). The subject matter of the Sequence Listing is incorporated herein by reference in its entirety.

### FIELD OF THE INVENTION

The present disclosure generally relates to circadian clock proteins and metabolic signaling.

### BACKGROUND

Metabolic diseases and hepatocellular cancer lack efficacious treatments. At the nexus of the portal and venous circulations, hepatocytes in the liver respond to an organism's glycemic status by coordinating substrate selection within the liver and across organ systems. Whereas the post-prandial state is marked by meal-derived carbohydrate flux into hepatocytes via the portal system and subsequent glycolysis and glucose oxidation through the tricarboxylic acid (TCA) cycle, the hallmarks of fasting include multiple adaptations to maintain glucose homeostasis in the absence of exogenous carbon. This includes a hepatocyte and whole-organism substrate switch from glycolytic flux and glucose oxidation toward fat oxidation, a concomitant decline in respiratory exchange ratio, activation of peripheral lipolysis, and storage of excess lipolyzed lipid as intrahepatic triglyceride for rapid subsequent on-site substrate mobilization. Fasting hepatocytes also induce autophagic flux as a means to recycle old or damaged organelles and glycoproteins as carbon substrate to fuel gluconeogenesis and ketogenesis. Finally, fasting hepatocytes secrete fibroblast growth factor 21 (FGF21)—an anti-diabetic hepatokine that communicates and integrates extrahepatic fat oxidation and promotes peripheral insulin sensitization to ensure efficient substrate absorption during the next refeed cycle. Together, these canonical hepatic fasting responses sustain carbon flux to key extrahepatic organs (e.g., heart and brain) for survival. These fasting responses can be leveraged to treat excess fat accumulation and insulin resistance therapeutically.

In addition to macronutrient flux control of hepatocyte metabolism, the hepatocyte harbors a cell-intrinsic clock that comprises genes Clock, Bmal1, cryptochrome (Cry) 1/2, and period homologs (Per) 1/2. These core circadian genes form a well-characterized transcriptional-translational feedback loop to maintain cyclic expressions. Deleting core circadian genes can result in arrhythmic liver transcriptomic profiles, and imposing time-restricted feeding or intermittent fasting incompletely restores circadian gene rhythmicity. In addition, genetic or environmental disruption of the core clock can induce metabolic disease in mice and humans, while metabolic disease itself alters rhythmic circadian gene expression. Yet, although a link between hepatic circadian rhythm and metabolism is apparent, the mechanisms by which clock genes integrate distinct metabolic inputs remains underappreciated. This is particularly highlighted by recent data indicating that the therapeutic metabolic response to time-restricted feeding occurs independent of the core clock. These dissonant findings led us to hypothesize that the hepatocyte harbors closely aligned-yet distinct-metabolic sensing mechanisms that drive mammalian fuel selection.

### SUMMARY

Among the various aspects of the present disclosure is the provision of a circadian probiotic system. The present disclosure describes compositions and methods that make use of the administration of a probiotic such as bacteria, yeast, or any other organism that has been modified to provide timed gene expression as a basis for the delivery of biologic drugs at prescribed times and frequencies.

At least in part, the present disclosure is directed to regulating glucose homeostasis in a subject in need thereof.

The present teachings include a method to protect against glucose intolerance in a subject by increasing a Per protein in the subject. In some embodiments, the subject has diabetes or obesity, the Per protein is Per1, and/or the Per protein is increased through administration of Per1, 6-TreAz, or IMCTA.

In one aspect of the present disclosure, a composition for inducing PER1 expression is provided, the composition comprising a deltaCBD fusion protein comprising a PER1 protein lacking a C-terminus CRY-1 binding domain (CBD).

In some embodiments, the composition further comprises trehalose or a trehalose analog. In some embodiments, the trehalose analog is selected from 6-azido trehalose (6-TreAz), 4-trehalosamine (4-TA), and IMCAT-C14 (IMCTA). In some embodiments, the deltaCBD fusion protein is packaged in an AAV8-vector under control of a TBG promoter.

In another aspect of the present disclosure, a composition for inducing PER1 expression is provided, the composition comprising at least one fusion protein selected from: a CRY-1 binding domain (CBD) fragment fusion protein comprising of a CBD region of a PER1 protein, wherein the CBD region lacks an N-terminus; a TAT-CBD fusion protein comprising of a CBD region of a PER1 protein fused to a viral TAT protein sequence YGRKKRRQRRR (SEQ ID NO: 1); an Fc-CBD fusion protein comprising a CBD region of a PER1 protein fused to an Fc portion of immunoglobulin; and an AAV8-native CBD fusion protein comprising a CBD region of a PER1 protein packaged in an AAV8-vector under control of a TGB promoter.

In some embodiments, the CBD region of the CBD fragment fusion protein is untagged or HA-tagged; the CBD region of the TAT-CBD fragment fusion protein is untagged or HA-tagged; the CBD region of the Fc-CBD fragment fusion protein is untagged or HA-tagged; and/or the CBD region of the AAV8-native CBD fragment fusion protein is untagged or HA-tagged. In some embodiments, the composition further comprises trehalose or a trehalose analog. In some embodiments, the trehalose analog is selected from 6-azido trehalose (6-TreAz), 4-trehalosamine (4-TA), and IMCAT-C14 (IMCTA).

In a further aspect of the present disclosure, a method for inducing PER1 expression in a subject in need thereof is provided, the method comprising: administering to the subject a composition comprising at least one fusion protein, wherein the at least one fusion protein is selected from: a deltaCBD fusion protein comprising a PER1 protein lacking a C-terminus CRY-1 binding domain (CBD); a CRY-1 binding domain (CBD) fragment fusion protein comprising of a CBD region of a PER1 protein, wherein the CBD region lacks an N-terminus; a TAT-CBD fusion protein comprising of a CBD region of a PER1 protein fused to a viral TAT protein sequence YGRKKRRQRRR (SEQ ID NO: 1); an Fc-CBD fusion protein comprising a CBD region of a PER1 protein fused to an Fc portion of immunoglobulin; and an AAV8-native CBD fusion protein comprising an untagged CBD region of a PER1 protein packaged in an AAV8-vector under control of a TGB promoter.

In some embodiments, the composition further comprises trehalose or a trehalose analog. In some embodiments, the trehalose analog is selected from 6-azido trehalose (6-TreAz), 4-trehalosamine (4-TA), and IMCAT-C14 (IMCTA). In some embodiments, the at least one fusion protein comprises the deltaCBD fusion protein packaged in an AAV8-vector under control of a TBG promoter. In some embodiments, the CBD fragment fusion protein is packaged in an AAV8-vector under control of a TBG promoter; the TAT-CBD fusion protein is packaged in an AAV8-vector under control of a TBG promoter; and the Fc-CBD fusion protein is packaged in an AAV8-vector under control of a TBG promoter. In some embodiments, the CBD region of the CBD fragment fusion protein is untagged or HA-tagged; the CBD region of the TAT-CBD fragment fusion protein is untagged or HA-tagged; the CBD region of the Fc-CBD fragment fusion protein is untagged or HA-tagged; and/or the CBD region of the AAV8-native CBD fragment fusion protein is untagged or HA-tagged. In some embodiments, administering the composition results in inhibition of CRY-1/PER1 interaction. In some embodiments, administering the composition results in at least one of: improved glucose tolerance; lowered fasting glucose; improved basal metabolic rate; and improved glucose homeostasis. In some embodiments, administering the composition comprising the TAT-CBD fusion protein results in greater cell permeability into multiple target tissues. In some embodiments, administering the composition comprising the Fc-CBD fusion protein results in a greater half-life of the fusion protein for dosing in the subject. In some embodiments, administering the composition comprising the AAV8-native CBD fusion protein results in inhibition of target tissue CRY/PER1 binding based on AAV tropism. In some embodiments, the subject has at least one of obesity, fatty liver disease, diabetes, type 2 diabetes mellitus, metabolic syndrome, liver cancer, and autophagic flux deficiency.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a schematic of the canonical core circadian regulatory network.

FIG. 1B is a graph showing the relative expression level of circadian genes (Per1, Per2, Cry1, Cry2, Bmal1, Clock) in liver from mice fasted for 0, 12, 16, or 24 h. Data expressed as mean±SEM. *p<0.05, **p<0.01, ***p<0.001 by one-way ANOVA n=3.

FIG. 1C is a schematic of the 24-h time-course harvest of tissues from mice either fed ad libitum or fasted for 16 h.

FIG. 1D is a graph showing the relative expression level of Per1 (normalized to mice fed ad libitum harvested at the same time) in liver harvested throughout the 24-h time course. Data expressed as mean±SEM. *p<0.05, **p<0.01, ***p<0.001 by Student's t test; n=3-4.

FIG. 1E is a graph showing the correlation between normalized expression level of Per1 in 16-h-fasted mice in (D) and the corresponding food consumption measured within the same period in the mice fed ad libitum. Dotted line denotes the 95% confidence interval for the simple linear regression calculation. Data expressed as mean±SEM by Pearson correlation test.

FIG. 1F is a schematic showing chair conformations of trehalose (Tre), 6-azido trehalose (6-TreAz), 4-trehalosamine (4-TA), and IMCAT-C14 (IMCTA).

FIG. 1G is a set of graphs showing the relative expression level of Per1 (left) and Per2 (right) from isolated wild-type primary hepatocytes treated for 24 h with regular growth medium (Control), Tre (100 mM), 6-TreAz (100 mM), 4-TA (100 mM), or IMCTA (100 mM) in complete culture medium. Data expressed as mean±SEM. *p<0.05, **p<0.01, ***p<0.001, ****p<0.0001 by Student's t test; n=3-6.

FIG. 1H is a graph showing relative expression level of Per1 from AML12 cells transduced with Ad-GFP or Ad-shPer1 for 48 h followed by a full medium change to either complete or starvation medium for 6 h. Data expressed as mean±SEM. ****p<0.0001 by two-way ANOVA; n=5.

FIG. 1I is a representative image of a western blot (top) and a corresponding set of graphs. Immunoblot analysis of AML12 cells described in (FIG. 1H) with additional treatment of dimethyl sulfoxide (DMSO) or bafilomycin A1 (BafA1) (200 nM) (left) during the medium change, and quantification of LC3A/B-II (right). Data expressed as mean±SEM. *p<0.05, **p<0.01 by two-way ANOVA; n=2.

FIG. 1J is a schematic of Per1^{fl/fl} mice design at the Per1 locus.

FIG. 1K is a schematic of the 14 h+2 h fast/refeed experimental design in Per1^{fl/fl} (Per1^{fl/f}l, AAV8-TBG-GFP) and Per1iLKO (Per1^{fl/fl}, AAV8-TBG-Cre) mice.

FIG. 1L is a set of graphs showing the concentration of serum non-esterified fatty acid (NEFA) (left), ketone body (middle), and hepatic triglyceride (TG) level from mice in (FIG. 1K). Data expressed as mean±SEM. *p<0.05, **p<0.01, ***p<0.001, ****p<0.0001 by two-way ANOVA; n=4-6.

FIG. 1M is a set of representative images from oil red O (ORO)-stained liver tissue of Per1^{fl/fl} (top) and Per1^{iLKO} (bottom) mice on a refeed (left) or fast (right) diet. Scale bar, 100 mm.

FIG. 1N is a heatmap showing unsupervised hierarchical clustering of all differentially regulated genes (p<0.05) from bulk RNA sequencing in liver harvested from (FIG. 1K); n=3.

FIG. 1O is a set of graphs showing the relative expression level of Per1 (top, left), Fgf21 (top, right), and Pdk4 (bottom) in liver harvested from mice in (FIG. 1K). Data expressed as mean±SEM. *p<0.05, **p<0.01, ***p<0.001, ****p<0.0001 by two-way ANOVA; n=4-6.

FIG. 1P is a graph of serum FGF21 protein level measured by ELISA from mice in (FIG. 1K). Data expressed as mean±SEM. ***p<0.001 by two-way ANOVA; n=4-6.

FIG. 2A is a set of uniform manifold approximation and projection for dimension reduction (UMAP) plots on single-nucleus multiome sequencing analysis of liver tissues from Per1^{fl/fl} and Per1^{cLKO} (Per1^{fl/fl}, Alb-Cre) fed ad libitum or fasted for 16 h (top). Nine major cell types were identified (bottom). In total, 9,871 (Per1^{fl/fl} Feed), 8,702 (Per1^{fl/fl} Fast), 9,494 (Per1^{cLKO} Feed), and 8,731 (Per1^{cLKO} Fast) nuclei were analyzed in each condition; n=3.

FIG. 2B is a Kyoto Encyclopedia of Genes and Genomes (KEGG) pathway analysis plot of differentially expressed genes (DEGs) between hepatocyte populations from Per1^{cLKO} Fast group and Per1^{fl/fl} Fast group.

FIG. 2C is a gene set enrichment analysis (GSEA) graph of circadian rhythm pathway between DEGs from hepatocytes in Per1^{cLKO} Fast group and Per1^{fl/fl} Fast group.

FIG. 2D is a set of KEGG pathway analysis plots showing activated pathways (top) and suppressed pathways (bottom) of differentially expressed peak signals between hepatocyte population from Per1^{cLKO} Fast group and Per1^{fl/fl} Fast group.

FIG. 2E is a schematic showing enriched motifs for Esrrg (top) and Atf1 (bottom) identified in hepatocyte population from Per1^{cLKO} Fast group.

FIG. 2F is a plot showing the quantification of normalized peak level at region chr7-45615005-45616587 in hepatocyte population from each group. In total, 9,267 (Per1^{fl/fl} Feed), 7,626 (Per1^{fl/fl} Fast), 5,445 (Per1^{cLKO} Feed), and 5,525 (Per1^{cLKO} Fast) nuclei were identified as hepatocytes and analyzed in each condition; n=3.

FIG. 2G is a coverage plot at Fgf21 locus from hepatocyte population. Functional units were identified based on the Encyclopedia of DNA Elements (ENCODE) database.

FIG. 2H is a set of graphs showing the relative expression of Per1 (top, middle left) and Fgf21 (middle right, bottom) in AML12 cells treated with siRNA targeting Ppara (top left, middle right), Esrrg (top right, bottom left), or Atf1 (middle left, bottom right) followed by medium change to complete or starvation medium. Data expressed as mean±SEM. *p<0.05, **p<0.01, ***p<0.001, ****p<0.0001 by two-way ANOVA; n=3.

FIG. 3A is a plot showing hepatocyte subpopulation (Fgf21^{high}) selection based on the normalized peak signal at chr7-45615005-45616587 region. Quantification of the normalized peak signal in each group. In total, of 144 (Per1^{fl/fl} Feed), 165 (Per1^{fl/fl} Fast), 145 (Per1^{cLKO} Feed), and 253 (Per1^{cLKO} Fast) nuclei were identified as Fgf21^{high} hepatocyte subpopulation and analyzed in each condition; n=3.

FIG. 3B is a UMAP plot of selected hepatocyte subpopulation distribution based on the treatment Feed or Fast for the mice Per1^{fl/fl} and Per1^{cLKO}.

FIG. 3C is a coverage plot at Fgf21 locus from selected hepatocyte subpopulation (Fgf21^{high}) and their corresponding Per1 expression level.

FIG. 3D is a farnesoid X receptor (FXR)/pregnane X receptor (PXR)/bile acid pathway analysis from Comprehensive Multi-omics Platform for Biological Interpretation (COMPBIO) from differentially regulated genes in Fgf21^{high} hepatocyte subpopulation from Per1^{cLKO} Fast group and Per1^{fl/fl} Fast group.

FIG. 3E is a KEGG pathway analysis plot of DEGs from Fgf21^{high} hepatocyte subpopulation between Per1^{cLKO} Fast group and Per1^{fl/fl} Fast group.

FIG. 3F is a gene ontology (GO) pathway analysis plot of DEGs from Fgf21^{high} hepatocyte subpopulation between Per1_{iLKO} Fast group and Per1^{fl/fl} Fast group.

FIG. 3G is a graph showing Seahorse XF Cell Mito Stress analysis (top) of AML12 cells and associated graph (bottom) of the maximal respiration AUC. AML12 cells were transduced with Ad-GFP or Ad-shPer1 for 48 h followed by medium change to either complete or starvation medium for 6 h (left), and area under the curve (AUC) quantification of the maximal respiration rate (right). Data expressed as mean±SEM. **p<0.01, ****p<0.0001 by two-way ANOVA; n=12.

FIG. 3H is a graph showing Seahorse XF glucose/pyruvate oxidation stress analysis (top) of AML12 cells and associated graph (bottom) of glucose/pyruvate inhibited maximal respiration AUC. AML12 cells were treated with Fgf21 antisense oligonucleotide (ASO) for 48 h followed by medium change to either complete or starvation medium for 6 h (left), and AUC quantification of the inhibited maximal respiration rate (right). Data expressed as mean±SEM. ****p<0.0001 by two-way ANOVA; n=12.

FIG. 3I is a graph showing Seahorse XF long-chain fatty acid oxidation stress analysis (top) of AML12 cells and associated graph of long-chain fatty acid inhibited maximal respiration AUC. AML12 cells were treated with Fgf21 ASO for 48 h followed by medium change to either complete or starvation medium for 6 h (left), and AUC quantification of the inhibited maximal respiration rate (right). Data expressed as mean±SEM. *p<0.05, **p<0.01, ****p<0.0001 by two-way ANOVA; n=12.

FIG. 4A is a schematic of in vitro feed/starve experiment in AML12 cells treated with either adenovirus or siRNA targeting Per1.

FIG. 4B is a set of graphs showing the relative expression level of Per1, Fgf21, and Pdk4 in AML12 cells treated with Ad-GFP or Ad-shPer1, fed or starved. Data expressed as mean±SEM. *p<0.05, **p<0.01, ***p<0.001, ****p<0.0001 by two-way ANOVA; n=5.

FIG. 4C is a schematic of in vitro feed/starve experiment in AML12 cells treated with either adenovirus or siRNA targeting Bmal1, Clock, or Cry1.

FIG. 4D is a set of graphs showing the relative expression level of Per1, Fgf21, and Pdk4 in AML12 cells treated with lipofectamine (Control), siBmal1, siClock, or siCryl, fed or starved. Data expressed as mean±SEM. *p<0.05, **p<0.01, ***p<0.001, ****p<0.0001 by Student's t test; n=3.

FIG. 4E is a schematic of ex vivo primary hepatocyte starvation experiment from Per2^{WT} and Per2^{KO} female mice.

FIG. 4F is a set of graphs showing the relative expression level of Per1, Fgf21, and Pdk4 in primary hepatocytes isolated from Per2^{WT} or Per2^{KO} mice, fed or starved. Data expressed as mean±SEM. *p<0.05, **p<0.01, ***p<0.001, ****p<0.0001 by two-way ANOVA; n=3-4.

FIG. 4G is a schematic of in vivo time-course experiment in Bmal1^{fl/fl} and Bmal1^{cLKO} (Bmal1^{fl/fl}, Alb-Cre) mice. Mice were either fed ad libitum or fasted for 16 h, and liver tissues were harvested every 4 h in a 24-h duration; n=3-4.

FIG. 4H is a set of plots showing the relative expression level of Per1, Fgf21, and Pdk4 (normalized to ZTO Bmal1^{fl/fl} feed) over time from liver of mice in FIG. 4G.

FIG. 4I is a set of plots showing correlation test result between normalized expression level of Per1 and Fgf21 (−8 h or +16 h) (left), Per1 and Pdk4 (−8 h or +16 h) (middle), and Fgf21 and Pdk4 (right) from FIG. 4H. Dotted line denotes the 95% confidence interval for the simple linear regression calculation. Data expressed as mean±SEM by Pearson correlation test.

FIG. 4J is a schematic of the 14 h+2 h fast/refeed experimental design in Bmal1^{fl/fl} (Bma/1^{fl/fl}, AAV8-TBG-GFP) and Bmal1^{iLKO} (Per1^{fl/fl}, AAV8-TBG-Cre) mice; n=3-5.

FIG. 4K is a set of graphs showing serum glucose levels (top left), Serum NEFA levels (top right), and hepatic TG levels (bottom) from the mice in FIG. 4J. Data expressed as mean±SEM. **p<0.01, ***p<0.001, ****p<0.0001 by two-way ANOVA.

FIG. 4L is a set of graphs showing the relative expression level of liver Bmal1 (top left), Per1 (top right), Fgf21 (middle), Pdk4 (bottom left), and Ppara (bottom right) from the mice described in FIG. 4J. Data expressed as mean±SEM. *p<0.05, **p<0.01, ***p<0.001, ****p<0.0001 by two-way ANOVA.

FIG. 5A is a schematic of the fasting-induced Pdk4-mediated inhibition of glucose oxidation.

FIG. 5B is a set of plots showing the respiratory exchange ratio (RER) measured during 16 h of fasting in Per1^{fl/fl} and Per1^{cLKO} (Per1^{fl/fl}, Alb-Cre) mice in indirect calorimetry (left), and quantification of RER during fasting from ZT12 to ZT20 and ZT20 to ZT4 (right). Data expressed as mean±SEM. *p<0.05, **p<0.01, ***p<0.001, ****p<0.0001 by two-way ANOVA; n=6.

FIG. 5C is a set of plots showing the glucose oxidation rate calculated based on VCO₂ and VO₂ during fasting. Shade denotes SEM (top) and quantification (bottom) from mice in (B). Data expressed as mean±SEM. *p<0.05, **p<0.01, ***p<0.001, ****p<0.0001 by Student's t test; n=6.

FIG. 5D is a graph showing the relative expression level of Per1 in liver from Per1^{fl/fl} or Per1^{cLKO} mice underwent 14 h+2 h fast/refeed. Data expressed as mean±SEM. *p<0.05, **p<0.01, ***p<0.001, ****p<0.0001 by two-way ANOVA; n=3.

FIG. 5E is an immunoblot image (top) and corresponding graph (bottom). Immunoblot analysis of liver tissues from FIG. 5D, quantification labeled on the top of each band (pPDHa1^{Ser293} was normalized to total PDH) (top), and quantification of pPDHa1^{Ser293} level normalized to Per1^{fl/fl} refeed (bottom). Data expressed as mean±SEM. *p<0.05, **p<0.01, ***p<0.001, ****p<0.0001 by two-way ANOVA; n=3.

FIG. 5F is a set of graphs showing relative expression level of Per1 and Pdk4 from AML12 cells transduced with Ad-GFP or Ad-shPer1 for 48 h followed by medium change to either complete or starvation medium for 48 h. Data expressed as mean±SEM. *p<0.05, **p<0.01, ***p<0.001, ****p<0.0001 by two-way ANOVA; n=3.

FIG. 5G is an immunoblot image (top) of AML12 cells as described in FIG. 5F (left), and quantification of pPDHa1^{Ser293} (bottom left) and PDK4 (bottom right). Data expressed as mean±SEM. *p<0.05, **p<0.01, ***p<0.001, ****p<0.0001 by two-way ANOVA; n=3.

FIG. 5H is an immunoblot image (top) of AML12 cells as described in FIG. 5F, with additional treatment of 25 μM CPI-613 2 h prior to harvest in Ad-shPer1-treated cells (top), and a corresponding graph quantifying pPDHa1 Ser293 (bottom). Data expressed as mean±SEM. *p<0.05, **p<0.01, ***p<0.001, ****p<0.0001 by two-way ANOVA; n=3.

FIG. 5I is a graph of pPDHa1^{Ser293} quantification from shPer1-treated AML12 cells with or without CPI-613 from FIG. 5G and FIG. 5H. pPDHa1 Ser293 level was normalized to corresponding Ad-GFP-treated cells cultured in complete medium. Data expressed as mean±SEM. *p<0.05, **p<0.01, ***p<0.001, ****p<0.0001 by two-way ANOVA; n=3.

FIG. 5J is a plot of RER measured during 16 h of fasting in Per1^{fl/fl} and Per1^{cLKO} mice in indirect calorimetry injected with either vehicle or 25 mg/kg CPI-613 at ZT20. Data expressed as mean±SEM. *p<0.05, **p<0.01, ***p<0.001, ****p<0.0001 by two-way ANOVA; n=3.

FIG. 6A is a schematic of the 14 h+2 h fast/refeed experimental design in Fgf21^{fl/fl} and Fgf21^{cLKO} (Fgf21^{fl/fl}, Alb-Cre) mice.

FIG. 6B is a diagram of differentially regulated gene (p<0.05) distribution in fasted liver found in Per^{iLKO} and Fgf21^{cLKO} mice compared to corresponding floxed control fasted mice; n=3.

FIG. 6C is a heatmap of the 53 overlapped genes found in FIG. 6B and their log fold change (log FC).

FIG. 6D is a set of graphs showing the relative expression level of Fgf21 and Pdk4 in liver harvested from the mice described in FIG. 6A. Data expressed as mean±SEM. *p<0.05, **p<0.01, ****p<0.0001 by two-way ANOVA; n=4-5.

FIG. 6E is a schematic of in vivo tracing experiment. In brief, [U-¹³C] glucose was infused to Per1^{fl/fl} or Perf^{iLKO} mice that underwent 14 h+2 h fast/refeed, with or without injection of 1 mg/kg FGF21; n=3.

FIG. 6F is a schematic of 13C-labeled (gray) and unlabeled carbon (white) distribution from [U-¹³C] glucose in glycolysis and PDH-mediated TCA cycle.

FIG. 6G is a set of graphs of the hepatic percent enrichment of correspondingly labeled metabolites involved in glycolysis (top) and PDH-mediated (bottom) TCA cycle in fasted mice described in FIG. 6E. Data expressed as mean±SEM. *p<0.05, **p<0.01 by two-way ANOVA; n=3.

FIG. 6H is a plot of RER measured in Per1^{WT} and Per1^{cLKO} (Per1^{fl/fl}, Alb-Cre) mice injected with either vehicle or mouse FGF21 recombinant protein (1 mg/kg) after 8 h of fasting (fasting started at ZT12); n=3.

FIG. 6I is a graph showing relative expression level of Pdk4 in liver harvested from mice described in FIG. 6H. Data expressed as mean±SEM. *p<0.05, **p<0.01, ***p<0.001, ****p<0.0001 by Student's t test; n=3.

FIG. 6J is a schematic of the regulatory pathway Per1-Fgf21-Pdk4-PDH during fasting.

FIG. 7 is a schematic showing the function of the circadian gene Period 1 during Feeding and Fasting. Per1 is upregulated during fasting in liver. Mice lacking hepatocyte Per1 fail to oxidize glucose and fatty acid properly during fasting, a defect rescued by supplying FGF21 or inhibiting pyruvate dehydrogenase activity.

FIG. 8A is a graph showing the relative expression level of Pgc1a in mice fasted for 0, 12, 16 or 24 h. Data expressed as mean±SEM. *p<0.05, **p<0.01 by student's t-test; n=3.

FIG. 8B is a set of graphs showing the relative expression level of Per1 and Per2 in culture media with different glucose concentration. Data expressed as mean±SEM. *p<0.05, **p<0.01, ***p<0.001, ****p<0.0001 by one-way ANOVA; n=3.

FIG. 8C is a set of graphs showing the relative expression level of Per1 and Pgc1a in culture media with different components (+/−ITS, +/−FBS). Data expressed as mean±SEM. **p<0.01, ****p<0.0001 by student's t-test; n=3.

FIG. 8D is a plot (top) and corresponding graph (bottom) showing individual total body weight change during 14 h+2 h fast/refeed in Per1^{fl/fl} and Perf^{iLKO} mice (top), and percent body weight change during 14 h fasting and 2 h refeeding/fasting (bottom). Data expressed as mean±SEM. ****p<0.0001 by two-way ANOVA; n=3.

FIG. 8E is a set of graphs of serum glucose (top) and hepatic NEFA (bottom) levels from mice described in FIG. 1K. Data expressed as mean±SEM. *p<0.05, ***p<0.001, ****p<0.0001 by two-way ANOVA; n=3-6.

FIG. 8F is a plot of KEGG pathway analysis on differentially expressed gene between fasted liver from Per1^{iLKO} and Per1^{fl/fl} mice described in FIG. 1K; n=3.

FIG. 8G is a graph showing the relative expression level of Ppara and its downstream targets from the mice described in FIG. 1K. Data expressed as mean±SEM. *p<0.05, **p<0.01, ***p<0.001, ****p<0.0001 by two-way ANOVA; n=3.

FIG. 9A is a UMAP plot showing 26 clusters identified in nuclei isolated from Per1^{fl/fl} and Per1^{cLKO} (Per1^{fl/fl}, Alb-Cre) liver either ad lib fed or 16 h-fasted; n=3

FIG. 9B is a plot showing genetic markers used for assigning major hepatic cell types in each cluster.

FIG. 9C is a heatmap of cell markers in each assigned hepatic cell type.

FIG. 9D is a set of plots showing relative expression level of Ppara and Pck1 in hepatocyte population from each group.

FIG. 9E is a plot of KEGG pathway analysis on differentially expressed genes, showing activated (left) and suppressed (right) pathways between Per1^{fl/fl} fasted and Per1^{fl/fl} ad lib fed hepatocytes.

FIG. 9F is a plot of KEGG pathway analysis on differentially expressed peak signals, showing activated (top) and suppressed (bottom) pathways between Per1^{fl/fl} fasted and Per1^{fl/fl} ad lib fed hepatocytes.

FIG. 9G is a coverage plot at the Pdk4 locus from the hepatocyte population.

FIG. 9H is a set of graphs showing relative expression level of Ppara and Atf1 in AML12 cells treated with siPpara or siAtf1 (FIG. 2H). Data expressed as mean±SEM. **p<0.01, ***p<0.001, ****p<0.0001 by two-way ANOVA; n=3.

FIG. 9I is a set of plots showing relative expression level of liver zone markers Cps1, Cyp2e1 and Glul in defined Fgf21^{High} and Fgf21^{Low} population.

FIG. 9J is a coverage plot at the Pdk4 locus from selected Fgf21^{High} hepatocyte subpopulation.

FIG. 9K is a graph showing relative expression level of Fgf21 in AML12 cells treated with Fgf21 ASO (FIG. 3H, FIG. 3I). Data expressed as mean±SEM. *p<0.05, **p<0.01 by two-way ANOVA; n=3.

FIG. 10A is a set of graphs showing relative expression level of Bmal1, Clock and Cry1 from AML12 cells treated with siRNA targeting Bmal1, Clock or Cry1. Data expressed as mean±SEM. *p<0.01, ***p<0.001, ****p<0.0001 by two-way ANOVA; n=3.

FIG. 10B is a set of graphs showing relative expression level of Dbp, Nr1d1 and Per1 in AML12 cells treated with either adenovirus or siRNA targeting Cry1. Data expressed as mean±SEM. *p<0.05, ****p<0.0001 by two-way ANOVA.

FIG. 10C is a set of graphs showing relative expression level of Dbp, Nr1d1 and Per1 in AML12 cells treated with either adenovirus or siRNA targeting Bmal1. Data expressed as mean±SEM. **p<0.01, ***p<0.001, ****p<0.0001 by two-way ANOVA.

FIG. 10D is a set of graphs showing relative expression level of Dbp, Nr1d1 and Per1 in AML12 cells treated with either adenovirus or siRNA targeting Clock. Data expressed as mean±SEM. **p<0.01, ***p<0.001, ****p<0.0001 by two-way ANOVA.

FIG. 10E is a set of plots showing relative expression level of Bmal1, Cry1, Clock and Per2 (normalized to ZTO Bmal1^{fl/fl} feed) from mice during 16 h-fasting timecourse as described in FIG. 4G; n=3-4

FIG. 10F is a set of plots showing correlation test result at different lag times (i.e. negative lag time represents a shift of expression towards left, and vice versa) between Per1 and Fgf21 (top left), Per1 and Pdk4 (top right) and Fgf21 and Pdk4 (bottom) expression when normalized to ZTO Bmal1^{fl/fl} feed. Data expressed as mean±SEM. *p<0.05, **p<0.01, ***p<0.001, ****p<0.0001 by Pearson correlation test.

FIG. 11A is a schematic of in vivo timecourse experiment in Per1^{fl/fl} and Perf^{iLKO} male mice. Mice were either ad lib fed or fasted for 16 h, and liver tissues were harvested every 4 h in a 24 h duration; n=3-5.

FIG. 11B is a set of plots showing relative expression level of Per1, Fgf21 and Pdk4 in male mice fed/fasted; n=3-5.

FIG. 11C is a set of plots showing relative expression level of Per1, Fgf21 and Pdk4 in male mice fed/fasted during dark phase. Data expressed as mean±SEM. *p<0.05, **p<0.01, ***p<0.001, ****p<0.0001 by two-way ANOVA; n=3-5.

FIG. 11D is a schematic of in vivo timecourse experiment in Per1^{fl/fl} and Perf^{iLKO} female mice that were fasted/fed during dark phase.

FIG. 11E is a set of graphs showing relative expression level of Per1, Fgf21 and Pdk4 in female mice fed/fasted. Data expressed as mean±SEM. *p<0.05, **p<0.01, ***p<0.001, ****p<0.0001 by two-way ANOVA and student's t-test; n=3-6.

FIG. 12A is a set of graphs or hepatic glycogen level in Per1^{fl/fl} and Per1^{cLKO} (Alb-Cre) mice 16 h-fasted/ad lib fed (left). Percent hepatic glycogen level change from 16 h-fasted mice compared to ad lib fed mice (right). Data expressed as mean±SEM. *p<0.05, *p<0.01, ***p<0.001, ****p<0.0001 by two-way ANOVA; n=3.

FIG. 12B is a plot of cumulative food consumption in Per1^{fl/fl} and Per1^{cLKO} mice; n=6.

FIG. 12C is a plot (top) and corresponding graph (bottom) of activity measured by beam counts during 24 h ad lib feeding in Per1^{fl/fl} and Per1^{cLKO} mice (left), and total activity quantification during 12 h dark and 12 h light phase (right). Data expressed as mean±SEM. *p<0.05, **p<0.01, ***p<0.001, ****p<0.0001 by two-way ANOVA; n=6.

FIG. 12D is a graph of total body weight of Per1^{fl/fl} and Per1^{cLKO} mice prior to fasting; n=6.

FIG. 12E is a plot (left) and corresponding graph (right) of activity measured by beam counts during 16 h fasting in Per1^{fl/fl} and Per1^{cLKO} mice (left), and total activity quantification (right); n=6.

FIG. 12F is a set of box plots showing fasting heat, 02 consumption and CO2 production in Per1^{fl/fl} and Per1^{cLKO} mice. Data expressed as mean±SEM. *p<0.05, **p<0.01, ***p<0.001, ****p<0.0001 by students t-test; n=6.

FIG. 13A is a graph showing relative expression level of liver Per1 from male Fgf21^{fl/fl} or Fgf21^{cLKO} (Fgf21^{fl/fl}, Alb-Cre) mice underwent 14 h+2 h fast/refeed. Data expressed as mean±SEM. *p<0.05, **p<0.01, ***p<0.001, **p<0.0001 by two-way ANOVA; n=5.

FIG. 13B is a set of graphs showing relative level of liver Per1 from female Fgf21^{fl/fl} or Fgf21^{cLKO} mice underwent 14 h+2 h fast/refeed. Data expressed as mean±SEM. *p<0.05, **p<0.01, ***p<0.001, ****p<0.0001 by two-way ANOVA; n=2-5.

FIG. 13C is a plot of KEGG pathway analysis on differentially expressed genes between fasted liver from Fgf21^{fl/fl} or Fgf21^{cLKO} mice in FIG. 6A; n=3.

FIG. 13D is a graph of serum glucose level in Fgf21^{fl/fl} or Fgf21^{cLKO} mice fasted/refed. Data expressed as mean±SEM. *p<0.05, **p<0.01, ***p<0.001, ****p<0.0001 by two-way ANOVA; n=3.

FIG. 13E is a set of graphs of serum NEFA (left) and ketone body (right) level in Fgf21^{fl/fl} or Fgf21^{cLKO} mice fasted/refed. Data expressed as mean±SEM. *p<0.05, *p<0.01, ***p<0.001, ****p<0.0001 by two-way ANOVA; n=4-5.

FIG. 13F is a set of graphs of hepatic NEFA (left) and TG (right) level in Fgf21^{fl/fl} or Fgf21^{cLKO} mice fasted/refed. Data expressed as mean±SEM. *p<0.05, **p<0.01, ***p<0.001, ****p<0.0001 by two-way ANOVA; n=4-5.

FIG. 13G is a representative image of ORO-staining in Fgf21^{fl/fl} or Fgf21^{cLKO} mice fasted/refed.

FIG. 13H is a graph of the percent enrichment in corresponding labelled metabolites involved in glycolysis in Per1^{fl/fl} and Perf^{iLKO} refeeding mice from FIG. 6E. Data expressed as mean±SEM. *p<0.05, **p<0.01, ***p<0.001, ****p<0.0001 by one-way ANOVA; n=3

FIG. 13I is a graph of percent enrichment in corresponding labelled metabolites involved in PDH-mediated TCA cycle in Per1^{fl/fl} and Perf^{iLKO} refeeding mice; n=3.

FIG. 14A is a diagram of 13C-labeled (red) and unlabeled carbon (grey) distribution from U¹³C-labeled glucose in glycolysis and pyruvate dehydrogenase (PDH)-mediated tricarboxylic acid (TCA) cycle.

FIG. 14B is a graph of percent enrichment of labeled metabolites involved in glycolysis pathway from isolated primary hepatocytes from Per1^{fl/fl} and Per1^{LKO} mice (n=3). Data are presented as the mean±SEM unless specified. Statistical tests were performed using student's t-test. *p<0.05, **p<0.01, ***p<0.001, ****p<0.0001.

FIG. 14C is a graph of percent Percent enrichment of labeled metabolites involved in PDH-mediated pathway (n=3). Data are presented as the mean±SEM unless specified. Statistical tests were performed using student's t-test. *p<0.05, **p<0.01, ***p<0.001, ****p<0.0001.

FIG. 14D is a diagram of 13C-labeled (blue) and unlabeled carbon (grey) distribution from U13C-labeled glucose in pyruvate carboxylase (PC)-mediated TCA cycle.

FIG. 14E is a set of graphs of percent enrichments of labeled metabolites involved in PC-mediated pathway (top) and ratio percentage between M+3 and M+2 labeled metabolites (bottom) (n=3). Data are presented as the mean±SEM unless specified. Statistical tests were performed using student's t-test. *p<0.05, **p<0.01, ***p<0.001, ****p<0.0001.

FIG. 14F is a schematic of experimental design of Per1^{fl/fl} and Per1^{iLKO} mice fed with normal chow or WD for 12 weeks.

FIG. 14G is a set of plots showing glucose tolerance test (GTT) and insulin tolerance test (ITT) in WD-fed Per1^{fl/fl} and Per^{iLKO} mice (n=4-7).

FIG. 14H is a graph of serum glucose level from the mice in FIG. 14F (n=4-7). Data are presented as the mean±SEM unless specified. Statistical tests were performed using two-way ANOVA. *p<0.05, **p<0.01, ***p<0.001, ****p<0.0001.

FIG. 14I is a set of graphs of liver cholesterol level (left) and liver triglyceride (TG) level (right) from the mice in FIG. 14F (n=4-7). Data are presented as the mean±SEM unless specified. Statistical tests were performed using two-way ANOVA. *p<0.05, **p<0.01, ***p<0.001, ****p<0.0001.

FIG. 14J is a set of immunohistochemistry images of Oil-Red-O (ORO) staining results from WD-fed Per1^{fl/fl} and Perf^{iLKO} liver tissues. Scale bar=50 μm.

FIG. 14K is a set of qRT-PCR graphs measuring liver Per1, Acc1, Scd1, Elovl6, Gpat and Mttp expression level from the mice in FIG. 14F (n=4-7). Data are presented as the mean±SEM unless specified. Statistical tests were performed using two-way ANOVA. *p<0.05, **p<0.01, ***p<0.001, ****p<0.0001.

FIG. 14L is a plot of hallmark gene sets significantly activated in WD-fed Perf^{iLKO} liver compared to WD-fed Per1^{fl/fl} liver from RNA-sequencing analysis.

FIG. 14M is a plot (top) and set of corresponding graphs (bottom) of heat production measured from indirect calorimetry (top), and quantifications during light phase (bottom left) and dark phase (bottom right). Whiskers represent 10-90 percentile. Data are presented as the mean±SEM unless specified. Statistical tests were performed using two-way ANOVA. *p<0.05, **p<0.01, ***p<0.001, ****p<0.0001.

FIG. 15A is a schematic of experimental design of mice overexpressed with GFP or Per1 followed by 12-week WD feeding.

FIG. 15B is a set of UMAP plots showing clustering of GFP- or Per1-overexpressed liver from single-nucleus RNA-sequencing.

FIG. 15C is a plot of cell type compositions in GFP and Per1-overexpressed liver tissues.

FIG. 15D is a plot of KEGG pathway analysis in Per1-overexpressed hepatocytes compared to GFP-treated hepatocytes.

FIG. 15E is a set of GSEA plots in NAFLD and unsaturated fatty acids biosynthesis pathways in Per1-overexpressed hepatocytes compared to GFP-overexpressed hepatocytes.

FIG. 15F is a set of feature plots in Gm42418, AY036118 and Cmss1 expression level.

FIG. 15G is a survival plot of hepatocellular carcinoma patients expressing high-CMSS1 or low-CMSS1 from The Cancer Genome Atlas (TCGA) dataset.

FIG. 16A is a schematic showing the structural design of wtPer1, Per1^{ΔCBD} and CBD vectors.

FIG. 16B is an immunoblot image showing in vitro pull-down and immunoblot targeting GFP-tag, CRY1 and HA-tag in Scrambled CBD (Scramble), wtPer1, Per1^{ΔCBD} and CBD overexpressed AML12 cells.

FIG. 16C is a schematic of experimental design in Bmal1^{fl/fl} and Bmal1^{LKO} mice overexpressing Scramble, wtPer1, Per1^{ΔCBD} and CBD through AAV8-TBG vector followed by 12-week WD-feeding.

FIG. 16D is a plot of glucose tolerance test result in Bmal1^{fl/fl} and Bmal1^{LKO} mice overexpressing Scramble and wtPer1 (top) and corresponding graph showing AUC analysis (bottom) (n=4-5). Data are presented as the mean±SEM. Statistical tests were performed using two-way ANOVA. *p<0.05, **p<0.01, ***p<0.001, ****p<0.0001.

FIG. 16E is a plot of glucose tolerance test result in Bmal1^{fl/fl} and Bmal1^{LKO} mice overexpressing Scramble and Per1^{ΔCBD} (top) and corresponding graph showing AUC analysis (bottom) (n=4-5). Data are presented as the mean±SEM. Statistical tests were performed using two-way ANOVA. *p<0.05, **p<0.01, ***p<0.001, ****p<0.0001.

FIG. 16F is a plot of glucose tolerance test result in Bmal1^{fl/fl} and Bmal1^{LKO} mice overexpressing Scramble and CBD (top) and corresponding graph showing AUC analysis (bottom) (n=4-5). Data are presented as the mean±SEM. Statistical tests were performed using two-way ANOVA. *p<0.05, **p<0.01, ***p<0.001, ****p<0.0001.

FIG. 16G is a set of plots of respiratory exchange ratio (RER) in Bmal1^{fl/fl} and Bmal1^{LKO} mice overexpressing Scramble and wtPer1 (n=4).

FIG. 16H is a set of plots of heat production in Bmal1^{fl/fl} and Bmal1^{LKO} mice overexpressing Scramble and wtPer1 (n=4).

FIG. 16I is a set of plots of RER in Bmal1^{fl/fl} and Bmal1^{LKO} mice overexpressing Scramble and Per1^{ΔCBD} (n=4-5).

FIG. 16J is a set of plots of heat production in Bmal1^{fl/fl} and Bmal1^{LKO} mice overexpressing Scramble and Per1^{ΔCBD} (n=4-5).

FIG. 16K is a set of plots of RER in Bmal1^{fl/fl} and Bmal1^{LKO} mice overexpressing Scramble and CBD (n=4-5).

FIG. 16L is a set of plots of heat production in Bmal1^{fl/fl} and Bmal1^{LKO} mice overexpressing Scramble and CBD (n=4-5).

FIG. 17A is a set of graphs of liver TG level in WD-fed Bmal1^{fl/fl} and Bmal1^{LKO} mice overexpressing Scramble, wtPer1, Per1^{ΔCBD} and CBD (n=4-5). Data are presented as the mean±SEM. Statistical tests were performed using two-way ANOVA. *p<0.05, **p<0.01, ***p<0.001, ****p<0.0001.

FIG. 17B is a set of graphs of serum low density lipoprotein cholesterol (LDLc) level in WD-fed Bmal1^{fl/fl} and Bmal1^{LKO} mice overexpressing Scramble, wtPer1, Per1^{ΔCBD} and CBD (n=4-5). Data are presented as the mean±SEM. Statistical tests were performed using two-way ANOVA. *p<0.05, **p<0.01, ***p<0.001, ****p<0.0001.

FIG. 17C is an illustration of genes involved in de novo lipogenesis pathway.

FIG. 17D is a set of qRT-PCR graphs measuring Srebp1c, Acc1, Fasn, Elovl6, Scd1 and Mttp in liver from Bmal1^{fl/fl} and Bmal1^{LKO} mice overexpressing Scramble and wtPer1 (n=4-5). Data are presented as the mean±SEM. Statistical tests were performed using two-way ANOVA. *p<0.05, **p<0.01, ***p<0.001, ****p<0.0001.

FIG. 17E is a set of qRT-PCR graphs measuring Srebp1c, Acc1, Fasn, Elovl6, Scd1 and Mttp in liver from Bmal1^{fl/fl} and Bmal1^{LKO} mice overexpressing Scramble and Per1^{ΔCBD} (n=4-5). Data are presented as the mean±SEM. Statistical tests were performed using two-way ANOVA. *p<0.05, **p<0.01, ***p<0.001, ****p<0.0001.

FIG. 17F is a set of qRT-PCR graphs measuring Srebp1c, Acc1, Fasn, Elovl6, Scd1 and Mttp in liver from Bmal1^{fl/fl} and Bmal1^{LKO} mice overexpressing Scramble and CBD (n=4-5). Data are presented as the mean±SEM. Statistical tests were performed using two-way ANOVA. *p<0.05, **p<0.01, ***p<0.001, ****p<0.0001.

FIG. 18A is a schematic of the workflow of in vitro and ex vivo pull-down and proteomic analysis in cells overexpressing GFP, wtPer1, Per1^{ΔCBD} and CBD.

FIG. 18B is a chart of pull-down and proteomics in AML12 cells.

FIG. 18C is a chart of pull-down and proteomics in primary hepatocytes.

FIG. 18D is an image of an immunoblot showing in vitro pull-down and immunoblot targeting PP2A subunit A (PP2A-A) in AML12 overexpressing GFP, wtPer1, Per1^{ΔCBD} and CBD.

FIG. 18E is a schematic of the workflow of in vitro starvation followed by glucose and insulin-stimulation in AML12 cells.

FIG. 18F is a set of qRT-PCR graphs measuring Per1, Elovl6 and Scd1 in AML12 cells treated with or without glucose or insulin (n=3). Data are presented as the mean±SEM. Statistical tests were performed using two-way ANOVA. *p<0.05, **p<0.01, ***p<0.001, ****p<0.0001.

FIG. 18G is a set of qRT-PCR graphs measuring Per1, Elovl6 and Scd1 in AML12 cells treated with different concentrations of PP2A inhibitor LB-100 (n=3). Data are presented as the mean±SEM. Statistical tests were performed using one-way ANOVA. *p<0.05, **p<0.01, ***p<0.001, ****p<0.0001.

FIG. 18H is a set of qRT-PCR graphs measuring Per1, Elovl6 and Scd1 in AML12 cells treated with PP2A activator DT-061 (n=3). Data are presented as the mean±SEM. Statistical tests were performed using student's t-test. *p<0.05, **p<0.01, ***p<0.001, ****p<0.0001.

FIG. 19A is a schematic of experimental design in Per1^{fl/fl} and Perf^{iLKO} mice subjected to Vehicle or LB-100 injection fed with 12-week WD.

FIG. 19B is a plot of normalized body weight of WD-fed Per1^{fl/fl} and Per1^{iLKO} mice treated with or without LB-100 (top) and a corresponding graph of percent body weight change between Week 10 and Week 1 post-diet change (bottom). Data are presented as the mean±SEM. Statistical tests were performed using two-way ANOVA. *p<0.05, **p<0.01, ***p<0.001, ****p<0.0001.

FIG. 19C is a graph of fat mass percentage measured by EchoMRI from the mice described in FIG. 19A (n=5-6). Data are presented as the mean±SEM. Statistical tests were performed using two-way ANOVA. *p<0.05, **p<0.01, ***p<0.001, ****p<0.0001.

FIG. 19D is a graph of lean mass percentage measured by EchoMRI from the mice described in FIG. 19A (n=5-6). Data are presented as the mean±SEM. Statistical tests were performed using two-way ANOVA. *p<0.05, **p<0.01, ***p<0.001, ****p<0.0001.

FIG. 19E is a graph of glucose tolerance test result (left) and AUC analysis (right) from the mice described in FIG. 19A (n=5-6). Data are presented as the mean±SEM. Statistical tests were performed using two-way ANOVA. *p<0.05, **p<0.01, ***p<0.001, ****p<0.0001.

FIG. 19F is a plot of insulin tolerance test (left) and corresponding graph of the AUC analysis (right) from the mice described in FIG. 19A (n=5-6). Data are presented as the mean±SEM. Statistical tests were performed using two-way ANOVA. *p<0.05, **p<0.01, ***p<0.001, ****p<0.0001.

FIG. 19G is a set of graphs of heat production measured by indirect calorimetry from the mice described in FIG. 19A subjected to vehicle (left) or LB-100 (right) (n=4).

FIG. 19H is a set of graphs of VO2 measured by indirect calorimetry from the mice described in FIG. 19A subjected to vehicle (left) or LB-100 (right) (n=4).

FIG. 19I is a set of graphs of VCO2 measured by indirect calorimetry from the mice described in FIG. 19A subjected to vehicle (left) or LB-100 (right) (n=4).

FIG. 20A is a set of images of livers from Per^{fl/fl} and Perf^{iLKO} mice subjected to Vehicle or LB-100 injection fed with 12-week WD (n=5-6).

FIG. 20B is a graph of liver to body ratio from Per1^{fl/fl} and Per1^{iLKO} mice subjected to Vehicle or LB-100 injection fed with 12-week WD (n=5-6).

FIG. 20C is a graph of liver TG levels from Per1^{fl/fl} and Perf^{iLKO} mice subjected to Vehicle or LB-100 injection fed with 12-week WD (n=5-6).

FIG. 20D is a set of images of ORO staining of liver tissues from Per1^{fl/fl} and Per1^{iLKO} mice subjected to Vehicle or LB-100 injection fed with 12-week WD (n=5-6).

FIG. 20E is a set of qRT-PCR graphs measuring expression of Per1, Acc1, Fasn, Elovl6, Scd1 and Mttp in liver tissues from Per1^{fl/fl} and Perf^{iLKO} mice subjected to Vehicle or LB-100 injection fed with 12-week WD (n=5-6).

FIG. 20F is a graph of serum PP2A activity in Per1^{fl/fl} and Perf^{iLKO} mice subjected to Vehicle or LB-100 injection fed with 12-week WD (n=5-6).

FIG. 20G is an image of an immunoblot measuring CHREBP, PCNA and ACTIN in isolated liver nuclei and total liver lysate (n=2).

FIG. 20H is a diagram illustrating the proposed mechanism of PER1-ChREBP-PP2A pathway.

FIG. 21A is a schematic of the workflow of phosphoproteomics anlaysis in liver tissues from Per1^{fl/fl} and Perf^{iLKO} mice subjected to Vehicle or LB-100 injection fed with 12-week WD

FIG. 21B is a principal component analysis (PCA) plot from Per1^{fl/fl} and Perf^{iLKO} mice subjected to Vehicle or LB-100 injection fed with 12-week WD as described in FIG. 21A.

FIG. 21C is an image of Hierarchical clustering of samples from Per1^{fl/fl} and Per1^{iLKO} mice subjected to Vehicle or LB-100 injection fed with 12-week WD (n=5).

FIG. 21D is a plot showing pathway enrichment analysis from differentially regulated phosphorylated sites among four groups.

FIG. 21E is a set of graphs of the normalized intensity of phosphorylated ChREBP level at Serine 25 (top) and total level (bottom) (n=5).

FIG. 21F is a schematic of signalome analysis of kinases in Perf^{iLKO}-Vehicle liver versus Per1^{fl/fl}-Vehicle liver (left) and Per1^{iLKO}-LB-100 versus Per1^{fl/fl}-LB-100 (right).

FIG. 22A is a set of plots of glucose (top) and insulin (bottom) tolerance testing results in chow-fed Per1^{fl/fl} and Perf^{iLKO} mice (n=4-5).

FIG. 22B is a set of graphs of GTT (left) and ITT (right) area under curve (AUC) analysis in chow- or WD-fed Per1^{fl/fl} and Per1^{iLKO} mice (n=4-7).

FIG. 22C is a plot of body weight normalized to Week 1 Per1^{fl/fl} and Per1^{iLKO} mice fed with normal chow or WD for 12 weeks as described in FIG. 14F (n=4-7).

FIG. 22D is a set of graphs of fat (left) and lean (right) mass percentage measured by EchoMRI from Per1^{fl/fl} and Per1^{iLKO} mice fed with normal chow or WD for 12 weeks as described in FIG. 14F (n=4-7).

FIG. 22E is a graph of liver to body weight ratio from Per1^{fl/fl} and Per1^{iLKO} mice fed with normal chow or WD for 12 weeks as described in FIG. 14F (n=4-7).

FIG. 22F is a plot and set of graphs of RER from mice as described in FIG. 14F. RER was measured by indirect calorimetry (top), RER quantification during light phase (bottom left) and RER quantification during dark phase (bottom right) (n=4-7). Whiskers represent 10-90 percentile.

FIG. 23A is a qRT-PCR graph measuring expression of liver Per1 in AAV8-TBG-GFP or AAV8-TBG-Per1 treated mice (n=5).

FIG. 23B is a UMAP plot identifying 17 clusters.

FIG. 23C is a heatmap of the UMAP clustering (FIG. 23B) assigned to 5 cell types by gene signature.

FIG. 23D is a set of feature plots showing expression of Scd1, Fasn and Fabp1.

FIG. 24A is an ITT plot (left) and corresponding graph (right) in Bma/1^{fl/fl} or Bmal1^{LKO} mice overexpressing hepatocyte wtPer1 (n=4-5)

FIG. 24B is an ITT plot (left) and corresponding graph (right) in Bma/1^{fl/fl} or Bmal1^{LKO} mice overexpressing hepatocyte Per1^{ΔCBD} (n=4-5).

FIG. 24C is an ITT plot (left) and corresponding graph (right) in Bma/1^{fl/fl} or Bmal1^{LKO} mice overexpressing hepatocyte CBD (n=4-5).

FIG. 24D is a set of graphs light (left) and dark (right) phase quantification of RER in Bmal1^{fl/fl} or Bmal1^{LKO} mice overexpressing hepatocyte wtPer1 (n=4-5). Whiskers represent 10-90 percentile.

FIG. 24E is a set of graphs light (left) and dark (right) phase quantification of heat production in Bmal1^{fl/fl} or Bmal1^{LKO} mice overexpressing hepatocyte wtPer1 (n=4-5). Whiskers represent 10-90 percentile.

FIG. 24F is a set of graphs light (left) and dark (right) phase quantification of RER in Bmal1^{fl/fl} or Bmal1^{LKO} mice overexpressing hepatocyte Per1^{ΔCBD} (n=4-5). Whiskers represent 10-90 percentile.

FIG. 24G is a set of graphs light (left) and dark (right) phase quantification of heat production in Bmal1^{fl/fl} or Bmal1^{LKO} mice overexpressing hepatocyte Per1^{ΔCBD} (n=4-5). Whiskers represent 10-90 percentile.

FIG. 24H is a set of graphs light (left) and dark (right) phase quantification of RER in Bmal1^{fl/fl} or Bmal1^{LKO} mice overexpressing hepatocyte CBD (n=4-5). Whiskers represent 10-90 percentile.

FIG. 24I is a set of graphs light (left) and dark (right) phase quantification of heat production in Bmal1^{fl/fl} or Bmal1^{LKO} mice overexpressing hepatocyte CBD (n=4-5). Whiskers represent 10-90 percentile.

FIG. 24J is a set of plots of heat and body weight correlations in mice from FIG. 16C.

FIG. 24K is a set of plots and corresponding graphs detailing the activity of the mice described in FIG. 16C.

FIG. 25A is a set of plots of body weight normalized to Week 1 from the mice in FIG. 16C (n=4-5).

FIG. 25B is a set of plots of cumulative food consumption per mouse from the mice in FIG. 16C (n=4-5).

FIG. 25C is a set of graphs of serum triglyceride (TG) levels from the mice in FIG. 16C (n=4-5).

FIG. 25D is a set of graphs of cholesterol levels from mice in FIG. 16C (n=4-5).

FIG. 25E is a set of graphs of albumin levels from mice in FIG. 16C (n=4-5).

FIG. 25F is a set of qRT-PCR graphs measuring Bmal1 expression level in liver tissues from the mice in FIG. 16C (n=4-5).

FIG. 25G is a set of qRT-PCR graphs measuring Per1 expression level in liver tissues from the mice in FIG. 16C (n=4-5).

FIG. 26A is a schematic detailing peptide selection criterion from pull-down proteomics data.

FIG. 26B is a diagram of the number of proteins bind to three PER1 constructs in AML12 (left) and isolated primary hepatocytes (right).

FIG. 26C is a diagram of common binding partners between AML12 and primary hepatocytes across all three constructs.

FIG. 26D is a set of qRT-PCR graphs measuring expression of Chrebp and Ppp2r1b in AML12 cells treated with siChrebp (left) or siPpp2r1b (n=3).

FIG. 26E is a set of qRT-PCR graphs measuring foldchange of Elovl6 and Scd1 in AML12 cells treated with either siChrebp or siPpp2r1b when stimulated with glucose and insulin compared to the starvation control (n=3).

FIG. 27A is an activity plot (left) and corresponding graph (right) quantifying activity by indirect calorimetry from the mice described in FIG. 19A (n=4).

FIG. 27B is a set of graphs of the light phase (left) and dark phase (right) heat production quantifications from FIG. 19G (n=4). Whiskers represent 10-90 percentile.

FIG. 27C is a graph quantifying heat and body weight correlation from the mice described in FIG. 19G (n=4).

FIG. 27D is a qRT-PCR graph measuring expression of Ucp1 in brown adipose tissues (BAT) from the mice described in FIG. 19G (n=5-6).

FIG. 27E is a set of plots of RER data from the mice described in FIG. 19A (n=4).

FIG. 27F is a set of graphs quantifying the light (left) and dark (right) phase RER (n=4).

FIG. 27G is a set of graphs quantifying the light (left) and dark (right) phase VO₂ (n=4).

FIG. 27H is a set of graphs quantifying the light (left) and dark (right) phase VCO₂ (n=4).

FIG. 28A is a set of whole-body images of Per1^{fl/fl} (top) and Perf^{iLKO} (bottom) mice treated with vehicle (left) or LB-100 (right) (FIG. 19A).

FIG. 28B is a set of graphs of fat (left) and lean (right) mass measured by EchoMRI from Per1^{fl/fl} and Perf^{iLKO} mice treated with vehicle or LB-100 (n=5-6).

FIG. 28C is a plot of cumulative food consumption amount per mouse from the mice described in FIG. 19A (n=5-6).

FIG. 28D is a set of graphs quantifying serum glucose (top left), LDLc (top right) and cholesterol (bottom) levels from the mice described in FIG. 19A (n=5-6).

FIG. 29A is a set of volcano plots of differentially phosphorylated sites (|log 2FC|>2, p.adj<0.01) in pairwise comparisons from liver samples in FIG. 19A (n=5).

FIG. 29B is a set of graphs of normalized intensity of phosphorylated ChREBP at Serine 195/196 (left) and phosphorylated BAD at Serine 113 (right).

FIG. 29C is a heat map of the kinase-substrate relation scores between vehicle-treated Per1^{iLKO} and Per1^{fl/fl} mice (n=5).

FIG. 29D is a heat map of the kinase-substrate relation scores between LB-100-treated Per1^{iLKO} and Per1^{fl/fl} mice (n=5).

### DETAILED DESCRIPTION

The present disclosure is based, at least, on the discoveries that Inhibiting cryptochrome circadian regulator binding domain interactions in the period circadian regulator 1 enhances PER1 function, and that Per1 regulates glucose homeostasis and protects against glucose intolerance in an obese subject.

It has been demonstrated that the hepatocyte fasting response is sufficient to convey the therapeutic effects of caloric restriction. As disclosed herein, one target found to be upregulated during fasting is the period circadian regulator 1. Overexpression of PER1 in hepatocytes of obese mice improved glucose and insulin tolerance, improved whole-body fat oxidation. In cultured liver cancer cells (hepatocellular carcinoma, mouse origin, HEPA1-6 cells) PER1 overexpression blocked cell proliferation in vitro. Finally, in patients with hepatocellular carcinoma, the high PER1 expressing tumors correlated with significantly improved survival when compared with low PER1 expressing tumors. Thus, PER1 expression induced autophagic flux pathways by transcriptomic analysis of livers in fasting and refed mice. Overall, the data demonstrated that PER1 protects against metabolic and proliferative disease.

When the structural determinants of PER1 protective effects were determined, the CRY1-binding domain (CBD) of PER1 was targeted. This is a regulatory domain that is required for PER1 to enter the nucleus and inhibit CLOCK/BMAL function. It was surprisingly found that PER1 (deltaCBD), a PER mutant lacking the CRY1 binding domain, exhibited enhanced insulin sensitizing and anti-proliferative effects in obese mice and in cancer cell lines, respectively. The conclusion is that PER1 exerts CRY-dependent and CRY-independent effects, and that the adaptive CRY1-independent functions of PER1 can be separated from its CLOCK/BMAL1 inhibitory (e.g., potentially maladaptive) functions by inhibiting CRY1/PER1 interactions.

To that end, four new compositions of matter constructs were generated, to be delivered as injectable peptide or viral delivery, to inhibit CRY1/PER1 interactions.
Protein 1) PER1 (deltaCBD). This protein is not known to exist in nature. It is PER1 lacking its C-terminus CBD. This in an AAV8-vector was packaged under control of a TBG promoter.
Protein 2) TAT-CBD. This is the C-terminal CBD fused to the viral TAT protein sequence (YGRKKRRQRRR). The effect of the fusion protein is greater cell permeability into multiple target tissues.
Protein 3) Fc-CBD. This is an Fc portion of immunoglobulin fused to CBD. The effect is greater half-life of the fusion protein for dosing in patients.
Protein 4) AAV8-native CBD (untagged). The native CBD is not known to exist in nature. The effect of this fusion protein is to inhibit target tissue CRY/PER1 binding based upon AAV tropism. This protein was packaged in an AAV8 under control of the TBG promoter.

The range of use for each of these is broad. In some embodiments, CBD constructs (including those disclosed herein) treat metabolic and proliferative diseases like type 2 diabetes mellitus, metabolic syndrome, and cancer. Efficacy of these fusion proteins can be used against any disease of autophagic flux deficiency, including Alpha 1 antitrypsin deficiency and neurodegenerative disease. Each of these fusion proteins can be used to treat metabolic diseases and hepatocellular cancer, which lack efficacious treatments.

Optimal, context-dependent substrate selection is critical for growth, adaptation, and long-term survival. A well-designed system should account for acute substrate flux and is primed by timing in anticipation of the organism's feeding/fasting behavior. Per1 is a candidate gene that may fulfill these functions. Per1 is a canonical clock gene, yet here it is demonstrates that Per1 is uniquely and acutely regulated by fasting/refeeding and glucose transporter blockade. It mediates fasting-induced autophagic flux and substrate selection and is required to drive a key fasting regulatory hepatokine, FGF21. Further elucidated is a control mechanism to link fasting to appropriate glucose and fatty acid oxidation in demonstrating that fasting-induced hepatocyte Fgf21 links Per1 upregulation to transcriptional Pdk4 activation and PDH phosphorylation to control substrate oxidation. This is supported by the absence of Fgf21 induction in Per1-deficient liver during fasting, and the fact that Fgf21_{LKO} mice partly phenocopy the Per1^{LKO} phenotype during fasting. This is mechanistically sensible, as this pathway provides a means to couple deficient carbohydrate content in hepatocytes to shunt whole-organism substrate utilization away from glucose metabolism via endocrine FGF21.

Data herein indicate that Per1's control over substrate selection, intriguingly, occurs autonomously and independently of the circadian clock. Equally important, however, circadian and metabolic inputs into Per1 expression are approximately additive. Shown herein is that genetic knockdown of other clock genes in multiple in vitro and in vivo model systems—in Per2, Cry1, Bmal1 and Clock-deficient hepatocytes, in Bmal1^{LKO} mice—fails to alter fasting-induced Per1 expression. Second, Bmal1^{LKO} mice are phenotypically normal during fasting, despite the canon that Bmal1 mediates Per1 transcription within the circadian context.

Per1 overexpression enhances glucose tolerance and glucose oxidation and promotes energy expenditure even in the absence of its CRY1/2 binding domain. Together, this data suggests that hepatocyte Per1 is regulated by circadian input and yet exerts its metabolic function independent of these circadian inputs. This coupling of distinct input-response within the same sensing factor (Per1) permits dynamic, finely tuned control over substrate selection that integrates, yet separately accounts for the organism's circadian and metabolic states. Moreover, when both inputs were altered, Per1 response amplitude was greater to metabolic signal over the canonical circadian regulation, which therefore opens the window for the well-regulated circadian feedback loop to adapt to exogenous nutrient changes.

Demonstrated herein, at a cellular level, is a model of how Per1 suppresses glucose oxidation by inhibiting PDH activity in hepatocytes, which is shown in the in vitro starvation and glucose tracing experiments. More importantly, the in vitro data is recapitulated in vivo, as highlighted by indirect calorimetry and in vivo glucose tracing data. Finally, beyond the demonstration of the Per1-Fgf21-Pdk4 axis in basal cellular and physiological conditions, these data are extended to verify multiple nodes in this pathway can rescue the oxidative defects observed in hepatocyte Per1-deficient mice. This includes exogenous treatment with recombinant FGF21 protein, CPI-613 targeting PDH both in vitro and in vivo, stabilized carbohydrate analogs that include IMCTA and 6-AzTre.

These findings define a Per1-Fgf21-Pdk4 regulatory axis in otherwise healthy mice. These data are not completely unprecedented, as at least one prior study in Siberian Hamsters has demonstrated that recombinant FGF21 can induce Pdk4 protein accumulation in liver. Paradoxically, previous studies also show Pdk4 expression is elevated in patients with non-alcoholic steatohepatitis (NASH), and its expression is blunted in Fgf21-treated obese mice. Similarly observed is a disrupted Per1-Fgf21-Pdk4 axis in the obese state, wherein WD-fed mice had elevated Fgf21 and Pdk4 expression levels in the absence of Per1 or Fgf21 in the liver, respectively. Subsequent work regarding how and why the obese state disrupts this axis will better define how (and at what ZT) to modify this pathway to treat metabolic liver disease.

The carbohydrate-specific sensing aspect of this pathway reveals translational avenues to these findings. Indeed, both generalized macronutrient withdrawal and hepatic GLUT blockade using trehalose to induce Per1. Herein is the identification of trehalose analogs 6-TreAz and IMCTA, each comprising distinct carbon structures, and each of which differentially, selectively induces Per1, Per2 and Per3. This supports prior data indicating a structure-activity relationship linking trehalose-like compounds to induction of circadian and fasting-induced genes.

In sum, a circadian-independent function for hepatocyte Per1 has been defined and an Fgf21-Pdk4-based mechanism was provided through which Per1 drives substrate selection. Shown here is that Per1 is necessary for canonical fasting responses and is sufficient to protect from glucose intolerance in obese animals independent of circadian input. Finally, pharmacological tools are also provided, through which to examine normal hepatocyte circadian interactions with metabolism and to augment glucose homeostasis in the obese state.

### Therapeutic Methods

Also provided is a process of treating, preventing, or reversing a disease, including but not limited to metabolic diseases in a subject in need of administration of a therapeutically effective amount of Per1, so as to protect from glucose intolerance and to augment glucose homeostasis.

Methods described herein are generally performed on a subject in need thereof. A subject in need of the therapeutic methods described herein can be a subject having, diagnosed with, suspected of having, or at risk for developing a disease, including but not limited to a metabolic disease. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a gene modulation agent including Per1, for example, an amount that would cause the desired therapeutic effect in a subject while minimizing undesired side effects. In various embodiments, an effective amount of the gene modulation agent described herein can regulate glucose homeostasis, prevent glucose intolerance, or limit the development of a metabolic disease.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a gene modulation agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a disease, including but not limited to inflammatory disease and osteoporosis.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a gene modulation agent such as Per1 described herein can occur as a single event or over a time course of treatment. For example, a gene modulation agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for a disease, including but not limited to inflammatory disease and osteoporosis.

A gene modulation agent can be administered simultaneously or sequentially with another agent, such as an antibiotic, an anti-inflammatory, or another agent. For example, a gene modulation agent, which can be a modified probiotic that drives production of therapeutic gene products can be administered simultaneously with another agent, such as an antibiotic or an anti-inflammatory. Simultaneous administration can occur through the administration of separate compositions, each containing one or more of a gene modulation agent, an antibiotic, an anti-inflammatory, or another agent. Simultaneous administration can occur through the administration of one composition containing two or more of a gene modulation agent, an antibiotic, an anti-inflammatory, or another agent. A gene modulation agent can be administered sequentially with an antibiotic, an anti-inflammatory, or another agent. For example, a gene modulation agent can be administered before or after the administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers. Solid drugs may be formulated as nanoparticles or spray-dry dispersions (SDD) that often contain added excipients to improve stability and aid processing.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, subcutaneous, intranasal, epidural, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about-2 to about 4). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a probiotic and materials to facilitate oral administration in a pill form. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Hepatocyte Period 1 Dictates Oxidative Substrate Selection Independent of the Core Circadian Clock

The present disclosure newly defines a critical circadian-independent function for hepatocyte Period 1 (Per1) and provides an Fgf21-Pdk4-based mechanism through which Per1 drives substrate selection. As shown herein, Per1 is necessary for canonical fasting responses and is sufficient to protect from glucose intolerance in obese animals independent of circadian input. Novel pharmacological tools are introduced herein through which to examine normal hepatocyte circadian interactions with metabolism and to augment glucose homeostasis in the obese state.

### INTRODUCTION

Proper fuel selection during fasting or famine is a matter of survival. Hepatocytes reside at the intersection of portal and venous circulations and thus moderate acute and long-term energy homeostasis by coordinating substrate selection within the liver and across organ systems. Whereas the post-prandial state is marked by meal-derived carbohydrate flux from the portal system into hepatocytes and subsequent glucose oxidation through the tricarboxylic acid (TCA) cycle, the hallmarks of fasting consist of adaptations that include a transition from exogenous glucose and glycogen-derived glucose oxidation to fatty acid β-oxidation. This elegantly coordinated process comprises peripheral lipolysis and lipid export to fuel hepatocyte oxidation, ketogenesis, and storage of excess lipid as intrahepatic triglyceride for rapid on-site mobilization. Fasting hepatocytes further compensate by inducing autophagic flux to salvage organelles and glycoproteins as substrate to fuel gluconeogenesis and ketogenesis. Finally, fasting hepatocytes communicate and integrate extrahepatic fat oxidation and promote peripheral insulin sensitization by secreting fibroblast growth factor 21 (FGF21), an anti-diabetic hepatokine that mediates efficient substrate absorption during the next refeeding cycle. Together, compensatory actions in the hepatocyte maintain survival during shorter and longer-term fasting by sustaining carbon flux to key organs and by altering chromatin structure to prepare for prolonged or repeated famine.

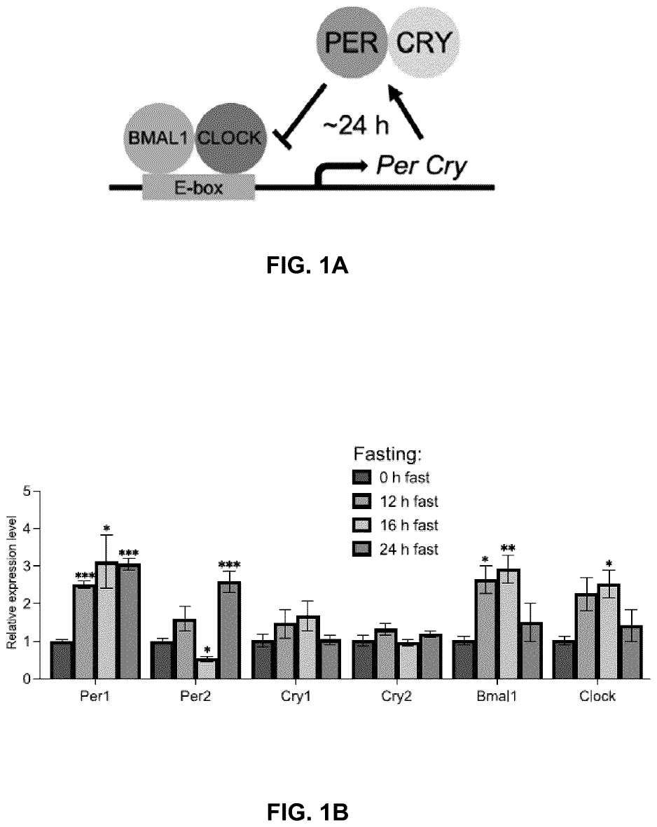

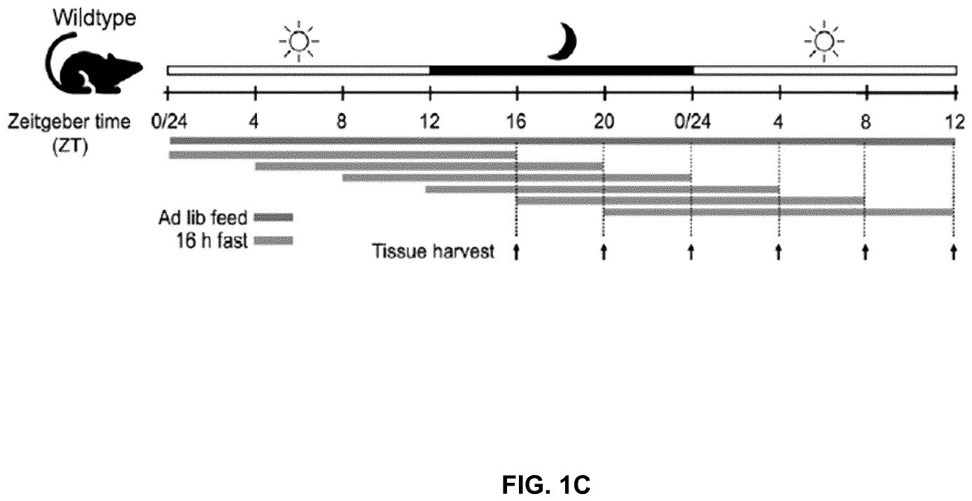

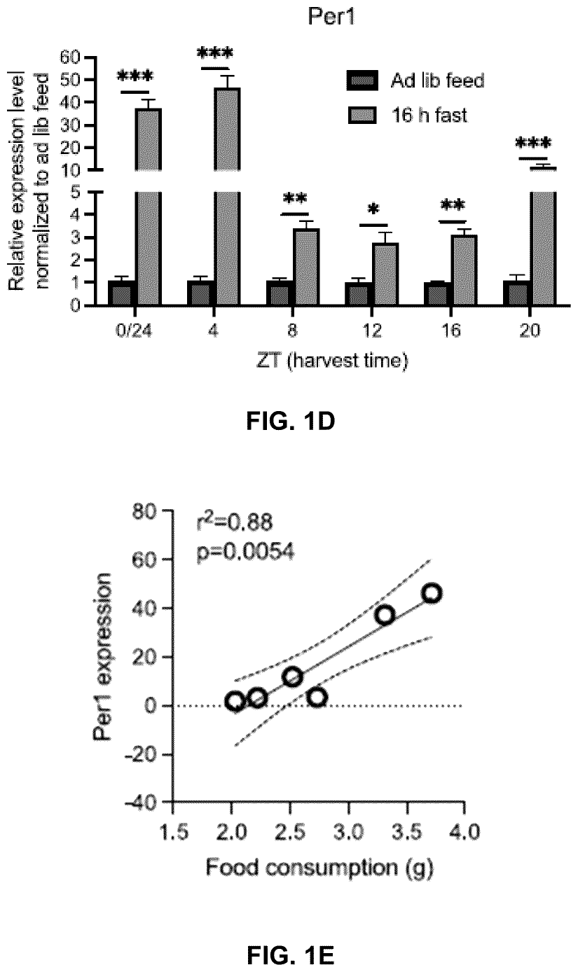

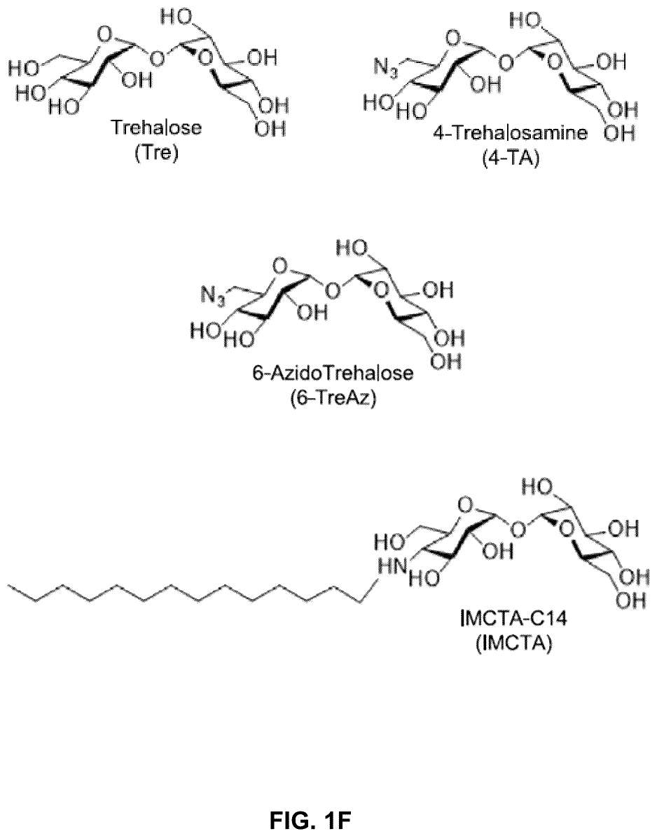

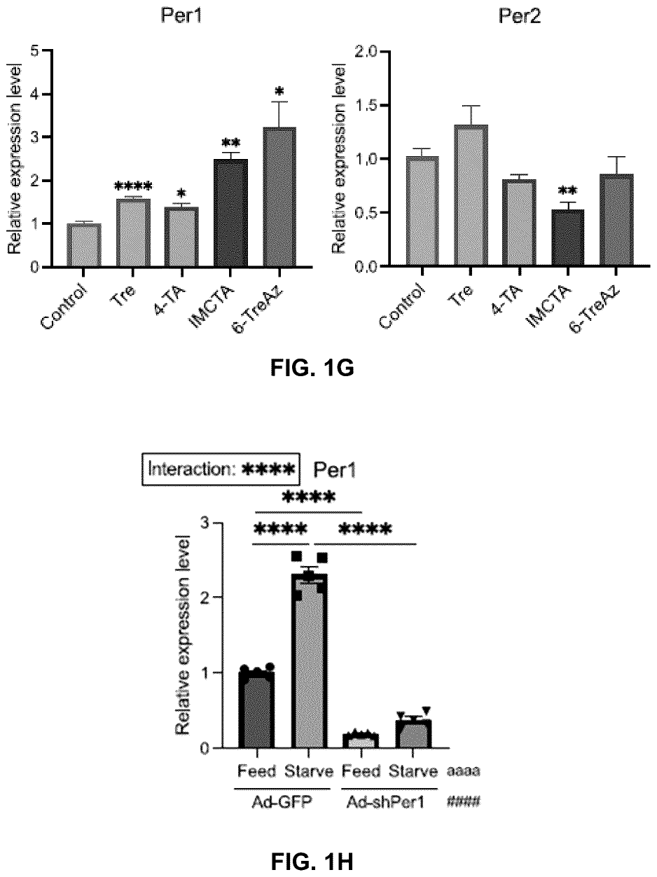

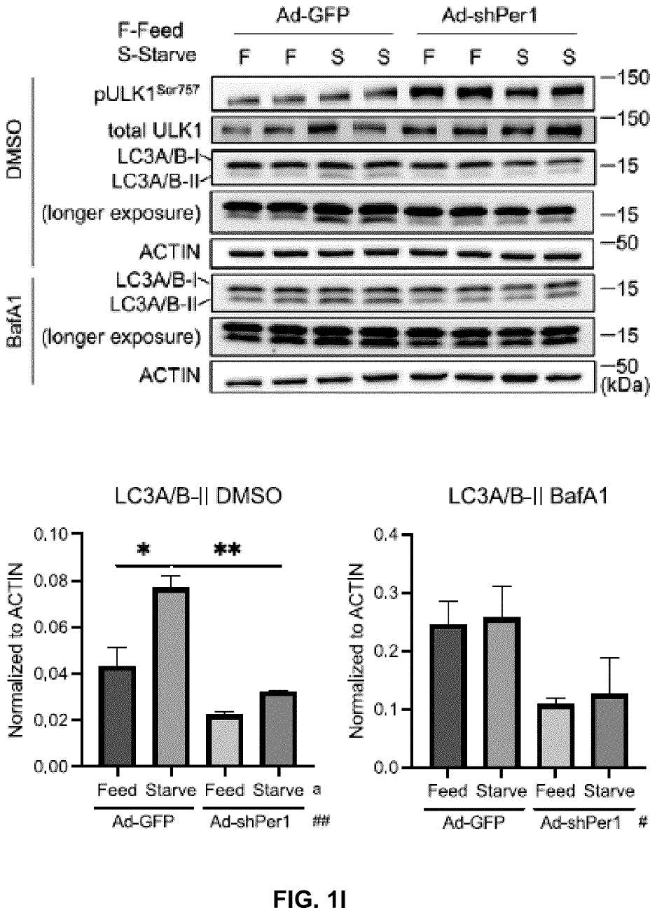

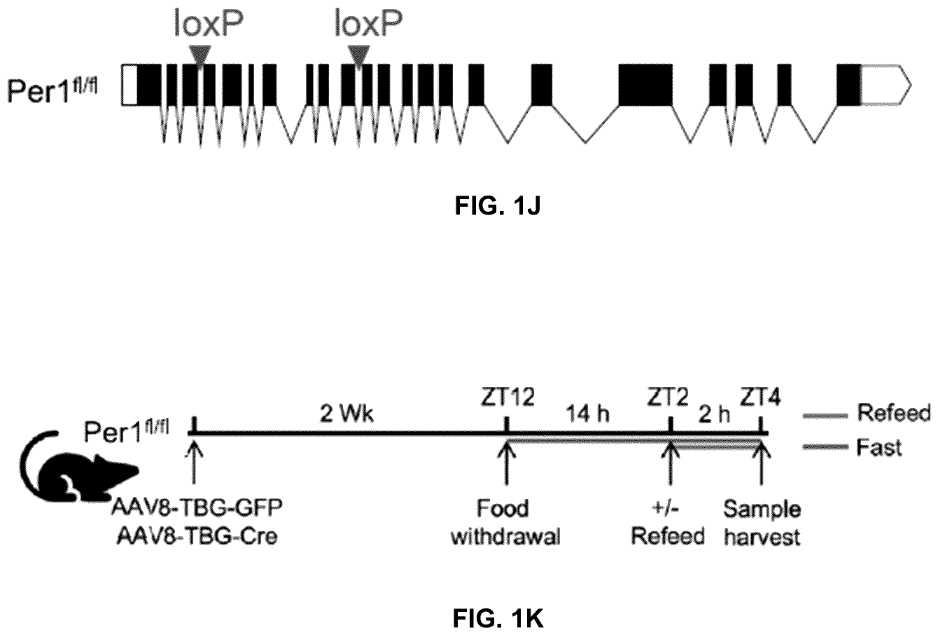

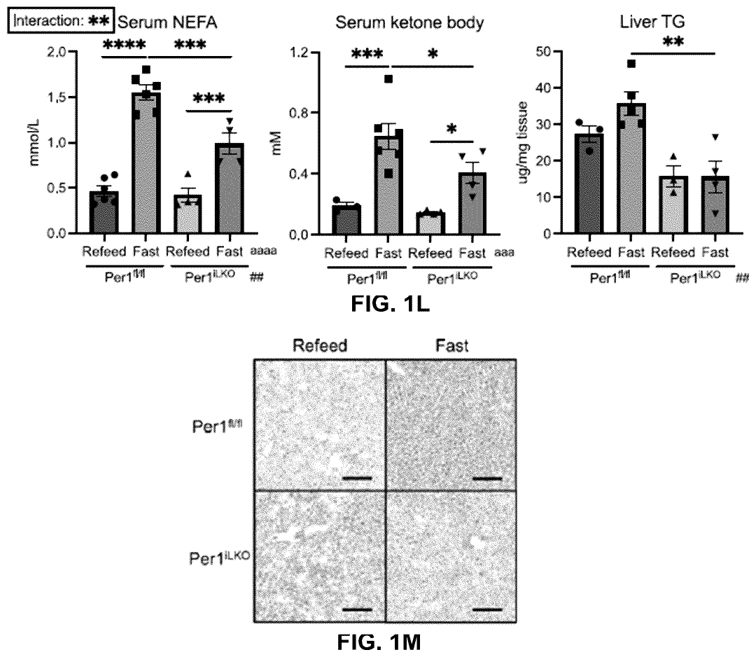

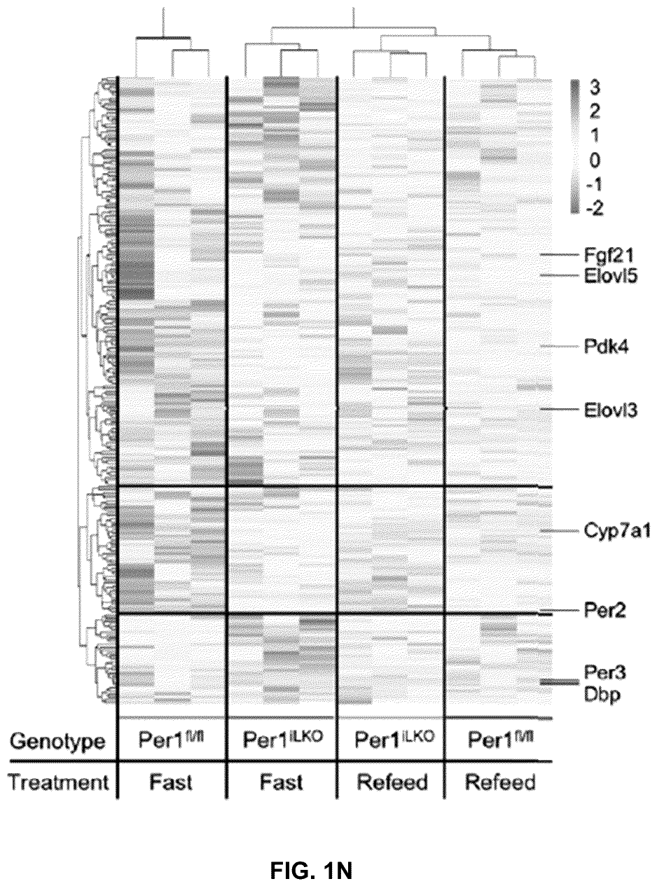

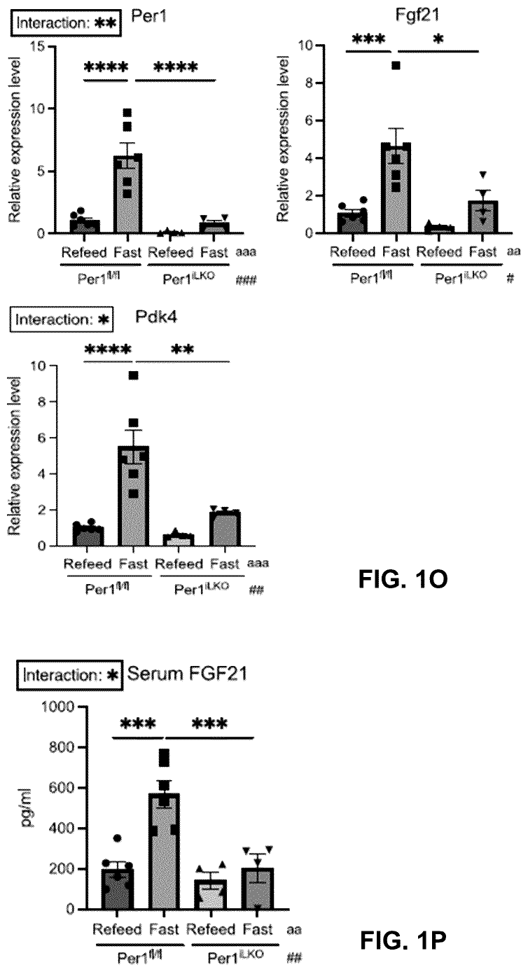

In addition, pending food availability, behaviorally complex organisms will fast, sleep, do both, or do neither, throughout the circadian clock. To account for this, the hepatocyte harbors a cell-intrinsic core clock that includes: Clock, Bmal1, cryptochrome 1 (Cry1) and Cry2, and period homolog gene 1 (Per1) and Per2. These form a well-characterized transcriptional-translational feedback loop to maintain cyclical expression (FIG. 1A). Genetic or environmental disruption of the core clock induces metabolic disease in mice and humans, while obesity alters rhythmic circadian gene expression. Recent data, however, suggest that the therapeutic metabolic response to time-restricted feeding is largely independent of the core clock. Thus, how and whether such signals interact to dictate fuel selection and survival remains incompletely addressed. Such dissonant findings led us to test the hypothesis that the hepatocyte integrates temporal and macronutrient sensing inputs to drive fuel selection.

Herein, this study identifies hepatic Per1 in multiple transcriptomic screens as a fasting-regulated, glucose-dependent factor in livers of fasting mice and in isolated hepatocytes. This study shows selective Per1 induction during short- and long-term fasting, even in the absence of an intact core clock. Indirect calorimetry and in vivo heavy-isotope metabolic labeling further demonstrate that Per1 drives the transition from glucose to fatty acid β-oxidation and manifold other fasting responses. Single-nucleus multiome sequencing and pharmacologic complementation experiments further demonstrate that Per1 mediates substrate transition through altering hepatocyte subpopulation chromatin accessibility, acute transcriptional changes, and post-transcriptional control of Fgf21, which modulates pyruvate dehydrogenase kinase 4 (Pdk4) signaling during fasting to shunt carbon flux away from pyruvate dehydrogenase (PDH)-mediated glucose oxidation. Finally, composition-of-matter hepatocyte carbohydrate flux inhibitors, 4-trehelosamine (4-TA), 6-azido trehalose (6-TreAz), and IMCTA-C14 (IMCTA), are nominated as translatable means by which to selectively induce Per1. These findings identify hepatocyte Per1 as a metabolic node that integrates temporal and nutritional input toward fuel selection and adaptation to shorter- and longer-term fasting.

### Results

### Hepatocyte Per1 Mediates the Adaptive Metabolic Response to Fasting

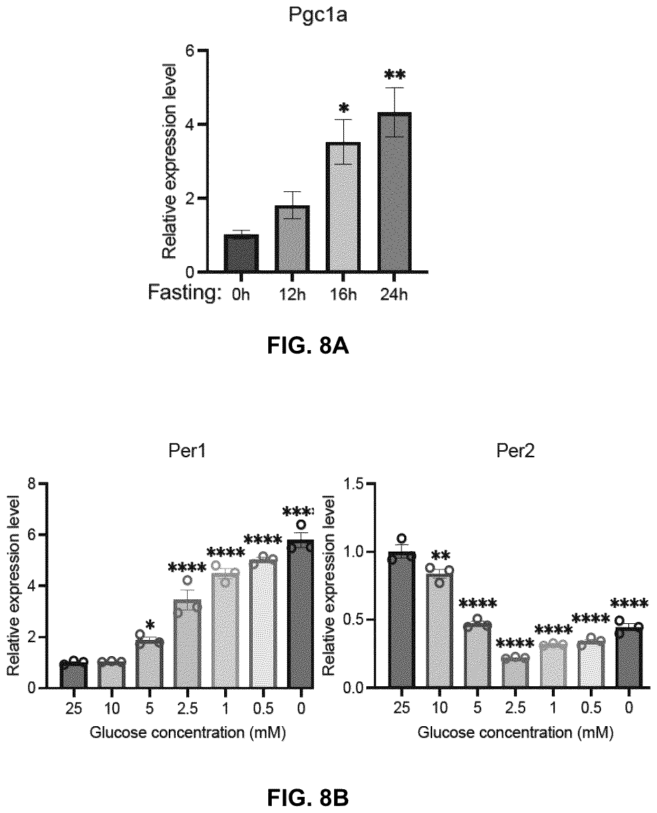

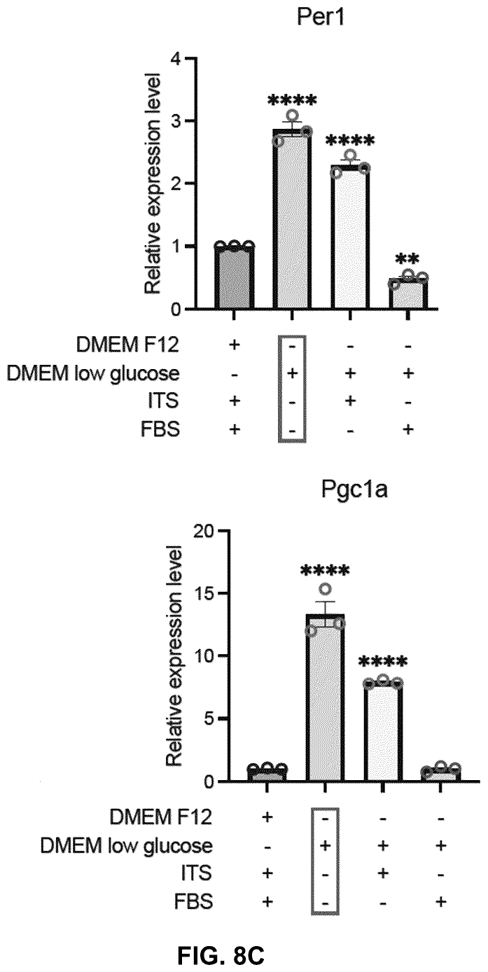

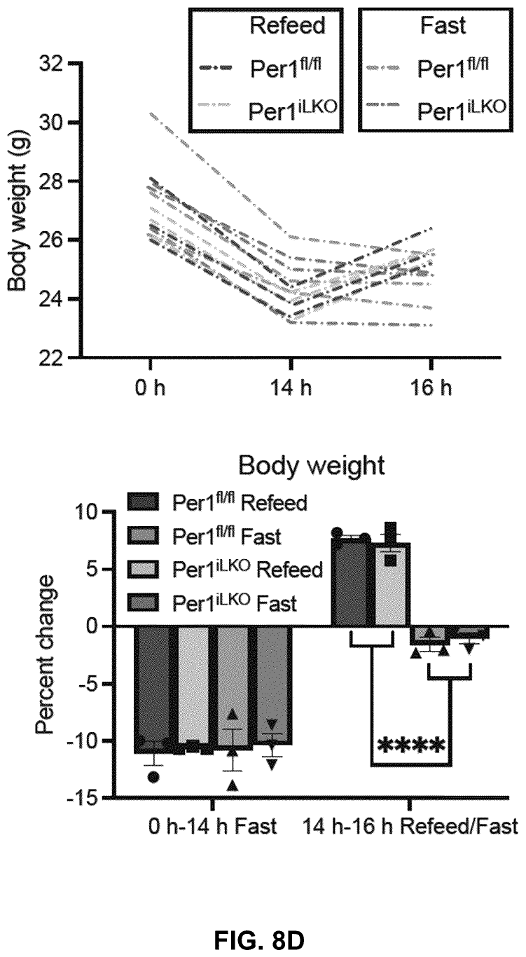

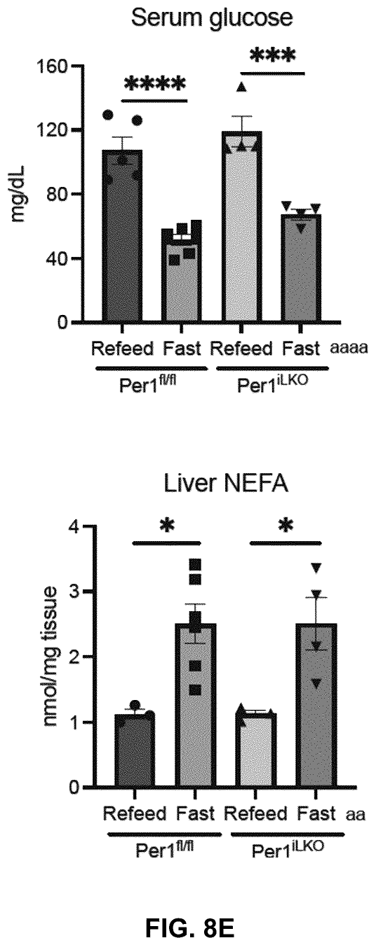

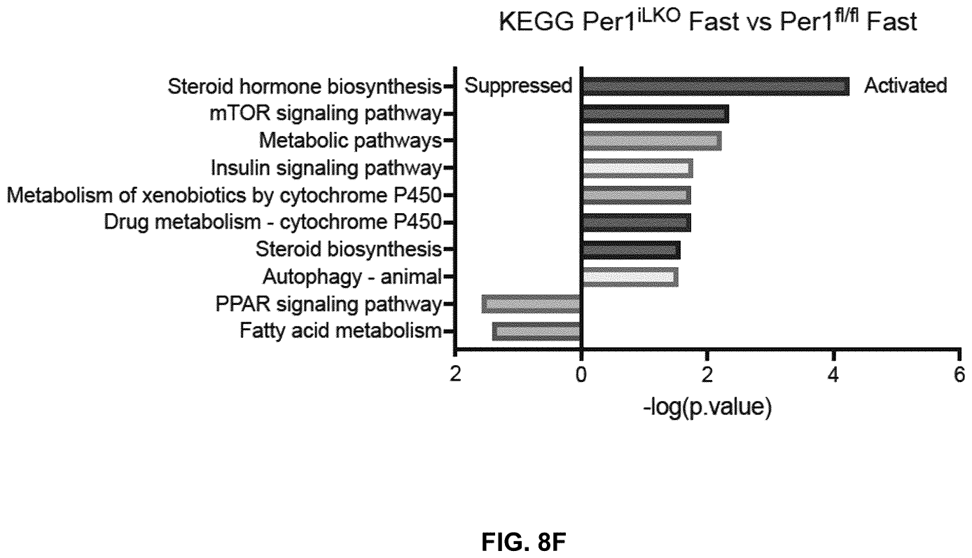

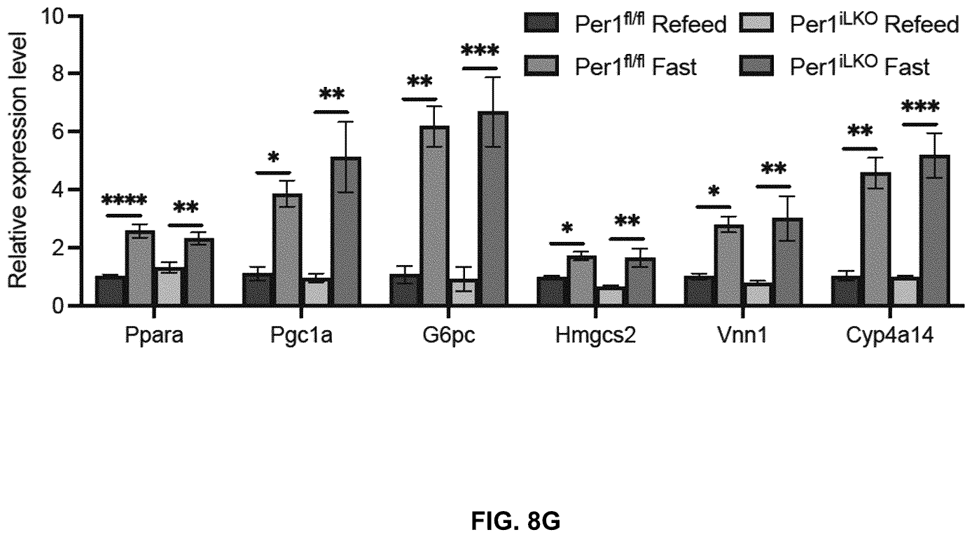

Per1 is a canonical circadian gene that is transcriptionally activated by Bmal1 and Clock (FIG. 1A). First examined were Per1, Pgc1a, and other core circadian gene expressions in liver in response to 12-h, 16-h, and 24-h starvation. This revealed significant induction of Per1 and the canonical fasting-induced Pgc1a gene during both shorter-term and prolonged fasting (FIG. 1B, FIG. 8A). In contrast, clock genes Per2, Cry1, Cry2, Bmal1, and Clock were not similarly induced and sustained throughout fasting for 12-24 h. Then the timing was defined for fasting initiation and termination interacted with Per1 induction in liver. A 16-h fasting was performed on wild-type mice and harvested tissues every 4 h throughout the 24-h time course (FIG. 1C). This revealed that fasting significantly induced Per1 across all time points, although fasting periods predominantly encompassing the dark phase (e.g., fasting termination at zeitgeber time 0/24 [ZT0/24] and ZT4) had the greatest stimulating effect on Per1 fasting induction (FIG. 1D). Because mice primarily eat throughout the active/dark phase, it was examined whether the magnitude of Per1 induction simply correlated with the magnitude of food deficit. Correlation of food mass consumed in mice fed ad libitum and Per1 induction during fasting at the same time point revealed a relationship between food deficit and Per1 induction (FIG. 1E). Next, it was tested whether glucose transporter (GLUT) blockade is sufficient to induce Per1 independent of full food withdrawal. This was performed using trehalose-derived GLUT inhibitors trehalose, 4-TA, 6-TreAz, and IMCTA (FIG. 1F). Each of these GLUT inhibitors induced Per1 up to 3.5-fold, without inducing Per2, in isolated primary hepatocytes (FIG. 1G). Consistent with this, the dose responsiveness of hepatocytes to glucose withdrawal was examined. Murine AML12 hepatocytes were treated with 0-25 mM glucose in regular growth medium and quantified Per1 and Per2 gene expression. Per1 but not Per2 increased in response to deceasing glucose exposure (FIG. 8B). Again, this indicated that glucose withdrawal is sufficient to induce hepatocyte Per1 and that Per1 regulation in response to glucose withdrawal is distinct from Per2 regulation.

To define broader Per1 functions during starvation, the effect of hepatocyte Per1 knockdown on autophagic flux and fasting signaling in vitro was quantified. Per1 expression was modified by adenovirus-driven short hairpin RNA (Ad-shPer1) in AML12 and starved the cells in nutrient-depleted medium (FIG. 1H, FIG. 8C). Then LC3B-II accumulation was quantified as a biomarker of autophagic flux, a process that is activated in liver during fasting. Per1 knockdown attenuated starvation-induced LC3B-II accumulation in hepatocytes treated with adenovirus encoding Per1 short hairpin RNA when compared with GFP-expressing hepatocytes (FIG. 1I). This occurred in the context of increased phosphorylation of the autophagy-inhibiting mammalian target of rapamycin (mTOR) complex 1 target site, PULK1Ser757, in fasting Per1-deficient hepatocytes (FIG. 1I).

This prompted the generation of mice harboring homozygous floxed hepatocyte-specific Per1 alleles using CRISPR-Cas9-mediated gene editing. LoxP sites were inserted flanking exons 4 and 10 of Per1 (FIG. 1J) and treated these mice with adeno-associated virus serotype 8 (AAV8)-encoding Cre recombinase under thyroxine-binding globulin promoter control (AAV8-TBG-Cre) to delete Per1 specifically in hepatocytes (Per1^{iLKO}, FIG. 1K). Two weeks after AAV8 treatment, these mice were subjected to 16-h fasting or fasting with refeeding (FIG. 1K). Fasting Per1^{iLKO} mice exhibited impaired fatty acid, ketone body, and intrahepatic triglyceride (TG) accumulation, and this was also confirmed by oil red O (ORO) staining (FIG. 1L, FIG. 1M). No significant difference in body-weight change, serum glucose, and liver free fatty acid level was observed between different genotypes (FIG. 8D, FIG. 8E).

Bulk transcriptomics in refed and fasting Per1^{fl/fl} and Perf^{iLKO} liver allowed us to identify potential pathways underlying defective substrate selection in Perf^{iLKO} mice. Unsupervised clustering demonstrated greater separation between fed and fasting Per^{fl/fl} liver transcriptome versus Per1^{iLKO} livers under the same conditions (FIG. 1N). Transcriptional pathways upregulated included mTOR and insulin signaling, whereas downstream peroxisome proliferator-activated receptor signaling and fatty acid oxidation were downregulated in fasting Per1^{iLKO} versus Per^{fl/fl} fasted liver (FIG. 8F). Among differentially expressed genes (DEGs) were the genes encoding both the fasting-induced hepatokine Fgf21 and Pdk4 (FIG. 1N). Deletion of hepatocyte Per1 in Per1^{iLKO} mice was verified and Fgf21 and Pdk4 gene expression was validated. Fgf21 peptide defects were verified by quantitative real-time PCR (real-time qPCR) (FIG. 1O) and ELISA (FIG. 1P). This confirmed impaired fasting-induced hepatic Fgf21 and Pdk4 expression and impaired fasting-induced FGF21 peptide in Per1^{iLKO} liver and serum, respectively (FIG. 1O, FIG. 1P). Given that Fgf21 and Pdk4 are both regulated by Ppara, Ppara expression was measured along with its downstream target genes (FIG. 8G). Fasting significantly induced Ppara and several target genes in both Per1^{fl/fl} and Per1^{iLKO} liver (FIG. 8G). Together, the data indicate that Per1 regulates Fgf21 and Pdk4 without major contribution by the Ppara pathway.

### Hepatocyte Per1 Mediates Transcriptional Changes and Chromatin Remodeling in a Fasting-Responsive Hepatocyte Subpopulation.

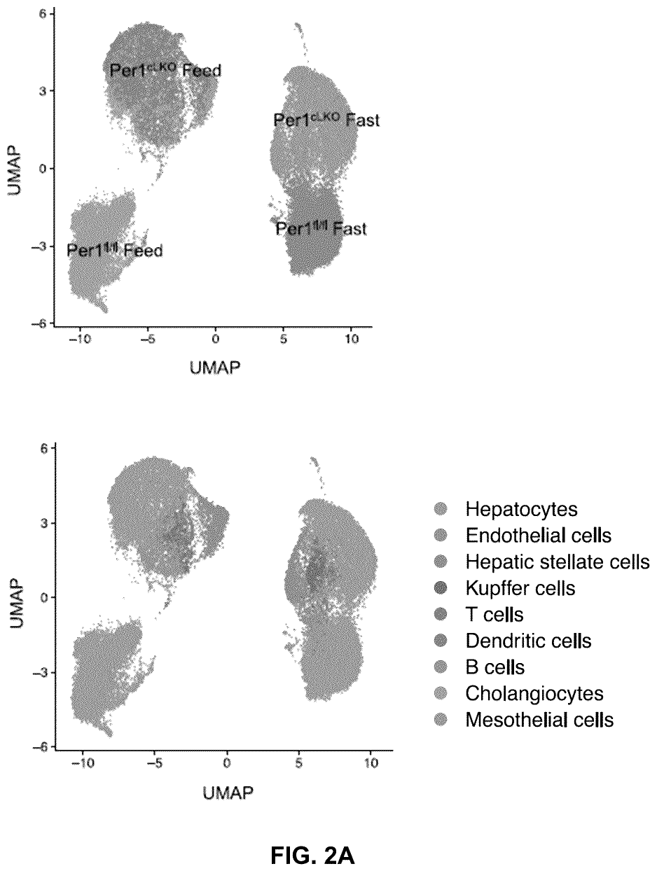

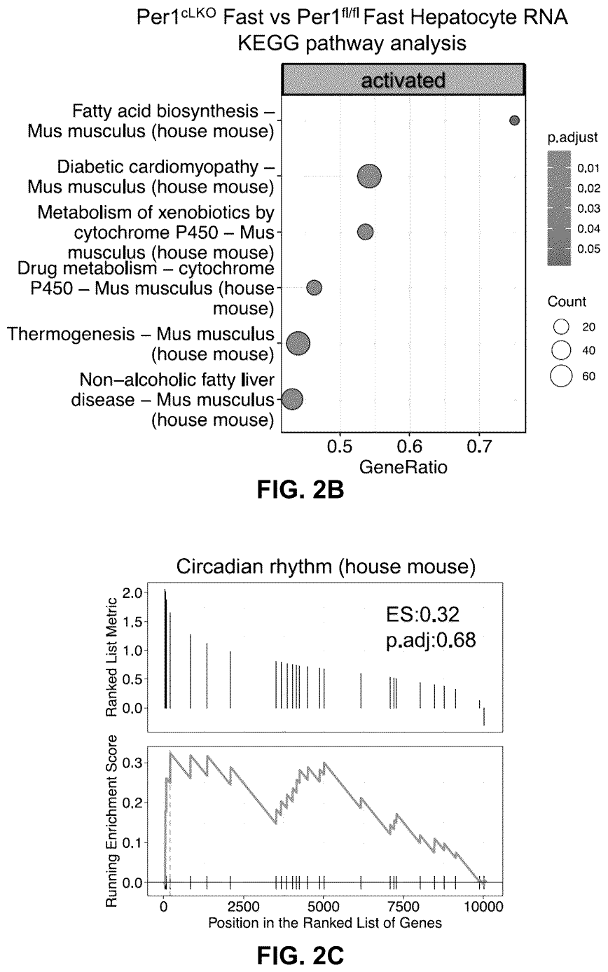

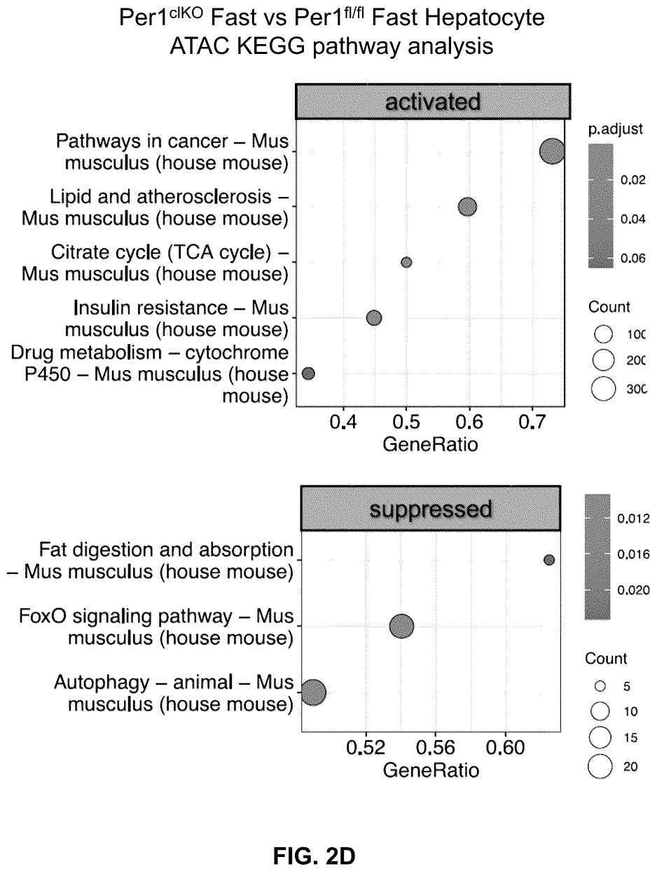

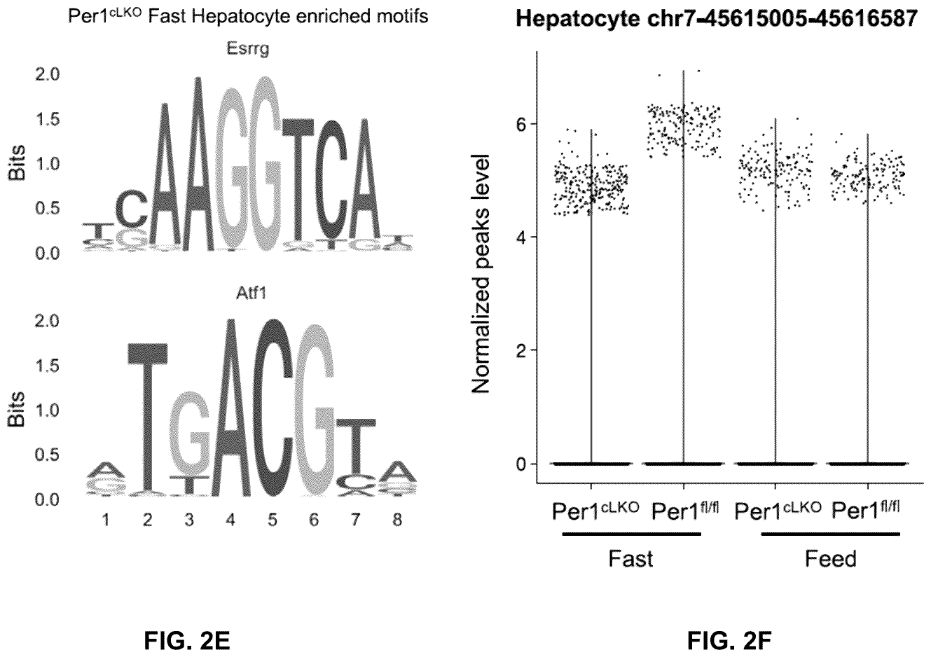

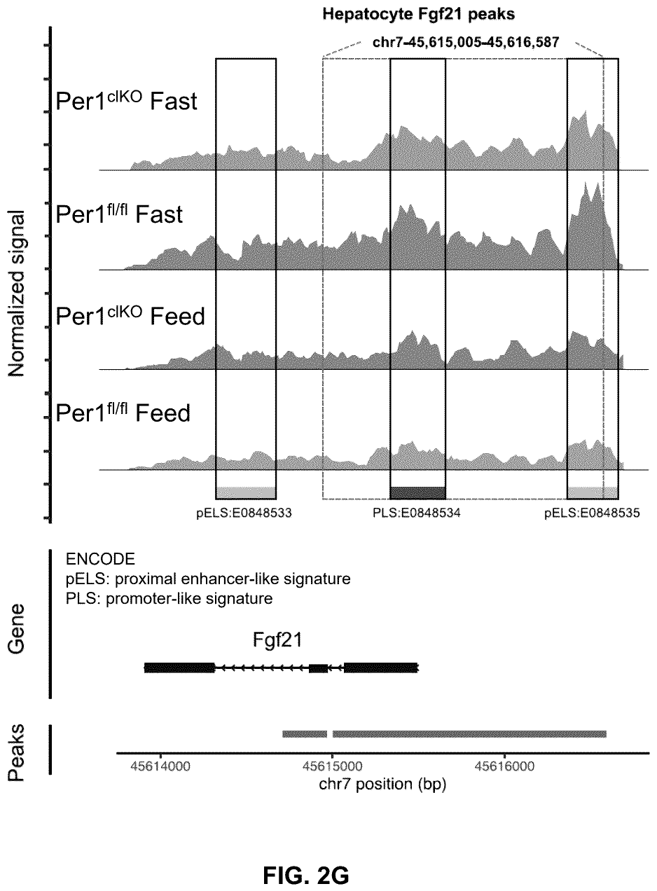

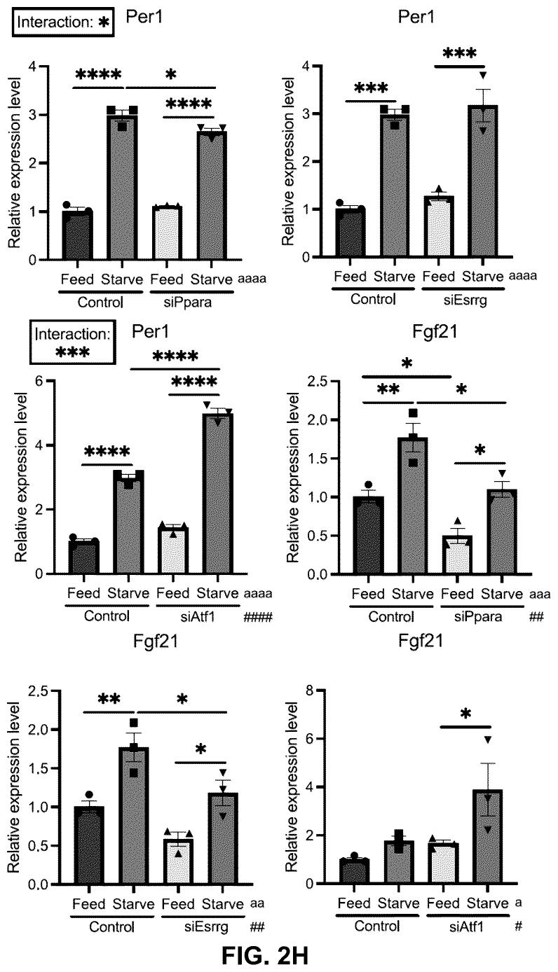

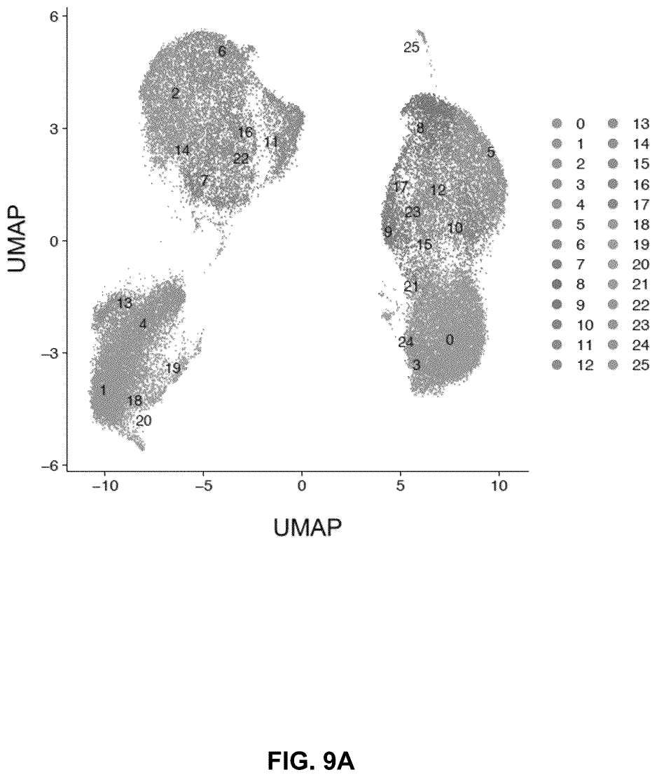

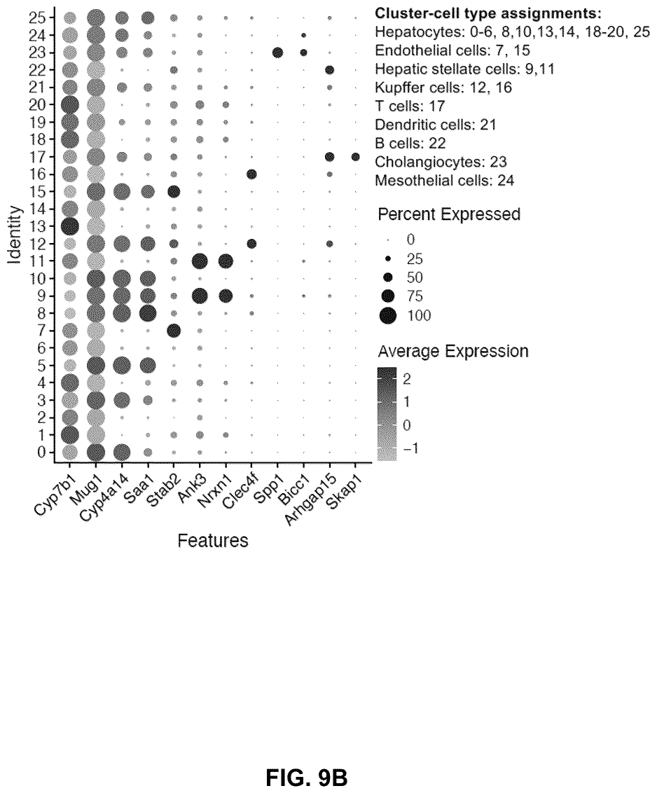

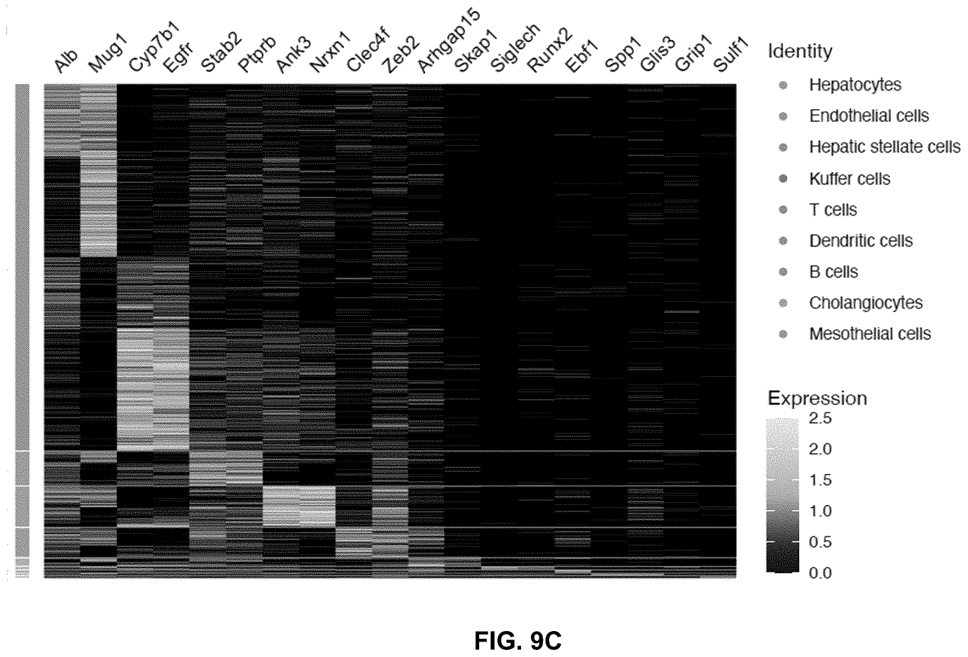

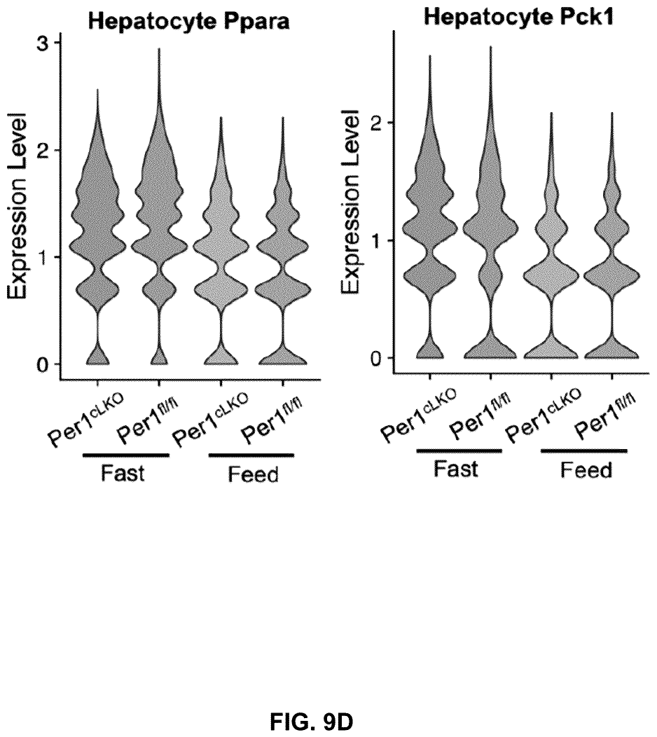

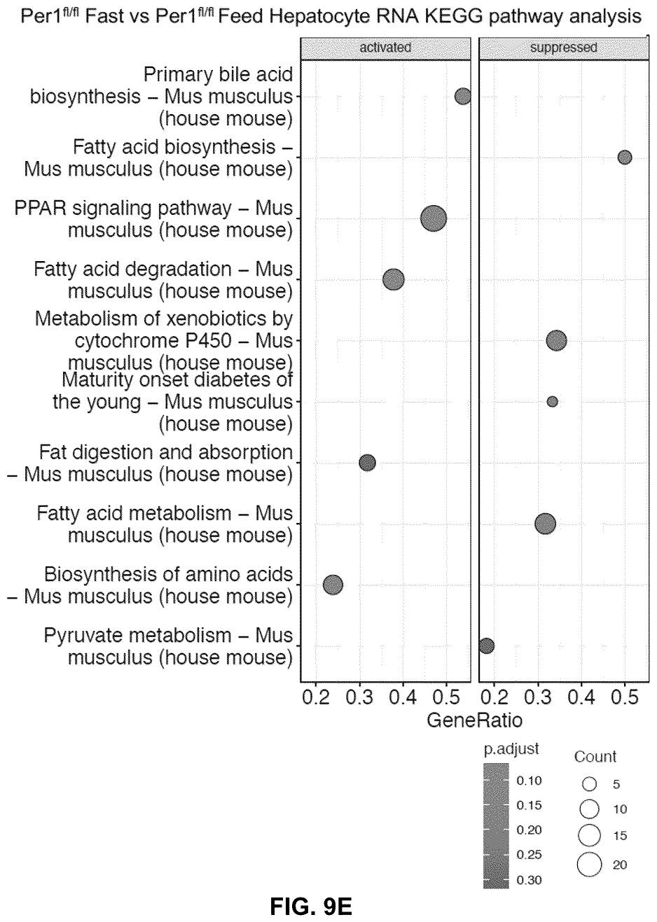

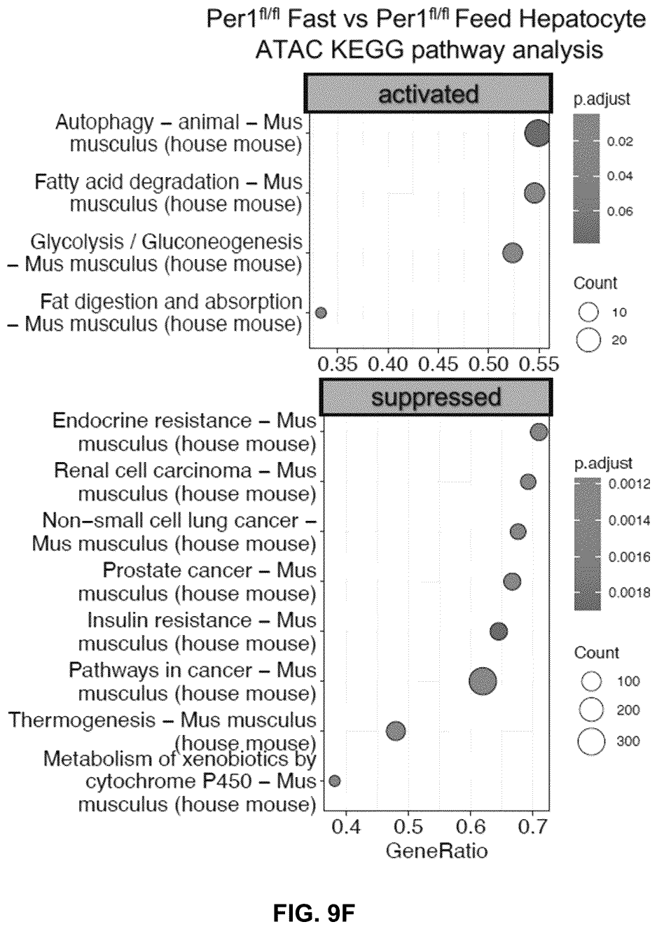

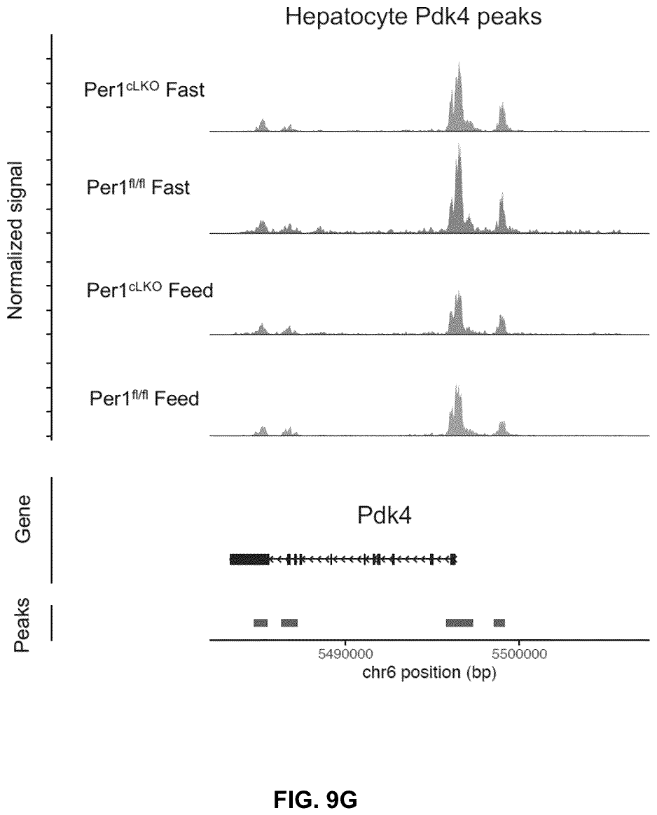

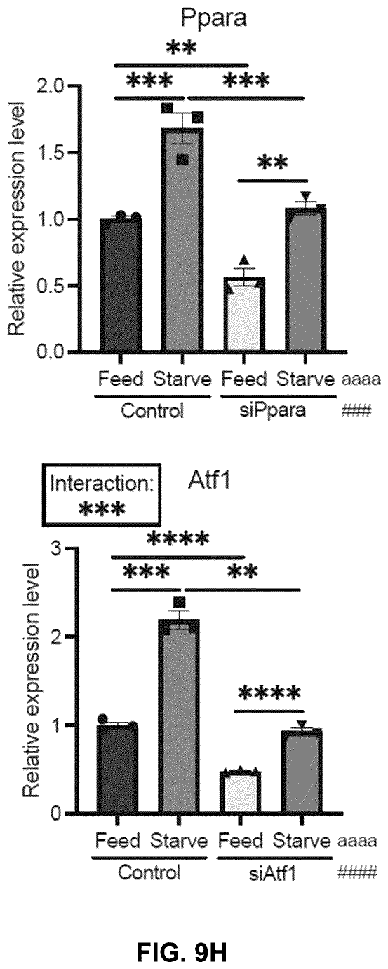

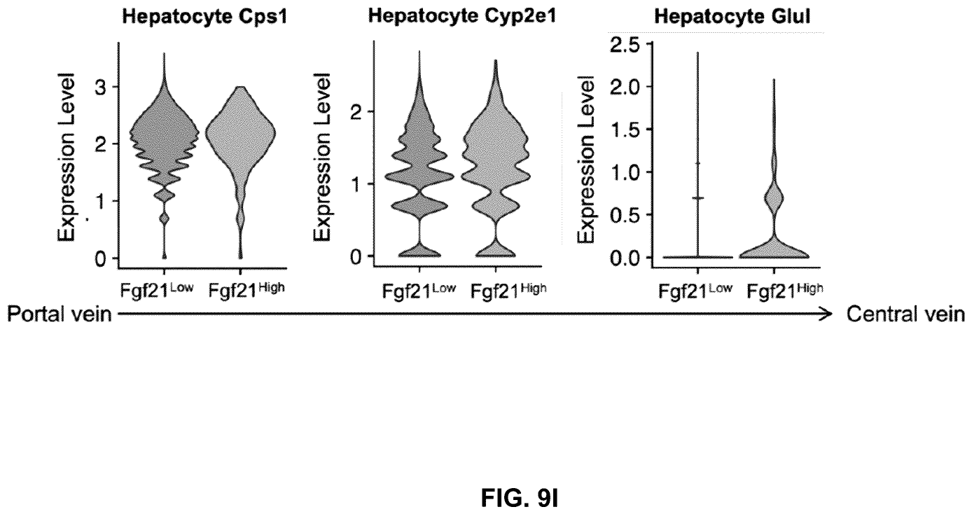

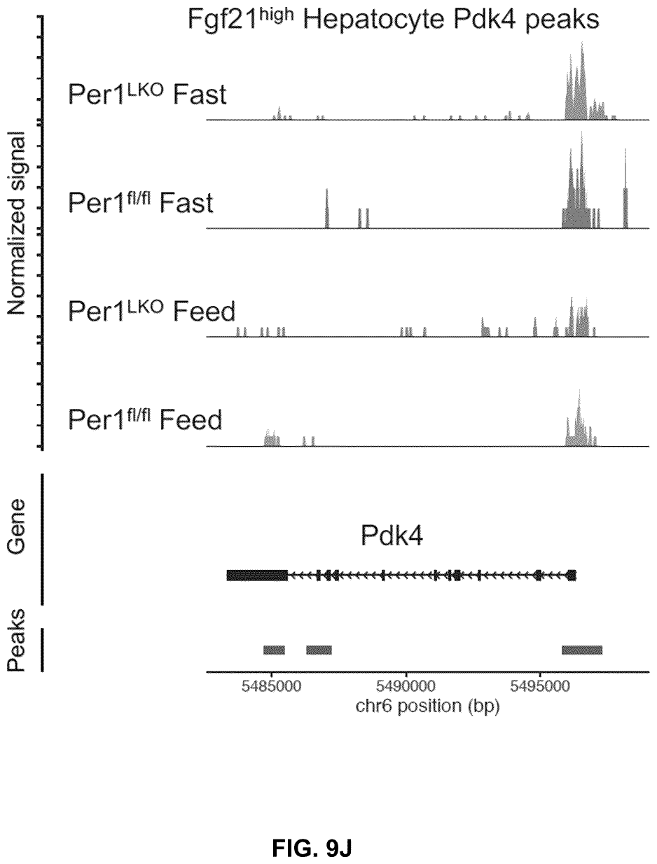

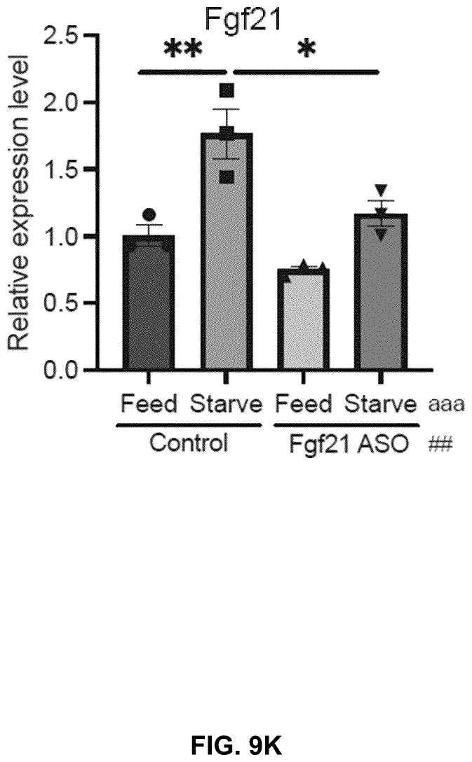

To gain deeper insight into hepatocyte-intrinsic defects during fasting in Per1^{fl/fl} or Per1^{fl/fl}; Alb-Cre (Per1^{cLKO}) liver, lever-aged single-nucleus multiome sequencing (e.g., single-nucleus RNA [snRNA] sequencing and single-nucleus assay for transposase-accessible chromatin [snATAC] sequencing) was used to define cellular subpopulations regulated by hepatocyte Per1 at the levels of RNA and chromatin (FIG. 2A). A stable germline hepatocyte-specific targeting approach was utilized to minimize acute gene expression and chromatin changes that might be observed due to an AAV8-mediated TBG-promoter-driven Cre targeting strategy. In this model, uniform manifold approximation and projection for dimension reduction (UMAP) plot-integrated snRNA-seq and snATAC-seq data revealed sharp demarcation between clusters of samples from each treatment and genotype (FIG. 2A). Cell-marker analysis verified identification of a broad complement of liver cells, including hepatocyte, endothelial, stellate, Kupffer cells, T cells, dendritic cells, B cells, cholangiocytes, and mesothelium cells (FIG. 9A, FIG. 9B, FIG. 9C). Expression of canonical hepatocyte genes Alb, Cyp7b1, Mug1, Cyp4a14, Egfr, and Saa1 defined the hepatocyte population in all groups (FIG. 9B, FIG. 9B). Transcriptomic signal of Fgf21 was not detected from the hepatocyte population in the single-nucleus multiome data. No prior groups have reported snRNA-seq Fgf21 expression in hepatocyte population. Upregulation of Ppara and Pck1 expression in fasting Per1^{fl/fl} mice when compared with fed Per1^{fl/fl} mice internally validated this studies fasting and analytical approaches (FIG. 9D). Kyoto Encyclopedia of Genes and Genomes (KEGG) pathway analysis comparing Per1^{fl/fl} and Per1^{cLKO} fasted hepatocytes revealed activation of genes involved in lipid deposition in the Per1^{LKO} fasted hepatocytes, without any detectable defect in the circadian rhythm pathway (FIG. 2B, FIG. 2C). snATAC-seq revealed enhanced chromatin accessibility in cancer, lipid, TCA-cycle, and insulin-resistance pathways in fasted Per1^{cLKO} hepatocytes. In addition, this analysis revealed suppressed chromatin accessibility in fat-metabolism- and autophagy-related pathways in fasted Per1^{cLKO} hepatocytes (FIG. 2D), which were oppositely regulated in Per1^{fl/fl} fasted hepatocytes when compared to Per1^{fl/fl} fed hepatocytes (FIG. 9E, FIG. 9F).

Motif analysis demonstrated a significant enrichment in Esrrg and Atf1 transcription factors in Per1^{cLKO} fasted hepatocytes (FIG. 2E). Since Esrrg is a known transcriptional activator that directly binds to the Fgf21 promoter, this study interrogated hepatocyte chromatin accessibility near the Fgf21 locus along chromosome 7 and made at least two key observations. First, fasting-induced chromatin accessibility at Fgf21, but not Pdk4, locus was reduced in Per1^{cLKO} hepatocytes during fasting at the promoter and enhancer regions (FIG. 2G, FIG. 9G). Overall, the data indicated cell subpopulation-intrinsic regulation of Fgf21 transcription. This study therefore directly examined Ppara, Esrrg, and Atf1 function in Per1-Fgf21 expression in fasting AML12 hepatocytes using a genetic loss-of-function experimental approach. This revealed that small interfering RNA (siRNA)-mediated Ppara and Esrrg knockdown impaired maximal Fgf21 induction during starvation (FIG. 2H, FIG. 9H). Although no motif was significantly enriched in Per1^{fl/fl} fasted hepatocytes, Esrrg was also enriched in hepatocytes from fed Per1^{cLKO} mice (Table 1). Direct functional and multiomic data together indicate that Per1 may regulate an Esrrg-Fgf21 axis.

TABLE 1
Enriched motifs in hepatocyte populations.

| Group | gene | RNA.auc | RNA.pval | Motif.feature | Motif.auc | Motif.pval | Avg.auc |
|---|---|---|---|---|---|---|---|
| Per1^{cLKO} | Esrrg | 0.526283 | 2.65E−17 | MA0643.1 | 0.561245 | 7.01E−48 | 0.543764 |
| Fast | | | | | | | |
| | Atf1 | 0.506561 | 2.73E−03 | MA0604.1 | 0.543743 | 2.96E−25 | 0.525152 |
| Per1^{cLKO} | Klf12 | 0.614698 | 8.69E−171 | MA0742.1 | 0.556446 | 1.82E−40 | 0.585572 |
| Feed | | | | | | | |
| | Nr5a2 | 0.594877 | 1.01E−148 | MA0505.1 | 0.559629 | 5.87E−45 | 0.577253 |
| | Sox6 | 0.600431 | 5.55E−154 | MA0515.1 | 0.513982 | 9.70E−04 | 0.557207 |
| | Rxra | 0.566399 | 9.27E−71 | MA0512.2 | 0.545022 | 2.34E−26 | 0.55571 |
| | Tcf12 | 0.540236 | 7.66E−30 | MA0521.1 | 0.552507 | 3.01E−35 | 0.546372 |
| | Rarb | 0.508603 | 4.39E−06 | MA0857.1 | 0.569374 | 3.21E−60 | 0.538989 |
| | Smad4 | 0.519885 | 5.68E−16 | MA1153.1 | 0.555565 | 2.88E−39 | 0.537725 |
| | Esrrg | 0.544217 | 1.88E−45 | MA0643.1 | 0.529594 | 2.90E−12 | 0.536905 |
| | Arnt | 0.521737 | 5.62E−15 | MA0004.1 | 0.550947 | 2.76E−33 | 0.536342 |
| | Nr2f6 | 0.5238 | 6.18E−34 | MA0677.1 | 0.545938 | 2.26E−27 | 0.534869 |
| | Zfx | 0.513888 | 7.77E−11 | MA0146.2 | 0.548948 | 7.46E−31 | 0.531418 |
| | Foxj3 | 0.51154 | 1.71E−08 | MA0851.1 | 0.548814 | 1.08E−30 | 0.530177 |
| | Arid3b | 0.505233 | 7.40E−05 | MA0601.1 | 0.547783 | 1.76E−29 | 0.526508 |
| | Prdm15 | 0.506894 | 6.87E−05 | MA1616.1 | 0.543111 | 2.65E−24 | 0.525002 |
| | Creb3l2 | 0.520085 | 1.27E−14 | MA0608.1 | 0.527824 | 5.21E−11 | 0.523954 |
| | Sox5 | 0.520736 | 5.85E−07 | MA0087.1 | 0.521105 | 6.37E−07 | 0.52092 |
| | Alx1 | 0.500407 | 5.97E−03 | MA0854.1 | 0.531817 | 6.05E−14 | 0.516112 |
| | Dlx3 | 0.500304 | 5.04E−03 | MA0880.1 | 0.528589 | 1.53E−11 | 0.514446 |
| | Dmrt1 | 0.500791 | 2.04E−03 | MA1603.1 | 0.527661 | 6.73E−11 | 0.514226 |
| | Foxj2 | 0.503512 | 2.19E−03 | MA0614.1 | 0.522312 | 1.41E−07 | 0.512912 |
| Per1^{fl/fl} | Foxj3 | 0.507639 | 5.03E−06 | MA0851.1 | 0.581532 | 2.74E−122 | 0.544585 |
| Feed | | | | | | | |
| | Sox6 | 0.539822 | 1.38E−37 | MA0515.1 | 0.542914 | 3.44E−35 | 0.541368 |
| | Stat2 | 0.512233 | 1.36E−07 | MA1623.1 | 0.564239 | 1.21E−76 | 0.538236 |
| | Npas2 | 0.504439 | 5.53E−11 | MA0626.1 | 0.519876 | 9.87E−09 | 0.512158 |
| Per1^{fl/fl} | N/A | | | | | | |
| Fast | | | | | | | |

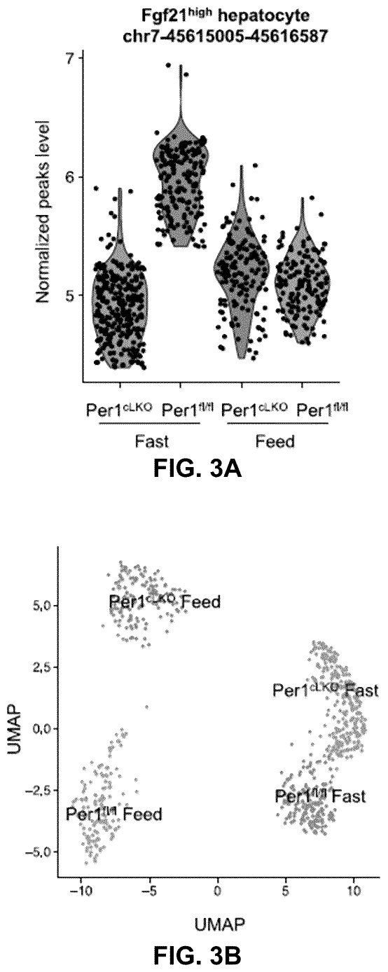

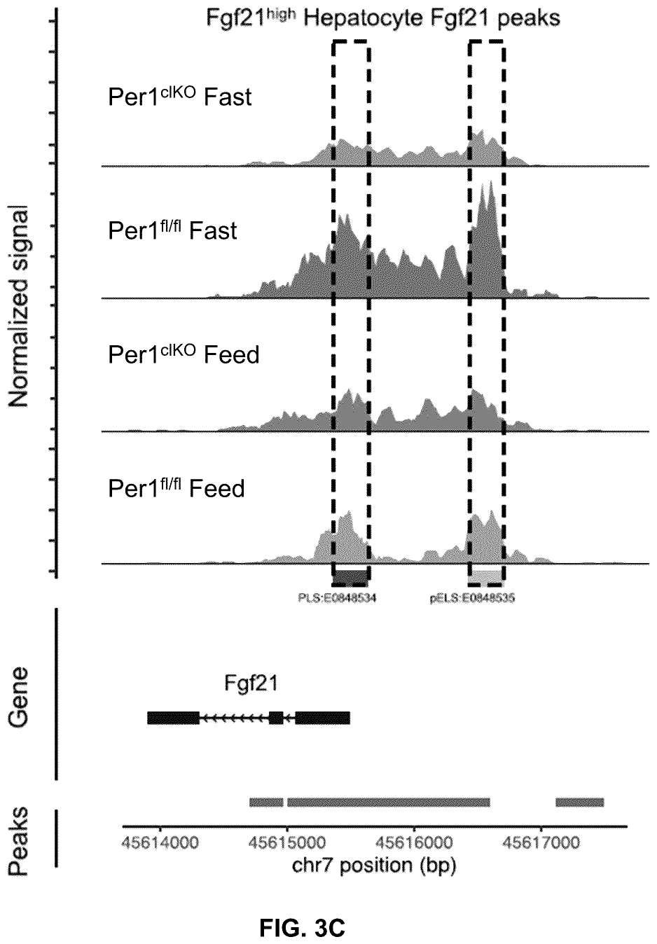

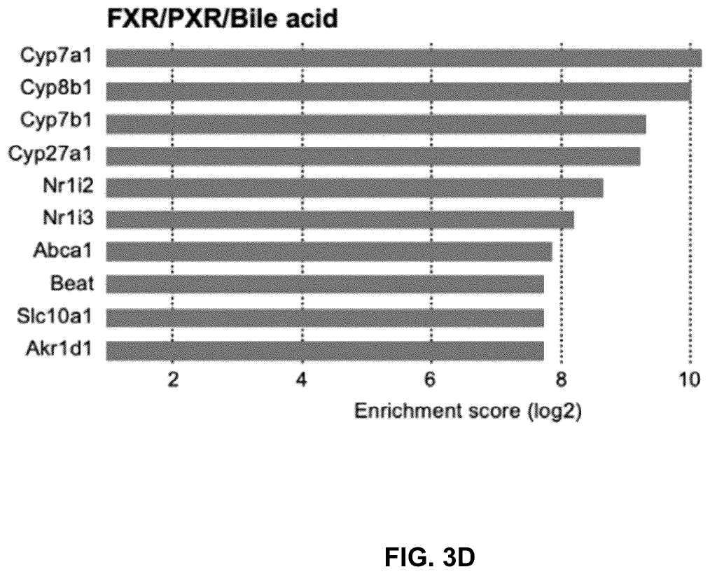

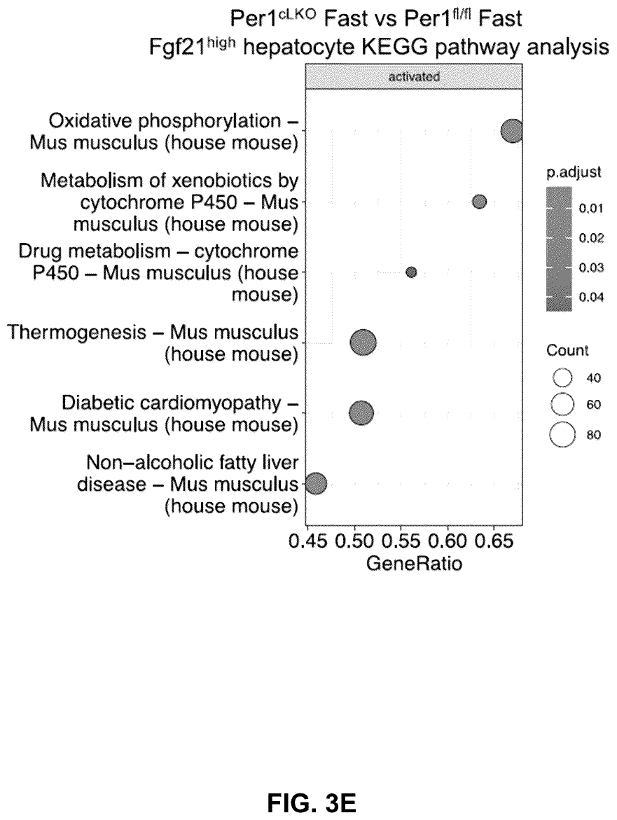

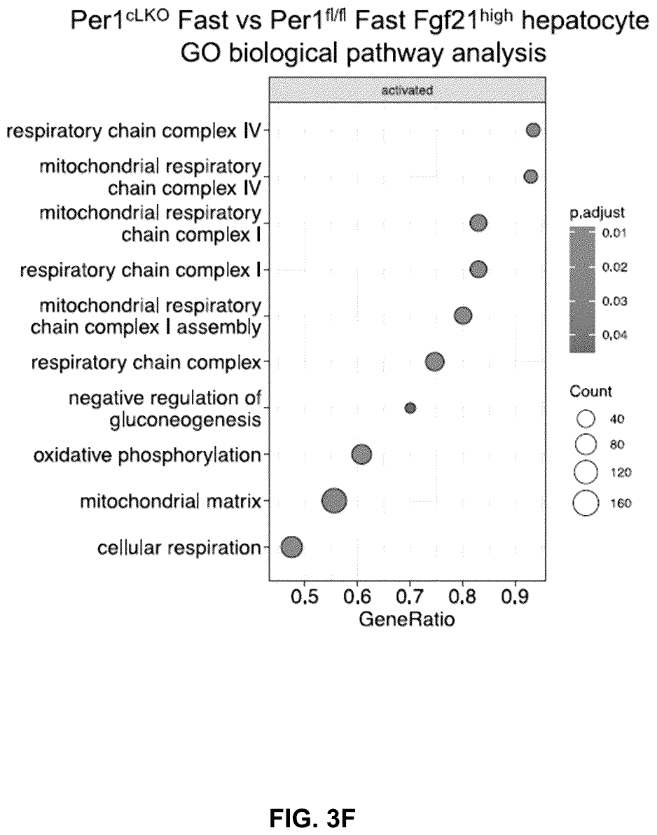

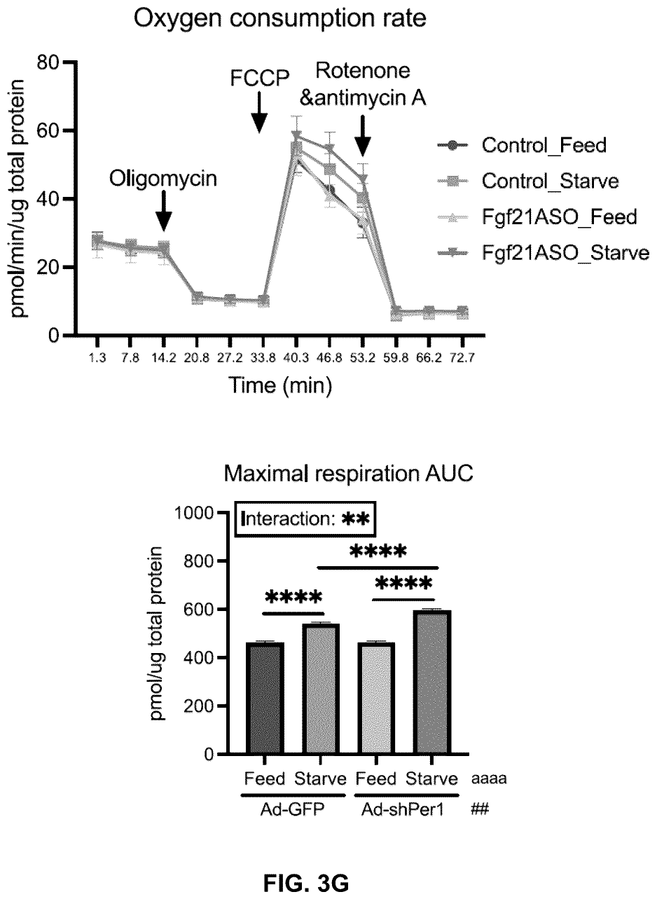

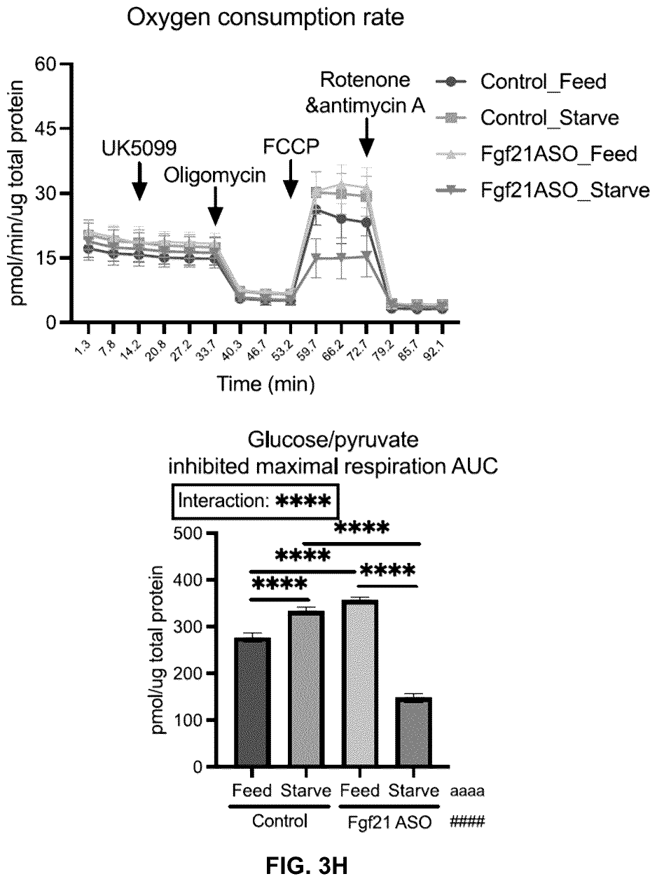

Second, a subpopulation of hepatocytes was identified that responded and maintained Fgf21 chromatin accessibility, as characterized by a normalized peak score >4 (e.g., Fgf21^{high} hepatocytes; FIG. 2F, FIG. 2G, FIG. 3A, FIG. 3B, FIG. 3C). These Fgf21^{high} hepatocytes comprised a relatively modest proportion of the overall hepatocyte population: Per1^{fl/fl} Feed, 1.6%; Per1^{fl/fl} Fast, 2.1%; Per1^{cLKO} Feed, 2.6%; Per1^{cLKO} Fast, 4.5%. The Fgf21^{high} hepatocyte subpopulation was dynamic and responded to fasting by increasing chromatin accessibility at the Fgf21 locus in fasting Per1^{fl/fl} mice but not in fasting Per1^{cLKO} mice (FIG. 3A, FIG. 3B, FIG. 3C). Isolated subpopulation analysis of the Fgf21^{high} hepatocyte transcriptomic profile further revealed enrichment of genes involved in the bile acid/farnesoid X receptor pathways in fasting Per1^{cLKO} and Fgf2^{high} hepatocytes (FIG. 3D, FIG. 9E, FIG. 9F; Table 2).

TABLE 2
Differentially expressed genes in
Fgf21^{High} versus Fgf21^{Low} hepatocytes.

| | | | |
|---|---|---|---|
| Gene | p_val | avg_log2FC | p_val_adj |

Per1^{fl/fl} Feed Hepatocyte Fgf21^{High} vs Fgf21^{Low}

| | | | |
|---|---|---|---|
| Zfp985 | 4.70E−15 | 5.985368 | 1.11E−10 |
| Rasl2-9 | 4.70E−15 | 5.985368 | 1.11E−10 |
| Gm47257 | 4.70E−15 | 5.985368 | 1.11E−10 |
| Ctrc | 3.61E−12 | 5.57033 | 8.55E−08 |
| Dipk1c | 3.63E−12 | 5.248402 | 8.60E−08 |
| Cdc7 | 3.07E−10 | 5.248402 | 7.28E−06 |
| Ptprh | 3.07E−10 | 5.248402 | 7.28E−06 |
| Gm48903 | 3.07E−10 | 5.248402 | 7.28E−06 |
| Gm36037 | 7.84E−10 | 3.985368 | 1.86E−05 |
| Ankrd35 | 7.43E−09 | 4.985368 | 0.000176 |
| Gm3764 | 7.46E−09 | 4.762975 | 0.000177 |
| A830082K12Rik | 1.35E−08 | 2.963 | 0.00032 |
| Platr16 | 2.62E−08 | 4.078477 | 0.000622 |
| MIf1 | 8.17E−08 | 4.762975 | 0.001936 |
| Galnt17 | 8.65E−08 | 3.284928 | 0.002051 |
| Tnip2 | 4.93E−07 | 1.985368 | 0.011682 |
| Hm629797 | 5.12E−07 | 3.897905 | 0.012146 |
| Gpr143 | 5.30E−07 | 4.57033 | 0.012567 |

Per1^{cLKO} Feed Hepatocyte Fgf21^{High} vs Fgf21^{Low}

| | | | |
|---|---|---|---|
| Fgf6 | 1.23E−17 | 6.77683 | 2.92E−13 |
| Klrb1a | 1.23E−17 | 6.77683 | 2.92E−13 |
| Gm44053 | 5.73E−12 | 5.77683 | 1.36E−07 |
| Gm6878 | 5.73E−12 | 5.77683 | 1.36E−07 |
| Gm35438.1 | 5.73E−12 | 5.77683 | 1.36E−07 |
| Gm15932 | 2.96E−10 | 4.191868 | 7.01E−06 |
| Dchs1 | 4.07E−09 | 5.191868 | 9.64E−05 |
| BC053393 | 4.07E−09 | 5.191868 | 9.64E−05 |
| Gm48898 | 4.07E−09 | 5.191868 | 9.64E−05 |
| Gm30655 | 4.07E−09 | 5.191868 | 9.64E−05 |
| Siah3 | 4.07E−09 | 5.191868 | 9.64E−05 |
| B130046B21Rik | 4.07E−09 | 5.191868 | 9.64E−05 |
| Fblim1 | 1.07E−08 | 4.384513 | 0.000254 |
| Eqtn | 2.14E−07 | 4.77683 | 0.005066 |
| Vmn2r60 | 2.14E−07 | 4.77683 | 0.005066 |
| Il17d | 2.14E−07 | 4.77683 | 0.005066 |
| Mgat3 | 2.14E−07 | 4.77683 | 0.005066 |
| Rmi2 | 2.14E−07 | 4.77683 | 0.005066 |
| Tmem47 | 2.14E−07 | 4.77683 | 0.005066 |
| Gm15046 | 2.14E−07 | 4.77683 | 0.005066 |
| Gpc3 | 3.90E−07 | 4.021943 | 0.009236 |
| Cd300c2 | 1.50E−06 | 3.869939 | 0.035671 |

Per1^{fl/fl} Fast Hepatocyte Fgf21^{High} vs Fgf21^{Low}

| | | | |
|---|---|---|---|
| Alox12b | 1.59E−14 | 6.498831 | 3.78E−10 |
| Gm5431 | 1.61E−14 | 5.498831 | 3.82E−10 |
| Ccdc190 | 1.61E−14 | 5.083794 | 3.82E−10 |
| 4930515L19Rik | 1.34E−13 | 5.176903 | 3.17E−09 |
| Gm47710 | 1.35E−13 | 4.913869 | 3.19E−09 |
| Mccc1os | 6.29E−12 | 5.013404 | 1.49E−07 |
| Gm50069 | 4.78E−11 | 5.913869 | 1.13E−06 |
| Gm9903 | 4.81E−11 | 5.498831 | 1.14E−06 |
| Gm33914 | 4.81E−11 | 5.498831 | 1.14E−06 |
| C330019F10Rik | 4.81E−11 | 5.498831 | 1.14E−06 |
| Gm20711 | 3.22E−10 | 4.498831 | 7.64E−06 |
| Rpa3 | 1.43E−09 | 4.176903 | 3.40E−05 |
| A630014C17Rik | 5.98E−09 | 5.498831 | 0.000142 |
| Tgfb3 | 6.02E−09 | 5.083794 | 0.000143 |
| Gm16553 | 5.35E−08 | 3.913869 | 0.001268 |
| Angpt4 | 1.52E−07 | 5.176903 | 0.003597 |
| Ckmt2 | 1.53E−07 | 4.761865 | 0.003626 |
| Dusp13 | 1.53E−07 | 4.761865 | 0.003626 |
| Epcam | 1.53E−07 | 4.761865 | 0.003626 |
| Gm34081 | 1.54E−07 | 4.498831 | 0.00364 |
| Tubb6 | 1.54E−07 | 4.498831 | 0.00364 |
| Gm14643 | 7.19E−07 | 3.59194 | 0.017044 |
| Styxl1 | 1.26E−06 | 3.020784 | 0.029904 |
| Nlrp9b | 1.54E−06 | 4.913869 | 0.03661 |
| Arhgap33 | 1.55E−06 | 4.691476 | 0.036735 |
| Mgst3 | 1.56E−06 | 4.498831 | 0.036923 |
| Chrnb2 | 1.97E−06 | 4.176903 | 0.046746 |

Per1^{cLKO} Fast Hepatocyte Fgf21^{High} vs Fgf21^{Low}

| | | | |
|---|---|---|---|
| Clvs1 | 1.59E−11 | 5.381141 | 3.78E−07 |
| Nsg1 | 1.59E−11 | 5.381141 | 3.78E−07 |
| Shisa6 | 1.01E−07 | 4.381141 | 0.002399 |
| Usp11 | 7.65E−07 | 3.018571 | 0.018129 |
| Gm14302 | 1.85E−06 | 3.533144 | 0.043754 |
| Npr3 | 1.85E−06 | 3.533144 | 0.043754 |

This study next asked whether zonal distribution could at least in part characterize the Fgf21^{high} hepatocyte population. This revealed significant enrichment and a trend toward significant enrichment of central vein zonal marker expression Cyp2e1 and Glul, respectively in the Fgf21^{high} population (FIG. 9I). This suggested potential zonal predilection associated with fasting sensitivity at the Fgf21 locus. Oxidative phosphorylation, thermogenic, and respiration pathways were further enriched in Per1^{cLKO} Fgf21^{high} hepatocytes that failed chromatin fasting induction (FIG. 3E, FIG. 3F). Again, no defect in chromatin accessibility was found at the Pdk4 locus (FIG. 9J), and this correlated with in vitro enhancement in mitochondrial respiration in starved Per1-deficient AML12 hepatocytes subjected to Seahorse mitochondrial respirometry (FIG. 3G).

In a distinct line of mitochondrial respiration experiments, this study next sought to define the effect of inhibiting fatty acid or glucose/pyruvate oxidation in hepatocytes with or without Fgf21 knockdown. Real-time mitochondrial respiration was measured in the presence or absence of pyruvate carrier inhibitor (UK5099) or fatty acid oxidation inhibitor (etomoxir) in AML12 hepatocytes with or without Fgf21 antisense oligonucleotide (FIG. 9K). Cells lacking Fgf21 exhibited significantly reduced mitochondrial respiration when pyruvate oxidation was inhibited. In contrast, inhibiting fatty acid oxidation in fasting Fgf21-deficient hepatocytes had no significant impact on maximal respiration when compared to fed Fgf21-deficient hepatocytes (FIG. 3H, FIG. 3I). This indicated that the absence of Fgf21 in hepatocytes causes greater reliance on glucose/pyruvate than fatty acid as their major energy source during fasting. Together, the data indicate a specific hepatocyte-intrinsic subpopulation of fasting non-responsive hepatocytes that is demarcated by failed Fgf21 accessibility, upregulated bile acid signaling, and increased oxidative phosphorylation.

### The Hepatocyte-Intrinsic Circadian Clock is Dispensable for Fasting-Induced Per1 Regulation

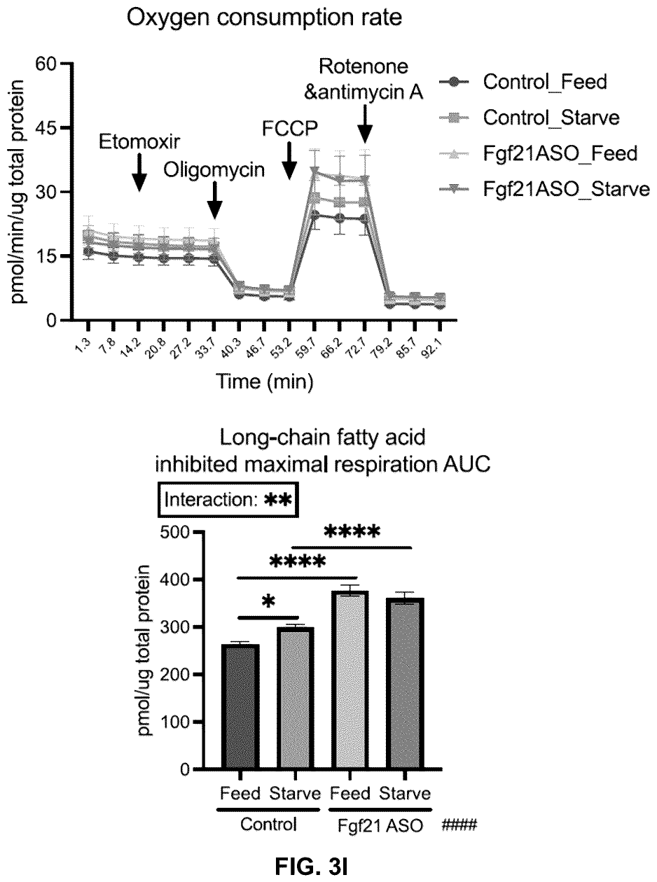

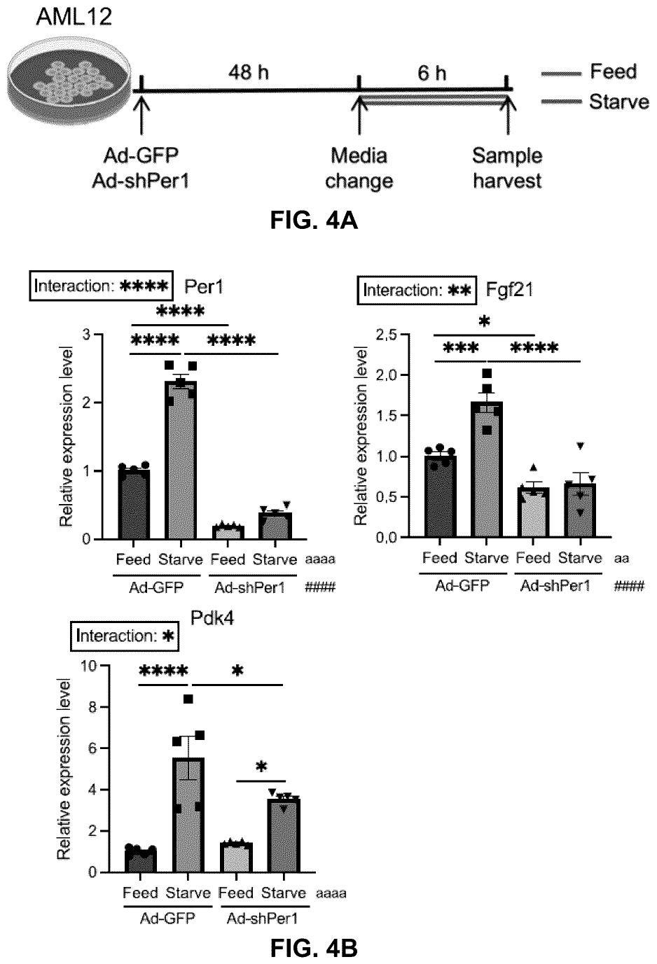

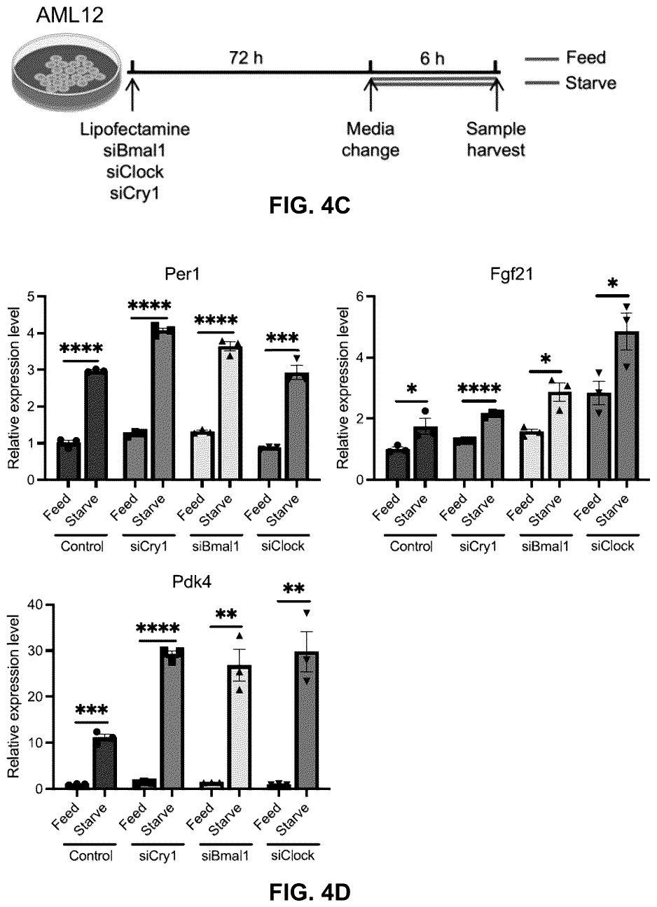

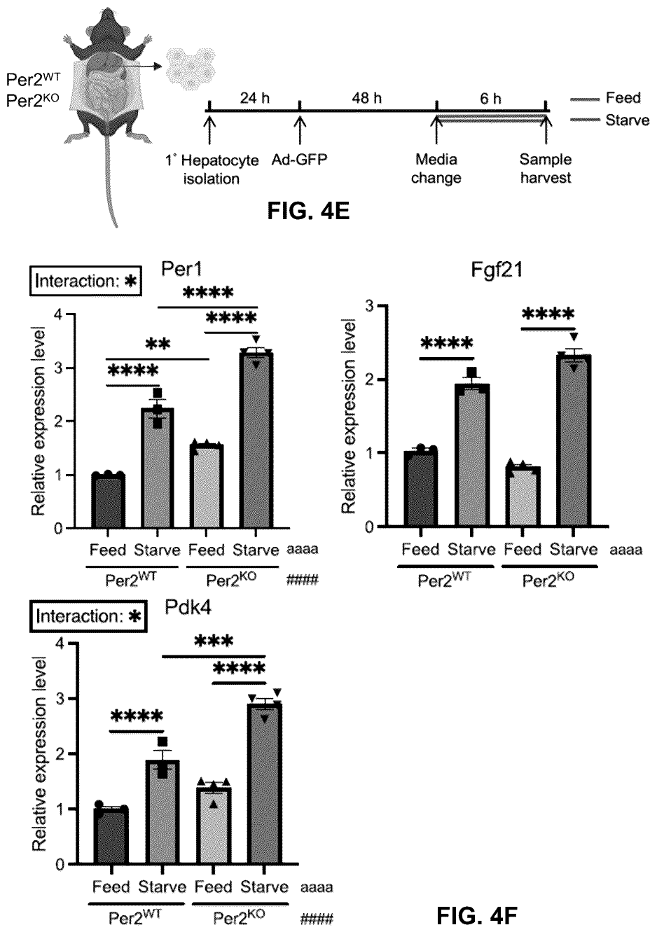

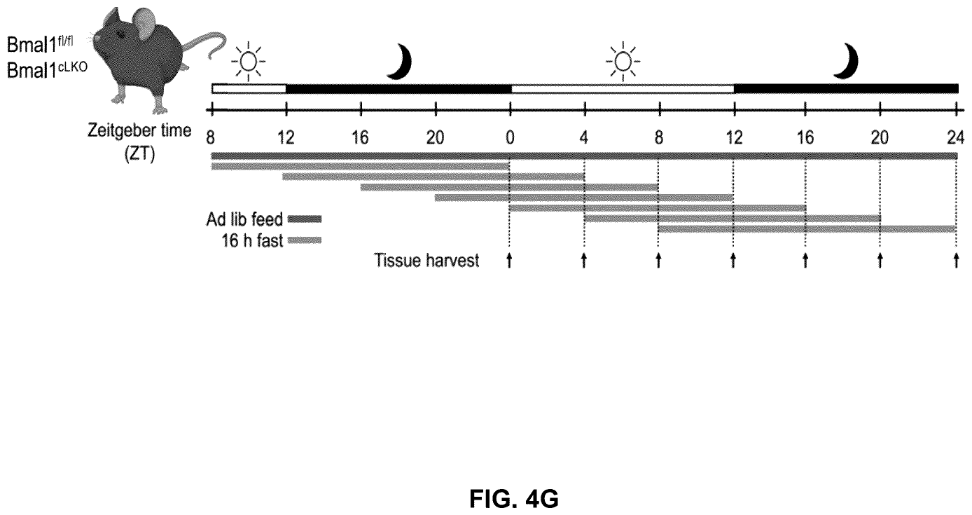

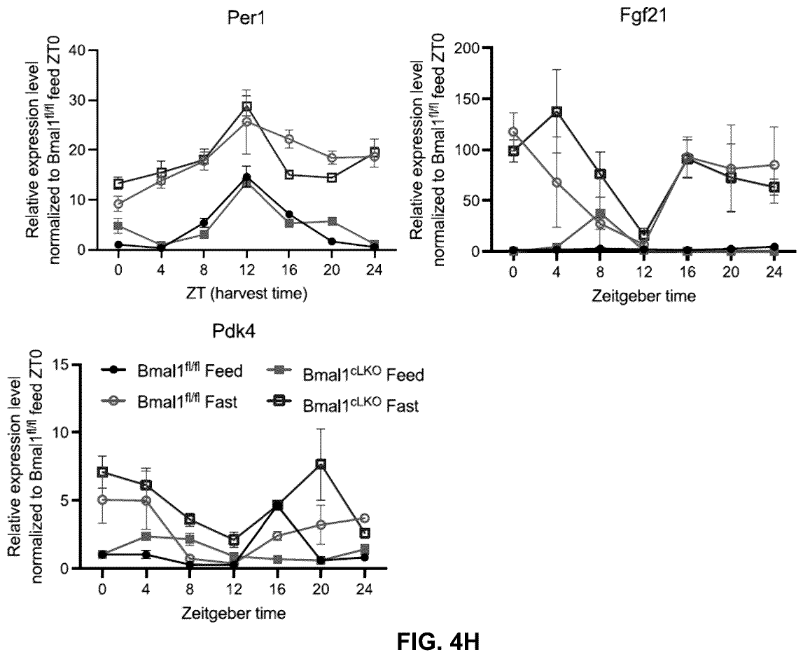

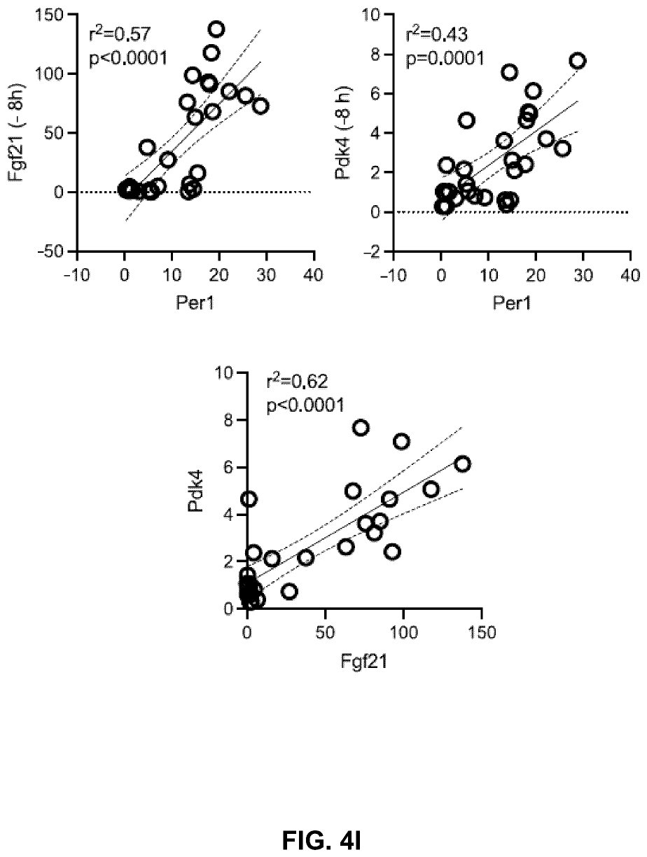

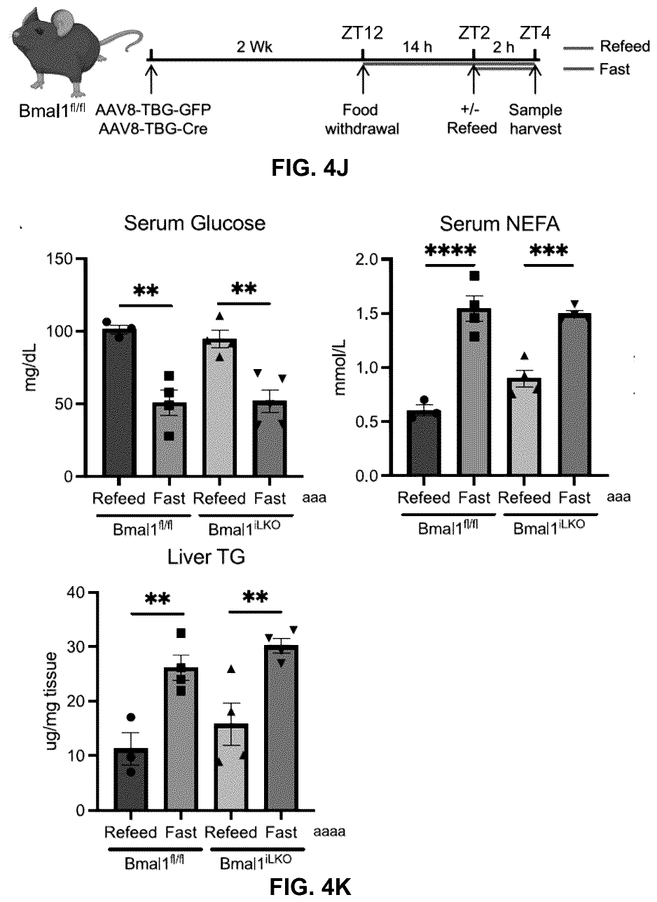

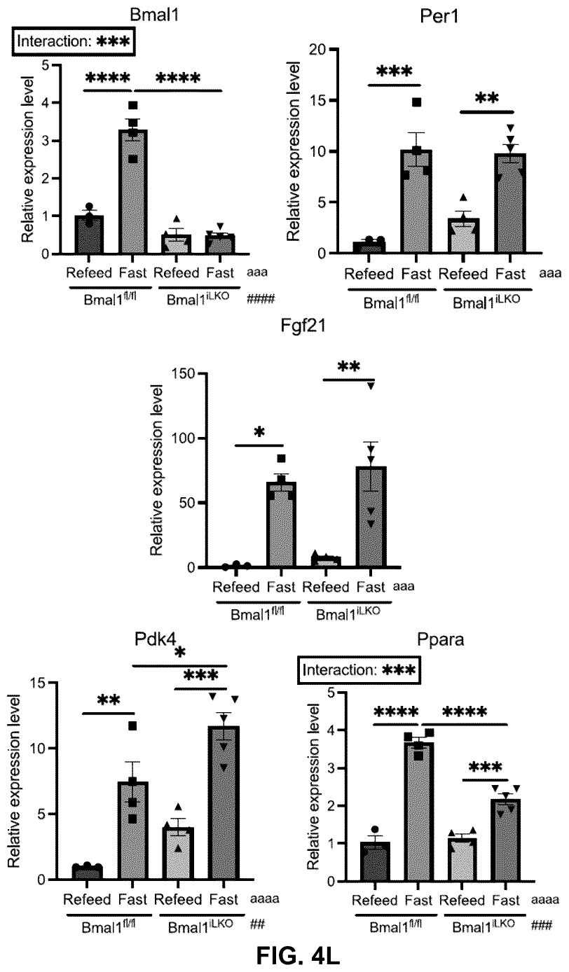

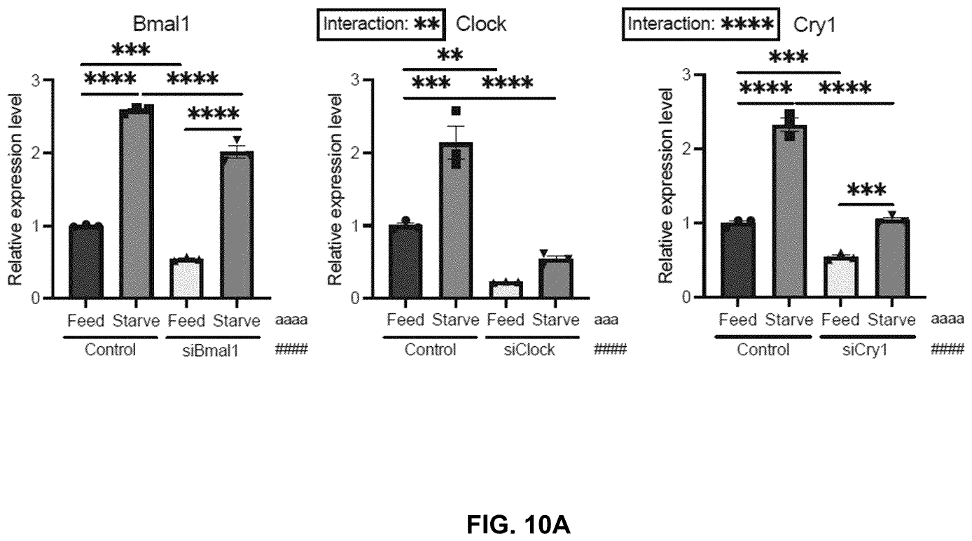

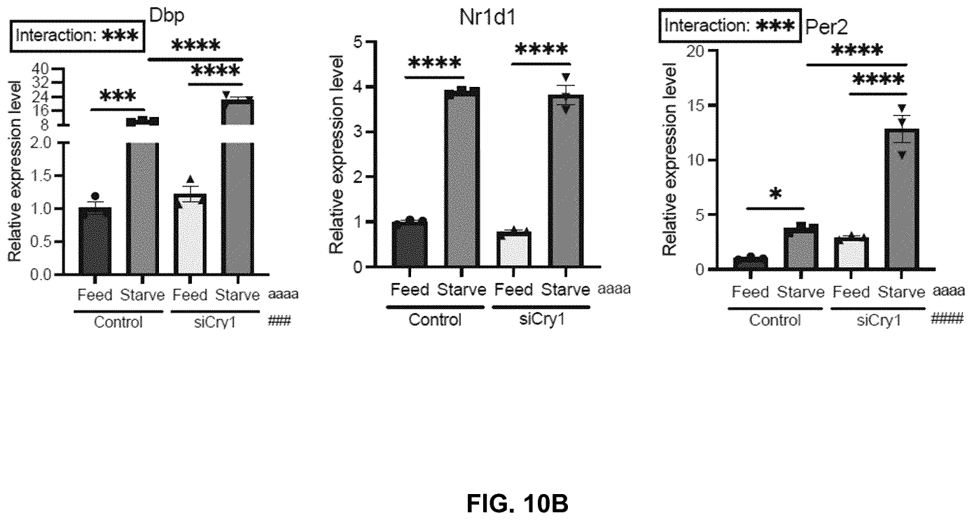

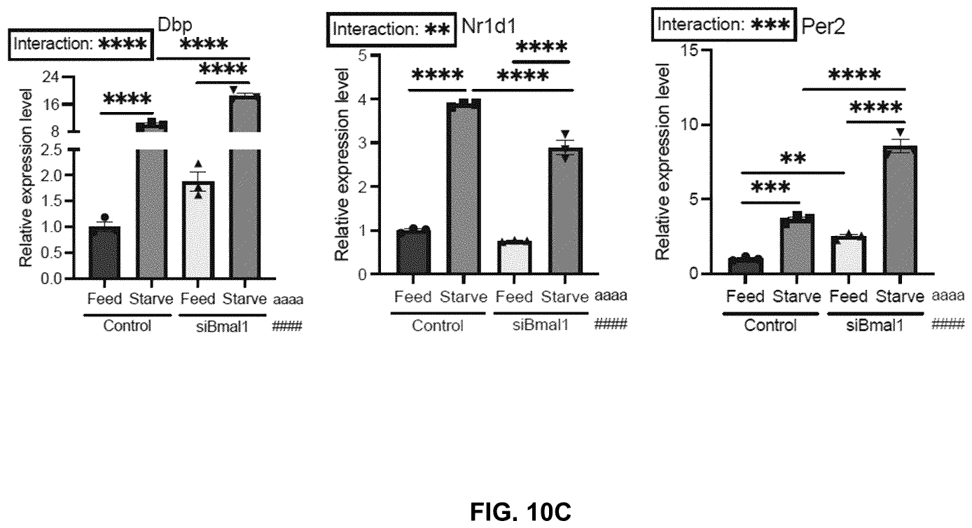

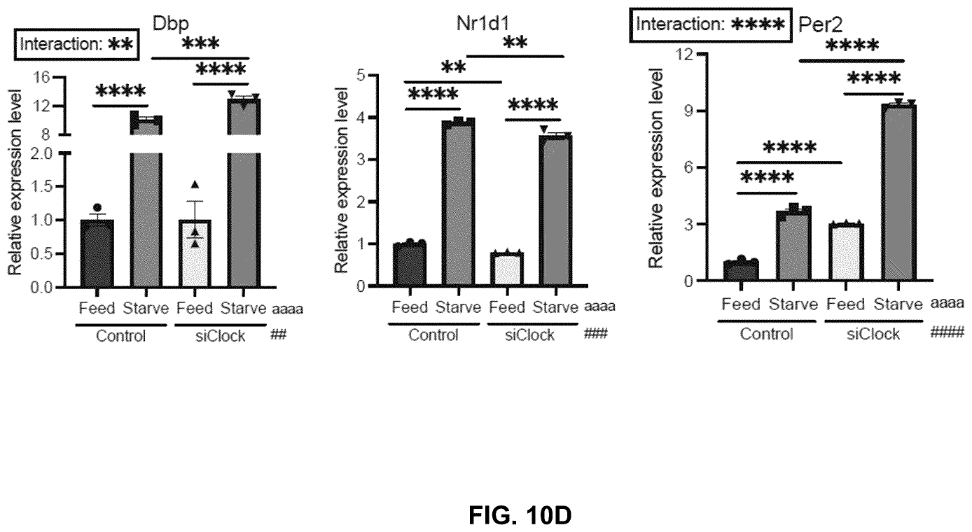

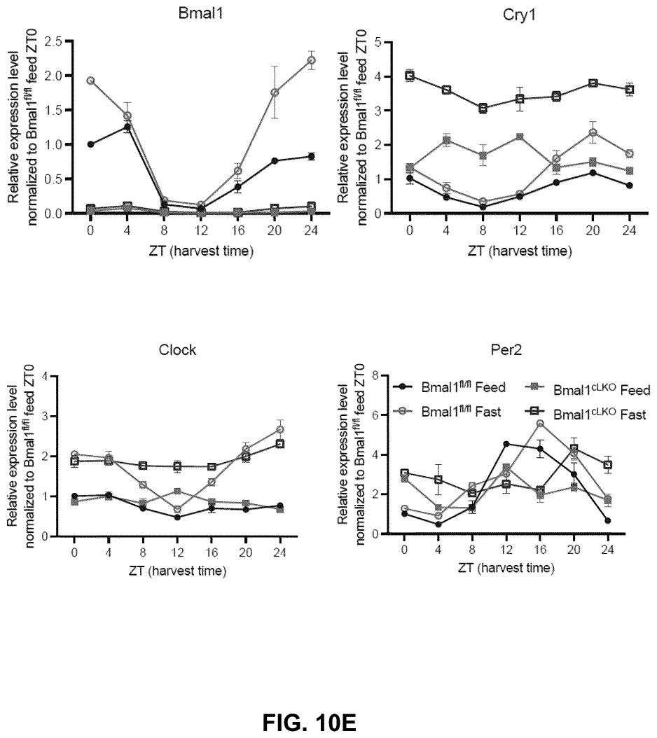

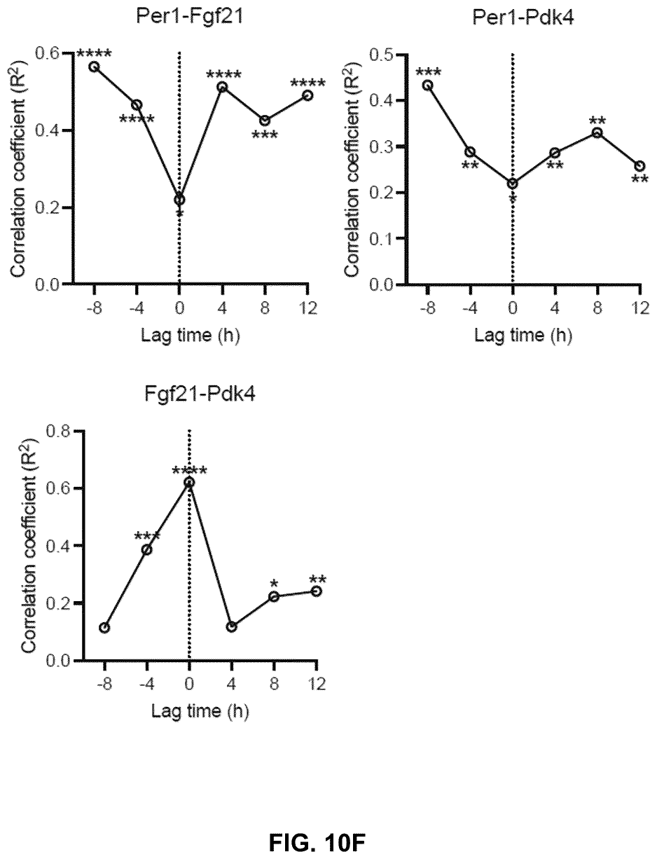

Per1 canonically functions in concert with other core circadian genes, including Per2, Cry1/2, Bmal1, and Clock. This study tested the extent to which Per1 regulatory effects on fasting require circadian regulation. First, Per1 knockdown followed by low-glucose, no-serum medium exposure in AML12 cells revealed that Per1 knockdown reduced Fgf21 and Pdk4 gene induction in response to nutrient withdrawal (FIG. 4A, FIG. 4B). In contrast, real-time qPCR confirmed intact transcriptional activation of Per1, Fgf21, and Pdk4 during starvation in cells deficient for any of the core clock genes-Cry1, Bmal1, and Clock, whereas several other circadian genes were disrupted when knocking down these core circadian genes. (FIG. 4C, FIG. 4D, FIG. 10A, FIG. 10B, FIG. 10C, FIG. 10D). Similarly, ex vivo primary hepatocytes isolated from Per2^{WT} and Per2^{KO} mice were challenged with starvation medium (FIG. 4E), and Per2^{KO} hepatocytes exhibited intact Per1, Fgf21, and Pdk4 induction during starvation when compared with Per2^{WT} hepatocytes (FIG. 4F).

To define circadian dependence of the Per1-Fgf21 pathway in vivo, Per1 activation was examined during fasting throughout the circadian clock in hepatocyte-specific Bmal1-deficient mice (FIG. 4G). Bmal1^{cLKO} (Bmal1^{fl/fl}, Alb-Cre) mice were selected as the model, because Bmal1 is required for diurnal behaviors in mice and rhythmic expression of Per1/2. Food was withdrawn every 4 h or fed mice ad libitum in Bmal1^{fl/fl} or Bmal1^{cLKO} mice (FIG. 4G). Mice were sacrificed 16 h after food withdrawal throughout ZTO-ZT24. Strikingly, Per1 expression maintained its rhythmicity and was upregulated in 16-h-fasted livers, independent of Bmal1 and independent of fasting initiation timing (FIG. 4H). In contrast, Per2 completely lost rhythmicity in the absence of hepatocyte Bmal1 and was again minimally responsive to fasting (FIG. 10E). Expression of other core circadian genes also depended on Bmal1 (FIG. 10E). Moreover, Fgf21 and Pdk4 expression was induced in Bmal1^{fl/fl} mice during fasting at most time points and was activated in Bmal1^{cLKO} mice independent of fasting initiation timing (FIG. 4H). It was noted that Per1-Fgf21-Pdk4 input response remained a significant linear stimulus-response relationship at most time points throughout the time course (FIG. 10F). However, a strong correlation was observed between Per1 and downstream Fgf21-Pdk4 expression 8 h later to yield the strongest correlation coefficient (FIG. 4I). No time lag was required to account for Fgf21 and Pdk4 alignment (FIG. 4I, FIG. 10F). The data indicate a Per1-Fgf21-Pdk4 association during fasting, consistent with the possibility that gene expression and chromatin remodeling constitute a key aspect of the Per1-mediated fasting response. It was also noted that in the standard fast/refeed model, Bmal1^{iLKO} (Bmal1^{fl/fl}, AAV8-TBG-Cre) maintained intact serum glucose, serum non-esterified fatty acid, and intrahepatic liver TG as well as intact Per1, Fgf21, and Pdk4 gene expression during fasting when compared with fed mice (FIG. 4J, FIG. 4K, FIG. 4L). Surprisingly, a significant defect in Ppara expression was identified in fasting mice lacking hepatocyte Bmal1, further suggesting a Ppara-independent activation of Fgf21-Pdk4 in fasting liver (FIG. 4L).

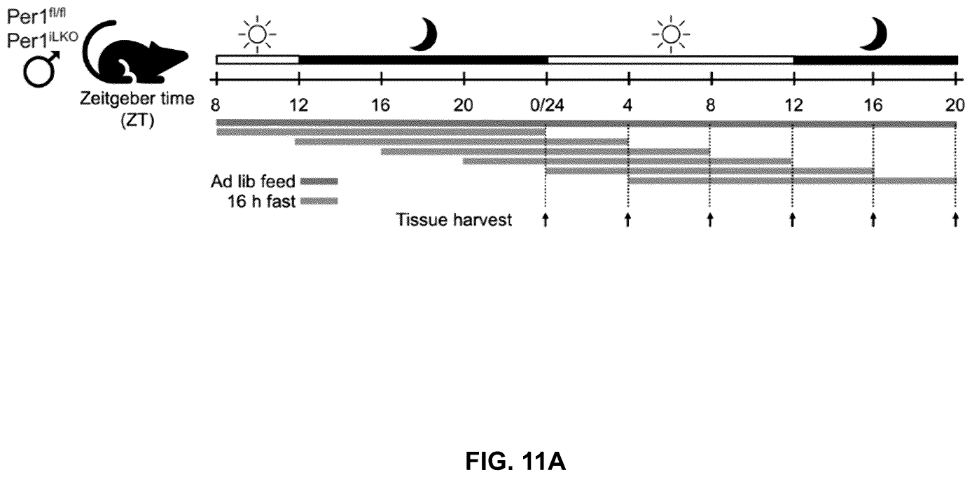

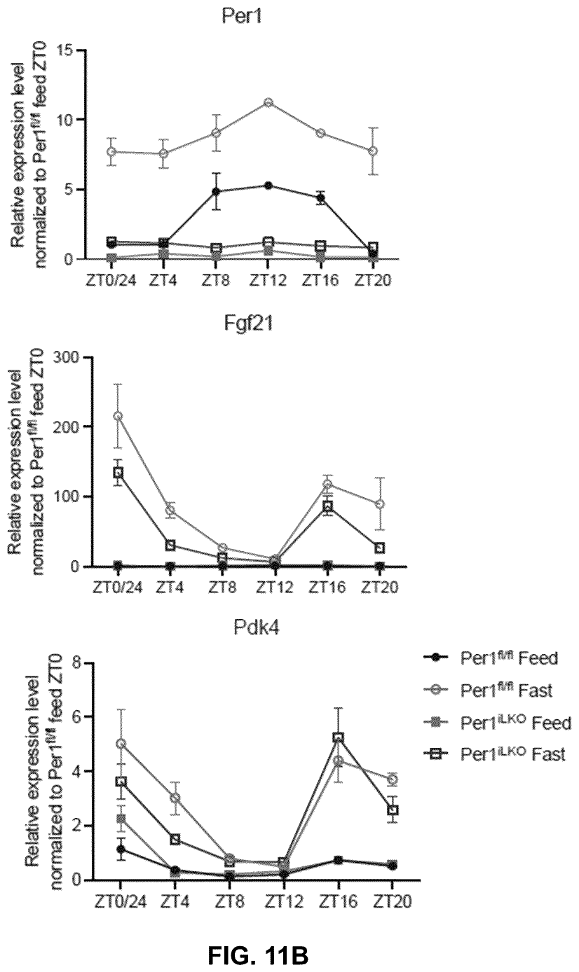

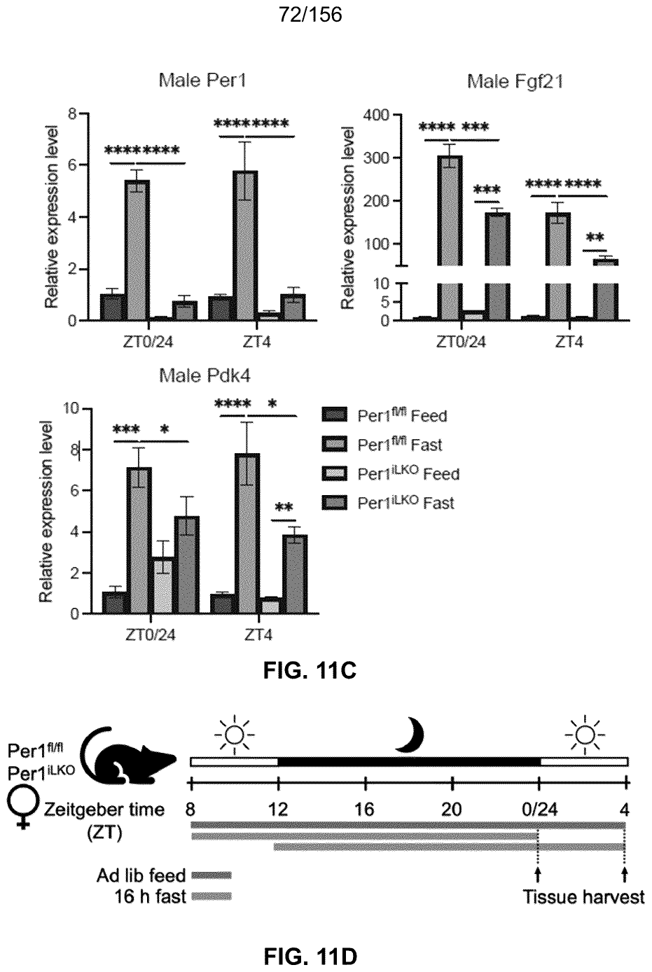

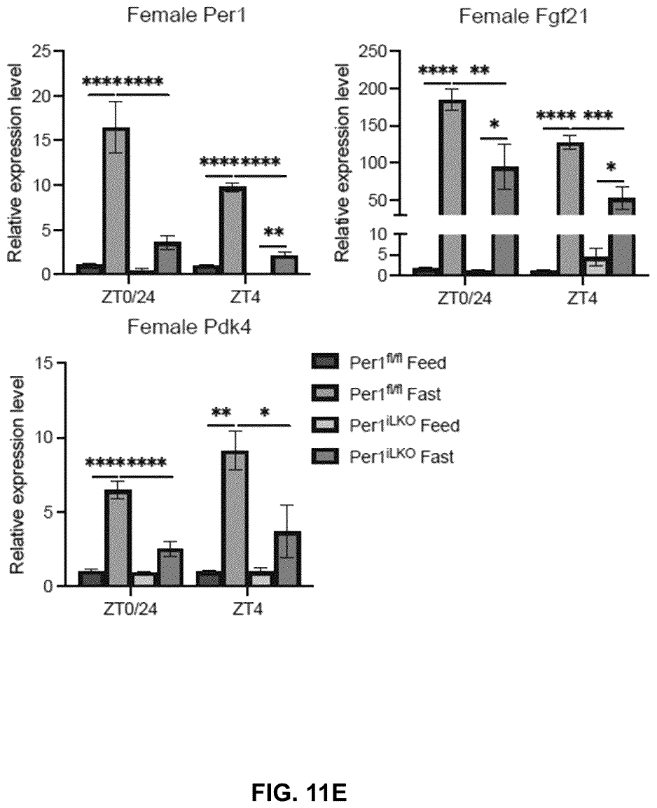

This study next sought to better understand temporal and nutritional interactions in male Per1^{fl/fl} and Per1^{iLKO} mice. Therefore a similar fast/feed time course was performed in Per1^{fl/fl} and Per1^{iLKO} mice (FIG. 11A). This again revealed that Fgf21 and Pdk4 gene expression were activated during fasting in Per1^{fl/fl} mice. Fgf21 and Pdk4 were significantly reduced in Per1^{iLKO} mice subjected to fasting that spans the entire dark phase (FIG. 11B, FIG. 11C). That is, 16-h-fasted Per1^{iLKO} mice analyzed at ZT0/24 and ZT4 exhibited the greatest magnitude of Fgf21-Pdk4 gene expression defect when compared with fasting Per1^{fl/fl} mice (FIG. 1D, FIG. 11B, FIG. 11C). To then test the extent to which Per1 regulates Fgf21 and Pdk4 in female mice, a 16-h fasting experiment was performed in female Per1^{fl/fl} mice treated with AAV8-TBG-GFP or AAV8-TBG-Cre (FIG. 11D). This showed that Per1 was significantly induced by fasting in control mice at both ZT0/24 and ZT4, and Fgf21 and Pdk4 were significantly reduced in fasting Per1^{iLKO} mice at the same time points (FIG. 11E).

### Pyruvate Dehydrogenase Links Per1 to Proper Fuel Selection During Fasting/Feeding

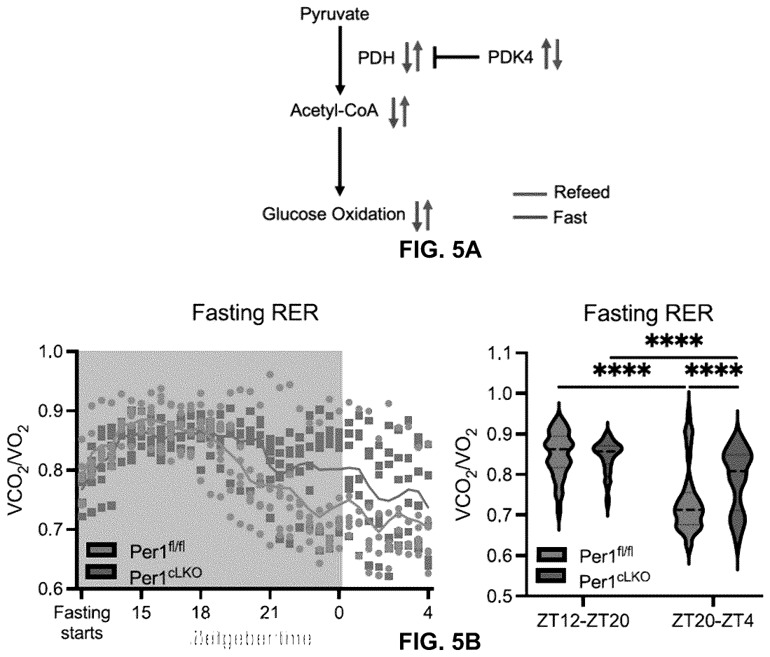

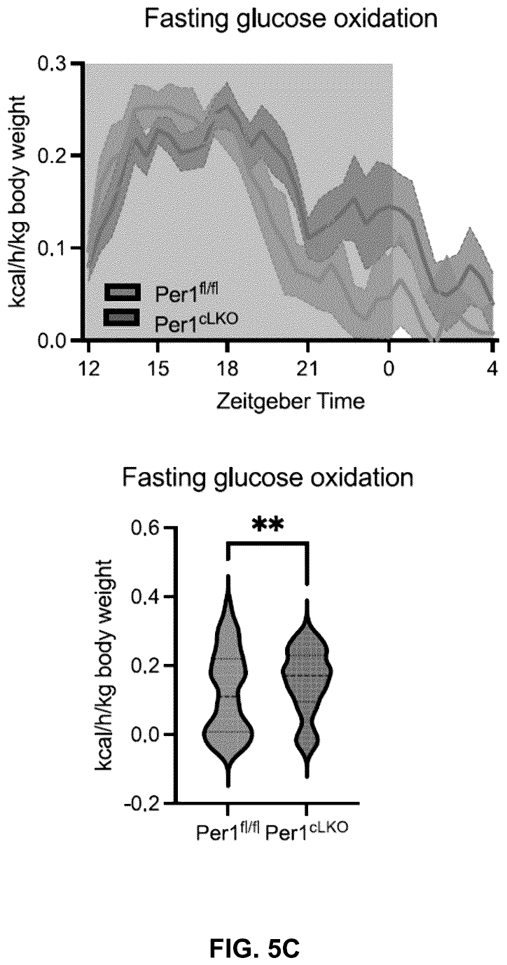

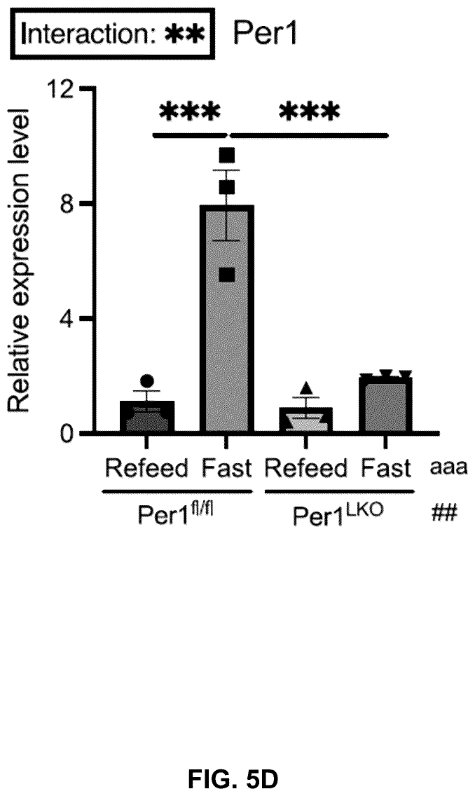

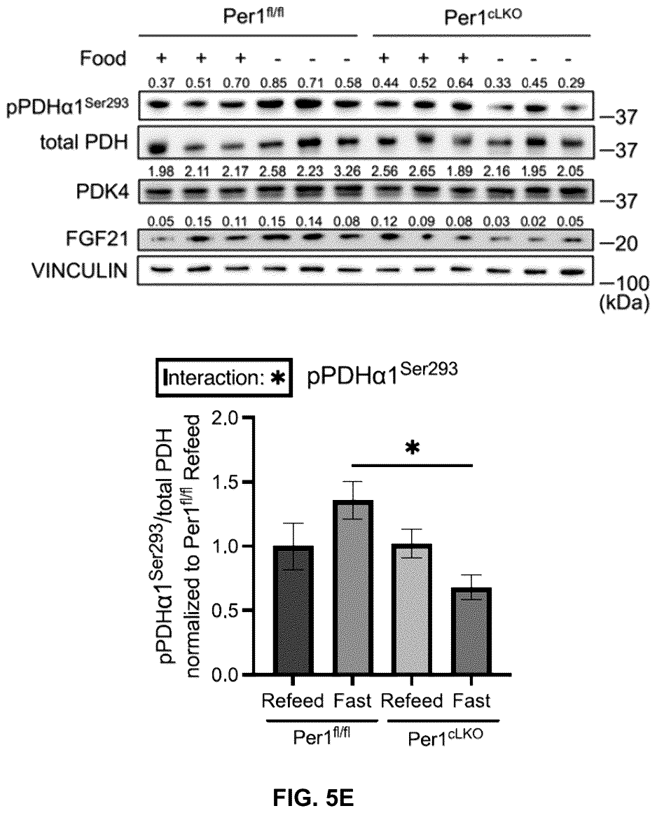

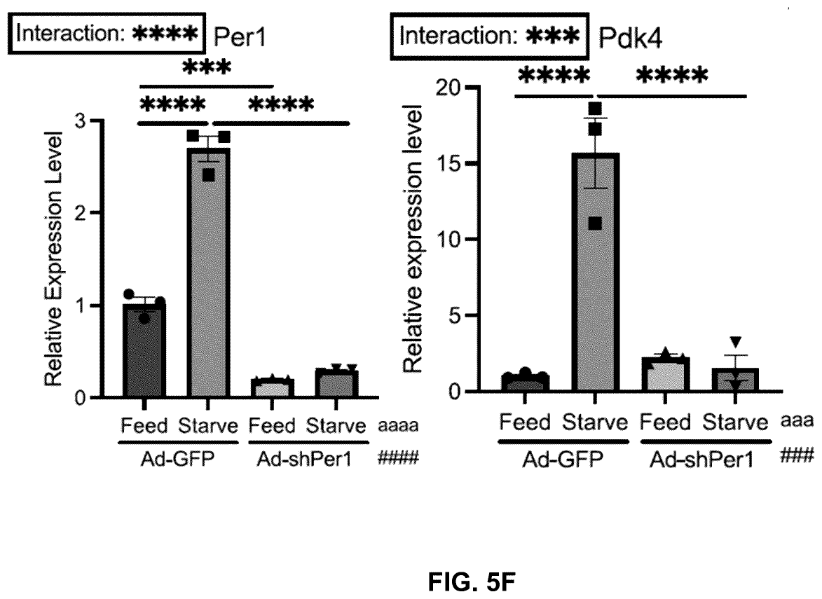

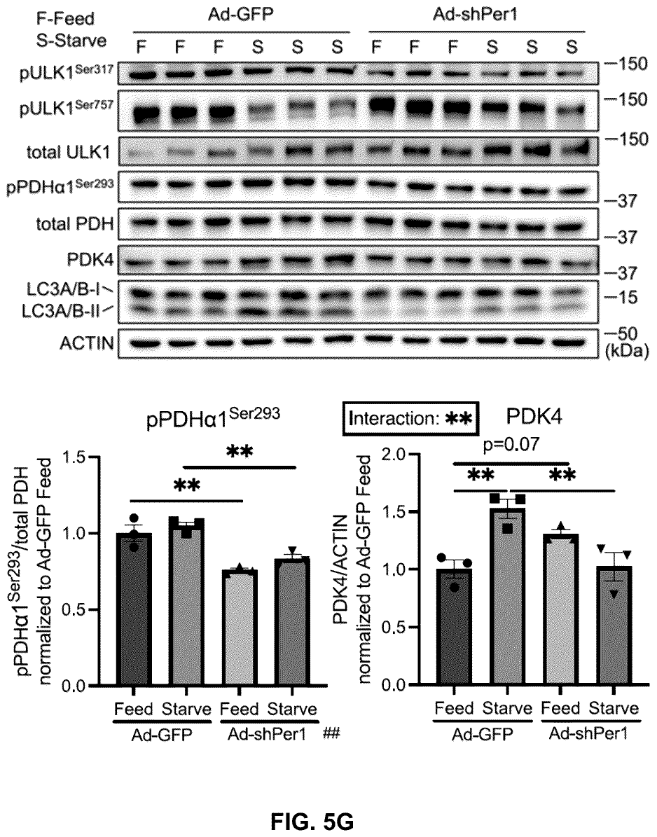

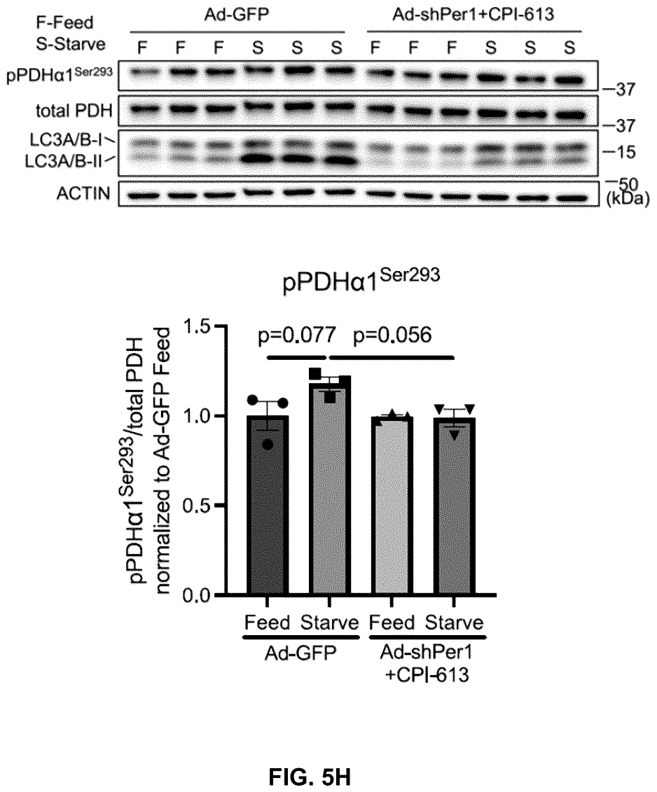

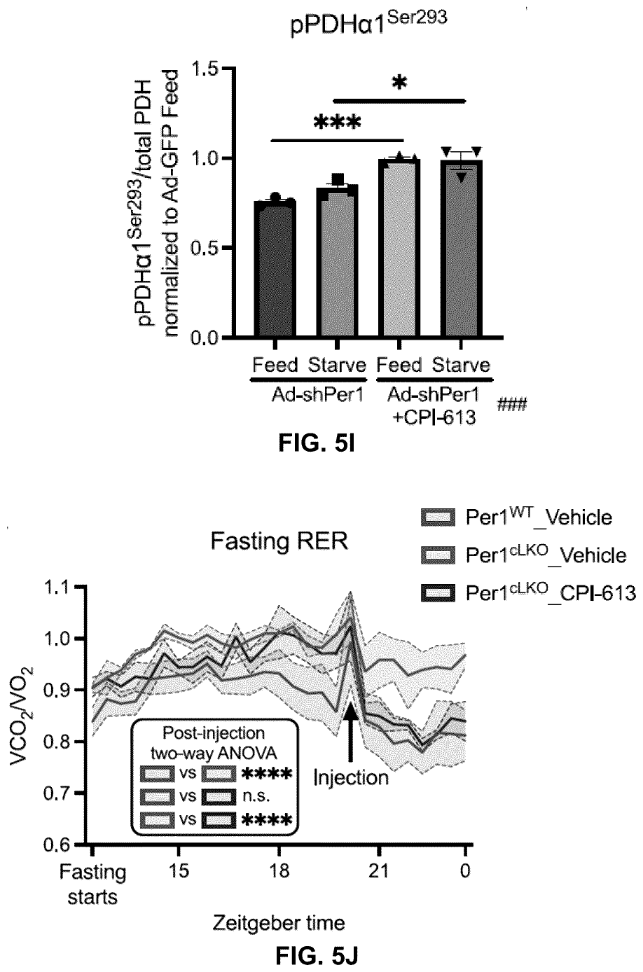

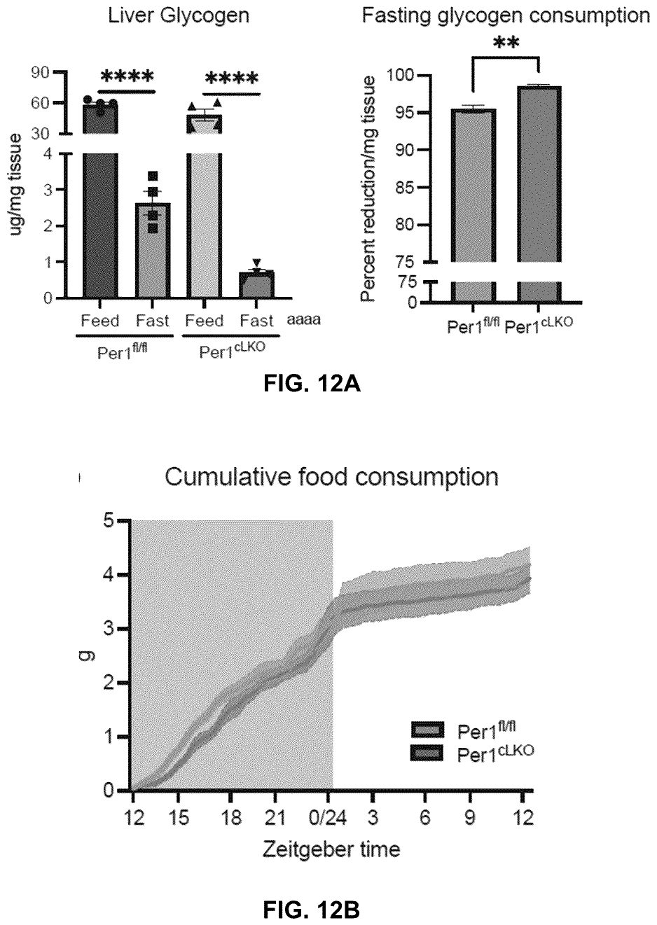

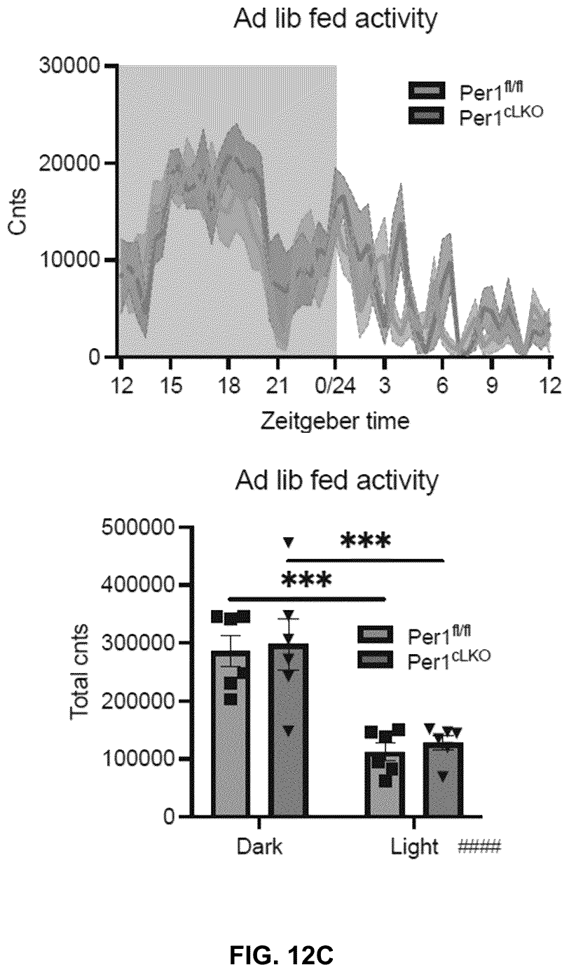

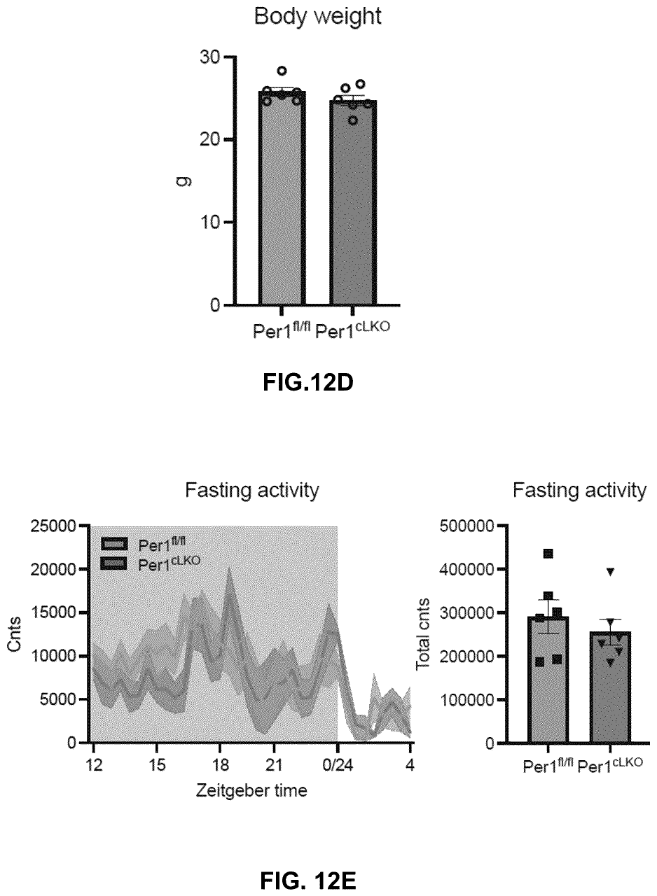

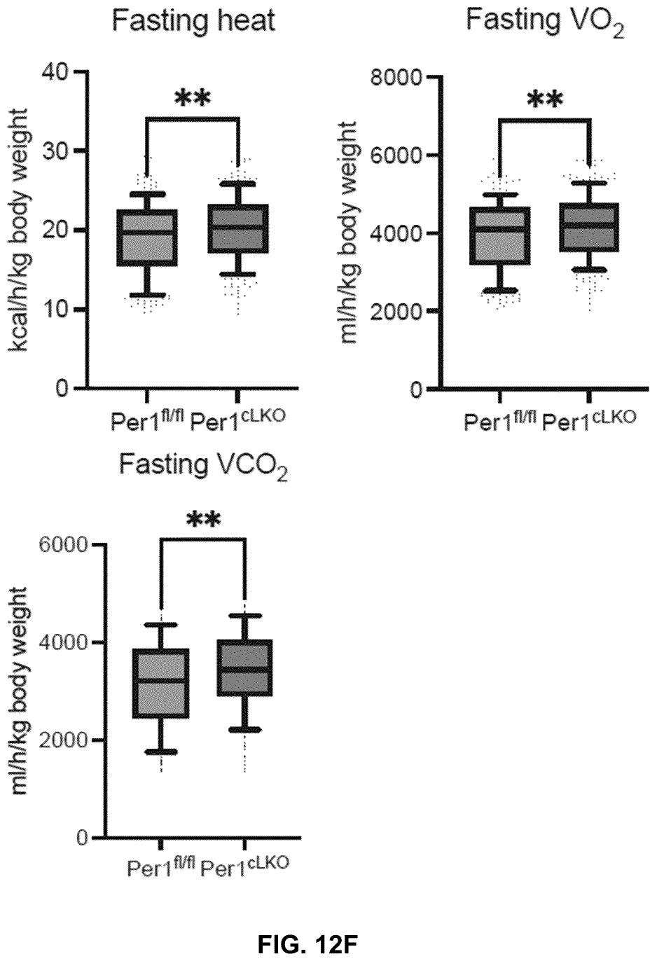

Pdk4 regulates fuel selection from glucose to fatty acid during fasting through phosphorylating and inhibiting PDH activity (FIG. 5A). Defective Pdk4 transcriptional activation and fatty acid oxidation in Perf^{iLKO} fasted liver prompted the hypothesis that hepatocyte Per1-deficient mice exhibit impaired fuel utilization from glucose to fatty acid oxidation during fasting. To test this, substrate selection was quantified in vivo by indirect calorimetry. Per1^{fl/fl} mice appropriately decreased glucose oxidative capacity during fasting, as indicated by a decrease in respiratory exchange ratio (RER). In contrast, Per1^{cLKO} mice exhibited an attenuated RER reduction (FIG. 5B, FIG. 5C) accompanied by a modestly higher hepatic glycogen utilization (FIG. 12A). Similarly, Per1^{cLKO} mice failed to completely suppress heat generation, oxygen uptake (VO2), and carbon dioxide output (VCO2) when compared with Per1^{fl/fl} mice during fasting. However, no genotype-driven differences were observed in food consumption, total activity, total body weight prior to fasting, or total activity during fasting (FIG. 12B, FIG. 12C, FIG. 12D, FIG. 12E, FIG. 12F).

This study then asked whether impaired fuel switching and enhanced glucose oxidation during fasting were due to failed inhibition of PDH. Liver pPDHa1^{Ser293} levels were measured in Per1^{fl/fl} and Per1^{cLKO} mice after 14 h+2 h fast/refeed. Per1^{cLKO} mice exhibited an increase in hepatic pPDHα1^{Ser293} during refeeding and a significant reduction in pPDHα1^{Ser293} during fasting (FIG. 5D, FIG. 5E). Again, the defective activation in FGF21 protein was observed in Per1^{cLKO} fasted liver (FIG. 5E). AML12 cells, in vitro, were subjected to 48 h of starvation and demonstrated impaired autophagic flux and decreased starvation-induced PDH phosphorylation, PDK4 protein, and gene expression (FIG. 5F, FIG. 5G), suggesting that Per1 drives the hepatocyte-intrinsic PDK4-PDH regulation.

Following the observation of a defective pPDHα1^{Ser293} level and abnormal glucose oxidation during fasting, this study tested whether pharmacologically inhibiting PDH activity using CPI-613 (devimistat) is sufficient to reverse dysregulated Per1^{cLKO} fasting glucose metabolism. Previously, CPI-613 treatment was shown to significantly increase the pPDH^{Ser293} level in leukemia K562 cell line. This study also verified that CPI-613 treatment significantly increased the level of pPDH^{Ser293} in shPer1-treated AML12 cells (FIG. 5H, FIG. 5I). In vivo injection of CPI-613 at ZT20 also reversed the inappropriately elevated RER observed in Per1^{LKO} mice during fasting (FIG. 5J). In summary, this study identified impaired substrate flexibility in fasting Per1^{cLKO} mice, and this was associated with reduced Pdk4 expression and lower pPDH^{Ser293} phosphorylation. Pharmacological PDH inhibition is sufficient to reverse the loss of substrate flexibility in Per1^{cLKO} mice.

### Hepatocyte Fgf21 Links Per1 to Pdk4-Mediated Fasting Glucose Oxidation Attenuation

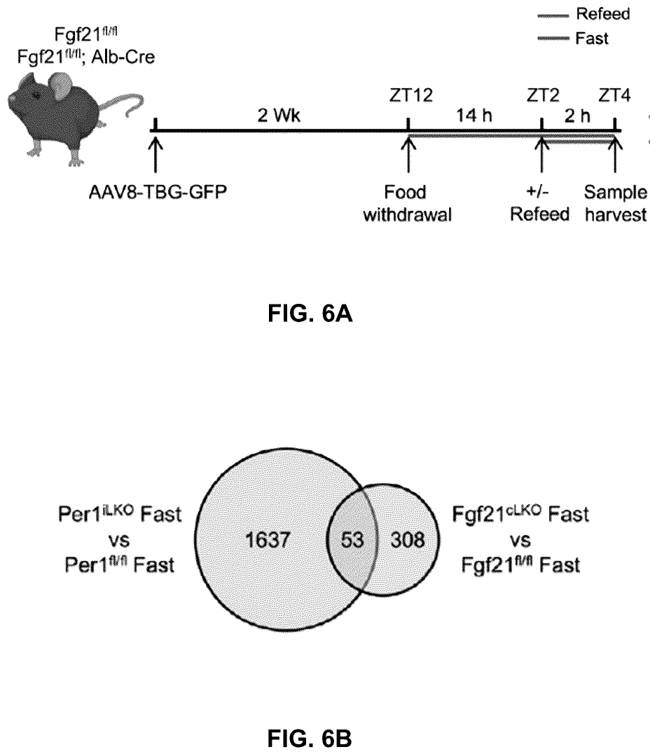

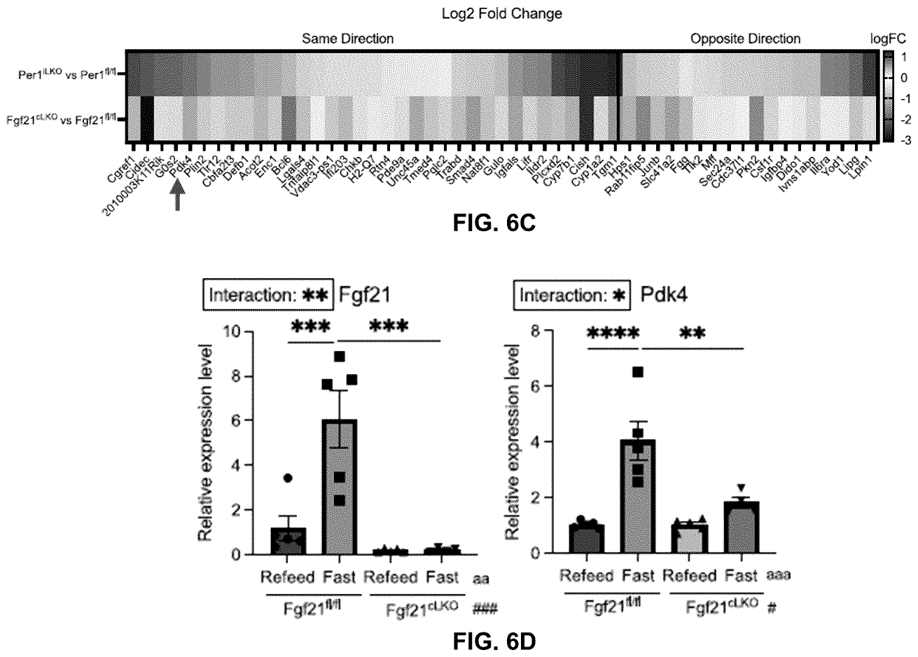

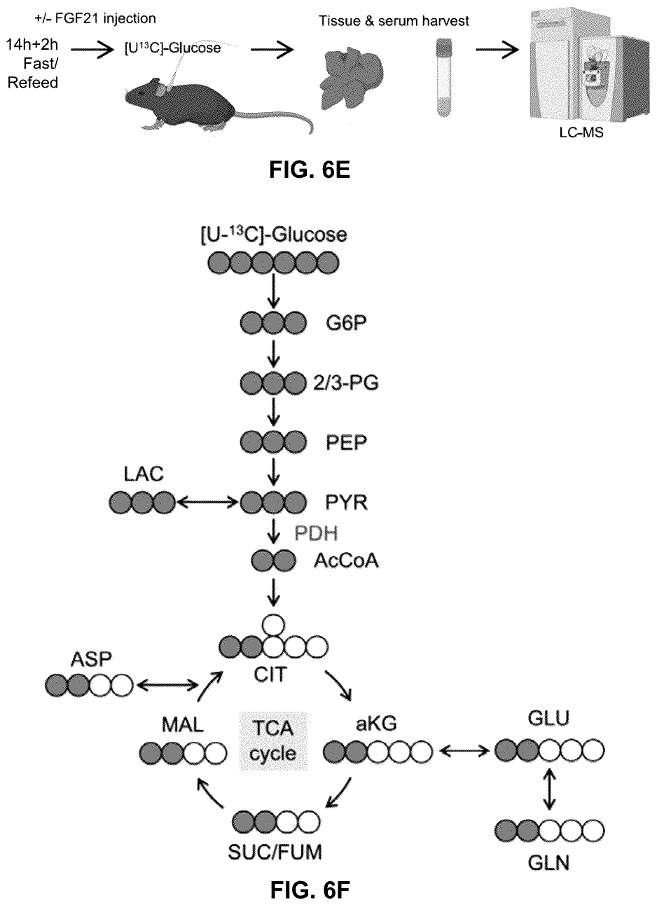

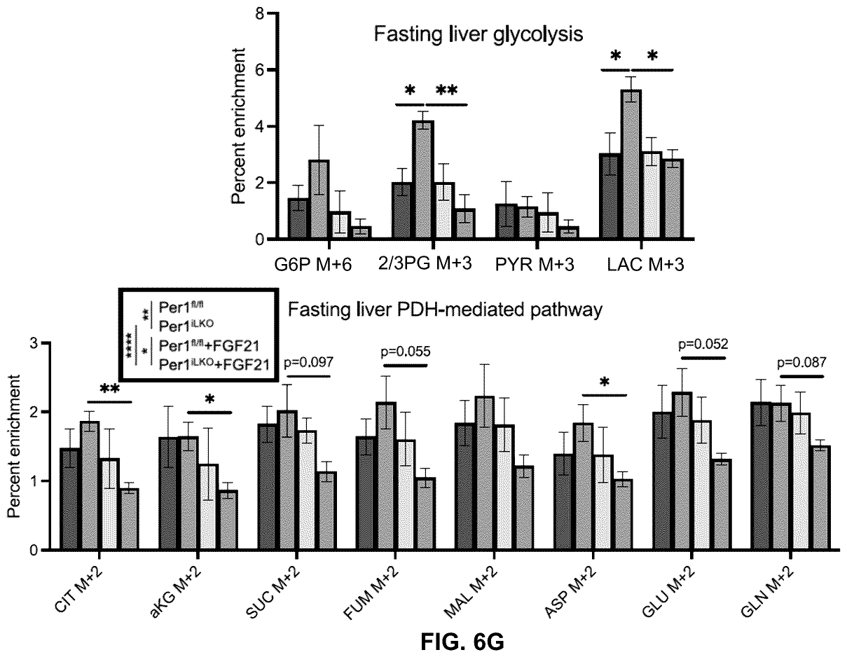

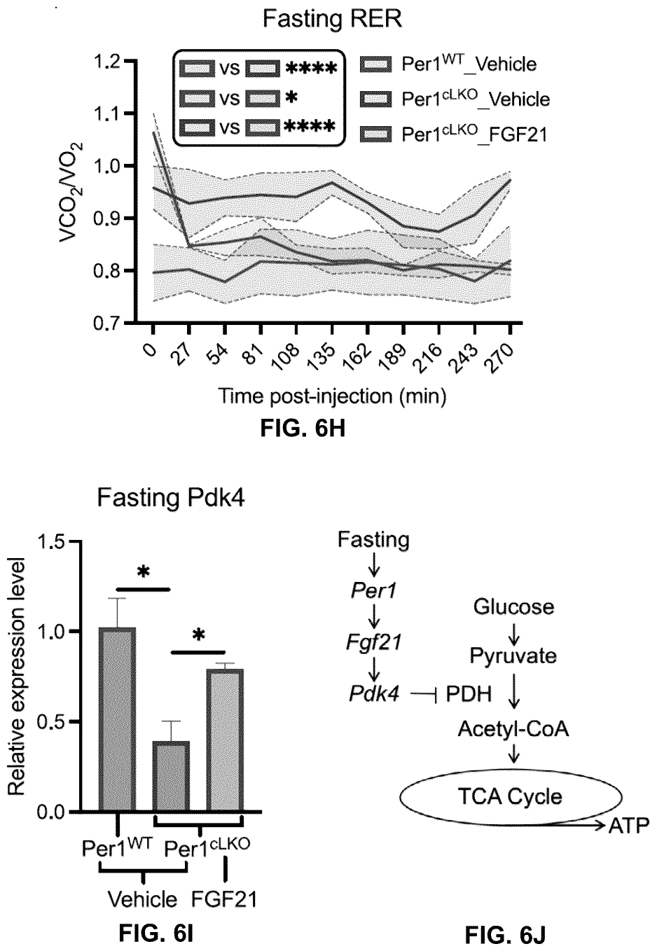

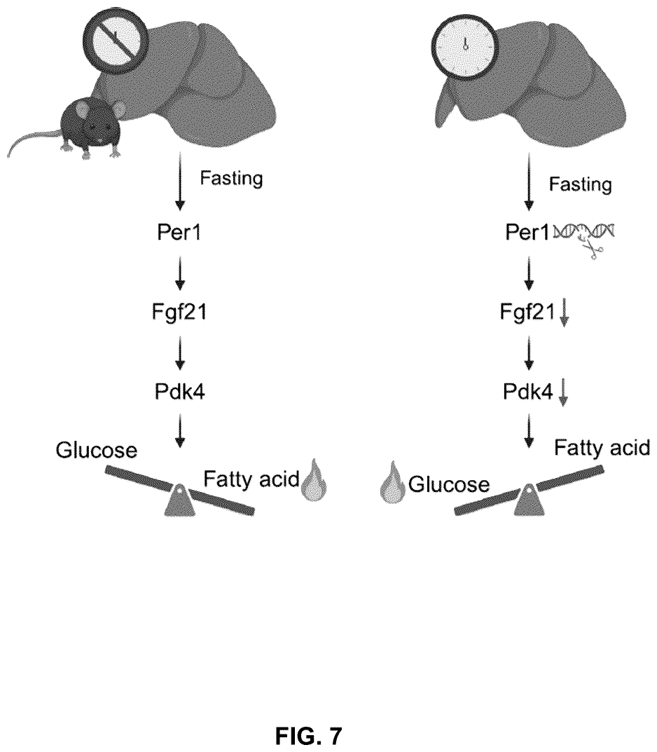

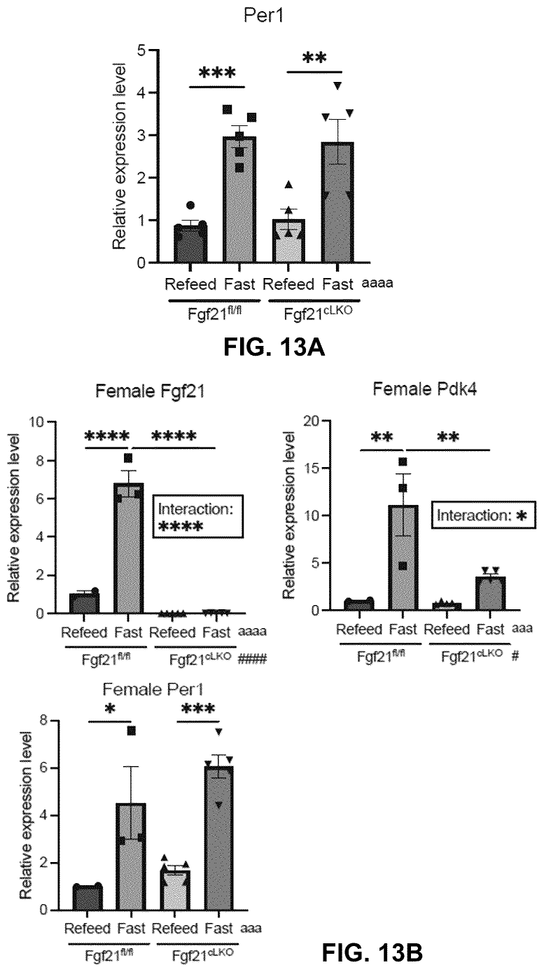

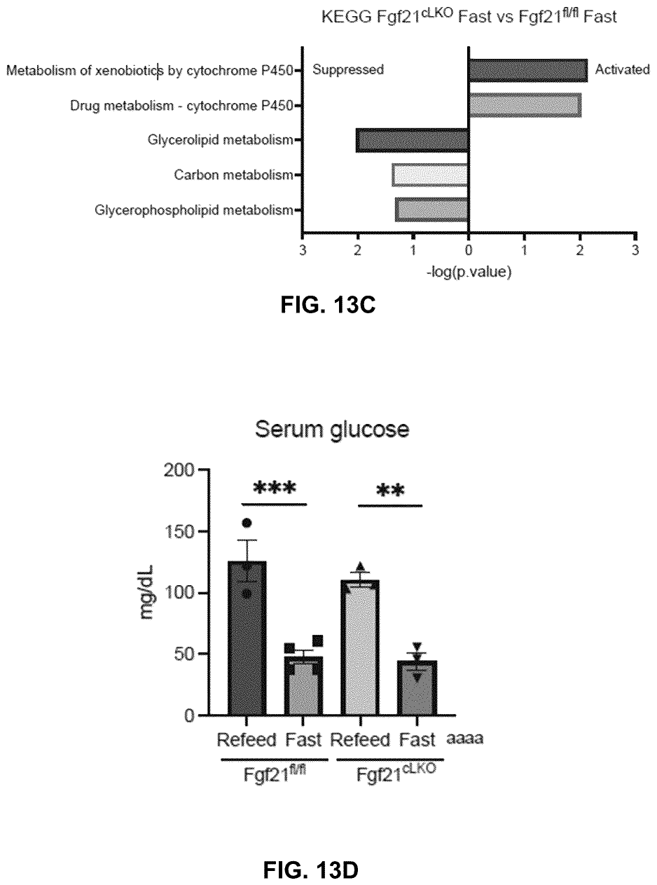

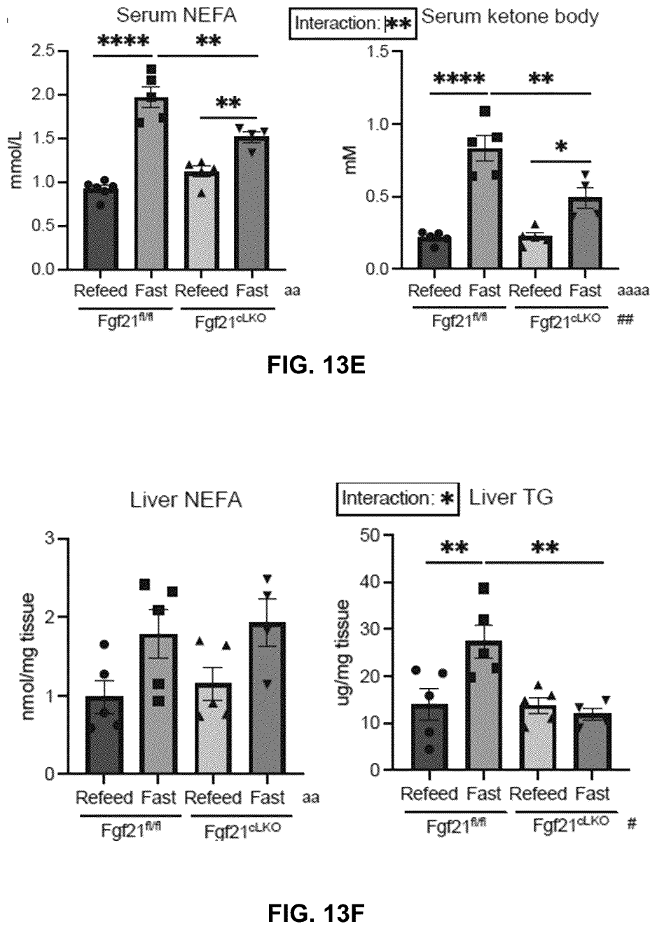

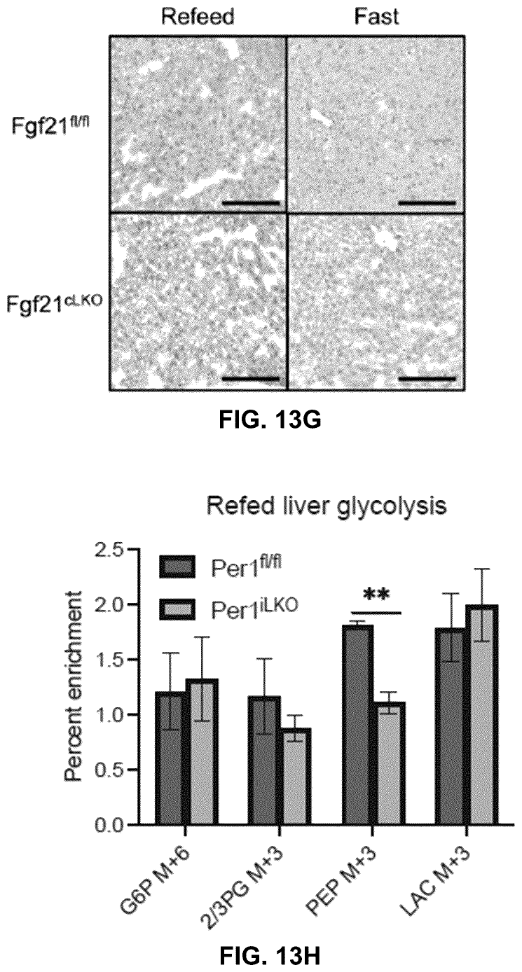

The strong correlation between Fgf21 and Pdk4 expression led us to quantify the degree to which fasting mice lacking hepatocyte-specific Fgf21 also exhibit aspects of impaired fasting signaling, as was observed in Per1^{LKO} mice. This study performed 14 h+2 h fast/refeed in Fgf21^{fl/fl} and Fgf21^{cLKO} (Fgf21^{fl/fl}, Alb-Cre) mice (FIG. 6A). Fasting Fgf21^{cLKO} mice upregulated Per1 to the same degree as in fasting Fgf21^{fl/fl} mice (FIG. 13A). Bulk RNA sequencing revealed 308 DEGs in liver from fasting Fgf21^{cLKO} mice versus Fgf21^{fl/fl} mice. Among them, 53 DEGs were differentially expressed when comparing Per1^{iLKO} fasted and Per1^{fl/fl} fasted mice. Thirty-five of 53 DEGs, including Pdk4, were similarly altered in the same direction in fasting Per1^{iLKO} and fasting Fgf21^{cLKO} versus fasting floxed control mice (FIG. 6B, FIG. 6C). The defect were validated in Pdk4 activation in Fgf21^{cLKO} liver from fasting mice in both males and females (FIG. 6D, FIG. 13B). KEGG pathway analysis also revealed similar fasting-induced transcriptomic pathway changes in Fgf21^{cLKO} and Per1^{iLKO} mice in comparison to those in control mice, including upregulation of cytochrome pathways (FIG. 13C). Moreover, fasting Fgf21^{cLKO} mice exhibited normal serum glucose and liver fatty acid accumulation during fasting, as well as impaired serum fatty acid, ketone body, ORO staining, and intrahepatic TG accumulation when compared with fasting Fgf21^{fl/fl} mice (FIG. 13D, FIG. 13E, FIG. 13F, FIG. 13G).

Finally, the extent to which exogenously administered FGF21 reconstitutes substrate selection observed in fasting Per1^{iLKO} mice was quantified. Per1^{fl/fl} or Perf^{iLKO} mice were treated with or without recombinant FGF21 protein after a 14 h+2 h fast/refeed and subjected them to heavy-isotope metabolic tracing in vivo (FIG. 6E). Although this study observed no significant defects found in total hepatic metabolites involved in glycolysis and PDH-mediated pathway in Per1^{iLKO} mice during refeeding (FIG. 13H, FIG. 13I), [¹³C₆] glucose tracing revealed increased labeling in the glycolytic and PDH-mediated TCA cycle in fasting Perf^{iLKO} mice (FIG. 6F, FIG. 6G). Moreover, treatment with recombinant FGF21 significantly reduced glycolytic and PDH-mediated TCA-cycle flux in Per1^{iLKO} mice but did not drive significant changes in fasting Per1^{fl/fl} mice (FIG. 6F, FIG. 6G).

At the physiological level, this study examined whether recombinant FGF21 administration would complement defective substrate selection in fasting Per1cLKO mice. Mice were treated at ZT21 (9 h post fasting), i.e., at the point of RER divergence observed previously when comparing Per1^{fl/fl} and Per1^{cLKO} fasted mice (FIG. 5B). This study quantified changes in substrate selection by RER throughout 5 h after injection (FIG. 6H). Indeed, FGF21 complementation suppressed fasting RER and reconstituted Pdk4 expression during fasting in Per1^{cLKO} mice (FIG. 6H, FIG. 6I). These data together validate a Per1-Fgf21 axis that mediates Pdk4 induction and fuel selection during fasting (FIG. 6J).

### DISCUSSION

Optimal substrate selection is critical for the growth, adaptation, and long-term survival of a species. A well-designed system accounts for acute substrate flux and yet is also temporally primed to anticipate the organism's feeding/fasting and nocturnal/diurnal behaviors. This study showed that Per1 is a candidate to execute both functions. Per1 is a canonical clock gene, yet it is shown that Per1 is both rhythmically expressed and induced by fasting, and each occurs independently of Bmal1 and several other clock genes. However, Per1 function extends beyond substrate selection, as observed, Per1 also drives autophagic flux and peripheral lipolysis in response to fasting. Finally, this study elucidated an oxidative control mechanism in observing that hepatocyte Fgf21 links Per1 upregulation to transcriptional Pdk4 activation and PDH phosphorylation to modulate TCA-cycle flux. This is supported by in vivo complementation data using both indirect calorimetry and in vivo substrate labeling after reconstituting FGF21 and blocking PDH. This pathway overall couples the carbohydrate-deficient hepatocyte to whole-organism shunting away from glucose metabolism.

Data herein indicate that Per1's control over substrate selection, intriguingly, occurs autonomously and independently of Bmal1 and other core clock genes. Equally importantly, however, circadian and metabolic inputs into Per1 expression are approximately additive (FIG. 4H). To that end, this study has shown that genetic knockdown of other clock genes in multiple in vitro and in vivo model systems—in Per2-, Cry1-, Bmal1—, and Clock-deficient hepatocytes and in Bmal1^{LKO} mice—fails to alter fasting-induced Per1 expression. Second, Bmal1^{LKO} mice are phenotypically normal during fasting, despite the traditional view that Bmal1 mediates Per1 transcription within the circadian context. These data indicate that suprachiasmatic nucleus control may not fully extend to peripheral clocks. Together, this data suggests that hepatocyte Per1 is regulated by circadian input and yet exerts its metabolic function independent of these circadian inputs. This coupling of distinct input response within the same sensing factor (Per1) permits rapid, dynamic, yet finely tuned substrate control that integrates, yet separately accounts for, an organism's circadian and metabolic states. Moreover, because the metabolic Per1 response amplitude is greater than its circadian response amplitude (FIG. 4H), this study postulates that the metabolic state is the dominant Per1 input. This is supported by data showing that exogenous treatment with recombinant FGF21 protein or PDH inhibitor CPI-613 can normalize the transcriptional and physiological phenotype of Per1^{LKO} mice. Addition work is should be performed to further this Per1-Fgf21-Pdk4 axis. This would include subsequent determination as to whether this signaling pathway invokes hepatocyte-intrinsic versus hepatocyte-extrinsic processes—or both—in executing its full physiological sequelae.

This study has also shown that excluding carbohydrate from the hepatocyte is sufficient to induce Per1. Thus, the carbohydrate-specific sensing aspect of this pathway reveals important translational applications. This study identified trehalose analogs 4-TA, 6-TreAz, and IMCTA, each comprising distinct carbon structures and each of which differentially and selectively induces Per1 but not Per2. The data support prior data indicating a structure-activity relationship linking trehalose-like compounds to induction of circadian and fasting-induced genes.

In sum, this study has identified a fundamental control mechanism that integrates temporal and metabolic inputs to dictate whole-organism substrate selection and overall fasting adaptations through Per1. Because intracellular carbohydrate is a key signal to suppress this pathway, this study introduced pharmacological tools that can be used to examine normal hepatocyte circadian interactions with metabolic control.

### Materials and Methods

### Mice

Wild-type C57BL/6J-strain mice (Jackson Laboratory, 000664), Fgf21^{fl/fl} mice (Jackson Laboratory, 022361) and Bmal1^{fl/fl} mice (Jackson Laboratory, 007668) were obtained directly from the Jackson Laboratory. Per1^{fl/fl} mice were generated by Genome Engineering & iPSC Center (GEiC) at Washington University. Upon arrival, mice were equilibrated for a minimum of 7 days in the specific pathogen-free vivarium prior to initiating metabolic measurements. Mice were kept under a 12 h alternating light/dark, temperature-controlled facility throughout the experimentation. All in vivo experimental procedures were performed in strict accordance with Institutional Animal Care and Use Committee (IACUC) guidelines at Washington University School of Medicine. Male mice were used unless specified. All experiments were done when mice are 8-week-old.

### AML12 Cell Line

AML12 cells (CRL-2254) were purchased directly from the American Type Culture Collection (ATCC) and propagated and maintained precisely per manufacturer specification. For in vitro feed/starve experiment, 1*10⁶ cells (per well) were seeded in 6-well plates. After overnight attachment, cells were treated with 10⁸ plaque-forming units (PFU) of Ad-GFP or Ad-shPer1 purchased directly from Vector Biolabs. 48 h post-transduction, culture media was switched to either complete media (Dulbecco's modified Eagle's medium/nutrient mixture F-12 (DMEM/F12; ThermoFisher, 11,320-033) supplemented with 10% fetal bovine serum (Gibco, 26140079), 40 ng/ml dexamethasone (Sigma Aldrich, D4902) and insulin-transferrin-selenium solution (Sigma, 11884)) or starvation media (DMEM low glucose, pyruvate; Gibco, 11885084 supplemented with 40 ng/ml dexamethasone). For autophagic flux quantification in FIG. 1I, media was supplemented with DMSO as vehicle control or 200 nM bafilomycin for 6 h. For in vitro experiment in FIG. 5, cells were transduced with Ad-GFP or Ad-shPer1 for 48 h and followed by 48 h media switch to complete or starvation media. Seahorse XF Cell Mito Stress (Agilent, 103015-100), XF Long Chain Fatty Acid Oxidation Stress (Agilent, 103672-100) and XF Glucose/Pyruvate Oxidation Stress (Agilent, 103673-100) tests were performed according to manufacturer's directions.

For in vitro experiment knocking down Fgf21 or other core circadian genes, cells were transfected with ASO purchased from INOS Pharmaceuticals (Fgf21 ASO, INO-256617), or siRNA purchased directly from Santa Cruz Biotechnology (siBmal1, sc-38166; si-Clock, sc-35075; and siCryl, sc-44835). Transfection was performed according to the RNAiMAX (Invitrogen, 13778150) transfection protocol. 72 h post-transfection, culture media was switched to either complete media or starvation media for 6 h. For in vitro treatment of CPI-613 in AML12 cells, 25 μM CPI-613 (Sigma-Aldrich, SML0404) was added 2 h prior to harvest.

### Primary Hepatocyte Isolation, Culture and Treatment

Primary murine hepatocytes obtained from wildtype, Per2^{KO[20]} mice were isolated and maintained in regular DMEM growth media (Sigma, D5796) containing 10% FBS. For in vitro starvation experiment in isolated primary hepatocytes, DMEM low glucose, pyruvate (Gibco, 11885084) was used.

### Virus Injection

Adeno-associated viruses under TBG promoter overexpressing GFP (AAV8-TBG-GFP) and Cre (AAV8-TBG-Cre) were obtained as ready-to-use viral stock from Vector Biolabs (Malvern, PA, USA). 10¹¹ viral particles were injected via tail vein 10-14 days prior to fast/refeed treatment. Standard rodent chow diet was used throughout the study.

### Serum and Hepatic Lipids, Indirect Calorimetry

For all serum analyses, submandibular blood was collected immediately prior to sacrifice and serum was separated. Triglycerides (ThermoFisher, TR22421), free fatty acids (Wako Diagnostics, 999-34691, 995-34791, 991-34891, 993-35191), glucose (Cayman, 10009582) and ketone body (Cayman, 700190) quantification were performed using commercially available reagents according to manufacturer's directions. Hepatic lipids, histology and indirect calorimetry analyses were done. Glucose oxidation was calculated using formula ((4.585*VCO2)-(3.226*VO2))*4. For in vivo injections of CPI-613 (Sigma-Aldrich, SML0404) and FGF21 (Bio-techne, 8409-FG/CF-MTO), mice were intraperitoneally injected 25 mg/kg and 1 mg/kg body weight correspondingly.

Quantitative Real-Time PCR (qRT-PCR)

Total RNA was prepared by homogenizing snap-frozen livers or cultured cells in Trizol reagent (Invitrogen, 15596026) according to the manufacturer's protocol. cDNA was prepared using Qiagen Quantitect reverse transcriptase kit (Qiagen, 205310). Real-time qRT-PCR was performed with Step-One Plus Real-Time PCR System (Applied Biosystems) using SYBR Green master Mix Reagent (Applied Biosystems) and specific primer pairs. Relative gene expression was calculated by a comparative method using values normalized to the expression of the internal control gene β-Actin. All primers were 10 custom-synthesized oligonucleotides obtained from Integrated DNA Technologies. Primer sequences are included in Table 3.

TABLE 3
Primer sequences

| Target | Species | Forward (5′-3′) | Reverse (5′-3′) |
|---|---|---|---|
| Actin | Mouse | GATTACTGCTCTGGCTCCTAG | GACTCATCGTACTCCTGCTTG |
| | | (SEQ ID NO: 1) | (SEQ ID NO: 2) |
| Atf1 | Mouse | GATTCCCACAAGAGTAACACG | CCTATGCTGTCAGATGAGTCCT |
| | | AC (SEQ ID NO: 3) | (SEQ ID NO: 4) |
| Bmal1 | Mouse | TGACCCTCATGGAAGGTTAGA | GGACATTGCATTGCATGTTGG |
| | | A (SEQ ID NO: 5) | (SEQ ID NO: 6) |
| Clock | Mouse | AGAACTTGGCATTGAAGAGTC | GTCAGACCCAGAATCTTGGCT |
| | | TC (SEQ ID NO: 7) | (SEQ ID NO: 8) |
| Cry1 | Mouse | CACTGGTTCCGAAAGGGACT | CTGAAGCAAAAATCGCCACCT |
| | | C (SEQ ID NO: 9) | (SEQ ID NO: 10) |
| Cyp4a14 | Mouse | TTTAGCCCTACAAGGTACTTG | GCAGCCACTGCCTTCGTAA (SEQ |
| | | GA (SEQ ID NO: 11) | ID NO: 12) |
| Esrrg | Mouse | AAGATCGACACATTGATTCCA | CATGGTTGAACTGTAACTCCCAC |
| | | GC (SEQ ID NO: 13) | (SEQ ID NO: 14) |
| Fgf21 | Mouse | CTGCTGGGGGTCTACCAAG | CTGCGCCTACCACTGTTCC (SEQ |
| | | (SEQ ID NO: 15) | ID NO: 16) |
| G6pc | Mouse | CGACTCGCTATCTCCAAGTGA | GTTGAACCAGTCTCCGACCA |
| | | (SEQ ID NO: 17) | (SEQ ID NO: 18) |
| Hmgcs2 | Mouse | CCGTATGGGCTTCTGTTCAG | AGCTTTGTGCGTTCCATCAG (SEQ |
| | | (SEQ ID NO: 19) | ID NO: 20) |
| Pdk4 | Mouse | AGGGAGGTCGAGCTGTTCTC | GGAGTGTTCACTAAGCGGTCA |
| | | (SEQ ID NO: 21) | (SEQ ID NO: 22) |
| Per1 | Mouse | TCCCCTATTCGCTTCTGTGC | GCAGCAATCGATGGATCTGC |
| | | (SEQ ID NO: 23) | (SEQ ID NO: 24) |
| Per2 | Mouse | GAAAGCTGTCACCACCATAGA | AACTCGCACTTCCTTTTCAGG |
| | | A (SEQ ID NO: 25) | (SEQ ID NO: 26) |
| Pgc1a | Mouse | ACACCGCAATTCTCCCTTGT | CGGCGCTCTTCAATTGCTTT (SEQ |
| | | (SEQ ID NO: 27) | ID NO: 28) |
| Ppara | Mouse | TGGTTCCTGGTGCCGATTTA | ACTAGCATCCCACTTAATTATGTA |
| | | (SEQ ID NO: 29) | TCT (SEQ ID NO: 30) |
| Vnn1 | Mouse | CTTTCCTCGCGGCTGTTTAC | CCTCCAGGTATGGGTAGATCGT |
| | | (SEQ ID NO: 31) | (SEQ ID NO: 32) |

### Immunoblotting

Protein from tissues and cells were harvested and proceeded. Antibodies information is provided in table 4.

TABLE 4
Key Resources

| | | |
|---|---|---|
| REAGENT | SOURCE | IDENTIFIER |

Antibodies

| | | |
|---|---|---|
| b-ACTIN | Cell Signaling Technology | Cat#3700 RRID:AB_2242334 |
| FGF21 | abcam | Cat#171941 RRID:AB_2629460 |
| GFP | Cell Signaling Technology | Cat#2956 RRID:AB_1196615 |
| LC3A/B | Cell Signaling Technology | Cat#12741 RRID:AB_2617131 |
| PDH | Cell Signaling Technology | Cat#2784 RRID:AB_2162928 |
| PDK4 | proteintech | Cat#12949-1-AP RRID:AB_2161499 |
| Pyruvate | Cell Signaling Technology | Cat#2784 RRID:AB_2162928 |
| Dehydrogenase | | |
| phospho- | Cell Signaling Technology | Cat#31866 RRID:AB_2799014 |
| PDHa1(Ser293) | | |
| phospho-ULK1(Ser317) | Cell Signaling Technology | Cat#37762 RRID:AB_2922992 |
| phospho-ULK1(Ser757) | Cell Signaling Technology | Cat#14202 RRID:AB_2665508 |
| ULK1 | Cell Signaling Technology | Cat#8054 RRID:AB_11178668 |
| VINCULIN | Cell Signaling Technology | Cat#13901 RRID:AB_2728768 |
| Anti-mouse IgG, HRP- | Cell Signaling Technology | Cat#7076 RRID:AB_330924 |
| linked | | |
| Anti-rabbit IgG, HRP- | Cell Signaling Technology | Cat#7074 RRID:AB_2099233 |
| linked | | |

Bacterial and virus strains

| | | |
|---|---|---|
| AAV8-TBG-GFP | Vector Biolabs | SKU#Vb1743 |
| AAV8-TBG-Cre | Vector Biolabs | SKU#VB1724 |
| Ad-GFP | Vector Biolabs | Cat#1060 |
| Ad-shPer1 | Vector Biolabs | SKU#shADV-268400 |

Chemicals, peptides, and recombinant proteins

| | | |
|---|---|---|
| U13C-Glucose | Cambridge Isotope | Cat#CLM-1396-10 |
| | Laboratories | |
| FGF21 recombinant | Bio-techne | Cat#8409-FG |
| protein | | |
| 6,8-Bis(benzylthio)- | Sigma-Arch | Cat#SML0404 |
| octanoic acid (CPI-613) | | |
| Commercial assays | | |
| Mouse/Rat Fibroblast | BioVendor R&D | Cat#RD291108200R |
| Growth Factor 21 ELISA | | |
| InfinityTM Triglycerides | Thermo | Cat#TR22421 |
| HR Series NEFA-HR | FUJIFILM | Cat#999-34691, 995-34791, |
| | | 991-34891, 993-35191 |
| Glucose Colorimetric | Cayman | Cat#10009582 |
| Assay Kit | | |
| b-Hydroxybutyrate | Cayman | Cat#700190 |
| (Ketone Body) | | |
| Colorimetric Assay Kit | | |
| Seahorse XF Cell Mito | Agilent Technologies | Cat#103015-100 |
| Stress Kit | | |
| Seahorse XF Long | Agilent Technologies | Cat#103672-100 |
| Chain Fatty Acid | | |
| Oxidation Stress Kit | | |
| Seahorse XF | Agilent Technologies | Cat#103673-100 |
| Glucose/Pyruvate | | |
| Oxidation Stress Kit | | |

Experimental models: Cell lines

| | | |
|---|---|---|
| AML12 | ATCC | Cat#CRL-2254 |

Oligonucleotide

| | | |
|---|---|---|
| siBmal1 | Santa Cruz Biotechnology | Cat#sc-38166 |
| siClock | Santa Cruz Biotechnology | Cat#sc-35075 |
| siCry1 | Santa Cruz Biotechnology | Cat#sc-44835 |
| siPpara | Santa Cruz Biotechnology | Cat#sc-36380 |
| siEsrrg | Santa Cruz Biotechnology | Cat#sc-44705 |
| siAtf1 | Santa Cruz Biotechnology | Cat#sc-29755 |
| Fgf21 antisense | IONIS Pharmaceuticals | Cat#ION-256,617 |
| oligonucleotide (ASO) | | |

### In Vivo Tracing and Metabolomic Analysis

All mouse studies were approved by the Institutional Care and Use Committee at Washington University in Saint Louis. To perform infusion studies, a catheter (Instech, C20PU-MJV1301) was placed in the right jugular vein and connected to a vascular access button (Instech, VABM1B/25) implanted subcutaneously in the back of the mice. All surgeries were performed at the Hope Center for Neurological Diseases, Washington University. Mice were allowed to recover from surgery for at least one week before tracer infusion.

U¹³C-Glucose (CIL, CLM-1396-PK) was freshly prepared in saline at a concentration of 200 mM. The mice were weighed to calculate the tracer infusion rate. To begin infusion, the vascular access button of individual mice was connected to the infusion line with a swivel (Instech, SMCLA), tether (Instech, KVABM1T/25), and infusion pump (CHEMYX, Fusion 100T). The infusion line was prefilled with 200 mM U13C-Glucose. Prime infusion was initiated at 1 μL/min/g for 2 min to clear the catheter locking solution, followed by continued infusion at 0.1 μL/min/g for 2 h. Following completion of the glucose infusion, mice were anesthetized, and blood was collected by cardiac puncture. Tissues were subsequently collected as quickly as possible (in 10 min or less) following euthanasia and snap-frozen in liquid nitrogen. Tissues were stored at −80° C. until processing for LC/MS analysis.

The liver tissue was mixed with ice-cold methanol:acetonitrile:water (2:2:1), and subjected to two cycles of 7 m/s (30 s/cycle) using an Omni Bead Ruptor Elute Homogenizer. For every 1 mg of tissue wet weight, 40 μL of extraction solvent was added. Samples were then incubated at −20° C. for 1 h to precipitate protein. Tissue extracts were centrifuged at 20,000 g and 4° C. for 10 min, and the supernatant was transferred into LC/MS vials.

Ultra-high-performance LC (UHPLC)/MS was performed with a Thermo Scientific Vanquish Horizon UHPLC system interfaced with a Thermo Scientific QExactive Plus Mass Spectrometer. Polar metabolites were separated on a HILICON iHILIC-(P)-Classic column (100×2.1 mm, 5 μm). The mobile-phase solvents were composed of: A=20 mM ammonium bicarbonate, 2.5 μM medronic acid, 0.1% ammonium hydroxide in water:acetonitrile 95:5; and B=water:acetonitrile 5:95. The column compartment was maintained at 40° C. The following linear gradient was applied at a flow rate of 0.25 mL min-1:0-1 min, 90% B; 12 min, 35% B; 12.5-14.5 min, 25% B; 15 min, 90% B followed by a re-equilibration phase of 10 column volumes. The injection volume was 4 μL for all polar experiments. Data was acquired in positive and negative ion mode with the following settings: spray voltage, 3.5 kV (positive) and −2.8 kV (negative); sheath gas, 45; auxiliary gas, 10; sweep gas, 2; capillary temperature, 250° C.; aux gas temperature, 300° C.; mass range, 65-975 Da; resolution, 140,000. LC/MS data were processed and analyzed with the open-source Skyline software. Natural-abundance correction of 13C was performed with AccuCor. Data can be found in Metabolomics Workbench Project PR002144.

RNA-Seq and snMultiome-Seq

RNA sequencing was performed by the Washington University Genome Technology Access Center (GTAC). Differentially expressed genes from the heatmap were analyzed by edgeR package, and the heatmap was generated by pheatmap package. Liver nuclei for snMultiome-seq were isolated using chromium nuclei isolation kit with RNase inhibitor (10× genomics, 1000494). Isolated nuclei were processed and sequenced by Washington University GTAC, and data were analyzed by Seurat and Signac package.

### Statistical Analysis

Data are presented as the mean±SEM. The number of independent biological samples (n) in each experiment is detailed in the figure description. The data were analyzed by unpaired two-sided Student's t test, one-way ANOVA or two-way ANOVA with GraphPad Prism 9 softwares. For all the analyses, */a/#p<0.05, **/aa/##p<0.01, ***/aaa/###p<0.001, ****/aaaa/####p<0.0001.

### Example 2: Hepatocyte Period 1 Shapes the Phosphoproteome to Link De Novo Lipogenesis to the Metabolic Clock

Hepatocyte Period 1 (Per1) integrates temporal and metabolic inputs to optimize substrate flux. However, both inputs are perturbed in context of obesity and insulin resistance. Here, this study has shown that inducible deletion of the hepatocyte clock gene Per1 in mice (Per1^{iLKO}) exacerbated diet-induced obesity, glucose intolerance, hepatic steatosis and de novo lipogenesis. Hepatocyte Per1 expression increased energy expenditure and reversed these metabolic derangements, largely independent of Cry1/2 binding and hepatocyte Bmal1 expression. Immunoprecipitation and proteomic analyses identified the phosphatase, PP2A, as a Per1 binding effector, and indeed, PP2A blockade in vivo reversed the enhanced de novo lipogenic gene expression, glucose intolerance, and hepatic steatosis in obese Per1^{iLKO} mice. Untargeted phosphoproteomics analyses unveiled profound disruption in the hepatic phosphoproteome and signalome network in Per1^{iLKO} mice, along with augmented carbohydrate response element (ChREBP) nuclear translocation and target gene expression, which were again reversed via PP2A blockade. Per1 restructures the hepatocyte phosphoproteome via PP2A blockade to link de novo lipogenesis to the metabolic clock.

### Introduction

Circadian and macronutrient inputs, among other signals, dictate substrate utilization and carbon fate. The liver plays a central role in dictating systemic substrate flux, owed partly to its anatomic position linking enterohepatic and venous systemic circulations. It is thus among the first to encounter and herald the complement of macronutrients vying for systemic access. Augmenting the liver's influence on systemic fuel utilization is the hepatocyte's intrinsic oscillatory circadian clock. Metabolic control is thus both intrinsically rhythmic and acutely modified by the presence or absence of exogenous macronutrients.

Prior data indicated that global rhythmic regulation of hepatocyte protein phosphorylation is a central metabolic control modality. However, the mechanisms governing control over the hepatocyte phosphoproteome are incompletely explored. In rodent liver, the vast majority (~90%) of total phosphatase activity is attributable to the promiscuous serine/threonine phosphatase, PP2A, which thereby broadly shapes carbon flux, cell signaling, and proliferation. One example of this includes recent data demonstrating that an IRF3-PP2A axis profoundly regulates body weight, adiposity, and glycemic control in mice and humans with obesity and metabolic dysfunction-associated steatotic liver disease (MASLD). The data underscore the importance of hepatocyte PP2A in hepatic and peripheral metabolic regulation.

Recently hepatocyte Period 1 (Per1) actions have been differentiated from other canonical circadian 41 genes. This study showed that hepatocyte Per1 independently and additively responds to distinct temporal and macronutrient inputs. This established a model whereby hepatocyte Per1 fine-tunes metabolic responses to optimize substrate selection in response to fasting, refeeding and zeitgeber time (ZT). Taken together, i) newly described functions for hepatocyte Per1 as a hybrid ‘metabolic clock’, ii) the existence of rhythmic global phosphorylation as a central hepatocyte metabolic control mechanism, and iii) the reliance of PP2A as a pivotal phosphatase that governs global hepatocyte phosphorylation status, prompted us to examine the hypothesis that hepatocyte Per1 optimizes hepatic metabolism through hierarchical control of the hepatocyte phosphoproteome. This hypothesis was evaluated in context of obesity and insulin resistance to understand the degree to which this hypothesis is translationally applicable.

Shown herein is data that hepatocyte-specific Per1 deletion predisposed mice to Western Diet-induced body weight gain, glucose intolerance, dyslipidemia, de novo lipogenic gene expression, and hepatic fat accumulation. Structure-function analysis revealed that hepatocyte-specific Per1 overexpression increased energy expenditure, blocked diet-induced glucose intolerance, hepatic fat accumulation and de novo lipogenic gene expression, independent of its CRY1/2 binding domain (CBD), and independent of hepatocyte Bmal1. Untargeted proteomic analysis identified PP2A as a direct PER1-binding effector, and subsequent PP2A blockade in Perf^{iLKO} mice reversed their predisposition to impaired energy utilization and exacerbated metabolic disease. Moreover, livers from obese Per1^{iLKO} mice exhibited a disrupted phosphoproteome and phosphor-signalome signature that was partly reverted by PP2A blockade. Specific sequelae of a deranged phosphoproteome included enhanced nuclear ChREBP translocation to the hepatic nucleus in Per1^{iLKO} mice, which was again reversible by PP2A blockade. This study concluded that PP2A executes Per1 control over the hepatocyte phosphoproteome to drive manifold metabolic processes, including ChREBP regulation of de novo lipogenesis in context of obesity. These findings nominate Per1—a canonical circadian gene—as an integrative sensor that links de novo lipogenesis to the metabolic clock.

### Results

### Hepatocyte-Specific Per1 Deletion Exacerbates Diet-Induced Glucose Intolerance, Lipid Accumulation and De Novo Lipogenic Gene Expression

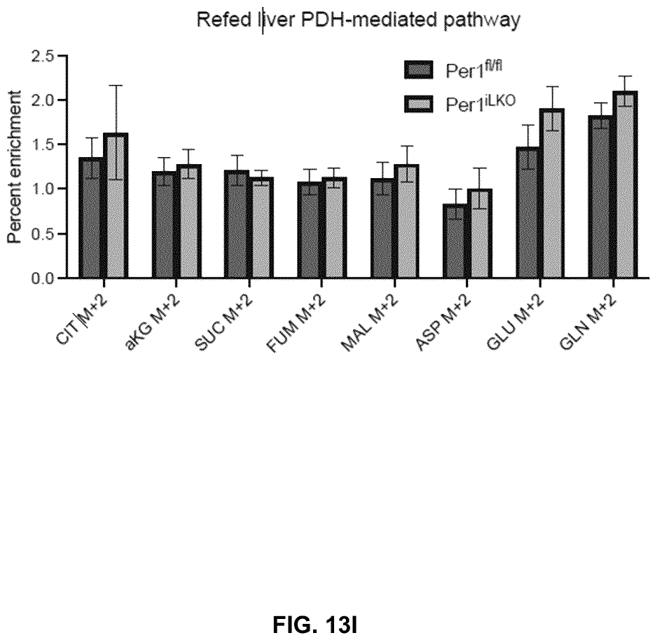

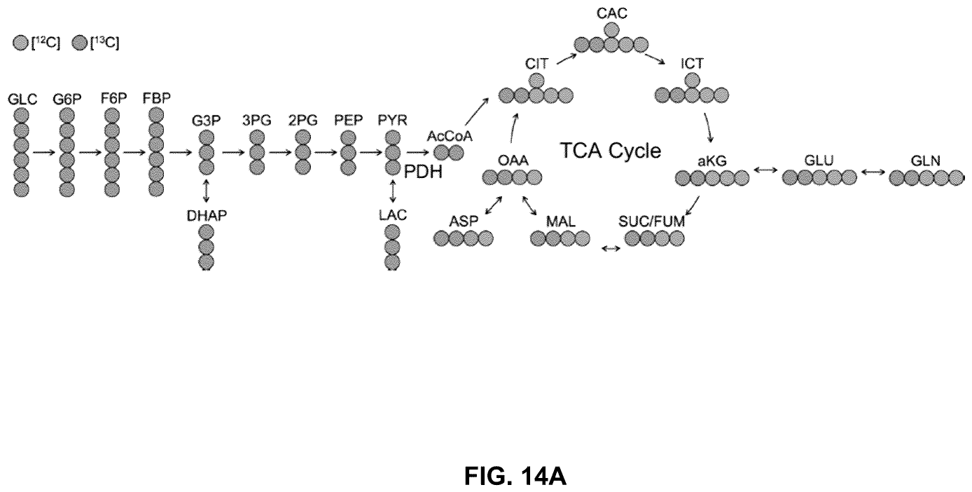

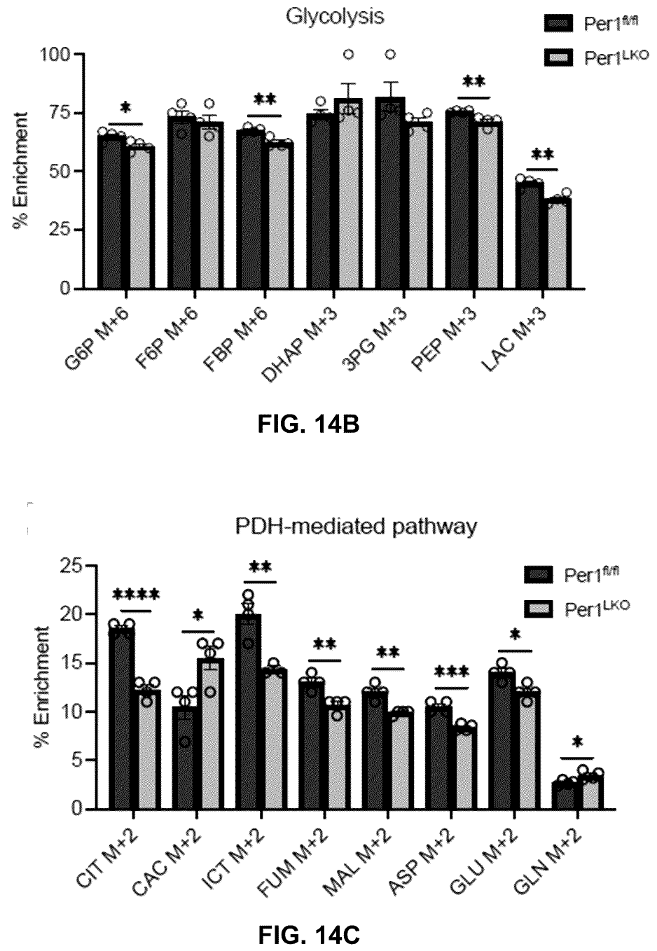

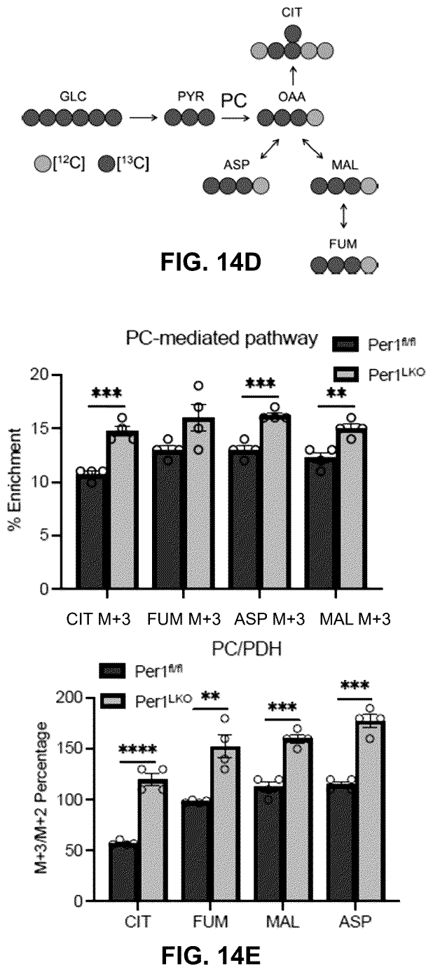

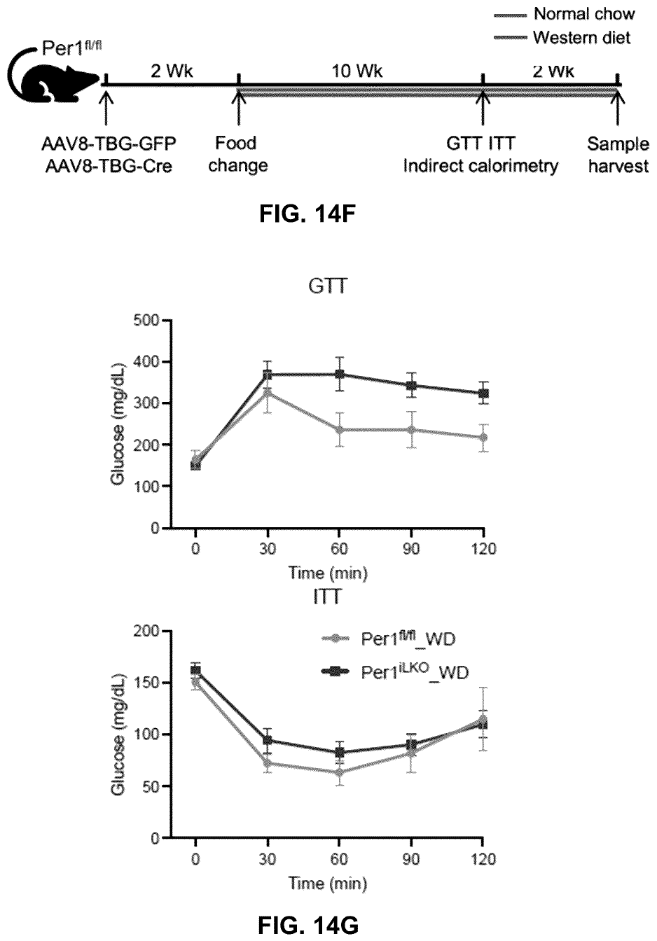

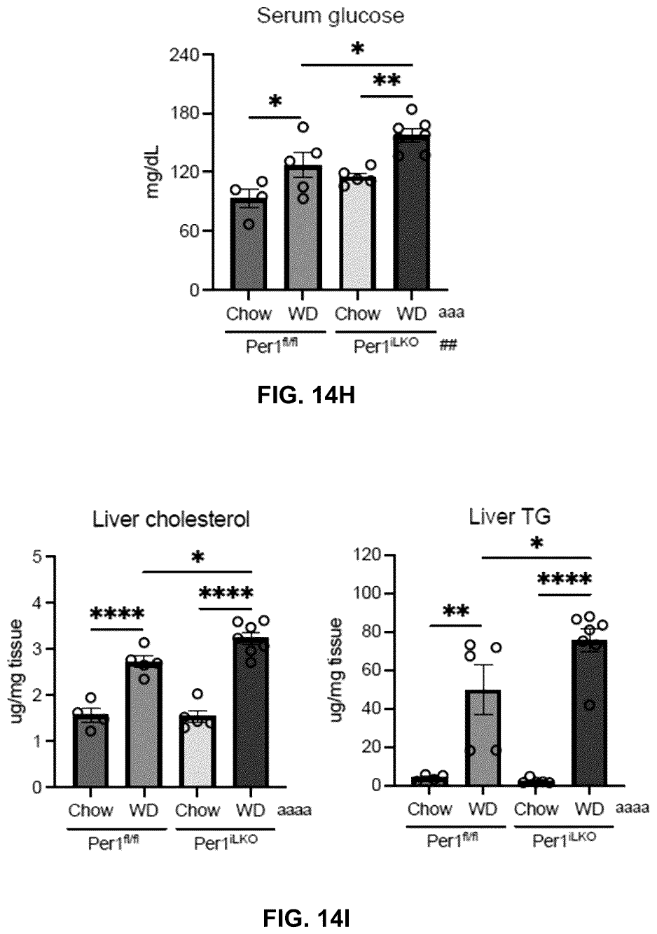

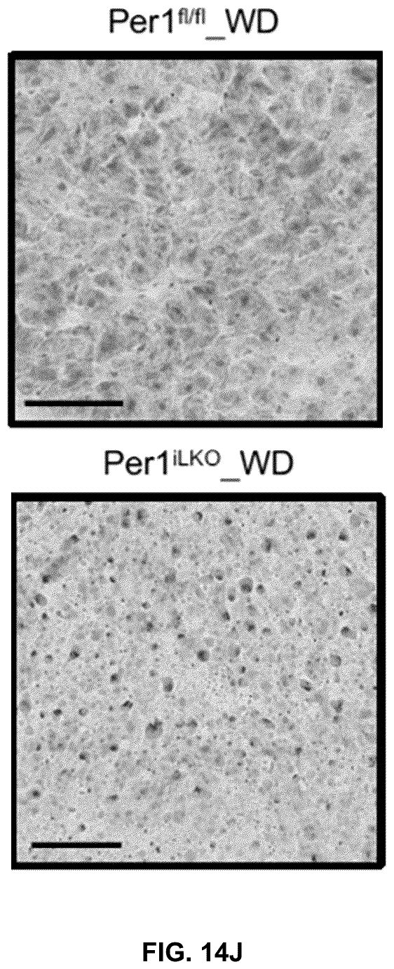

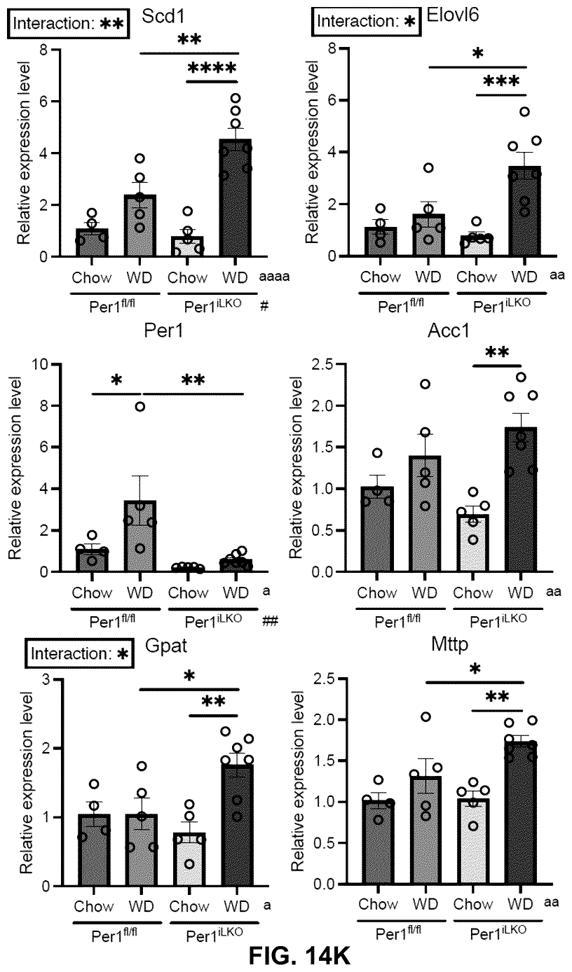

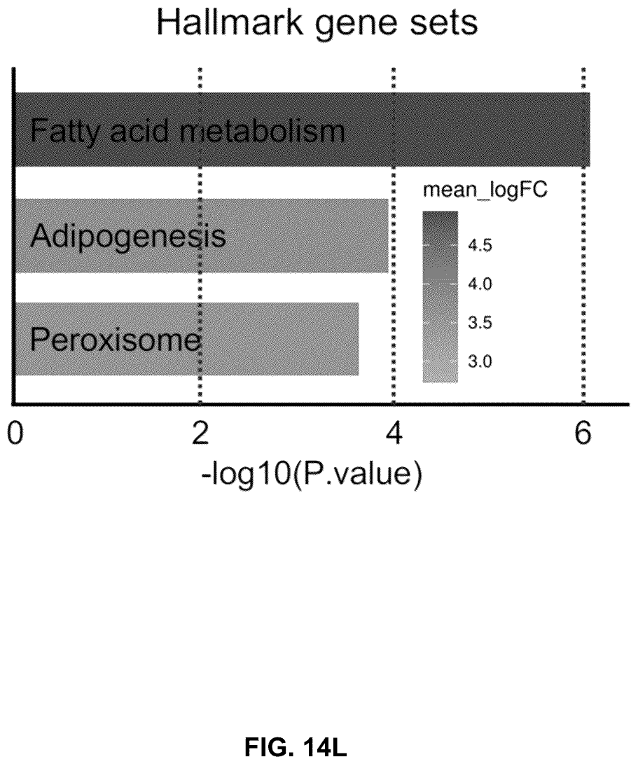

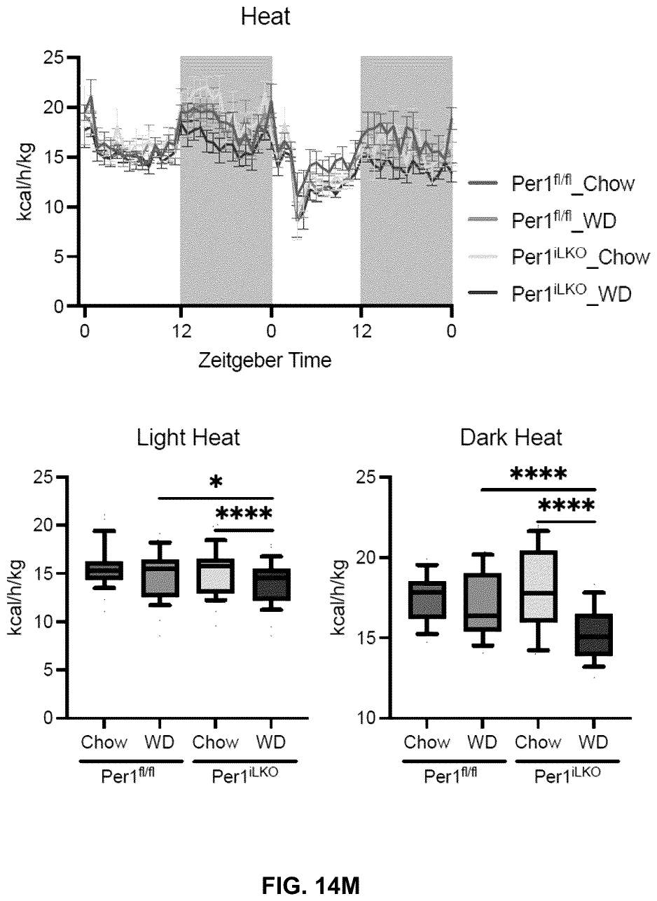

Previously, shown is that Per1 mediates fasting-induced Fgf21 and Pdk4 activation to facilitate the transition from glucose oxidation to fatty acid oxidation. To investigate how Per1 mediates glucose oxidation under nutrient replete conditions, in vitro isotope tracing was performed in isolated primary hepatocytes. Primary hepatocytes were isolated from mice with LoxP sites flanking the Per1 locus (Per1^{fl/fl}) and mice lacking hepatocyte Per1 (Per1^{LKO}, Albumin-Cre). It was found that nutrient-replete Per1^{LKO} hepatocytes exhibited lower glycolysis and TCA cycle intermediary labeling generated through the pyruvate dehydrogenase (PDH) pathway, suggesting an impaired glucose oxidation (FIG. 14A, FIG. 14B, FIG. 14C). In contrast, labelled metabolites generated through the pyruvate carboxylase (PC)-mediated pathway was increased in Per1^{LKO} hepatocytes (FIG. 14D, FIG. 14E). This predilection towards PC over PDH-mediated glucose flux through the TCA cycle in Per1^{LKO} hepatocytes indicates a predominantly anabolic state over the oxidative state (FIG. 14E).

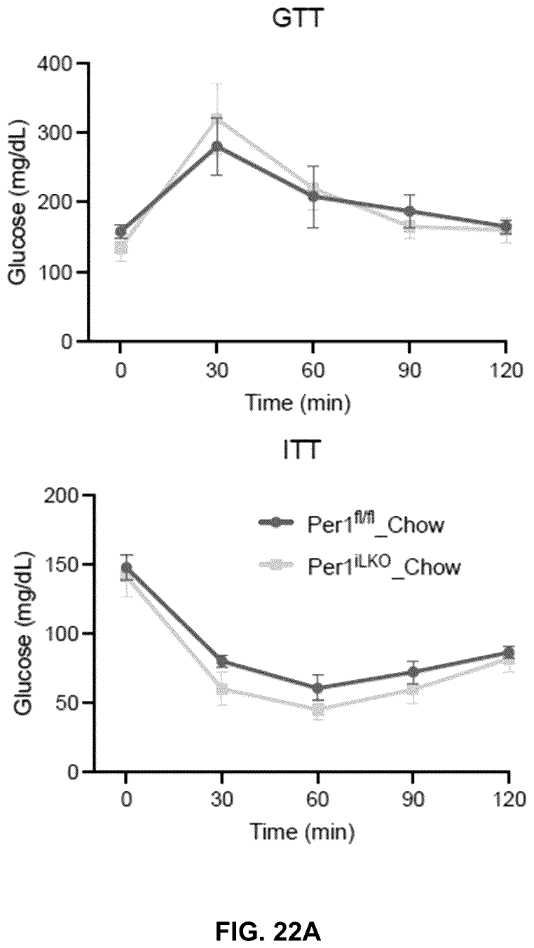

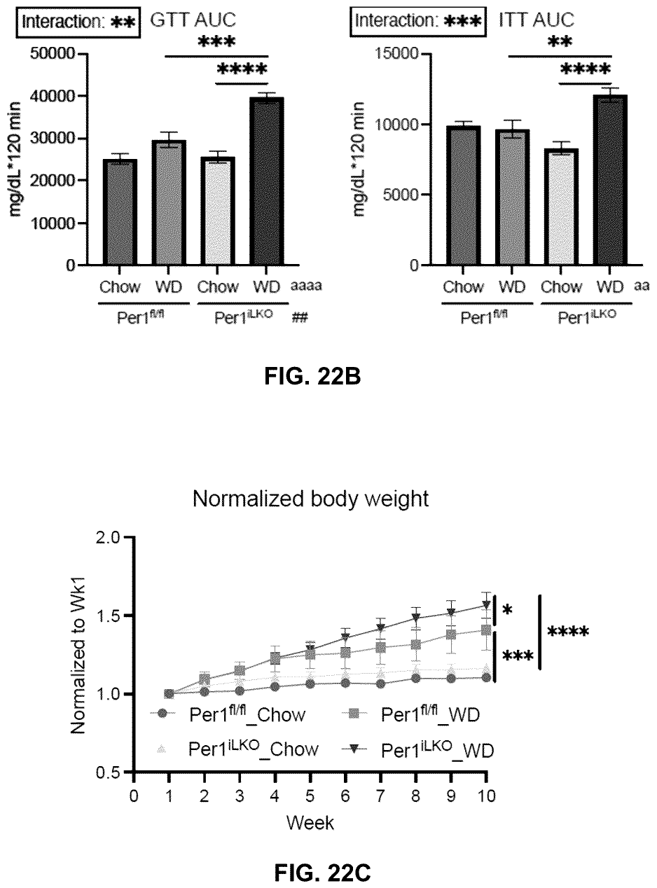

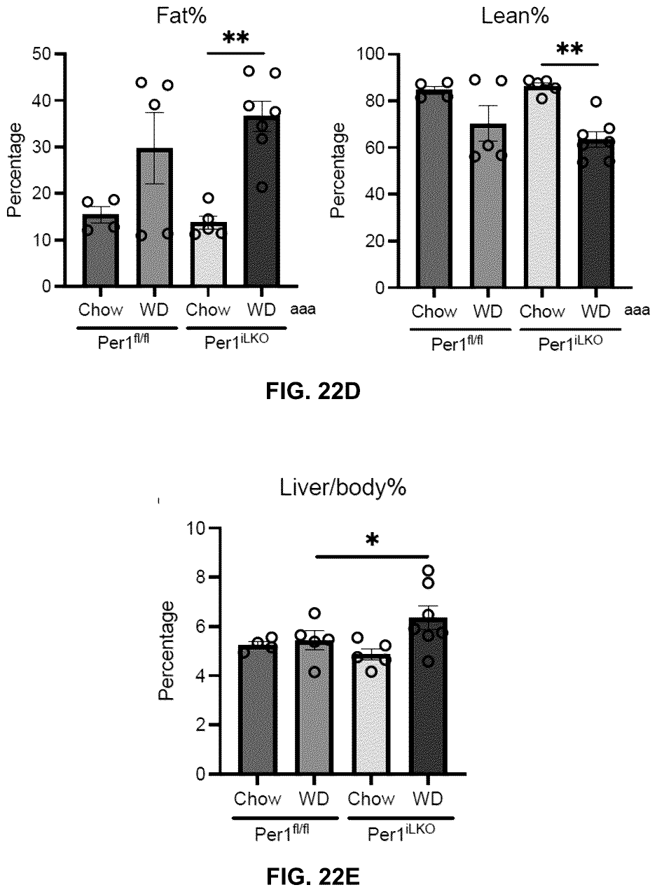

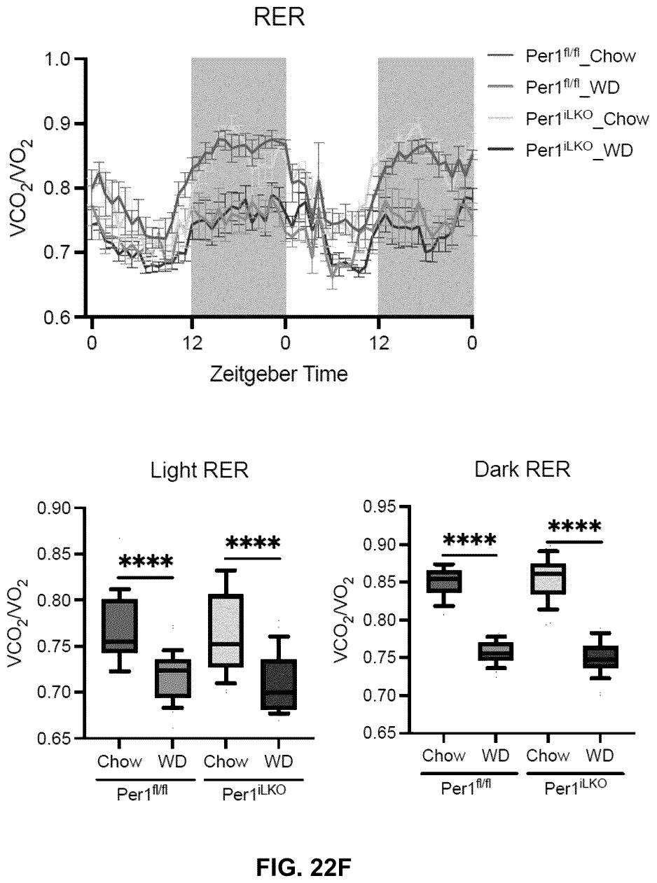

To define how hepatocyte Per1 dictates the response to overnutrition in vivo, this study next utilized mice with inducible hepatocyte-specific Per1 deletion (Per1^{iLKO}) by treating Per1^{fl/fl} mice with adeno-associated virus serotype 8 (AAV8) expressing either GFP or Cre under hepatocyte-specific thyroxine binding globulin (TBG) promoter control followed by challenge with chow or Western Diet (WD, 12 wk; FIG. 1F). Glucose and insulin tolerance testing (GTT and ITT) revealed significantly higher area-under-curve (AUC) values in WD-fed Per1^{iLKO} (FIG. 1G, FIG. 22B), but not in chow-fed Per1^{iLKO} mice when compared with Per1^{fl/fl} controls (FIG. 22A). WD-fed Per1^{iLKO} mice also gained more weight than WD-fed Per1^{fl/fl} mice without genotypic differences in fat and lean mass percentage (FIG. 22C, FIG. 22D). However, WD-fed Per1^{iLKO} mice developed a higher liver weight-to-body weight ratio compared to WD-fed Per1^{fl/fl} mice (FIG. 22E). This associated with significantly higher serum glucose, hepatic cholesterol and triglyceride (TG) levels in Perf^{iLKO} mice (FIG. 14H, FIG. 14I). Excess lipid accumulation was corroborated histologically in Perf^{iLKO} mice by Oil-Red-O (ORO) staining (FIG. 14J). Quantitative real-time PCR (qRT-PCR) validated lower Per1 expression in Per1^{iLKO} mice, whereas de novo lipogenic (DNL) genes—Acc1, Scd1, Elovl6, Gpat and Mttp—were significantly upregulated in WD-fed Per1^{iLKO} livers (FIG. 14K). Transcriptomic analysis confirmed significant activation in fatty acid metabolic, adipogenic, and peroxisomal gene pathways (FIG. 14L). Indirect calorimetry in these mice revealed lower heat generation in WD-fed mice versus chow-fed control mice. Obese Per1^{iLKO} mice had significantly lower heat production in both light and dark phases, suggesting a thermogenic defect in Perf^{iLKO} mice (FIG. 14M) with a trend in lower respiratory exchange ratio (RER; FIG. 22F). Collectively, loss-of-function data indicate that hepatocyte Per1 is required to protect liver from diet-induced obesity, hepatic lipid accumulation, DNL gene expression, and while driving oxidative metabolism.

### Single-Nucleus RNA-Sequencing Reveals Hepatocyte-Specific Per1 Suppression of Translationally Relevant MAFLD and HCC Disease-Associated Genes

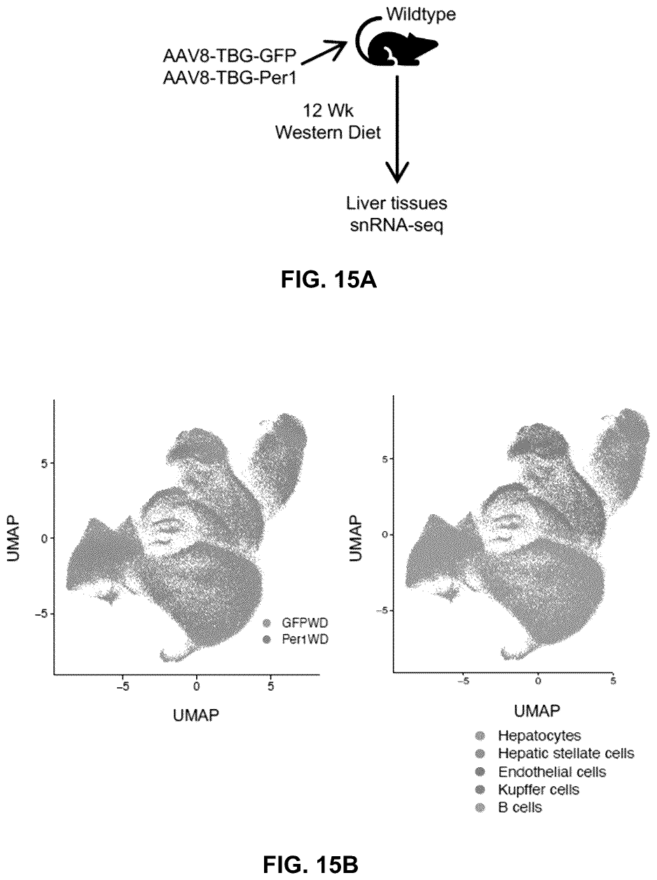

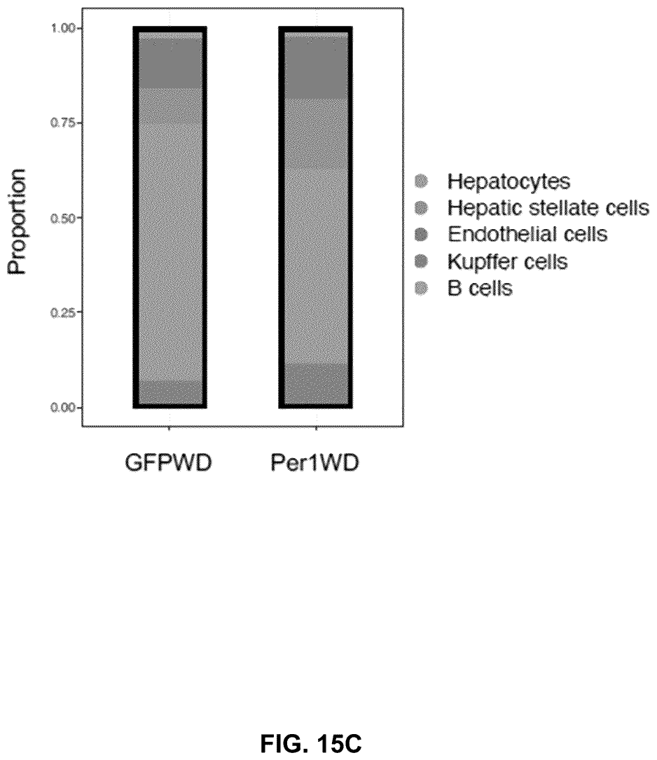

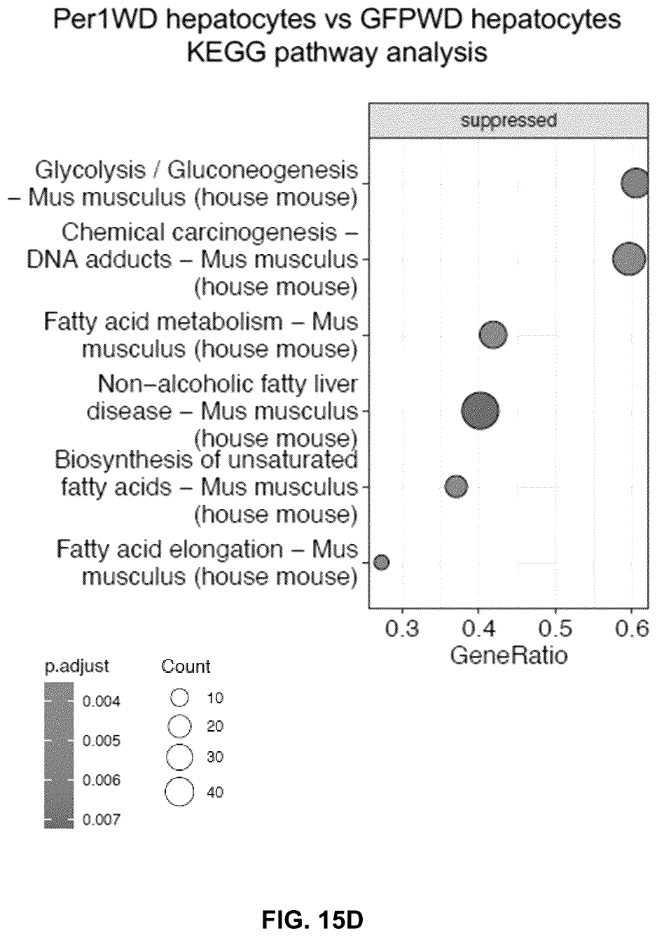

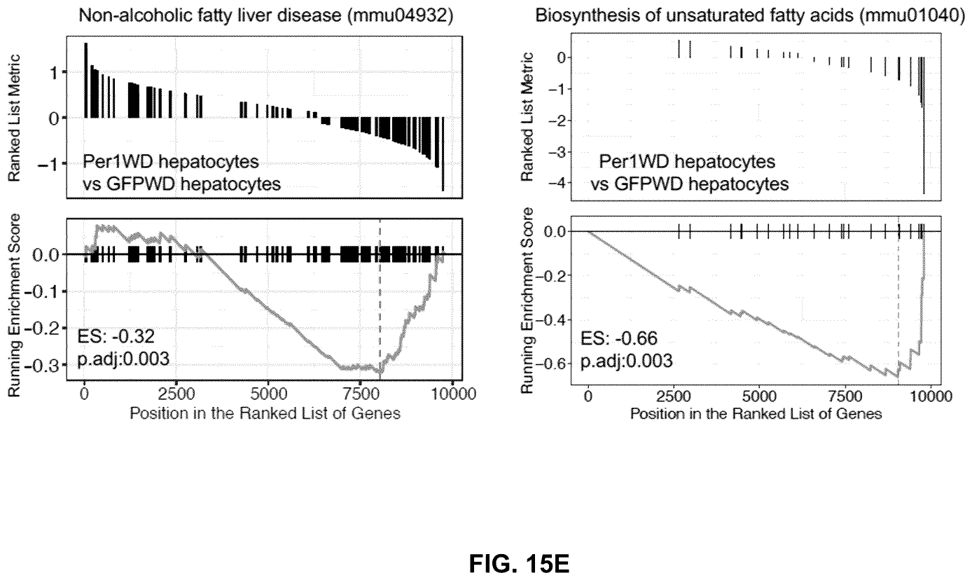

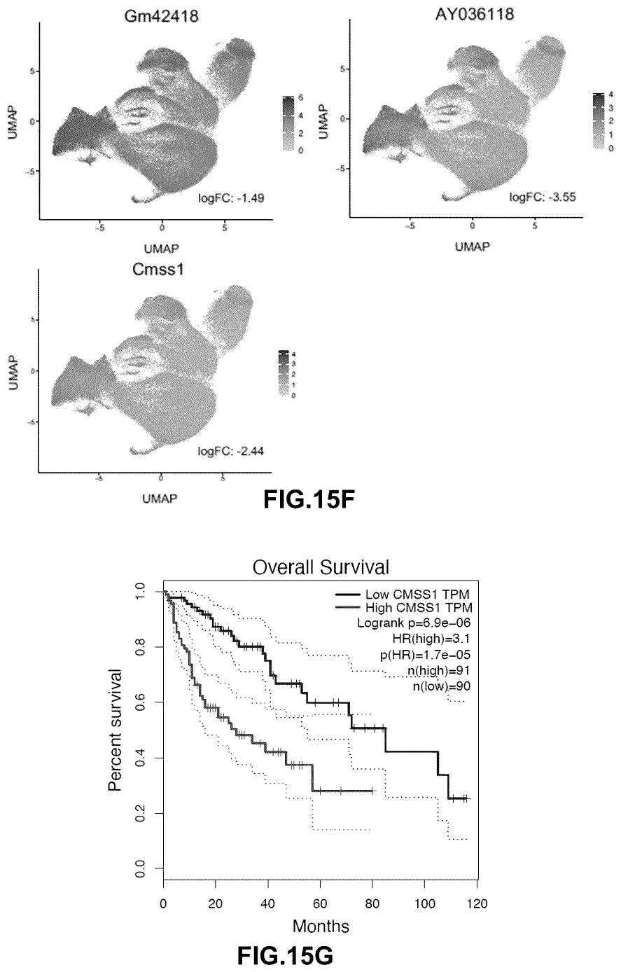

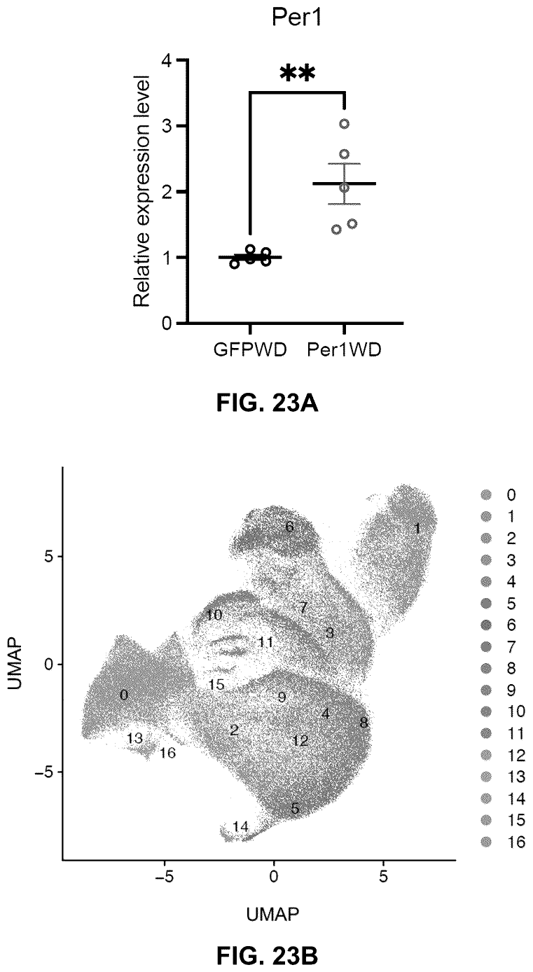

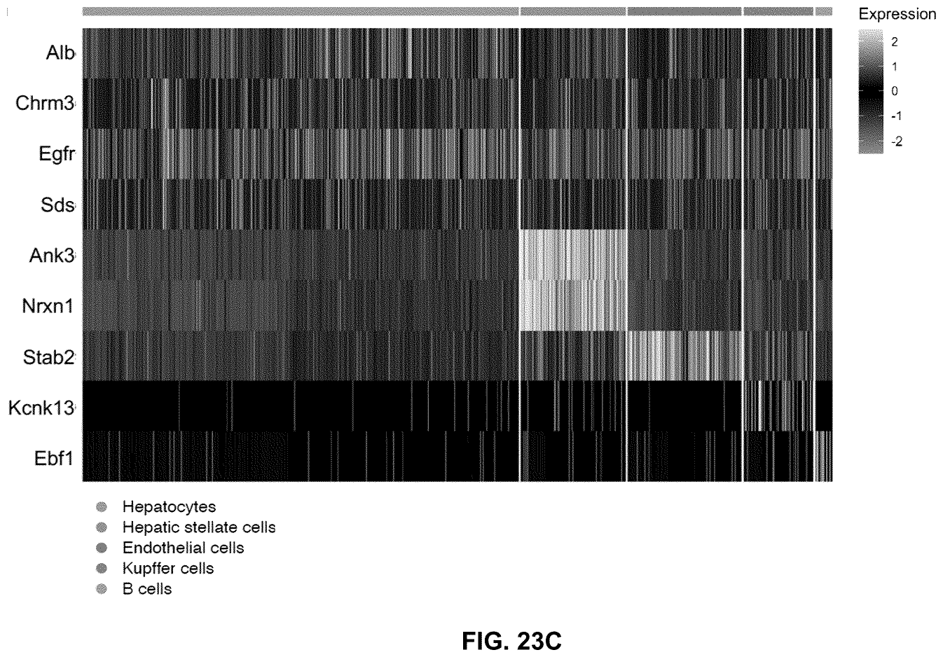

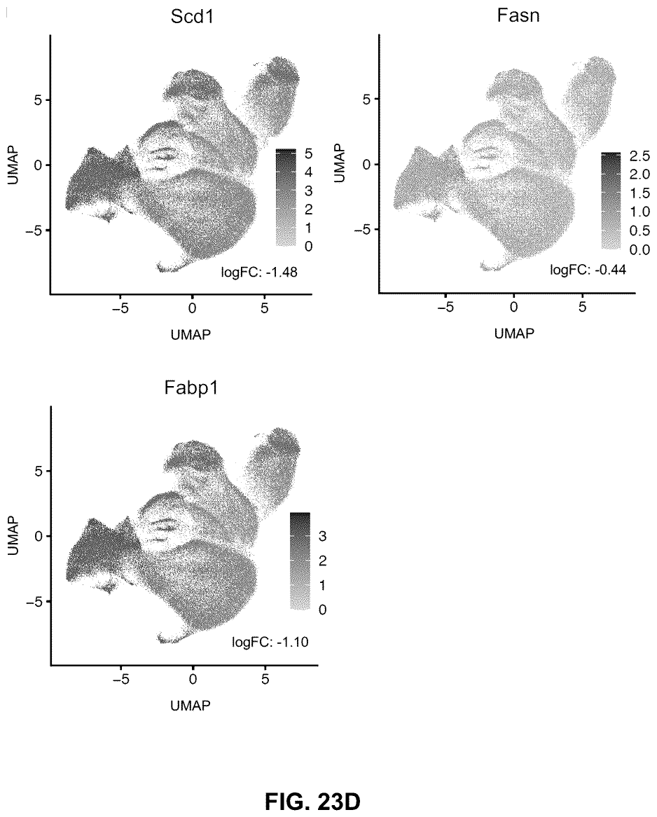

Next, this study examined the degree to which forced Per1 expression in hepatocytes drives transcriptional changes in livers from obese mice. To resolve heterogeneous cell types in the liver, single-nucleus RNA sequencing (snRNA-seq) was performed in WD-fed wild-type mice expressing GFP or Per1 (FIG. 15A, FIG. 23A). Five major liver cell types were assigned to the 17 clusters (FIG. 15B, FIG. 15C, FIG. 23B, FIG. 23C). Kyoto Encyclopedia of Genes and Genomes (KEGG) pathway analysis of differentially expressed genes revealed significant suppression in pathways involved in fatty acid synthesis, non-alcoholic fatty liver disease (MASLD), gluconeogenesis and carcinogenesis (FIG. 15D, FIG. 15E). Differential expression analysis identified the three significantly downregulated genes in hepatocytes overexpressing Per1: AY036119, Gm42418, and Cmss1 (FIG. 15F). Although the functions of these three genes are not fully understood, AY036119 and Gm42418 are previously identified as MASLD-associated genes. Genes involved in lipogenesis and development of fatty liver disease were also significantly suppressed in Per1-overexpressed liver (FIG. 23D). Moreover, data from patients with hepatocellular carcinoma (HCC) revealed that lower hepatic CMSS1 expression correlates with a significantly improved survival outcome (FIG. 15G). Hepatocyte Per1-overexpression in obese mice significantly reduced hepatocyte-specific genes associated with MASLD and HCC, and these transcriptional changes portend translationally relevant consequences in human disease.

Structure-Function Analyses in Clock-Deficient Mice Reveal Per1 Sub-Domains that Drive Glucose Tolerance and Thermogenesis

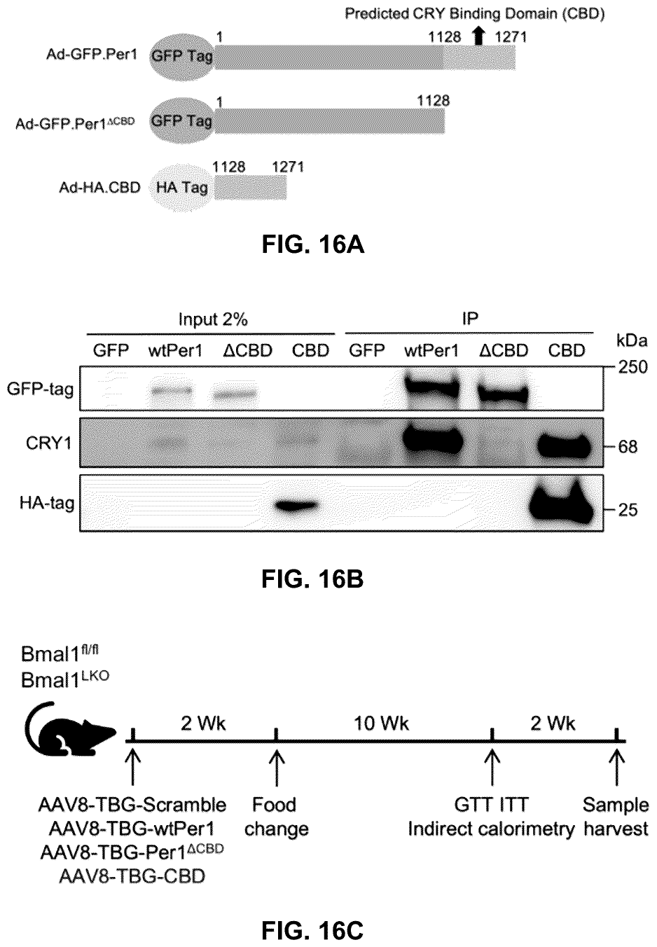

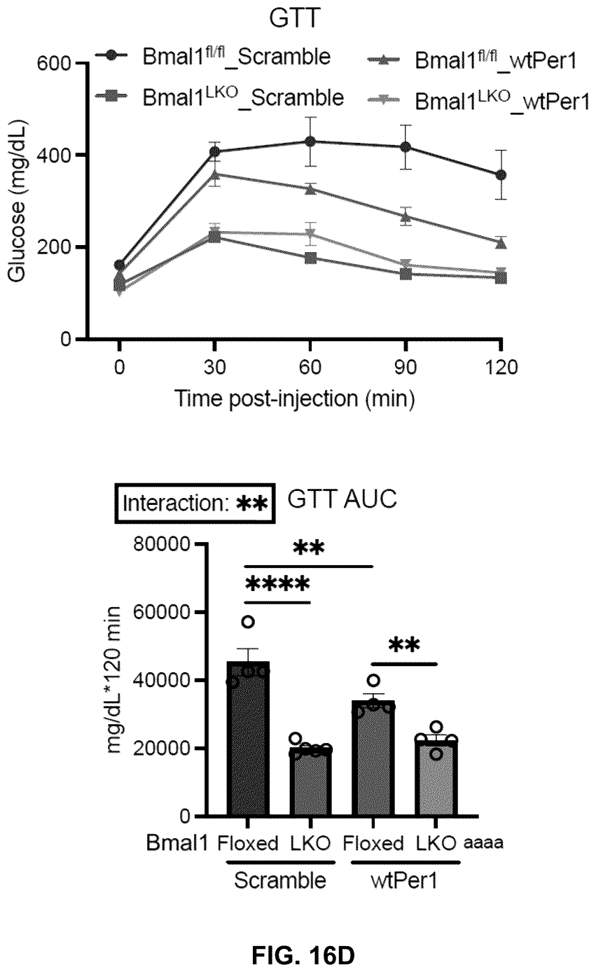

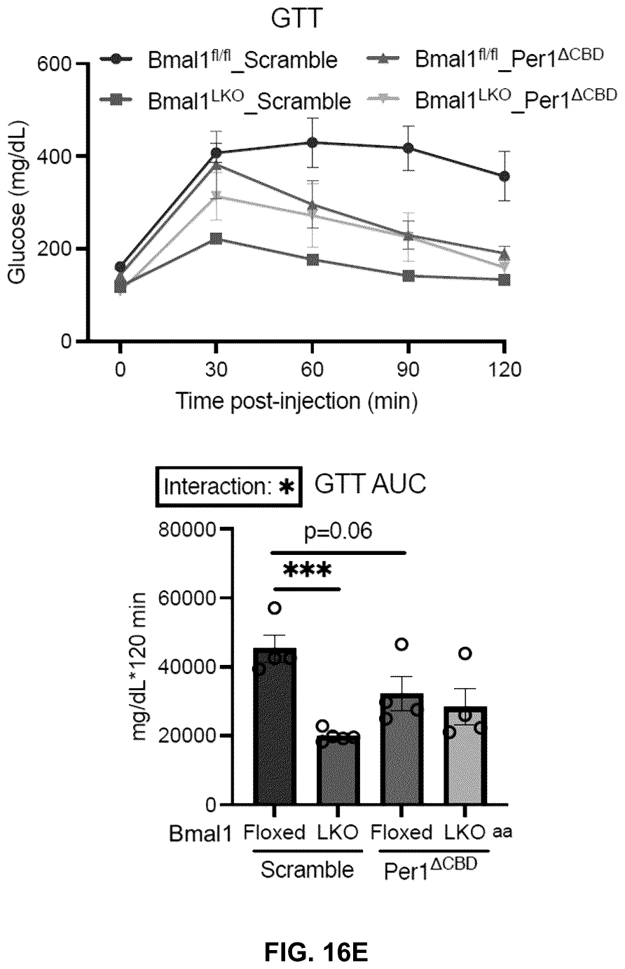

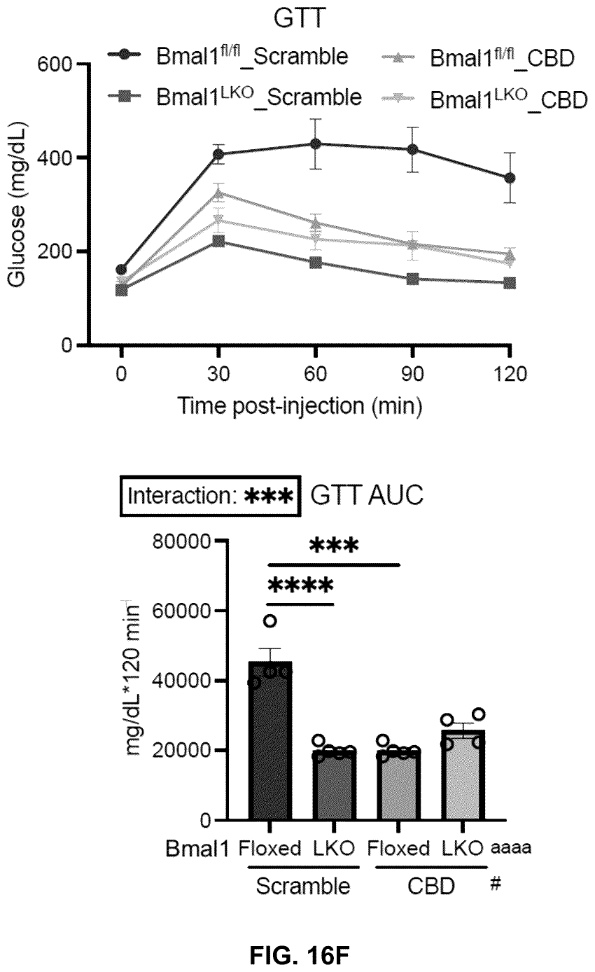

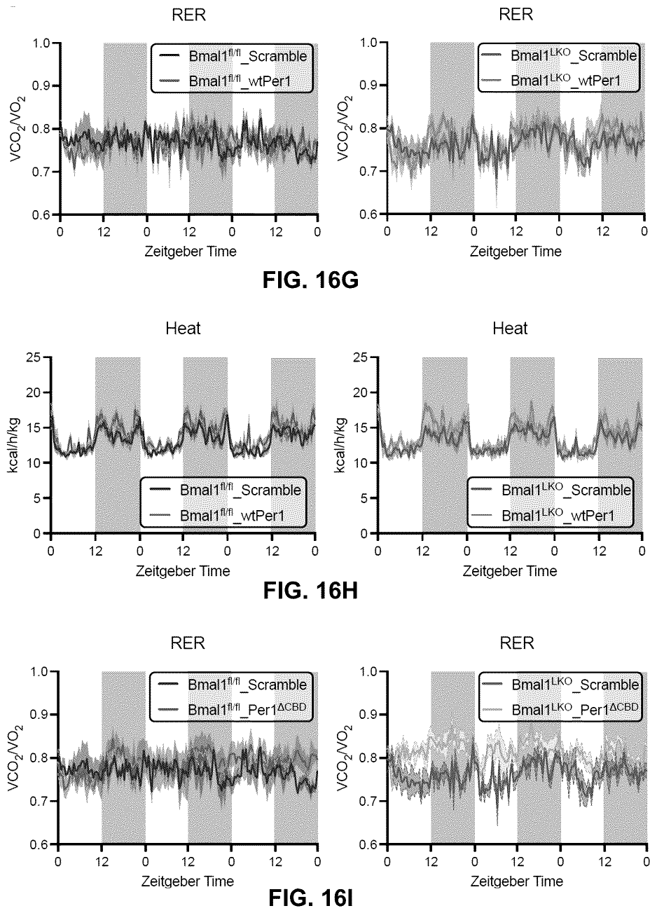

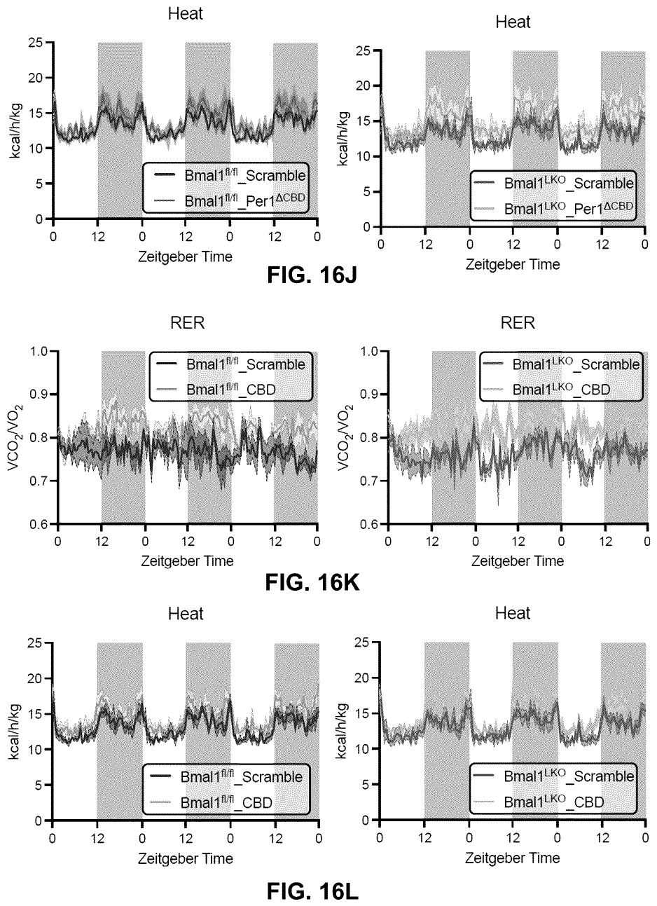

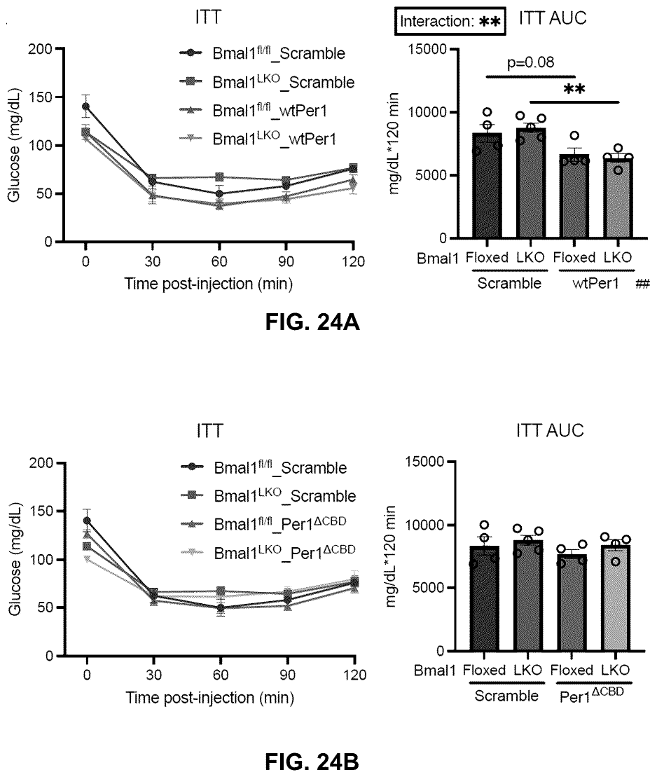

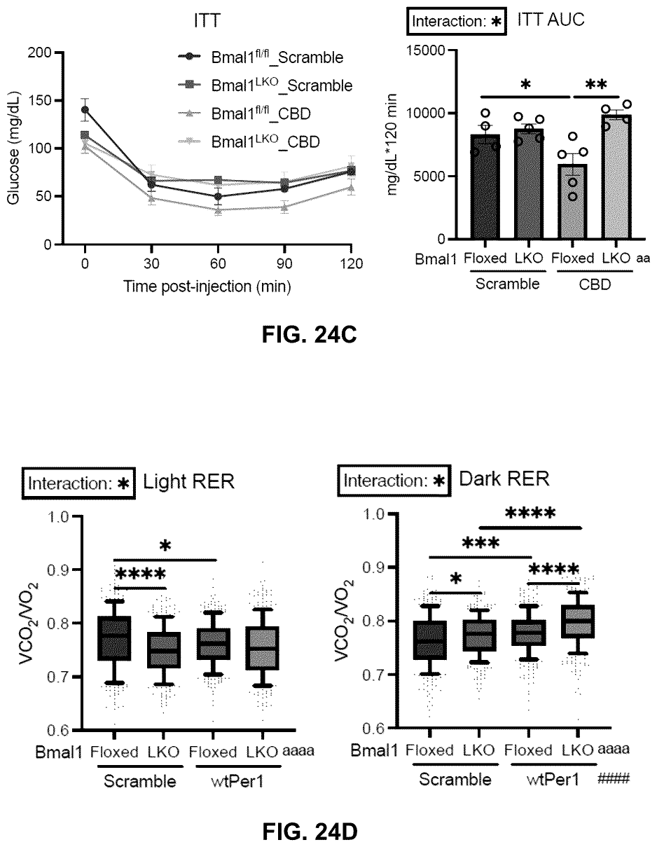

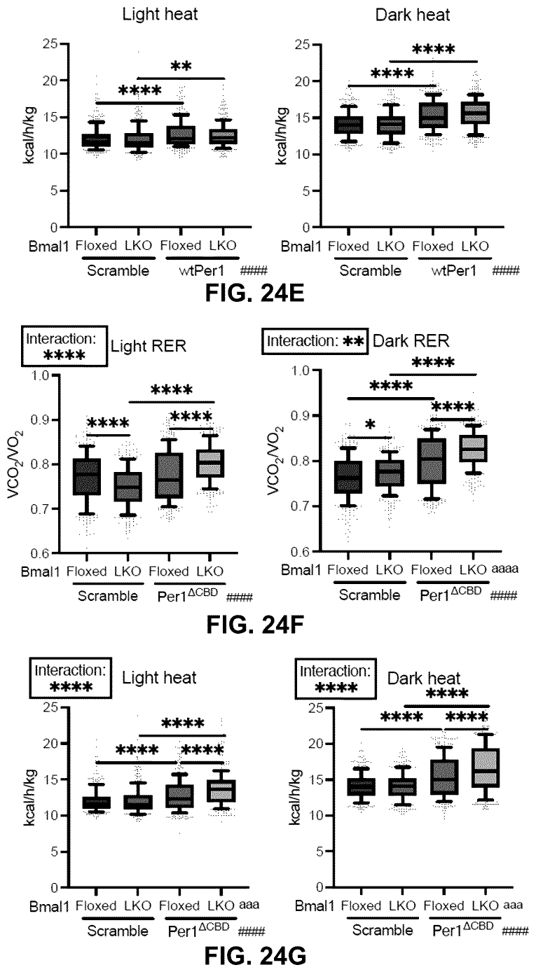

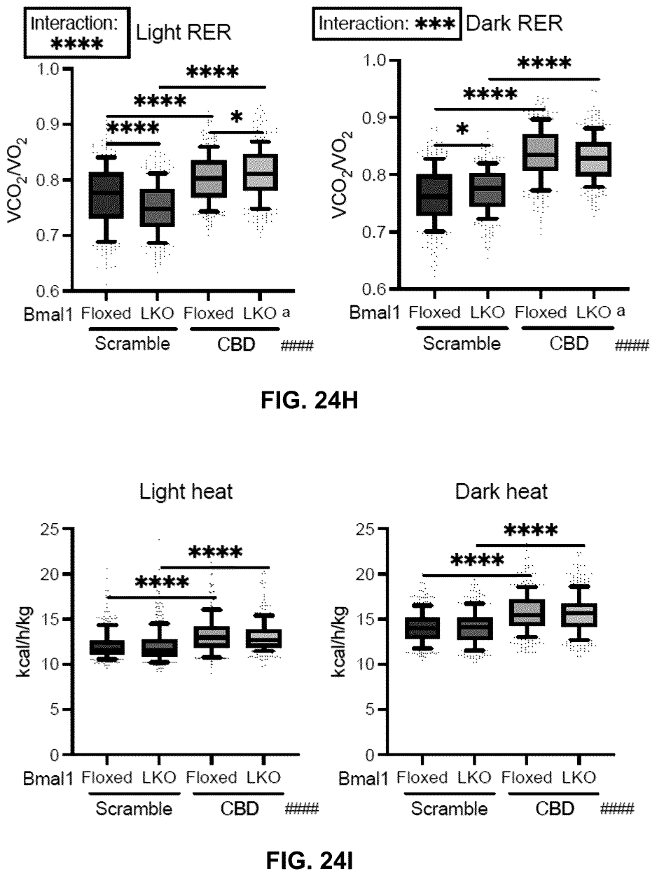

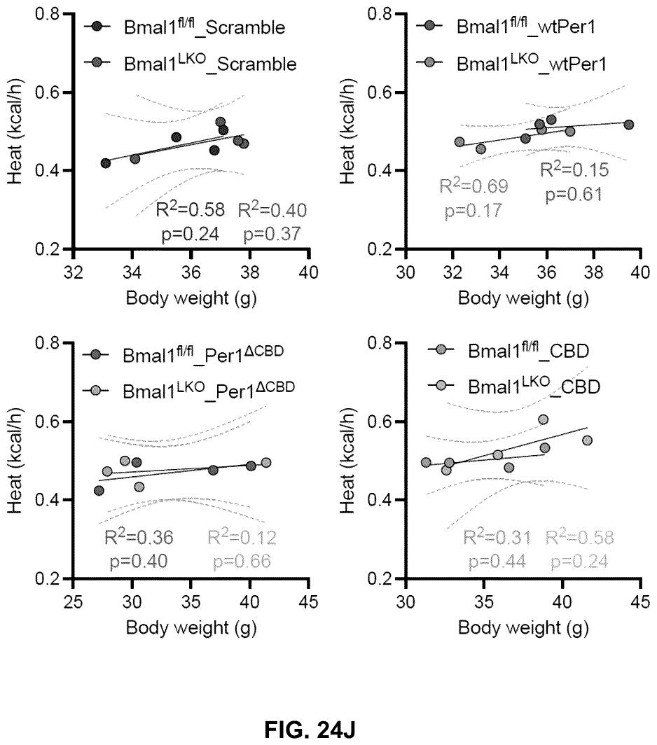

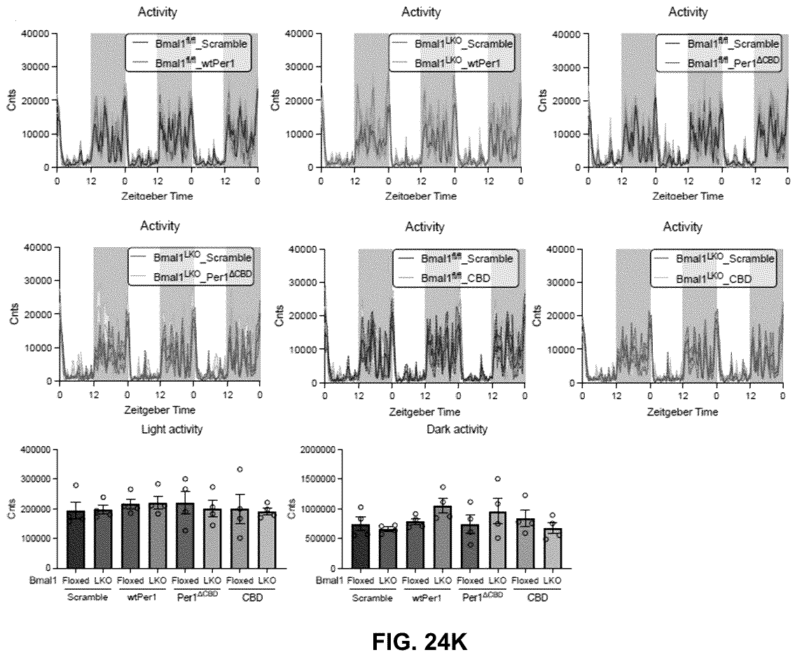

Per1 primarily executes its known circadian function through direct binding to CRY1/2 to function as a heterodimer. To examine the extent to which metabolic function of hepatocyte Per1 requires CRY-binding activity, two mutant constructs were generated. The first lacks the putative CRY-binding domain (Per1^{ΔCBD}) and the second construct expresses only the CRY-binding domain (CBD) without the N-terminus (FIG. 16A). In vitro expression of full-length Per1 and its two mutant constructs followed by co-immunoprecipitation validated that full-length PER1 and CBD domain alone bind CRY1, but PER1^{ΔCBD} does not (FIG. 16B). To control for peptide expression in hepatocytes, a control construct was generated expressing the peptide containing scrambled sequence amino acids reflected in the CBD fragment. The efficacy of each construct in modulating the systemic energy metabolism in vivo was examined in the presence or absence of a fully functional liver circadian clock. Therefore Per1 or its mutant constructs was overexpressed in Bmal1^{fl/fl} or Bmal1^{LKO} (Bmal1^{fl/fl}×Albumin-Cre) mice and fed the mice with WD for 12 weeks (FIG. 16C). GTT and ITT demonstrated that mice lacking hepatocyte Bmal1 maintained lower blood glucose during GTT but not ITT (FIG. 16D, FIG. 16F, FIG. 24A, FIG. 24B, FIG. 24C). Bmal1^{fl/fl} mice overexpressing Per1, Per1^{ΔCBD} and CBD exhibited trending or significant reductions in glucose tolerance AUC (FIG. 16D, FIG. 16E, FIG. 16F). This indicates that Per1, Per1^{ΔCBD} and CBD are each sufficient to improve glucose homeostasis in obese mice. No further improvement in GTT or ITT was observed in Bmal1^{LKO} mice overexpressing Per1 or its two mutant constructs.

Gas exchanges quantification in these mice via indirect calorimetry demonstrated that wtPer1 overexpression in Bmal1^{fl/fl} and Bmal1^{LKO} mice significantly increased RER during the dark phase (FIG. 16G, FIG. 24D). Mice overexpressing hepatocyte wtPer1 had significantly higher heat production in both Bma/1^{fl/fl} and Bmal1^{LKO} mice independent of light/dark phase (FIG. 16H, FIG. 24E). Overexpressing Per1^{ΔCBD} mutant construct in Bmal1^{fl/fl} mice increased RER in dark phase and had higher heat production in both light and dark phases. Bmal1^{LKO} mice overexpressing Per1^{ΔCBD} had significantly higher RER and heat production in both light/dark conditions (FIG. 16I, FIG. 16J, FIG. 24F, FIG. 24G). CBD fragment uniquely increased heat and RER in both dark and light phases in both Bmal1^{fl/fl} and Bmal1^{LKO} mice (FIG. 16K, FIG. 16L, FIG. 24H, FIG. 24I). No correlation was observed between body weight and heat production in all conditions, and no difference was observed in total locomotor activity across groups (FIG. 24J, FIG. 24K). In summary, wtPer1 increases glucose oxidation and heat production independent of its CRY-binding activity, and CBD itself also exerts a similar effect as wtPer1 and Per1^{ΔCBD} constructs. In addition, a more complex interaction was observed when comparing RER in Bmal1^{fl/fl} mice and Bmal1^{LKO} mice expressing each construct. This indicates that Per1's function in energy expenditure is Bmal1 and Cry-independent, yet Per1's action in substrate oxidation may not be completely Bmal1-independent.

### Per1 Suppresses Lipogenesis and Triglyceride Accumulation Independent of Bmal1

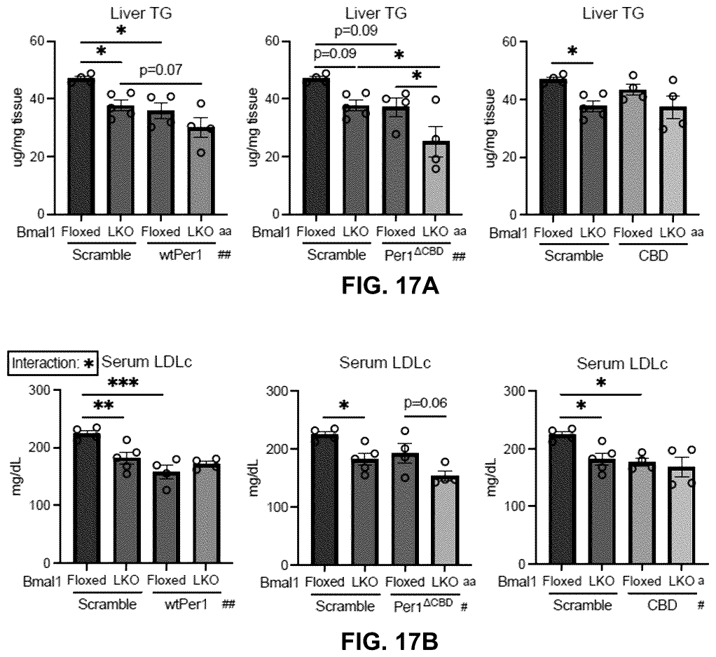

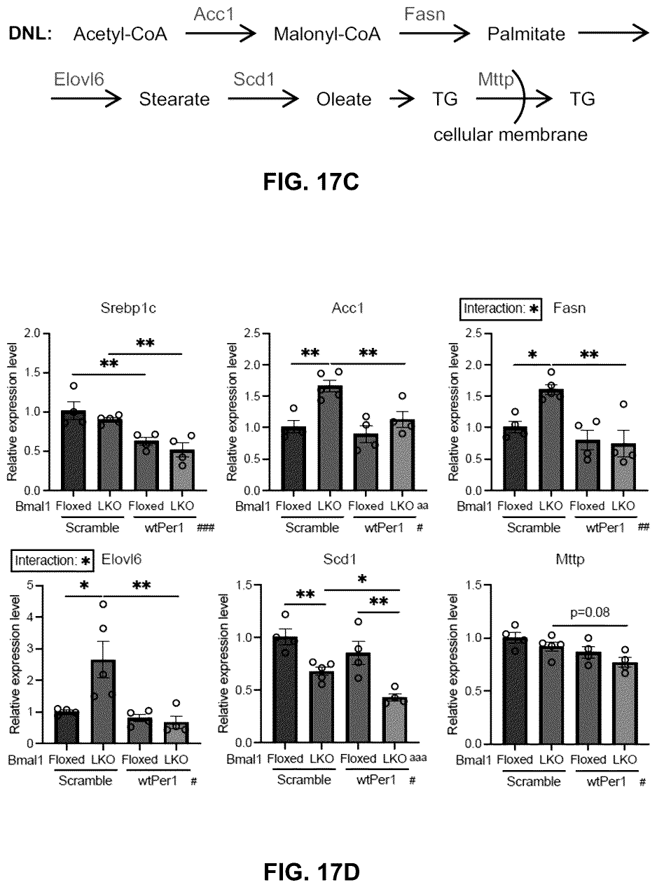

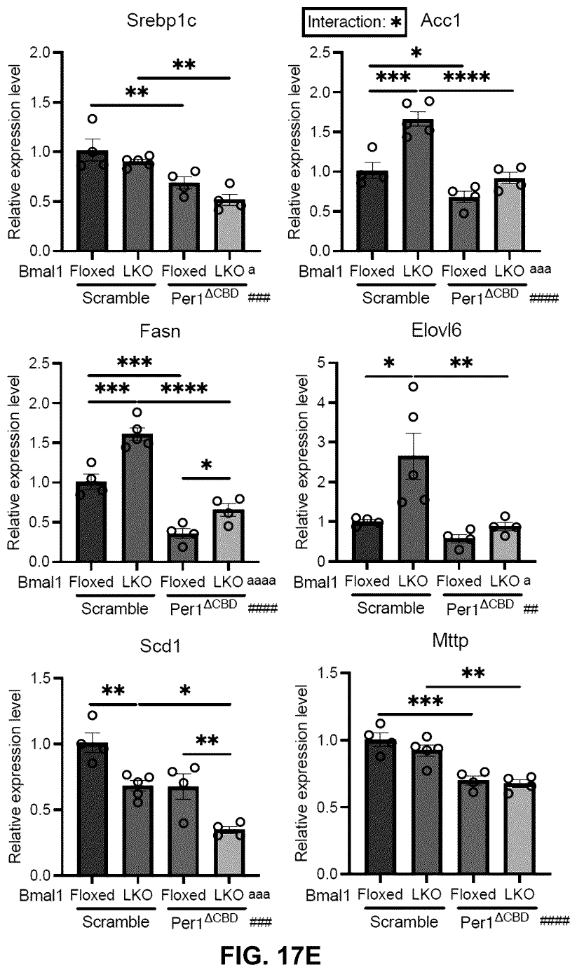

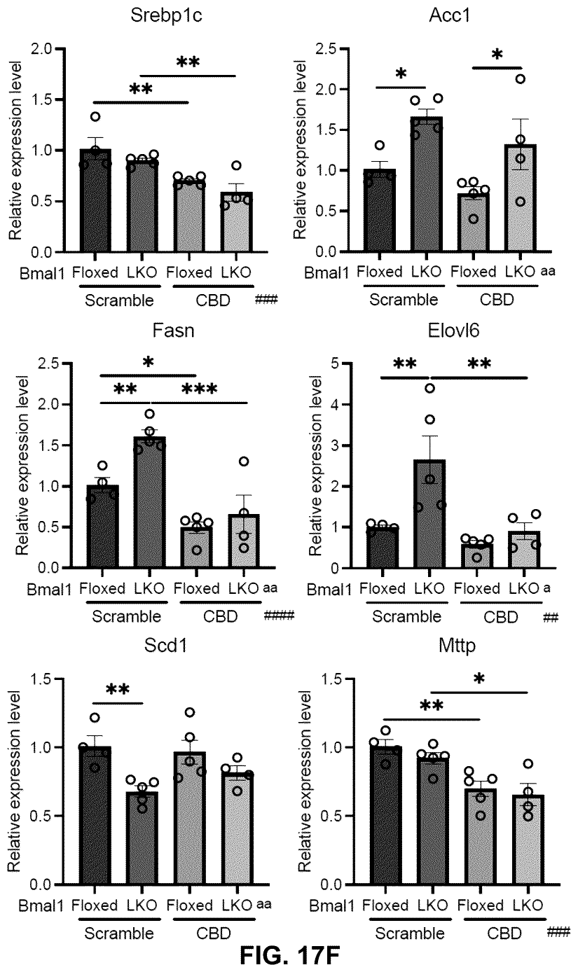

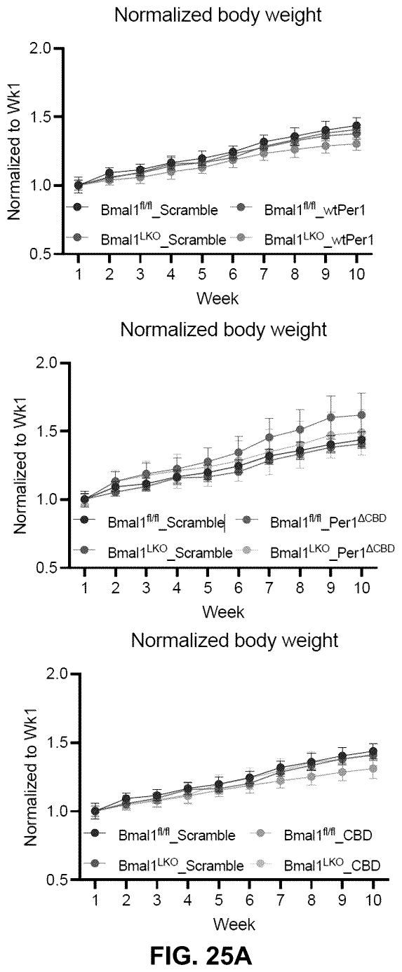

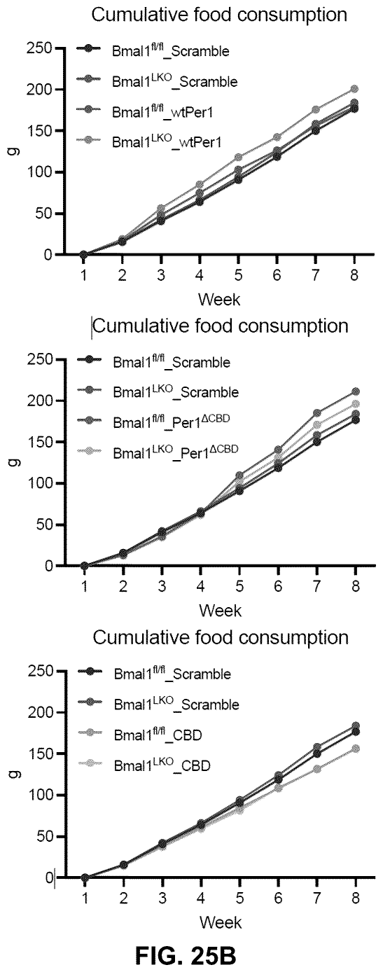

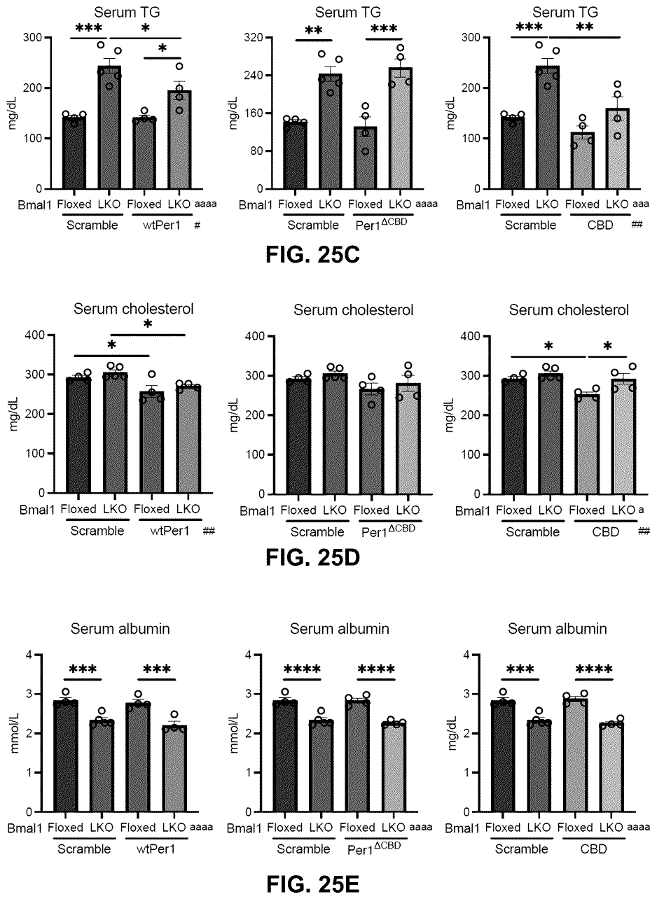

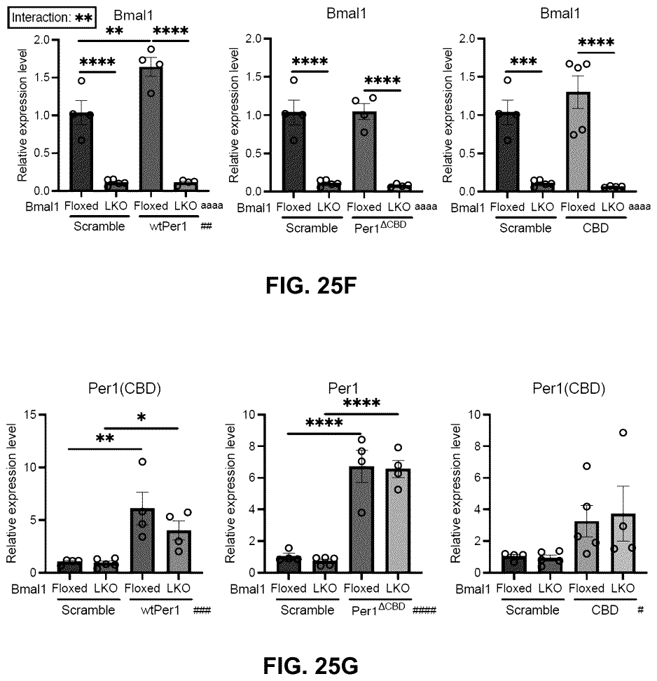

This study next quantified the efficacy of wtPer1, Per1^{ΔCBD} and CBD on hepatic and serum lipids. After twelve-week WD exposure, no significant difference were observed in normalized body weight or total food consumption among different groups (FIG. 25A, FIG. 25B). Bmal1^{LKO} mice had lower TG in liver tissue but significantly higher serum TG in scramble-expressing mice (FFIG. 17A, FIG. 25C). wtPer1 reduced hepatic TG in WD-fed Bmal1^{fl/fl} mice, with trends lower in Bmal1-deficient liver, and also upon expressing either of the truncated mutants (FIG. 17A). In contrast both wtPer1 and CBD were each sufficient to lower total cholesterol and low-density lipoprotein cholesterol (LDLc) in Bmal1^{fl/fl} mice, but not in Bmal1^{LKO} mice for LDLc level (FIG. 17B, FIG. 25D). Per1 and Per1 mutant overexpression did not affect serum albumin level (FIG. 25E). The data confirm an anti-steatotic and anti-dyslipidemic effect of full-length Per1 in liver, and indicate a partial interaction between Cry1/2 binding and Bmal1 function in Per1's effects on lipid homeostasis.

Given that DNL gene expression is increased in WD-fed Per1^{iLKO} liver, therefore it was asked if overexpressing Per1 or its mutant constructs blocks lipogenic pathway gene expression (FIG. 17C). Overexpression of wtPer1 along and its two mutant constructs were sufficient to supress expression of lipogenic gene transcription factor Srebp1c in both Bmal1^{fl/fl} and Bmal1^{LKO} mice (FIG. 17D, FIG. 17E, FIG. 17F). wtPer1 overexpression significantly reduced the expression of lipogenic genes Acc1, Fasn, Elovi6 and Mttp in Bmal1^{LKO} mice, with a trend towards lower expression in Mttp (FIG. 17D). Similarly, Per1^{ΔCBD} overexpression significantly reduced Acc1, Fasn and Mttp in both Bmal1^{fl/fl} and Bmal1^{LKO} mice, with a significant reduction of Scd1 in Bmal1^{LKO} mice (FIG. 17E). Overexpressing CBD fragment significantly suppressed Fasn and Mttp in both Bmal1^{fl/fl} and Bmal1^{LKO} mice. It also suppressed Elovi6 in Bmal1^{LKO} mice (FIG. 17F). qRT-PCR further validated a successful knockout of Bmal1 in Bmal1^{LKO} mice, and successful overexpression of each construct (FIG. 25F, FIG. 25G). Together, Per1 generally suppresses de novo lipogenic gene in both Bmal1^{fl/fl} and Bmal1^{LKO} mice, and this does not fully require its CRY binding function.

In Vitro Pull-Down Nominates PP2A as a Direct PER1 Binding Effector that Mediates Hepatocyte De Novo Lipogenic Gene Expression

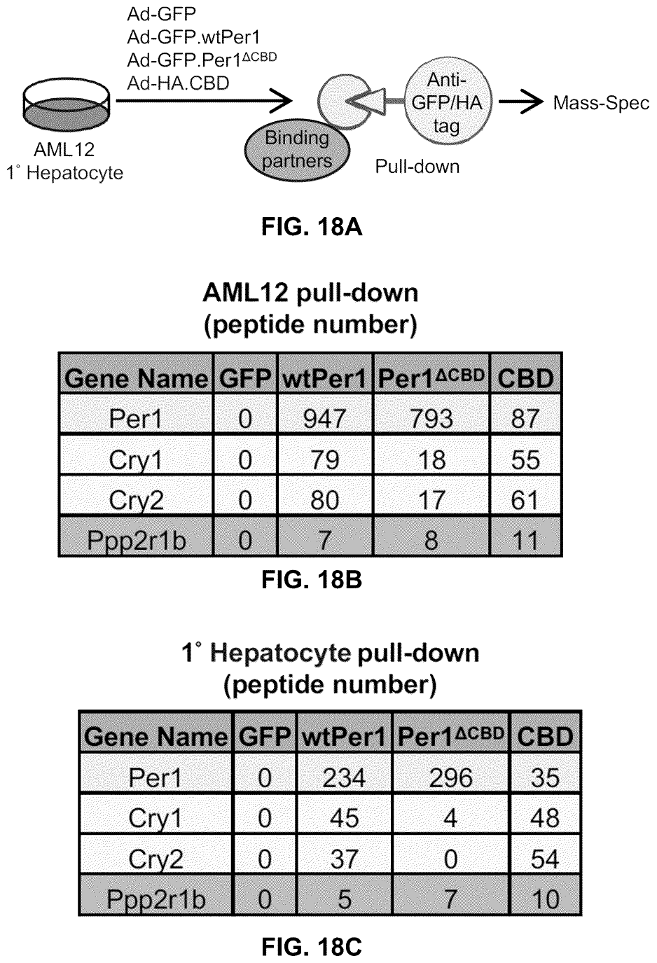

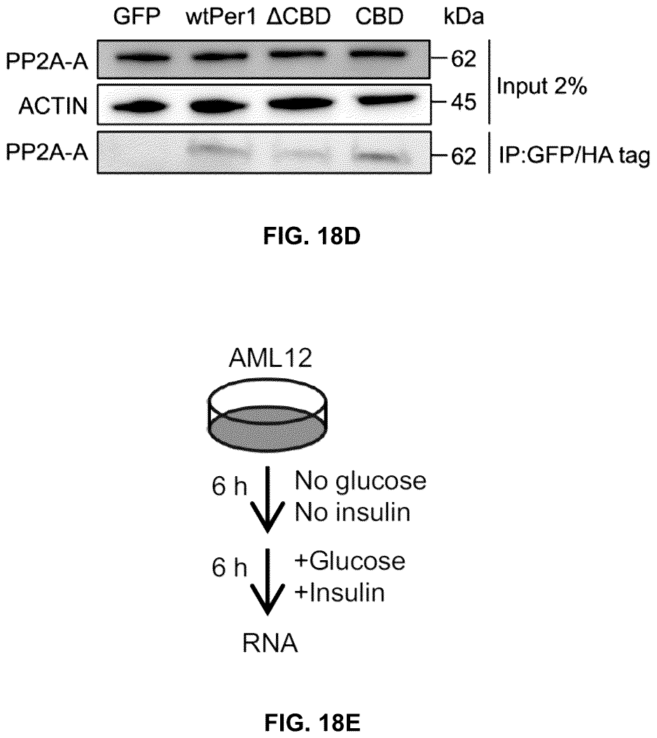

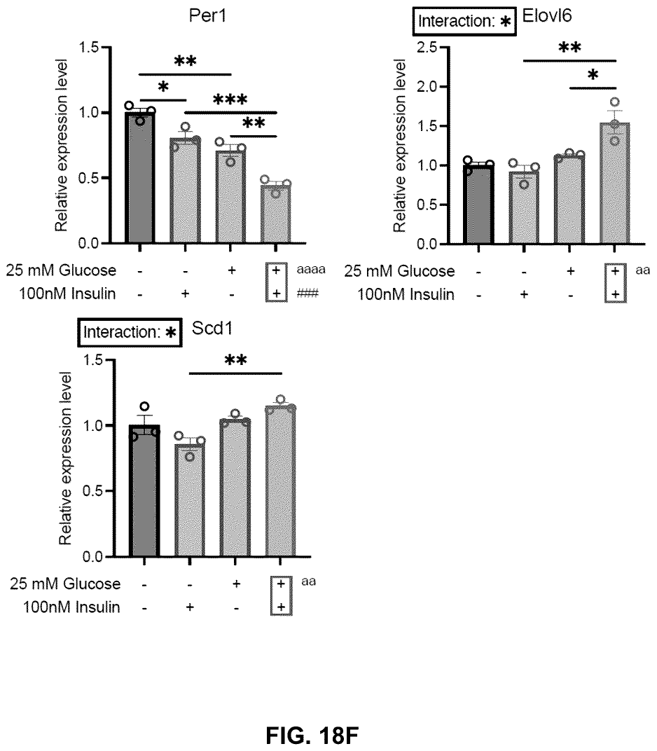

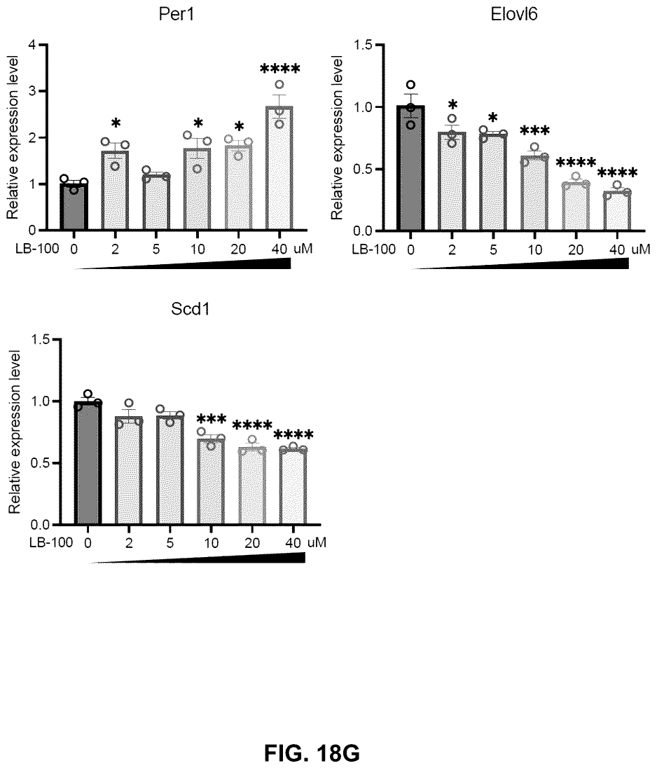

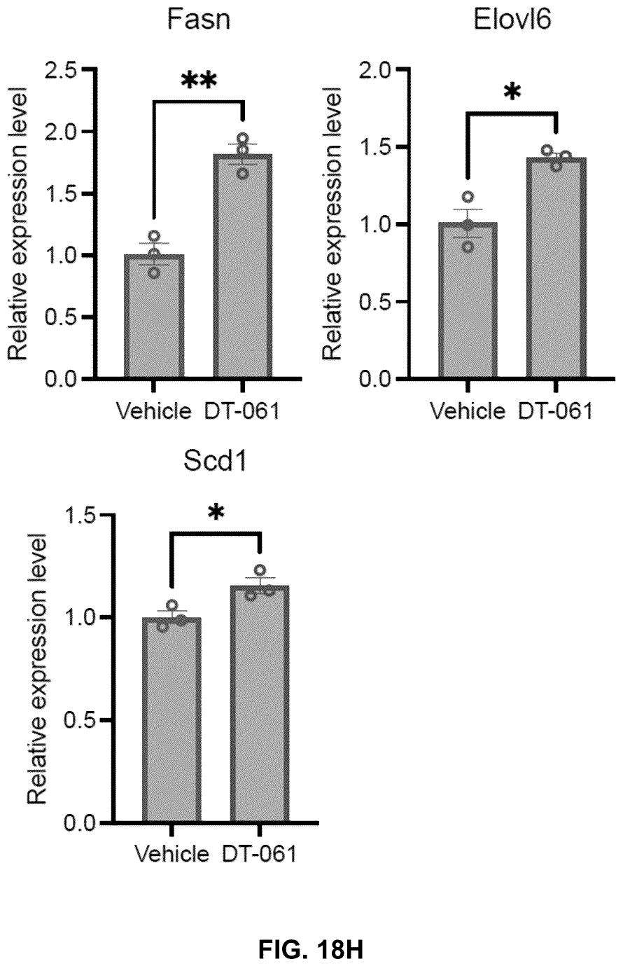

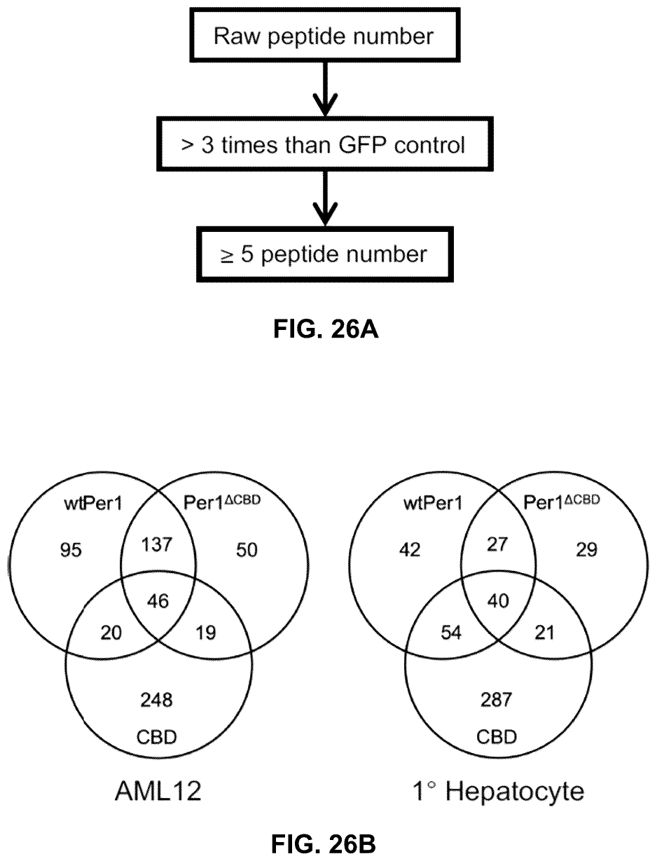

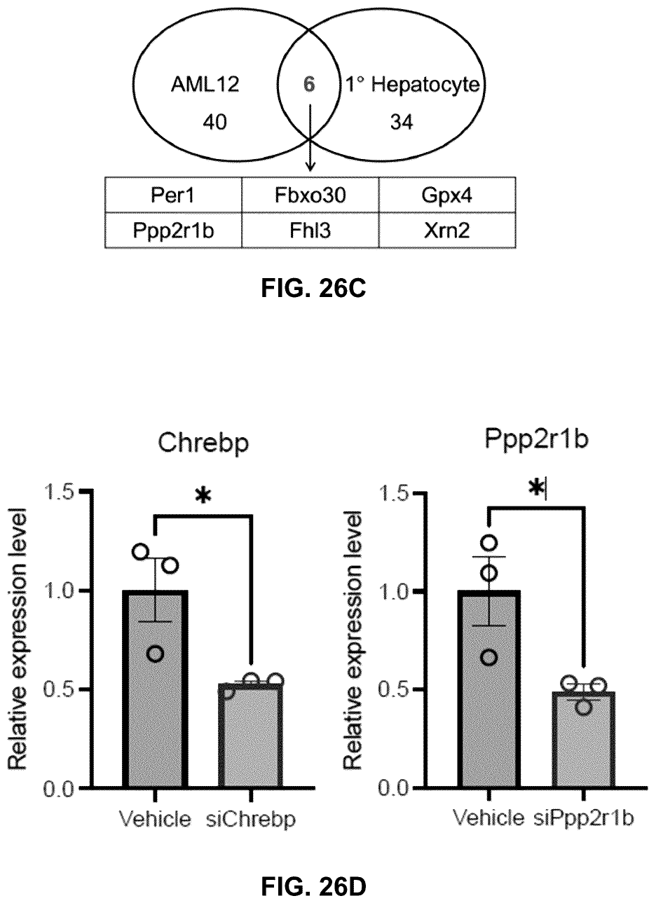

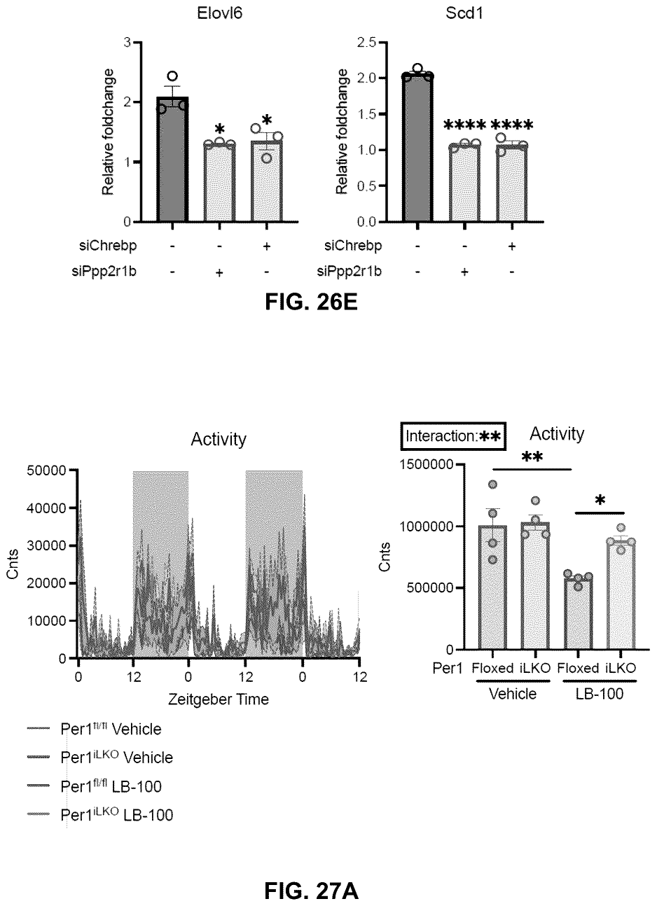

Per1, Per1^{ΔCBD} and CBD have analogous effects suppressing DNL gene expression and enhancing glucose oxidation. Therefore, immunoprecipitation was performed using Per1, Per1^{ΔCBD} and CBD as bait. This was followed by proteomic analysis to define binding partners common to each of the three constructs. This study tested the hypothesis that the effects of Per1 on lipid and energy homeostasis are reflected by Per1 effectors that bind across PER1 domains. Using an adenoviral vector, tagged Per1, Per1^{ΔCBD}, CBD, or GFP alone was overexpressed in AML12 hepatocytes and isolated primary hepatocytes from wild-type mice (FIG. 18A). Proteomic analysis validated that Per1^{ΔCBD} has minimum binding activity with CRY1/2, and that CBD fragment binds to CRY1/2 (FIG. 18B, FIG. 18C). Among all peptides that met the screening criteria, CBD fragment had the highest number of binding partners among all three constructs in both AML12 and primary hepatocyte models (FIG. 26A, FIG. 26B).

Surprisingly, 46 peptides were common binding partners across all 180 three constructs in AML12, and 40 peptides were common binding partners across all three constructs in primary hepatocytes (FIG. 26B). Among these, 6 of them were shared between the in vitro AML12 and ex vivo primary hepatocyte models (FIG. 26C). One of them—Ppp2r1b—is a gene encoding protein phosphatase 2 A subunit A (PP2A-A) (FIG. 18B, FIG. 18C, FIG. 26C). The proteomic data was validated through in vitro pull-down in AML12 and immunoblot targeting PP2A-A, which showed a clear co-immunoprecipitation between PP2A-A and PER1, Per1^{ΔCBD} and CBD constructs (FIG. 18D).

PP2A dephosphorylates the pro-lipogenic transcription factor carbohydrate-responsive element binding protein (ChREBP) to promote its nuclear entry and DNL gene transcription. This study tested if in vitro PP2A manipulation is sufficient to drive changes in lipogenic gene expression. To that end, AML12 hepatocytes (0 mM glucose, 0.5% FBS, 6 h) were serum- and glucose-starved, followed by addition of 25 mM glucose with 100 nM insulin to induce DNL (FIG. 18E). As showed previously, adding glucose suppresses Per1, and insulin further inhibits Per1 expression. In contrast, supplementing glucose and insulin were sufficient to induce lipogenic genes Elovl6 and Scd1 (FIG. 18F). To evaluate how PP2A affects lipogenic gene expression, it was demonstrated that both Elovl6 and Scd1 gene expression were dose-dependently inhibited by the PP2A small-molecule inhibitor, LB-100 (FIG. 18G). To next evaluate if PP2A activation is sufficient to promote lipogenic gene expression, cells were treated with 20 μM DT-061, a PP2A activator. DT-061 induced Fasn, Elovl6 and Scd1, indicating a positive regulation of PP2A towards lipogenesis (FIG. 18H). When Chrebp or Ppp2r1b were knocked down through small interfering RNA (siRNA), it was noticed that the activation in Elovl6 and Scd1 were blunted under glucose and insulin supplemented condition, suggesting that PP2A and ChREBP are necessary for activating lipogenic gene expression (FIG. 26D, FIG. 26E).

### PP2A Mediates Per1 Hepatic and Inter-Organ Regulation of Energy Homeostasis in Per1^{iLKO} Mice

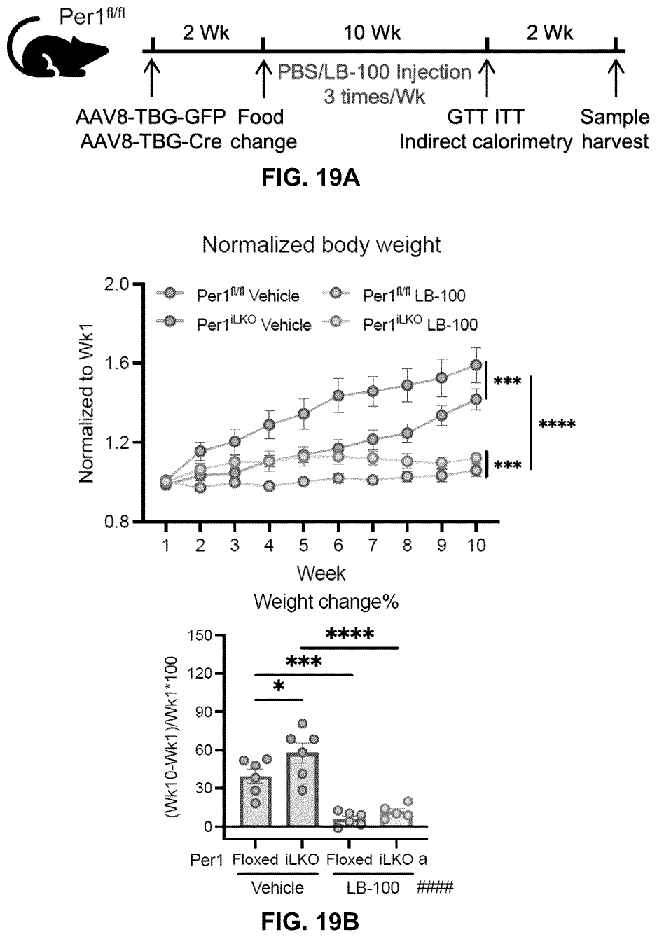

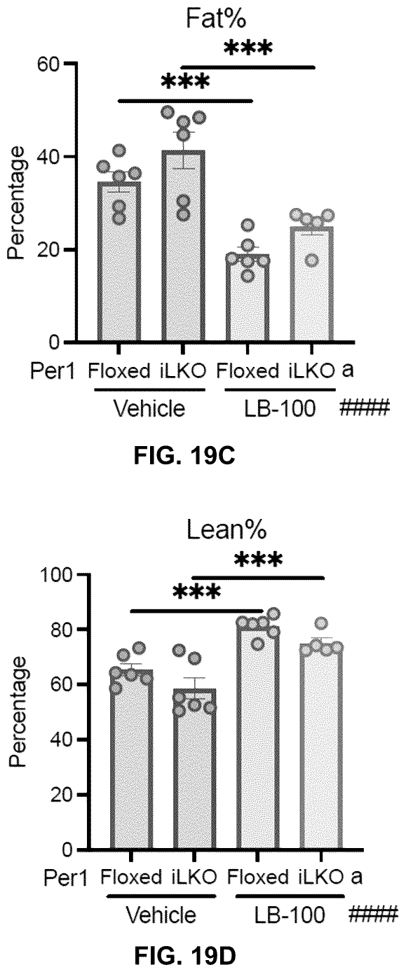

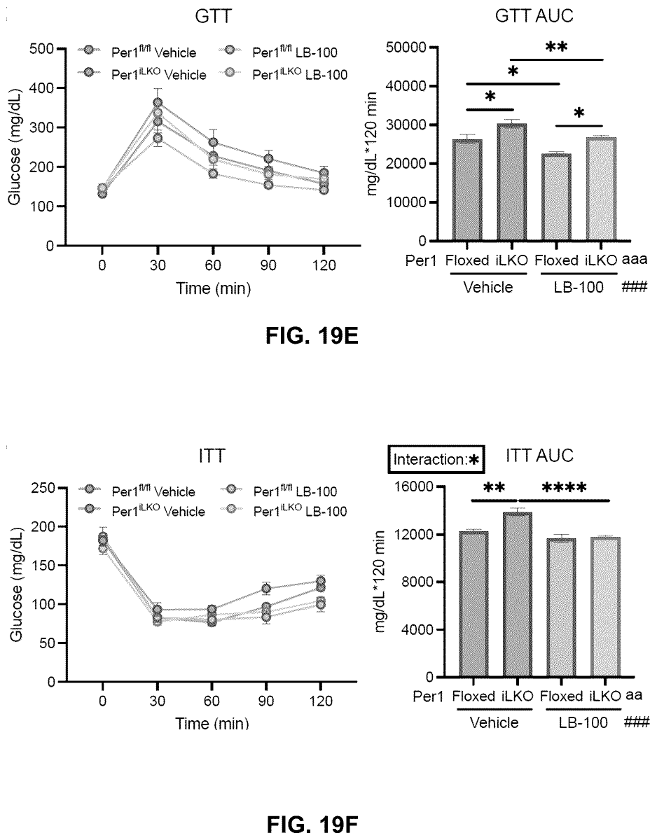

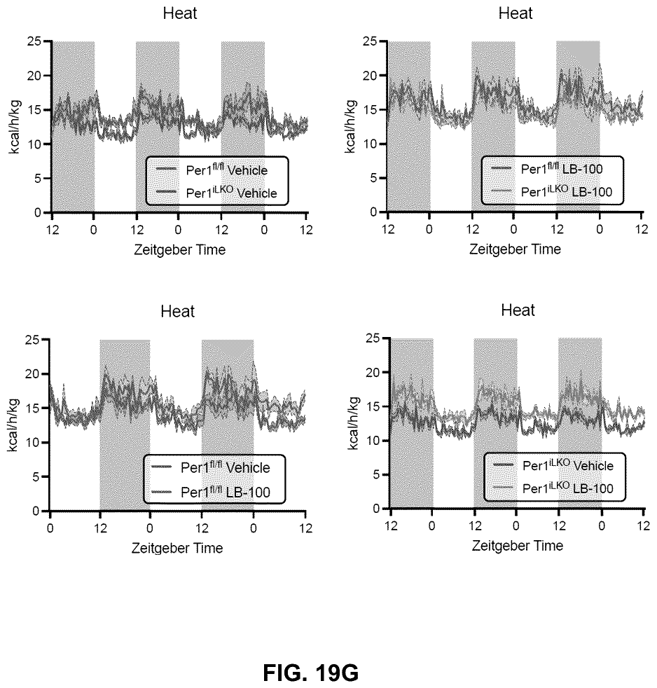

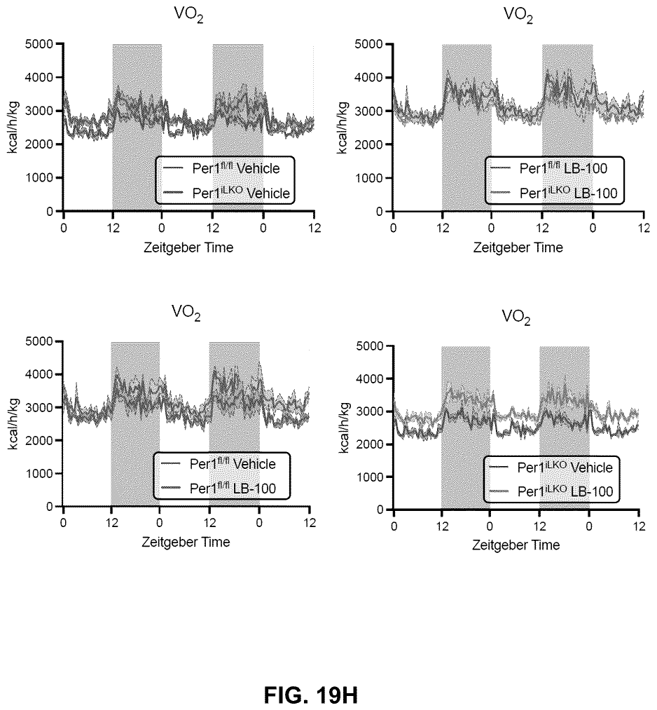

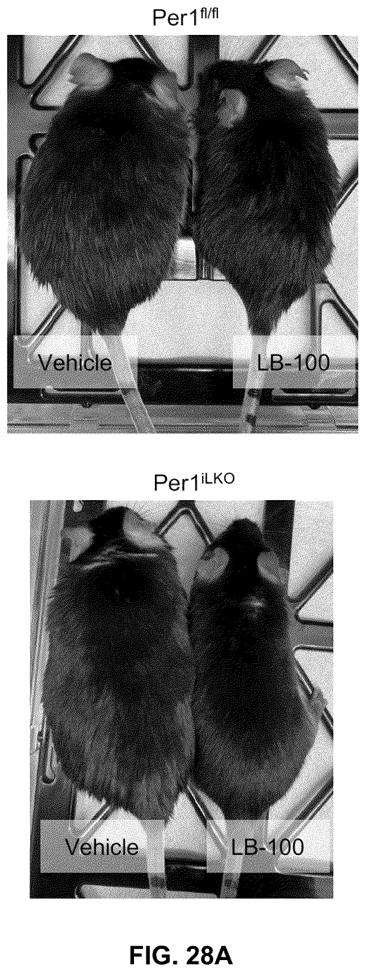

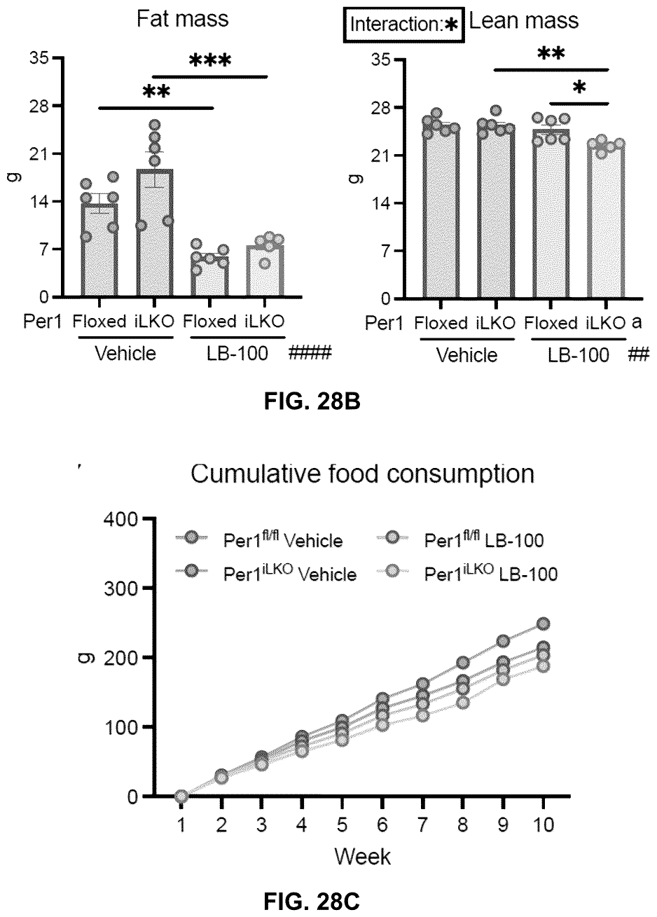

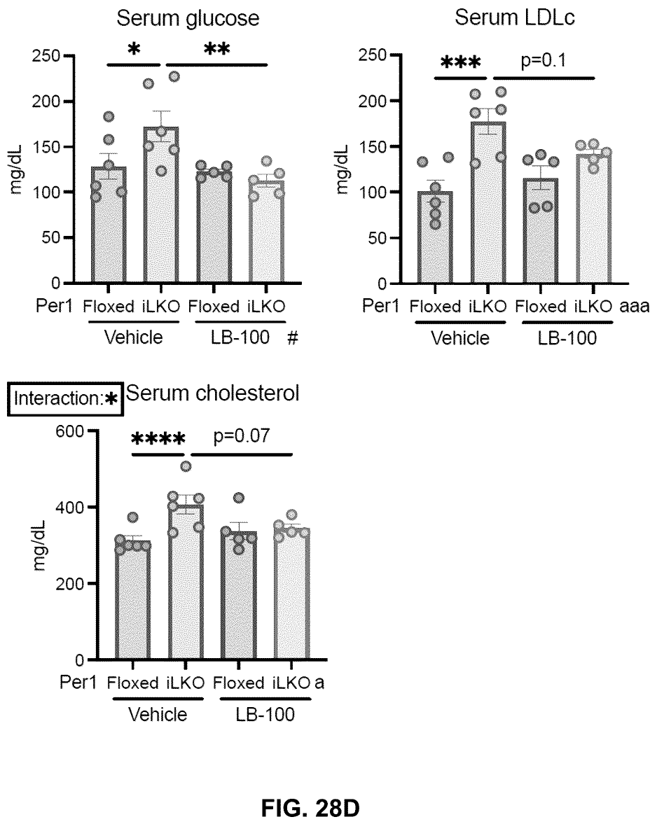

Prior data indicate that inhibiting PP2A activity ameliorates obesity induced by high fat diet. Based on the finding that Per1 and its two mutant constructs Per1^{ΔCBD} and CBD each bind to PP2A, and PP2A blocks lipogenic gene expression in hepatocytes, it was tested if LB-100 treatment in Per1^{iLKO} mice can prevent body weight gain and promote energy expenditure. PP2A was inhibited in Per1^{fl/fl} and Perf^{iLKO} mice using LB-100 (3×/wk, 12 weeks; FIG. 19A). Both Per1^{fl/fl} and Per1^{iLKO} treated with LB-100 gained less weight throughout the treatment (FIG. 19B). No significant difference was observed in total food consumption (FIG. 28C). However, at 10 wk WD exposure, Perf^{iLKO} mice treated with vehicle control gained significantly more weight than control Per1^{fl/fl} mice, whereas PP2A blockade reversed the excess weight gain when comparing the two genotypes (FIG. 19B, FIG. 28A). PP2A blockade also significantly reduced fat mass and fat percentage, and increased lean mass percentage in both genotypes, with a mild reduction in lean mass in Per1^{iLKO} mice (FIG. 19C, FIG. 19D, FIG. 28B). Glucose and insulin tolerance testing revealed that Per1^{iLKO} mice treated with vehicle again exhibited higher glucose throughout the measurement, and PP2A blockade in Per1^{iLKO} mice significantly reduced glucose levels in both assays (FIG. 19E, FIG. 19F).

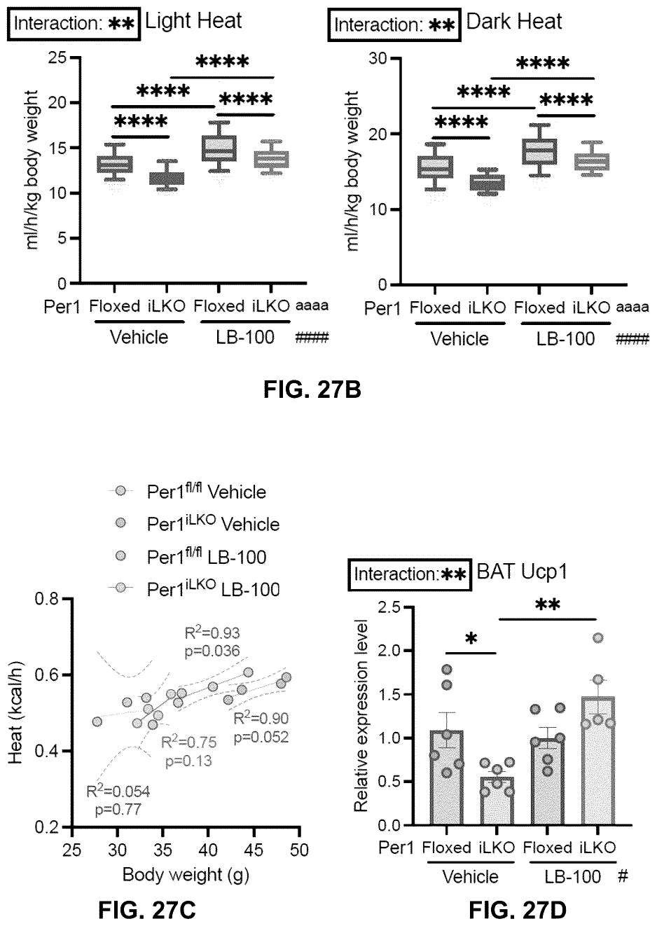

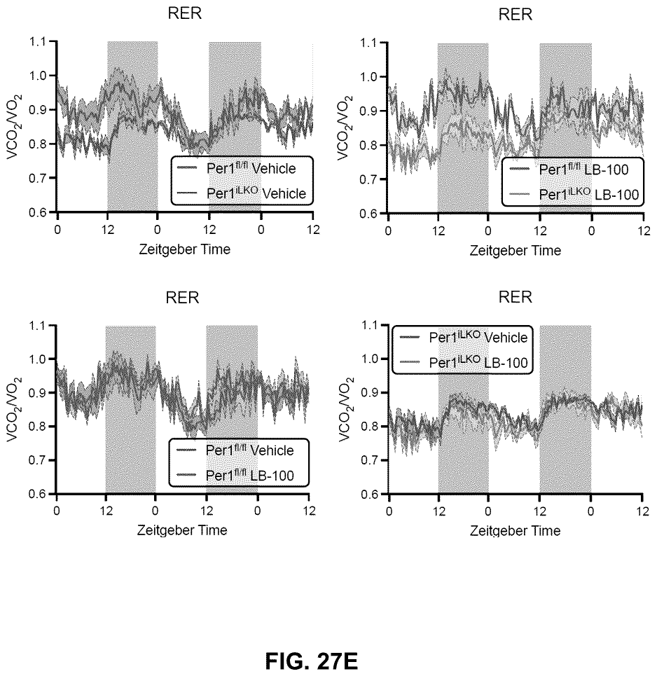

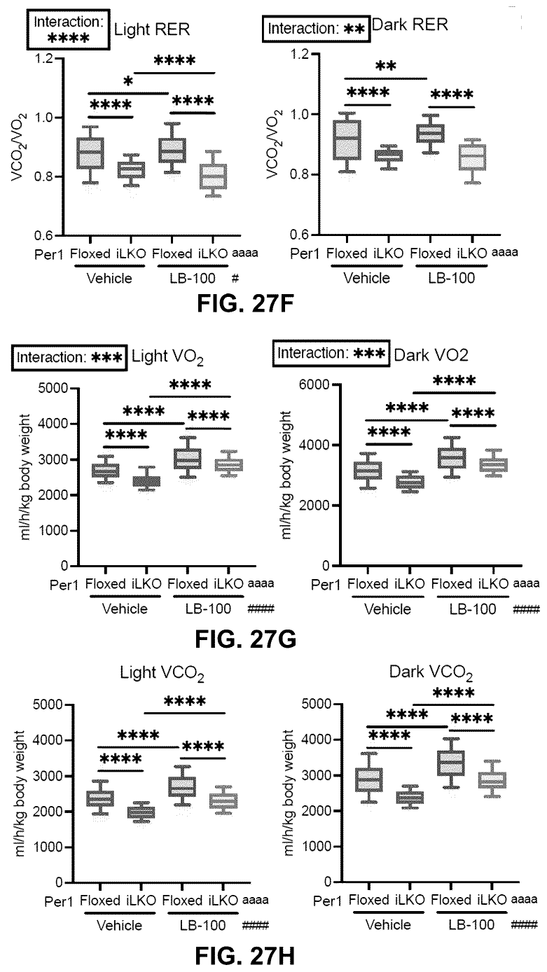

To define changes in whole-body oxidative metabolism, energy expenditure was quantified by indirect calorimetry. PP2A blockade in Per1^{fl/fl} mice lowered total locomotion when compared with mice in other groups (FIG. 27A). Vehicle-treated Per1^{iLKO} again showed significantly lower heat production than Per1^{fl/fl} mice in both light and dark phases, whereas LB-100 treatment significantly increased energy expenditure in both genotypes in both phases (FIG. 19G, FIG. 27B). A strong correlation between body weight and head production was found in vehicle treated mice but not in LB-100-treated mice (FIG. 27C). Then, expression of mitochondrial thermogenic gene Ucp1 was measured in brown adipose tissue (BAT) from these mice. It was found that indeed Perf^{iLKO} mice under vehicle treatment had lower Ucp1 expression than control Per1^{fl/fl} mice, and LB-100 treatment significantly increased Ucp1 expression in Per1^{iLKO} mice (FIG. 27D). PP2A blockade in Per1^{fl/fl} mice also significantly increased RER in both light and dark cycles, with slightly reduced RER in Perf^{iLKO} mice during light phase without apparent changes in dark phase RER (FIG. 27E, FIG. 27F). LB-100 treatment in both genotypes also significantly induced in VO2 and VCO2 (FIG. 19H, FIG. 19I, FIG. 27G, FIG. 27H). Overall, inhibiting PP2A induces whole body oxidative metabolism and thermogenesis, and significantly reverses impaired energy expenditure observed in Per1^{iLKO} mice.

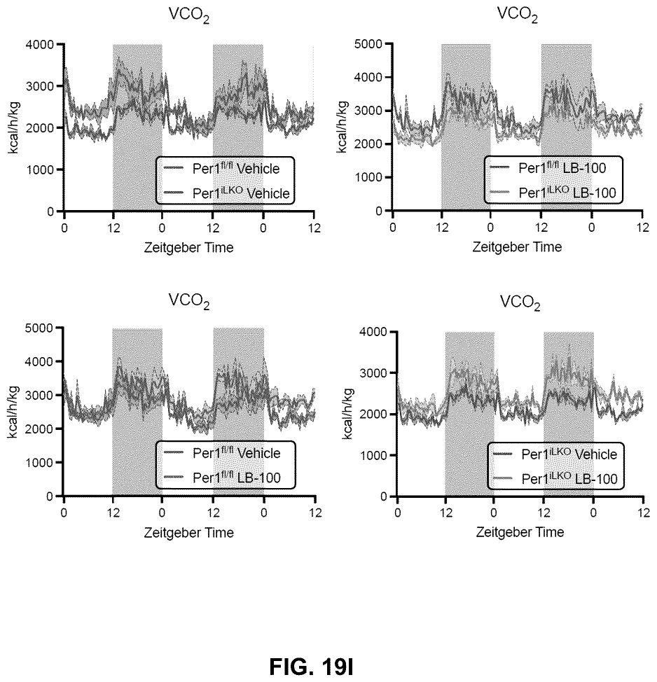

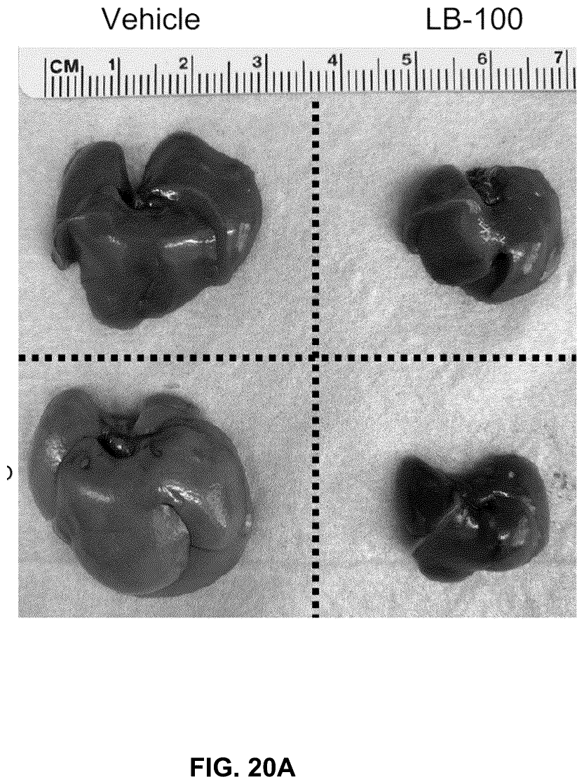

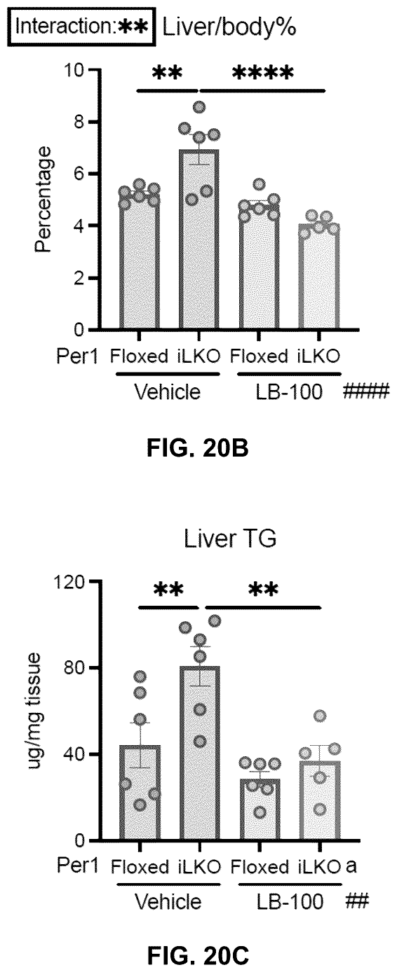

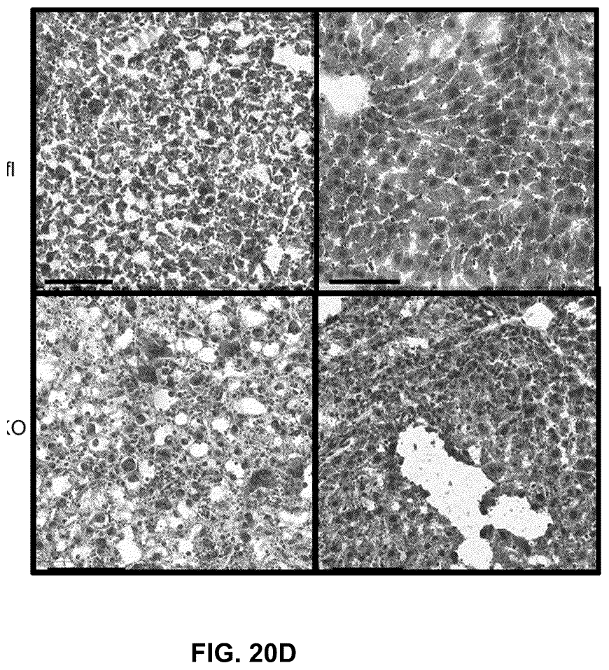

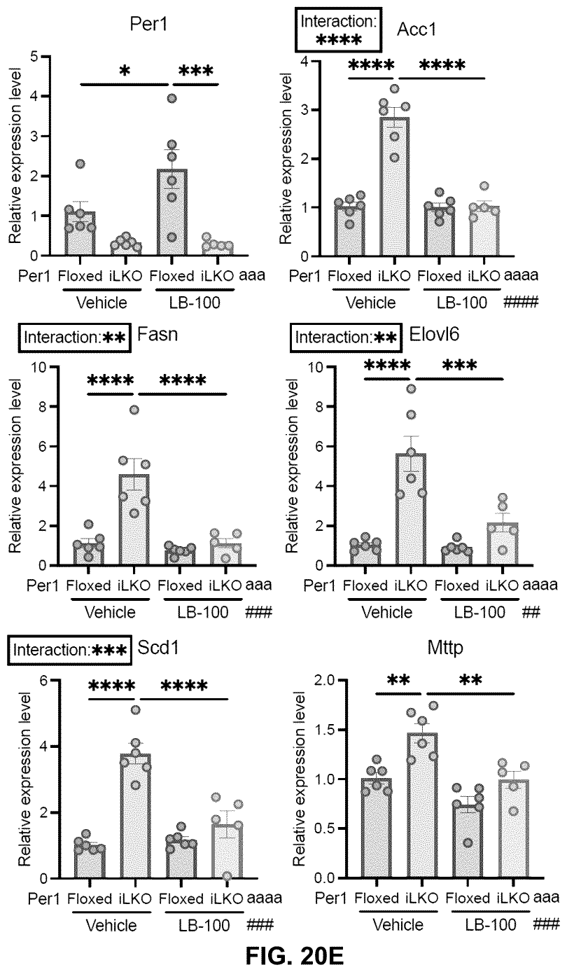

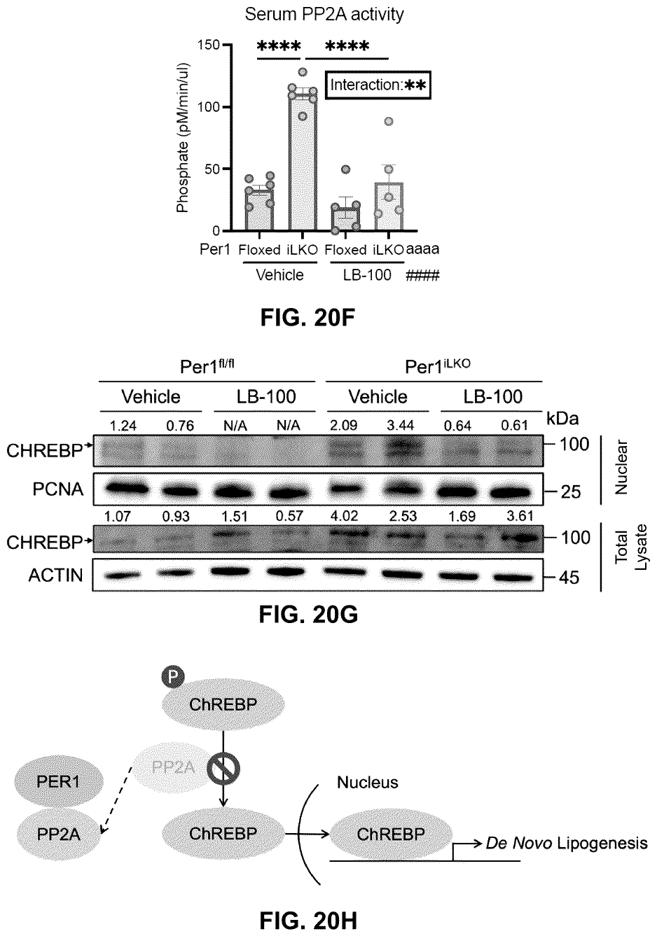

Next, hepatic lipids and DNL gene expression were examined in mice treated PP2A inhibitor. This study first observed larger, paler liver tissue in Perf^{iLKO} mice compared to Per1^{fl/fl} mice injected with vehicle control. However, PP2A inhibition reduced the liver size in both genotypes, and Perf^{iLKO} mice treated with PP2A inhibitor resulted in a darker and healthier liver tissue (FIG. 20A). Associated with a smaller liver size in PP2A inhibitor-treated Per1^{iLKO} mice, it was also found that the liver/body weight percentage in Per1^{iLKO} mice treated with PP2A inhibitor was significantly lower than vehicle control (FIG. 20B). Additionally, Per1^{iLKO} mice under vehicle control accumulated more liver TG than control Per1^{fl/fl} mice. PP2A blockade significantly reduced liver TG in Perf^{iLKO} mice, and this was further corroborated by ORO staining (FIG. 20C, FIG. 20D). Serum analysis revealed that PP2A inhibitor lowered glucose in Per1^{iLKO} mice, and produced a trend toward lower LDLc and cholesterol when compared with vehicle treated Perf^{iLKO} mice (FIG. 28D).

Gene expression in these mice first validated the knockout in hepatocyte Per1. Similar to the in vitro data, PP2A inhibition significantly induced Per1 in Per1^{fl/fl} mice (FIG. 20E). In addition, PP2A blockade significantly suppressed DNL gene expressions, including Acc1, Fasn, Elovl6, Scd1, Mttp and Gpat, which were significantly activated in vehicle-treated Per1^{iLKO} mice (FIG. 20E). To evaluate the PP2A activity in vehicle control mice, and to validate if LB-100 successfully inhibits PP2A, PP2A activity was quantified from serum of Per1^{fl/fl} mice and Per1^{iLKO} mice. In vehicle-treated controls, Per1^{iLKO} mice demonstrated significantly higher PP2A activity than Per1^{fl/fl} mice, whereas LB-100 blocked PP2A activity in Per1^{iLKO} mice (FIG. 20F). Given the previously established role of PP2A in promoting ChREBP nuclear localization, nuclear protein was extracted from liver tissues in Per1^{fl/fl} and Per1^{iLKO} mice with or without PP2A blockade. Vehicle-treated Per1^{iLKO} mice had higher nuclear ChREBP level than Per1^{fl/fl} mice, which matches with the finding in hyperactive PP2A in Perf^{iLKO} mice. In contrast, PP2A inhibitor reduced nuclear ChREBP level in both genotypes (FIG. 20G). Vehicle-treated Per1^{iLKO} mice also had higher total ChREBP level in their liver tissues, but PP2A inhibition did not significantly change the total ChREBP abundance (FIG. 20G). In summary, it was found that mice lacking hepatocyte Per1 exhibit hyperactive PP2A activity, and pharmacological inhibition of PP2A is sufficient to reverse the predisposed development of obesity and fatty liver in Per1^{iLKO} mice (FIG. 20H).

### A Hepatocyte PER1 to PP2A Axis Reshapes the Hepatic Phosphoproteome and Key Metabolic Pathways

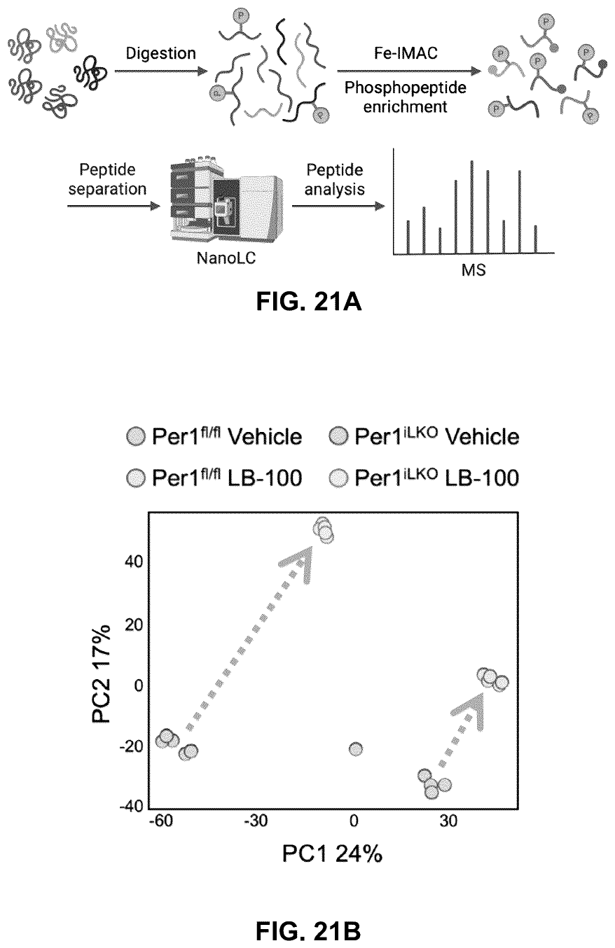

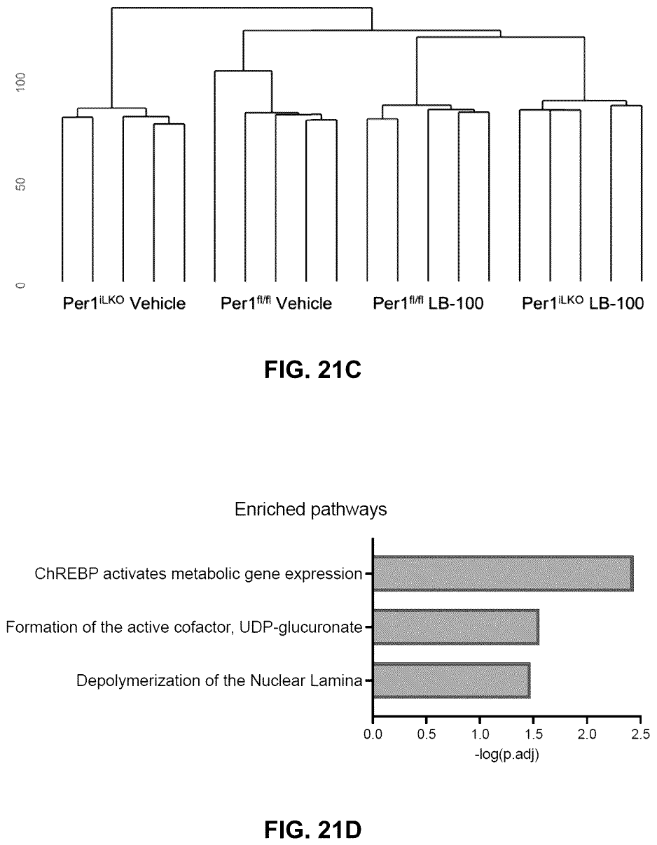

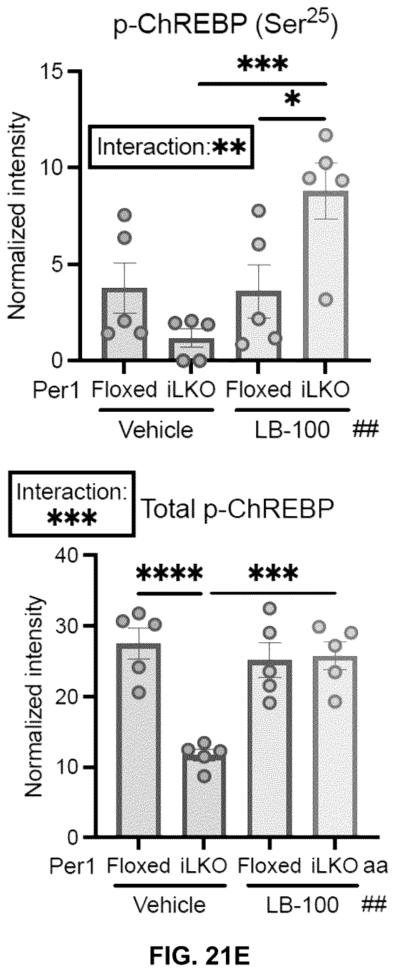

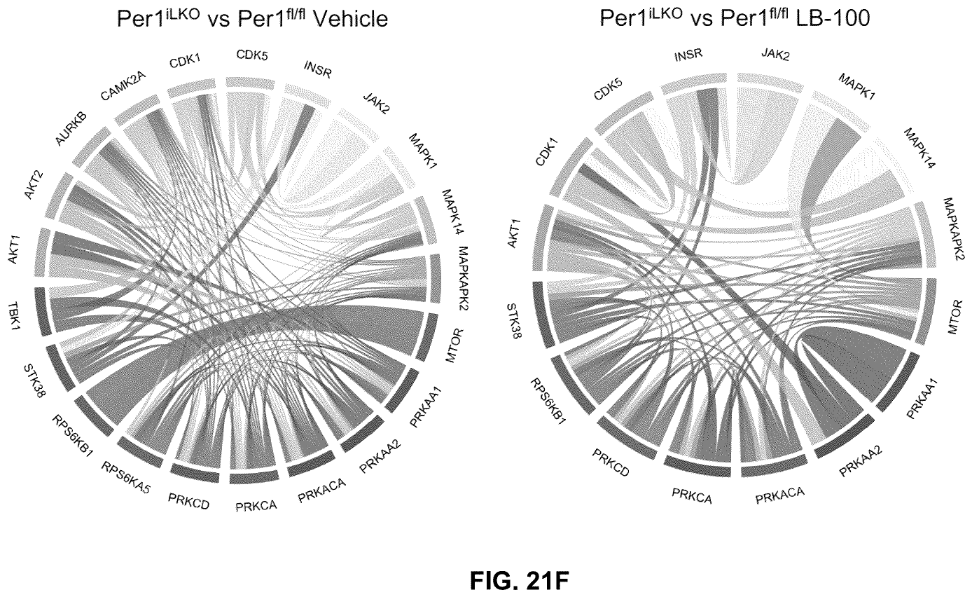

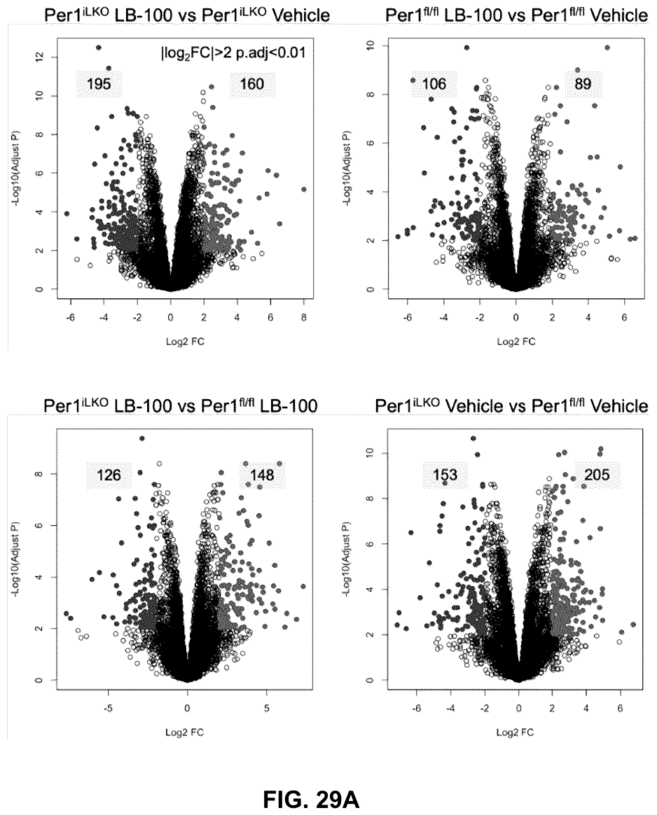

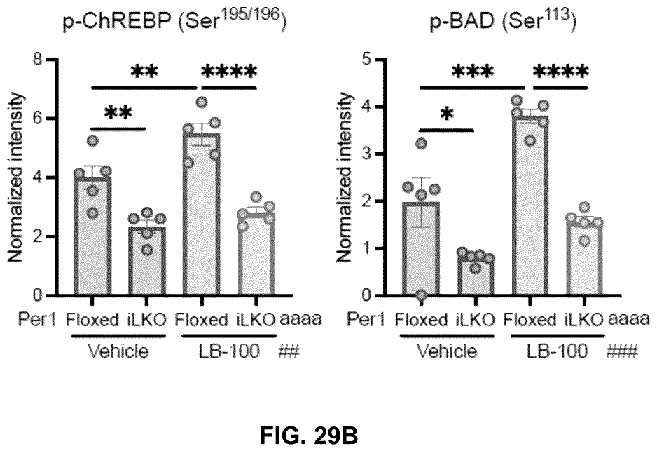

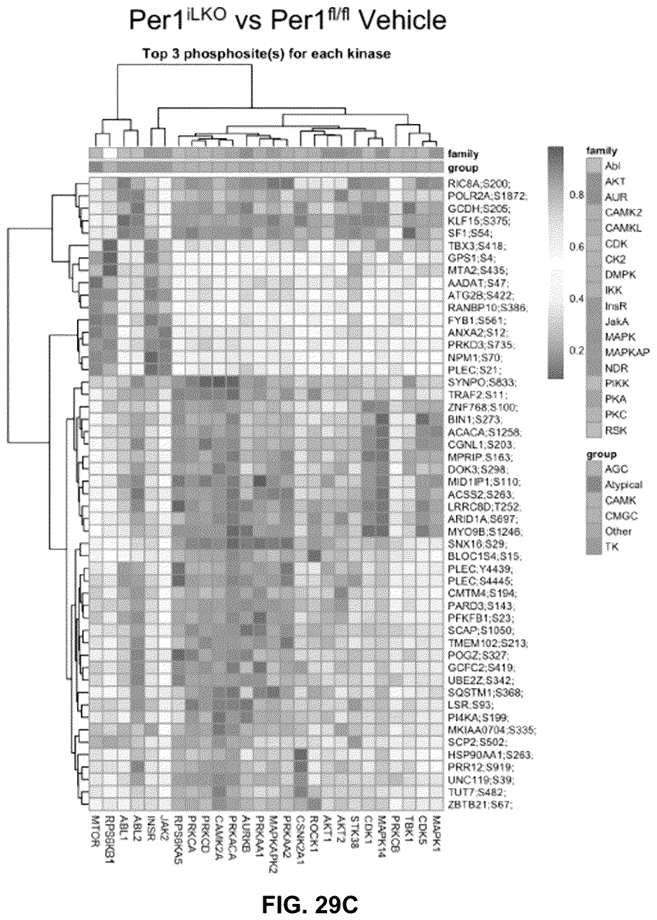

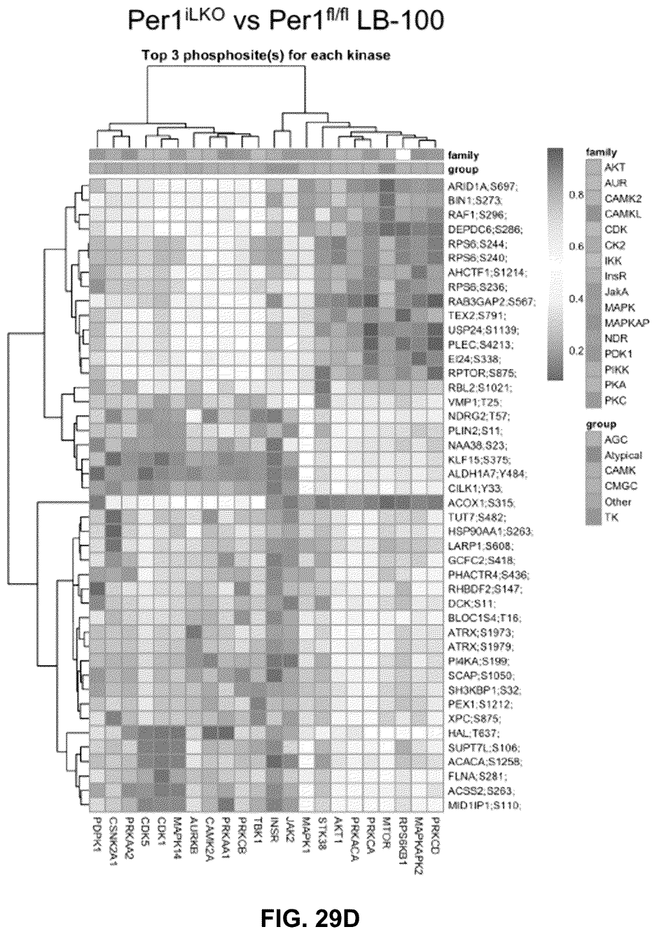

PP2A functions as a serine/threonine phosphatase and mediates phosphorylation of multiple targets. Therefore, it was hypothesized that antagonistic PER1-PP2A relationship reshapes the phosphoproteome and signalome beyond ChREBP phosphorylation and localization. To test this, livers from Per1^{fl/fl} and Per1^{iLKO} mice were subjected with or without LB-100 treatment to Fe-IMAC phosphopeptide enrichment and Nano Liquid Chromatography (NanoLC) peptide analysis (FIG. 21A). Consistent with this hypothesis, the basal hepatic phosophoproteome was easily distinguished, with 153 down- and 205 upregulated phosphorylated peptides in control livers from WD-fed Per1^{iLKO} versus Per1^{fl/fl} mice. PP2A inhibition reduced these two numbers to 126 and 148 correspondingly between Per1^{iLKO} and Per1^{fl/fl} liver (FIG. 29A). Principal component analysis (PCA) revealed four distinct clusters across genotypes and treatments, and hierarchical clustering showed that both genotypes under LB-100 treatment were more closely related than the vehicle condition (FIG. 21B, FIG. 21C). Pathway enrichment analysis using differentially regulated phosphor-targets across all four groups identified ChREBP-activated metabolic gene expression as the most significantly changed pathway (FIG. 21D). Then, phosphorylation was examined at two established PP2A target sites (ChREBPSer195/196 and BADSer113) to confirm the effectiveness of LB-100. It was found that LB-100 treatment significantly increased the level of p-ChREBPSer195/196 and p-BADSer113 level in Per1^{fl/fl} mice, and more importantly, Per1^{iLKO} mice under vehicle treatment had significantly lower p-ChREBPSer195/196 and p-BADSer113 when compared to vehicle-treated Per1^{fl/fl} mice (FIG. 29B). These data further support a higher PP2A activity in Per1^{iLKO} liver, and validate the inhibition of PP2A by LB-100. Among all phosphorylated sites detected in ChREBP, ChREBPSer25 was more phosphorylated in LB-100-treated Per1^{iLKO} mice than the vehicle control, and total phosphorylated ChREBP level was the lowest in vehicle-treated Per1^{iLKO} mice but was raised to the same level as in Perfil/fl mice after PP2A blockage (FIG. 21E).

More specific analysis revealed interacting kinase/target dyads relationships that were reversed upon PP2A inhibition. This included key fed/fasting pathways signals, such as the mTOR-S6K and AMPK pathway. Strikingly the phosphor-signalome was heavily intertwined in untreated WD-fed Perf^{iLKO} versus Per1^{fl/fl} liver, but PP2A blockade muted the number and breadth of connections in this signalome (FIG. 21F, FIG. 29C, FIG. 29D), and specifically restricted the AMPK connection while dispersing the mTOR signaling away from anabolic function (S6K) toward insulin signalling (AKT1) and autophagic flux induction (e.g. MAPK1) (FIG. 21F). Together the data indicate that PP2A reshapes the hepatocyte phosphoproteome to link hepatocyte Per1 to hepatic and peripheral metabolic regulation in obesity.

### DISCUSSION

The circadian clock was first established in the suprachiasmatic nucleus in brain as a transcriptional-translational feedback loop composed of a group of genes namely the core circadian genes. Subsequent studies have identified a similar rhythmic behavior of the core circadian genes—Clock, Bmal1, Per1/2, Cry1/2 and Rev-erb-a/b—in other peripheral tissues including the liver, kidney, heart and muscle. Given the central function in these peripheral tissues to maintain metabolic homeostasis, multiple studies have focused on dissecting the interconnection between the circadian clock in these organs and their metabolic consequences. Mice challenged with high-fat diet developed a dampened circadian rhythm in the liver, and reversely, mice lacking key circadian components in the liver are more prone to the development of metabolic diseases including obesity and MASLD. These studies collectively demonstrate a reciprocal regulation between the liver clock and metabolism. However, the degree to which members of the core circadian clock genes integrate circadian and metabolic inputs remains incompletely elucidated.

Beyond transcriptional regulation, the circadian clock mediates intracellular signaling events through post-translational modifications, including phosphorylation and acetylation. More than a quarter of hepatic proteins maintain a rhythmic phosphorylation in a daily basis, including central metabolic mediators AKT and mTOR, and also other core circadian genes, such as the PERIOD protein family and BMAL1/CLOCK complex. Together, the data highlight how global phosphorylation maintains a functional liver circadian and metabolic clock.

Period 1 is a canonical circadian gene within the Period family. It functions through direct binding to CRY1/2 and translocate to nucleus to inhibit BMAL1:CLOCK binding to its promoter. Prior work has shown that Per1 and its homolog Per2 are not functionally redundant, and Per2 but not Per1 is required for normal circadian clock rhythmicity. Example 1 reported a circadian-independent function of hepatocyte Per1 during fasting. This study showed that fasting induces hepatocyte Per1, and this drives the glucose-to-fat substrate selection that is required for optimal fasting metabolism in the absence of glucose. Notably, this metabolic regulation proceeds independently of time of day, and independently of a fully functioning molecular clock. Per1 thus acts both as a rhythmic circadian and metabolic gene that separately and additively responds to fasting and carbohydrate exclusion from the hepatocyte to optimize fuel selection. Again, its function in the context of obesity and insulin resistance remains unclear.

Here, it was hypothesized that the systemic sequelae of diet-induced obesity are predicated on metabolic regulation by hepatocyte Per1. This is based on the observation that Per1 is a key fasting-responsive hepatic control node. Loss of hepatocyte Per1 function predisposed mice to diet-induced obesity, glucose intolerance, hepatic steatosis and de novo lipogenic gene expression. This occurs through direct PP2A phosphatase binding and phosphor-regulation. The data are important because they identify Per1 action beyond transcriptional regulation, and extend its action to post-translational control over PP2A. Second, Per1 integrates nutritional and circadian signals to block metabolic liver and peripheral disease. Most importantly, the data support the hypothesis that the hepatocyte phosphoproteome is labile to both circadian and macronutrient control, and that Per1 integrates these signals. Nevertheless, one definitive arm of this global regulation that was shown is the PER1-PP2A-ChREBP axis, which blocked diet-induced hepatic fat accumulation. Specifically, Per1 deletion exacerbated, whereas hepatocyte Per1 overexpression blocked diet-induced hepatic TG accumulation and hepatocyte DNL gene expression. Aligned with this new finding, prior data revealed that Per1 shifts substrate predilection toward fat oxidation. This dual function of hepatocyte Per1 effectively shunts fatty acids away from storage as triglycerides to maintain substrate availability for oxidation. This is adaptive both during physiological fasting and the obese/insulin resistant state.

This study extend the findings translationally to show that a minimal PP2A binding domain—the CRY binding domain peptide of PER1—is sufficient to exert much of the effect of full-length of Per1 on hepatocyte DNL, dyslipidemia and energy expenditure. These data again support a transcriptionally independent function for Per1 in metabolic control. Also, the use of the CRY binding domain peptide may be a translatable biologic agent to abate DNL. Finally, the data raise the possibility that PER1, CRY1/2 and PP2A compete for binding and function, and that Per1 induction during fasting or throughout the clock fine-tunes the phosphoproteome and phosphor-signalome.

### Materials and Methods

### Animals

Bmal1^{fl/fl} mice (Jackson Laboratory, 007668) were obtained directly from the Jackson Laboratory. Per1^{fl/fl} mice were generated by Genome Engineering & iPSC Center (GEiC) at Washington University. Upon arrival, mice were equilibrated for a minimum of 7 days in the specific pathogen-free vivarium prior to initiating metabolic measurements. Mice were kept under a 12 h alternating light/dark, temperature-controlled facility throughout the experimentation. All in vivo experimental procedures were performed in strict accordance with Institutional Animal Care and Use Committee (IACUC) guidelines at Washington University School of Medicine. Male mice were used unless specified. All experiments were done when mice are 8-week-old. For in vivo LB-100 treatment, LB-100 (Selleck Chemistry, S7537) was dissolved in PBS and 1.5 mg of the drug per kg of total body weight was i.p. injected to mice three times a week.

### Cell Culture

AML12 cells (CRL-2254) were purchased directly from the American Type Culture Collection (ATCC) and propagated and maintained precisely per manufacturer specification. Cells were cultured in complete media (Dulbecco's modified Eagle's medium/nutrient mixture F-12 (DMEM/F12; ThermoFisher, 11,320-033) supplemented with 10% fetal bovine serum (Gibco, 26140079), 40 ng/ml dexamethasone (Sigma Aldrich, D4902) and insulin-transferrin-selenium solution (Sigma, 11884)). Primary murine hepatocytes obtained from wildtype, Per1^{fl/fl} and Per1^{LKO} mice were isolated and maintained in regular DMEM growth media (Sigma Aldrich, D5796) containing 10% FBS.

### Virus Injection

Adeno-associated viruses under TBG promoter overexpressing GFP (AAV8-TBG-GFP), Cre (AAV8-TBG-Cre), wtPer1, Per1^{ΔCBD} and CBD were obtained as ready-to-use viral stock from Vector Biolabs (Malvern, PA, USA). 10¹¹ viral particles were injected via tail vein 10-14 days prior to diet change. Standard rodent chow diet or Western Diet (Inotiv, TD.88137) were used as specified in the results.

### Serum and Hepatic Lipids, Indirect Calorimetry

For all serum analyses, submandibular blood was collected immediately prior to sacrifice and serum was separated. Triglycerides (ThermoFisher, TR22421), LDLc (FUJIFILM, 990-28011, 993-00404, 999-00504), free fatty acids (FUJIFILM, 999-34691, 995-34791, 991-34891, 993-35191), glucose (Cayman, 10009582), albumin (Sigma Aldrich, MAK124) quantification were performed using commercially available reagents according to manufacturer's directions. Serum PP2A activity measurement was done using Ser/Thr Phosphatase Assay Kit 1 (K-R-pT-I-R-R) upon manufacture's instruction (EMD Millipore Corporation, 17-127). Hepatic lipids, histology and indirect calorimetry analyses were performed.

### Glucose and Insulin Tolerance Testing

Glucose tolerance test was performed in mice fasted for 6 hours. Mice were weighted and injected with 30% glucose solution dissolved in saline 2 g/kg body weight. Blood glucose level was measured using glucometer every 30 minutes. Similarly, insulin solution was injected in mice fasted for 4 hours 0.75 units/kg body weight (Humulin R, NDC 0002-8215-17). Blood glucose level was measured using glucometer every 30 minutes.

Quantitative Real-Time PCR (qRT-PCR)

Total RNA was prepared by homogenizing snap-frozen livers or cultured cells in Trizol reagent (Invitrogen, 15596026) according to the manufacturer's protocol. cDNA was prepared using Qiagen Quantitect reverse transcriptase kit (Qiagen, 205310). Real-time qRT-PCR was performed with Step-One Plus Real-Time PCR System (Applied Biosystems) using SYBR Green master Mix Reagent (Applied Biosystems) and specific primer pairs. Relative gene expression was calculated by a comparative method using values normalized to the expression of the internal control gene β-Actin. All primers were custom-synthesized oligonucleotides obtained from Integrated DNA Technologies. Primer sequences are included in Table 5.

TABLE 5
qRT-PCR primer sequences

| Target | Forward (5′-3′) | Reverse (5′-3′) |
|---|---|---|
| Actin | GATTACTGCTCTGGCTCCTAG | GACTCATCGTACTCCTGCTTG |
| | (SEQ ID NO: 1) | (SEQ ID NO: 2) |
| Acc1 | TGTCCGCACTGACTGTAACCA | TGCTCCGCACAGATTCTTCA |
| | (SEQ ID NO: 33) | (SEQ ID NO: 34) |
| Bmal1 | TGACCCTCATGGAAGGTTAGAA | GGACATTGCATTGCATGTTGG |
| | (SEQ ID NO: 5) | (SEQ ID NO: 6) |
| Chrebp | CTGGGGACCTAAACAGGAGC | GAAGCCACCCTATAGCTCCC |
| | (SEQ ID NO: 35) | (SEQ ID NO: 36) |
| Elov16 | GGAAAGCAGTTCAACGAGAACG | AGATGCCGACCACCAAAGATA |
| | (SEQ ID NO: 37) | (SEQ ID NO: 38) |
| Fasn | CCTGGATAGCATTCCGAACCT | AGCACATCTCGAAGGCTACACA |
| | (SEQ ID NO: 39) | (SEQ ID NO: 40) |
| Gpat | CAACACCATCCCCGACATC | GTGACCTTCGATTATGCGATCA |
| | (SEQ ID NO: 41) | (SEQ ID NO: 42) |
| Mttp | CTCTTGGCAGTGCTTTTTCTCT | GAGCTTGTATAGCCGCTCATT |
| | (SEQ ID NO: 43) | (SEQ ID NO: 44) |
| Per1 | CGGATTGTCTATATTTCGGAGC | TGGGCAGTCGAGATGGTGTA |
| | A (SEQ ID NO: 45) | (SEQ ID NO: 46) |
| Per1 | ATCCTGGCCACTCTGATGAC | TTCTTCCTCCATGGCAGAGT |
| (CBD) | (SEQ ID NO: 47) | (SEQ ID NO: 48) |
| Ppp2r1b | TCGCGGTTTTAATCGACGAG | CTACCCCGAGTGCTAGAGCTA |
| | (SEQ ID NO: 49) | (SEQ ID NO: 50) |
| Scd1 | ACGCCGACCCTCACAATTC | CAGTTTTCCGCCCTTCTCTTT |
| | (SEQ ID NO: 51) | (SEQ ID NO: 52) |
| Srebp1c | CCATGGATTGCACATTTGAA | GAAGTCACTGTCTTGGTTG (SEQ |
| | (SEQ ID NO: 53) | ID NO: 54) |
| Ucp1 | AGGCTTCCAGTACCATTAGGT | CTGAGTGAGGCAAAGCTGATTT |
| | (SEQ ID NO: 55) | (SEQ ID NO: 56) |

### In Vitro Lipogenic Activation

For in vitro de novo lipogenesis experiment, 1*10⁶ cells (per well) were seeded in 6-well plates. After overnight attachment, media were switched to DMEM no glucose (Gibco, 11966025) supplemented with 0.5% FBS for 6 hours. After 6-hour starvation, to induce de novo lipogenesis, cells were supplemented with 25 mM glucose with 100 nM insulin (Gibco, 11965092; Humulin R, NDC 0002-8215-17) for 6 hours. For in vitro experiment knocking down Chrebp and Ppp2r1b, cells were transfected with siRNA purchased directly from Thermo Fisher Scientific (sMIxipl, ID181945; siPpp2r1b, ID284489). Transfection was performed according to the Lipofectamine 3000 (Invitrogen, L3000015) transfection protocol. For in vitro treatment of LB-100 and DT-061 in AML12 cells, 0-40 uM LB-100 dissolved in PBS (Selleck Chemistry, S7537) or 20 uM DT-061 dissolved in DMSO (Selleck Chemistry, S8774) were supplemented in media with 25 mM glucose and 100 nM insulin for 6 hours.

### Immunoprecipitation

For in vitro and ex vivo pull-down assays in AML12 cells and primary hepatocytes, cells were cultured in complete media as described previously. Adenovirus overexpressing GFP (Cat #1060) or N-terminal GFP-tagged wtPer1, 1 Per1^{ΔCBD} or N-terminal HA-tagged CBD constructs under CMV promoter were designed and acquired from Vector Biolab (Malvern, PA, USA). 1×10⁸ PFU/ml or 1×10⁷ PFU/ml were added to culture media in AML12 and primary hepatocytes, respectively. 48 h post-transfection in AML12 or 24 h in primary hepatocytes, proteins were harvested in cell lysis buffer (Cell Signaling Technology, #9803) and quantified using BCA kit (Thermo Scientific, 23225). Pull-down for GFP-tagged constructs was performed using biotinylated GFP antibody (abcam, ab6658) and streptavidin magnetic beads (Thermo Scientific, 88816). Pull-down for HA-tagged CBD was performed using Pierce Magnetic HA-Tag IP/Co-IP Kit (Thermo Scientific, 88838). Pull-down was performed according to streptavidin magnetic beads protocol. Briefly, 500 ug of total lysate was incubated with 1 ul of biotinylated antibody overnight on 4° C. shaker and transferred to 20 ul streptavidin magnetic beads. Mixture of samples and beads were incubated 1 hour on 4° C. shaker, and beads were washed three times with cold PBS using magnetic stand. Beads were then incubated with 50 ul loading buffer and heated at 99° C. for 5 minutes. Supernatants were then assayed and analyzed through immunoblot. Pull-down proteomic (data not shown).

### Immunoblotting

Protein from tissues and cells were harvested using RIPA buffer (Cell Signaling Technology, 9806) supplemented with protease of phosphatase inhibitor (Thermo Fisher Scientific, A32961). Protein concentration was determined by BCA. Extracted protein samples were mixed with 2× Laemmli Sample Buffer (Biorad, 1610737) supplemented with 2-Mercaptoethanol in 1:1 volume ratio. Mixed samples were heated at 99° C. for 5 minutes and then analyzed using SDS-PAGE Gels (Biorad).

RNA-seq and snRNA-seq

RNA sequencing was performed by the Washington University Genome Technology Access Center (GTAC). Differentially expressed genes analysis was performed using edgeR package. Liver nuclei for snRNA-seq were isolated using chromium nuclei isolation kit with RNase inhibitor (10× genomics, 1000494). Isolated nuclei were processed and sequenced by Washington University GTAC, and data were analyzed by Seurat package.

### Ex Vivo Tracing

For ex vivo glucose tracing experiments, primary hepatocytes from Per1^{fl/fl} and Per1^{LKO} (Per1^{fl/fl}, Alb-Cre) male mice were isolated. 24 h post-seeding, cells were treated with 10⁷ PFU Ad-GFP for 24 h, and media was switched to U¹³C-glucose complete media composed of DMEM no glucose no glutamine (Gibco, A1443001) supplemented with 25 mM U¹³C-glucose (Cambridge Isotope Laboratories, CLM-1396-10) and 4 mM unlabeled glutamine (Sigma, G8540-100G). 24 h after media change, cells were fixed, and metabolites were extracted according to the protocol from the Human Metabolome Technologies (HMT) ver.ACB.1.0.0. Samples were frozen and stored in −80° C. upon shipment to HMT for CE-MS analysis.

### Phosphoproteomic Analysis

Frozen liver samples were sent to Creative Proteomics (New York, USA) for processing and downstream analysis. Briefly, samples were removed from −80° C. and homogenized in 200 μL lysis buffer (8 M urea, 100 mM tris-HCl, pH 8.0, 1% protease inhibitor, 1% phosphatase inhibitor) by sonication. Protein concentration was determined using a BCA kit and 40 μg protein was used for each sample. Disulfide bridges were reduced by 10 mM TCEP at 56° C. for 1 h. Reduced cysteine residues were then alkylated by 20 mM iodoacetamide (IAA) in the dark at room temperature for 30 min. Six volumes of pre-chilled (−20° C.) acetone was added allow protein to precipitate overnight. The precipitated protein was reconstituted in 200 μL of 100 mM TEAB. The solution was then subject to overnight digestion at 37° C. with trypsin (Promega) using an enzyme to substrate ratio of 1:50 (w/w). Purification of peptides is performed at room temperature on C18 reversed-phase columns. The lyophilized peptide was reconstituted in 0.1% TFA any insoluble was removed by centrifugation at 1,780 g for 15 min. The columns were conditioned by 100% ACN followed by 0.1% TFA, 80% ACN and then equalized by 0.1% TFA. The acidified and cleared digest were then loaded onto column. After wash by 0.1% TFA, the peptides were eluted from the column by 0.1% TFA, 80% ACN. Phosphopeptide enrichment was done using Fe-IMAC beads according to manufacturer's protocol.

Nanoflow UPLC was performed using following settings: Nanoflow UPLC:Ultimate 3000 nano UHPLC system (ThermoFisher Scientific, USA). Nanocolumn: trapping column (PepMap C18, 100A, 100 μm×2 cm, 5 μm) and an analytical column (PepMap C18, 100A, 75 μm×50 cm, 2 μm). Loaded sample volume: 1 μg. Mobile phase: A: 0.1% formic acid in water; B: 0.1% formic acid in 80% acetonitrile. Total flow rate: 250 nL/min. LC linear gradient: from 2 to 8% buffer B in 3 min, from 8% to 20% buffer B in 60 min, from 20% to 40% buffer B in 23 min, then from 40% to 90% buffer B in 4 min. The full mass spectrometry scan was performed between 300-1,650 m/z at the resolution 60,000 at 200 m/z, the automatic gain control target for the full scan was set to 3e6. The MS/MS scan was operated in Top 20 mode using the following settings: resolution 15,000 at 200 m/z; automatic gain control target 1e5; normalized collision energy at 28%; isolation window of 1.4 Th; charge sate exclusion: unassigned, 1, >6; dynamic exclusion 30 s.

Raw MS files were analyzed and searched against Mus musculus protein database according to sample species using Maxquant (1.6.2.6). The parameters were set as follows: the protein modifications were carbamidomethylation (C) (fixed), oxidation (M) (variable), Phospho (STY) (variable); the enzyme specificity was set to trypsin; the maximum missed cleavages were set to 2; the precursor ion mass tolerance was set to 10 ppm, and MS/MS tolerance was 0.02 Da. Normalized signals of each phosphorylated sites on aligned proteins were further analyzed using PhosR package. Phosphoproteomic normalized signal (data not shown).

### Statistical Analysis

Data are presented as the mean±SEM unless otherwise specified. The number of independent biological samples (n) in each experiment is detailed in the figure descriptions. The data were analyzed by unpaired two-sided student's t test, one-way ANOVA or two-way ANOVA with GraphPad Prism 9 software. For all the analyses, */a/#p<0.05, **/aa/##p<0.01, ***/aaa/###p<0.001, ****/aaaa/####p<0.0001.

What is claimed is:

1. A composition for inducing PER1 expression, the composition comprising a deltaCBD fusion protein comprising a PER1 protein lacking a C-terminus CRY-1 binding domain (CBD).

2. The composition of claim 1, further comprising trehalose or a trehalose analog.

3. The composition of claim 2, wherein the trehalose analog is selected from 6-azido trehalose (6-TreAz), 4-trehalosamine (4-TA), and IMCAT-C14 (IMCTA).

4. The composition of claim 1, wherein the deltaCBD fusion protein is packaged in an AAV8-vector under control of a TBG promoter.

5. A composition for inducing PER1 expression, the composition comprising at least one fusion protein selected from:
a CRY-1 binding domain (CBD) fragment fusion protein comprising of a CBD region of a PER1 protein, wherein the CBD region lacks an N-terminus;
a TAT-CBD fusion protein comprising of a CBD region of a PER1 protein fused to a viral TAT protein sequence YGRKKRRQRRR (SEQ ID NO: 1);
an Fc-CBD fusion protein comprising a CBD region of a PER1 protein fused to an Fc portion of immunoglobulin; and
an AAV8-native CBD fusion protein comprising a CBD region of a PER1 protein packaged in an AAV8-vector under control of a TGB promoter.

6. The composition of claim 5, wherein:
the CBD region of the CBD fragment fusion protein is untagged or HA-tagged;
the CBD region of the TAT-CBD fragment fusion protein is untagged or HA-tagged;
the CBD region of the Fc-CBD fragment fusion protein is untagged or HA-tagged; and
the CBD region of the AAV8-native CBD fragment fusion protein is untagged or HA-tagged.

7. The composition of claim 5, further comprising trehalose or a trehalose analog.

8. The composition of claim 6, wherein the trehalose analog is selected from 6-azido trehalose (6-TreAz), 4-trehalosamine (4-TA), and IMCAT-C14 (IMCTA).

9. A method for inducing PER1 expression in a subject in need thereof, the method comprising:
administering to the subject a composition comprising at least one fusion protein, wherein the at least one fusion protein is selected from:
a deltaCBD fusion protein comprising a PER1 protein lacking a C-terminus CRY-1 binding domain (CBD);
a CRY-1 binding domain (CBD) fragment fusion protein comprising of a CBD region of a PER1 protein, wherein the CBD region lacks an N-terminus;
a TAT-CBD fusion protein comprising of a CBD region of a PER1 protein fused to a viral TAT protein sequence YGRKKRRQRRR (SEQ ID NO: 1);
an Fc-CBD fusion protein comprising a CBD region of a PER1 protein fused to an Fc portion of immunoglobulin; and
an AAV8-native CBD fusion protein comprising an untagged CBD region of a PER1 protein packaged in an AAV8-vector under control of a TGB promoter.

10. The method of claim 9, wherein the composition further comprises trehalose or a trehalose analog.

11. The method of claim 10, wherein the trehalose analog is selected from 6-azido trehalose (6-TreAz), 4-trehalosamine (4-TA), and IMCAT-C14 (IMCTA).

12. The method of claim 9, wherein the at least one fusion protein comprises the deltaCBD fusion protein packaged in an AAV8-vector under control of a TBG promoter.

13. The method of claim 9, wherein:
the CBD fragment fusion protein is packaged in an AAV8-vector under control of a TBG promoter;
the TAT-CBD fusion protein is packaged in an AAV8-vector under control of a TBG promoter; and
the Fc-CBD fusion protein is packaged in an AAV8-vector under control of a TBG promoter.

14. The method of claim 9, wherein:
the CBD region of the CBD fragment fusion protein is untagged or HA-tagged;
the CBD region of the TAT-CBD fragment fusion protein is untagged or HA-tagged;
the CBD region of the Fc-CBD fragment fusion protein is untagged or HA-tagged; and
the CBD region of the AAV8-native CBD fragment fusion protein is untagged or HA-tagged.

15. The method of claim 9, wherein administering the composition results in inhibition of CRY-1/PER1 interaction.

16. The method of claim 9, wherein administering the composition results in at least one of:
improved glucose tolerance;
lowered fasting glucose;
improved basal metabolic rate; and
improved glucose homeostasis.

17. The method of claim 9, wherein administering the composition comprising the TAT-CBD fusion protein results in greater cell permeability into multiple target tissues.

18. The method of claim 9, wherein administering the composition comprising the Fc-CBD fusion protein results in a greater half-life of the fusion protein for dosing in the subject.

19. The method of claim 9, wherein administering the composition comprising the AAV8-native CBD fusion protein results in inhibition of target tissue CRY/PER1 binding based on AAV tropism.

20. The method of claim 9, wherein the subject has at least one of obesity, fatty liver disease, diabetes, type 2 diabetes mellitus, metabolic syndrome, liver cancer, and autophagic flux deficiency.


SEQUENCE LISTING (56 sequences)

SEQ ID NO: 1 - RNA, 21 nt, synthetic construct
  source 1..21: mol_type = other RNA
gattactgctctggctcctag

SEQ ID NO: 2 - RNA, 21 nt, synthetic construct
  source 1..21: mol_type = other RNA
gactcatcgtactcctgcttg

SEQ ID NO: 3 - RNA, 23 nt, synthetic construct
  source 1..23: mol_type = other RNA
gattcccacaagagtaacacgac

SEQ ID NO: 4 - RNA, 22 nt, synthetic construct
  source 1..22: mol_type = other RNA
cctatgctgtcagatgagtcct

SEQ ID NO: 5 - RNA, 22 nt, synthetic construct
  source 1..22: mol_type = other RNA
tgaccctcatggaaggttagaa

SEQ ID NO: 6 - RNA, 21 nt, synthetic construct
  source 1..21: mol_type = other RNA
ggacattgcattgcatgttgg

SEQ ID NO: 7 - RNA, 23 nt, synthetic construct
  source 1..23: mol_type = other RNA
agaacttggcattgaagagtctc

SEQ ID NO: 8 - RNA, 21 nt, synthetic construct
  source 1..21: mol_type = other RNA
gtcagacccagaatcttggct

SEQ ID NO: 9 - RNA, 21 nt, synthetic construct
  source 1..21: mol_type = other RNA
cactggttccgaaagggactc

SEQ ID NO: 10 - RNA, 21 nt, synthetic construct
  source 1..21: mol_type = other RNA
ctgaagcaaaaatcgccacct

SEQ ID NO: 11 - RNA, 23 nt, synthetic construct
  source 1..23: mol_type = other RNA
tttagccctacaaggtacttgga

SEQ ID NO: 12 - RNA, 19 nt, synthetic construct
  source 1..19: mol_type = other RNA
gcagccactgccttcgtaa

SEQ ID NO: 13 - RNA, 23 nt, synthetic construct
  source 1..23: mol_type = other RNA
aagatcgacacattgattccagc

SEQ ID NO: 14 - RNA, 23 nt, synthetic construct
  source 1..23: mol_type = other RNA
catggttgaactgtaactcccac

SEQ ID NO: 15 - RNA, 19 nt, synthetic construct
  source 1..19: mol_type = other RNA
ctgctgggggtctaccaag

SEQ ID NO: 16 - RNA, 19 nt, synthetic construct
  source 1..19: mol_type = other RNA
ctgcgcctaccactgttcc

SEQ ID NO: 17 - RNA, 21 nt, synthetic construct
  source 1..21: mol_type = other RNA
cgactcgctatctccaagtga

SEQ ID NO: 18 - RNA, 20 nt, synthetic construct
  source 1..20: mol_type = other RNA
gttgaaccagtctccgacca

SEQ ID NO: 19 - RNA, 20 nt, synthetic construct
  source 1..20: mol_type = other RNA
ccgtatgggcttctgttcag

SEQ ID NO: 20 - RNA, 20 nt, synthetic construct
  source 1..20: mol_type = other RNA
agctttgtgcgttccatcag

SEQ ID NO: 21 - RNA, 20 nt, synthetic construct
  source 1..20: mol_type = other RNA
agggaggtcgagctgttctc

SEQ ID NO: 22 - RNA, 21 nt, synthetic construct
  source 1..21: mol_type = other RNA
ggagtgttcactaagcggtca

SEQ ID NO: 23 - RNA, 20 nt, synthetic construct
  source 1..20: mol_type = other RNA
tcccctattcgcttctgtgc

SEQ ID NO: 24 - RNA, 20 nt, synthetic construct
  source 1..20: mol_type = other RNA
gcagcaatcgatggatctgc

SEQ ID NO: 25 - RNA, 22 nt, synthetic construct
  source 1..22: mol_type = other RNA
gaaagctgtcaccaccatagaa

SEQ ID NO: 26 - RNA, 21 nt, synthetic construct
  source 1..21: mol_type = other RNA
aactcgcacttccttttcagg

SEQ ID NO: 27 - RNA, 20 nt, synthetic construct
  source 1..20: mol_type = other RNA
acaccgcaattctcccttgt

SEQ ID NO: 28 - RNA, 20 nt, synthetic construct
  source 1..20: mol_type = other RNA
cggcgctcttcaattgcttt

SEQ ID NO: 29 - RNA, 20 nt, synthetic construct
  source 1..20: mol_type = other RNA
tggttcctggtgccgattta

SEQ ID NO: 30 - RNA, 27 nt, synthetic construct
  source 1..27: mol_type = other RNA
actagcatcccacttaattatgtatct

SEQ ID NO: 31 - RNA, 20 nt, synthetic construct
  source 1..20: mol_type = other RNA
ctttcctcgcggctgtttac

SEQ ID NO: 32 - RNA, 22 nt, synthetic construct
  source 1..22: mol_type = other RNA
cctccaggtatgggtagatcgt

SEQ ID NO: 33 - RNA, 21 nt, synthetic construct
  source 1..21: mol_type = other RNA
tgtccgcactgactgtaacca

SEQ ID NO: 34 - RNA, 20 nt, synthetic construct
  source 1..20: mol_type = other RNA
tgctccgcacagattcttca

SEQ ID NO: 35 - RNA, 20 nt, synthetic construct
  source 1..20: mol_type = other RNA
ctggggacctaaacaggagc

SEQ ID NO: 36 - RNA, 20 nt, synthetic construct
  source 1..20: mol_type = other RNA
gaagccaccctatagctccc

SEQ ID NO: 37 - RNA, 22 nt, synthetic construct
  source 1..22: mol_type = other RNA
ggaaagcagttcaacgagaacg

SEQ ID NO: 38 - RNA, 21 nt, synthetic construct
  source 1..21: mol_type = other RNA
agatgccgaccaccaaagata

SEQ ID NO: 39 - RNA, 21 nt, synthetic construct
  source 1..21: mol_type = other RNA
cctggatagcattccgaacct

SEQ ID NO: 40 - RNA, 22 nt, synthetic construct
  source 1..22: mol_type = other RNA
agcacatctcgaaggctacaca

SEQ ID NO: 41 - RNA, 19 nt, synthetic construct
  source 1..19: mol_type = other RNA
caacaccatccccgacatc

SEQ ID NO: 42 - RNA, 22 nt, synthetic construct
  source 1..22: mol_type = other RNA
gtgaccttcgattatgcgatca

SEQ ID NO: 43 - RNA, 22 nt, synthetic construct
  source 1..22: mol_type = other RNA
ctcttggcagtgctttttctct

SEQ ID NO: 44 - RNA, 21 nt, synthetic construct
  source 1..21: mol_type = other RNA
gagcttgtatagccgctcatt

SEQ ID NO: 45 - RNA, 23 nt, synthetic construct
  source 1..23: mol_type = other RNA
cggattgtctatatttcggagca

SEQ ID NO: 46 - RNA, 20 nt, synthetic construct
  source 1..20: mol_type = other RNA
tgggcagtcgagatggtgta

SEQ ID NO: 47 - RNA, 20 nt, synthetic construct
  source 1..20: mol_type = other RNA
atcctggccactctgatgac

SEQ ID NO: 48 - RNA, 20 nt, synthetic construct
  source 1..20: mol_type = other RNA
ttcttcctccatggcagagt

SEQ ID NO: 49 - RNA, 20 nt, synthetic construct
  source 1..20: mol_type = other RNA
tcgcggttttaatcgacgag

SEQ ID NO: 50 - RNA, 21 nt, synthetic construct
  source 1..21: mol_type = other RNA
ctaccccgagtgctagagcta

SEQ ID NO: 51 - RNA, 19 nt, synthetic construct
  source 1..19: mol_type = other RNA
acgccgaccctcacaattc

SEQ ID NO: 52 - RNA, 21 nt, synthetic construct
  source 1..21: mol_type = other RNA
cagttttccgcccttctcttt

SEQ ID NO: 53 - RNA, 20 nt, synthetic construct
  source 1..20: mol_type = other RNA
ccatggattgcacatttgaa

SEQ ID NO: 54 - RNA, 19 nt, synthetic construct
  source 1..19: mol_type = other RNA
gaagtcactgtcttggttg

SEQ ID NO: 55 - RNA, 21 nt, synthetic construct
  source 1..21: mol_type = other RNA
aggcttccagtaccattaggt

SEQ ID NO: 56 - RNA, 22 nt, synthetic construct
  source 1..22: mol_type = other RNA
ctgagtgaggcaaagctgattt